SEQUENCE LISTING (15 sequences)

SEQ ID NO: 1 - protein, 497 aa, Homo sapiens
  source 1..497: note = GBA1 (wildtype)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 2 - DNA, 13625 bp, Homo sapiens
  source 1..13625: mol_type = genomic DNA; note = GBA1 (wildtype)
acagaagtacaatgctgattatgatctgtcagctcagcaaggggcagacaccctggccttcatgtctctcctggaggagaagttgatcccggtgctggtgagtgtgcccagacctcccagcatccatggccagccggggaggggacgggaacacacagacccacacagagactcaggagagcatggaggtcagaagcccaccttgaatcagacaggtgcactggctcagacctgcctgtttcttcctgcccacccaatccaggtacatactttttggatagacaccaagaactacgtagaagtgacccggaagtggtatgcagaggctatgccctttcccctcaacttcttcctgcctggccgcatgcagcggcagtacatggaacggctacagctgctgactggggagcacaggcctgaggacgaggaagagctggagaaggaggtagctctgagaccgggggctattgtatgagatgagccccaaggatgctggccaggaatgggagtgcttaggtgcggaggtggcactgttcccgcagctgcaagcctacctgtgtcgcccctacagctgtaccgagaggctcgggagtgtctgaccctgctctctcagcgcctgggctctcaaaagttcttctttggagatgcgtgagtctgactccaagagggtaatgggtggcttggaagaagatacaggttcagatggagcagctggagctggggctggggctggggctggctcaggctctggataggaggtccctgagacagatactggccctggtgacagtggggctgtgcgtggggccagagccttctcagaggtacaaaagggtagggtgggagggcagccaggcacaggaagggcctgaagagctgtggggcactgagtgtgccctttatgcagccctgggatagagccctattcagggccaggctggcgccacctggggatctctccccataccaggtctagaactgtgtgtcctgtccttccctggtggccgcctgctgcccagagcccacctcccaaggctgactcttcctccagctccatctttaccccttctaccccagtggttctcctccatcccacccttctctctctgctccagccctgcctccttggacgccttcgtcttcagctacttggccctgctgctgcaggcaaagctgcccagtgggaagctgcaggtccacctgcgtgggctgcacaacctctgtgcctattgtacccacattctcagtctctacttcccctgggatggaggtaaggggcagatgggaggggcagccctggggagagtgggcagggatccaagaactagttctcctaacacaccttccttccttgaccctcagctgaggtaccaccgcaacgccagacaccagcaggcccagagactgaggaggagccataccggcgccggaaccagatcctatctgtgctggcaggactggcagccatggtgggctacgccttgctcagcggcattgtctccatccagcgggcaacgcctgctcgggccccaggcacccggaccctgggcatggctgaggaggatgaagaggaatgatttgtcctcacgctcccaagactggtttttctactctcatgcattccagaggcccccgtgcctcctcgttgttggtacagccggacacggggtgctgccacccagaataaagccactcacactgactgggctcaaacattttctcctttaagagctgccatttttcctggctggtgccataggaatcatctgggtgcctgggcacacccgctgctgctttaaggcttccgccctgatgctgacactgctgctccacgggcccagttctgcatctccaggaaagacaaacagtctccagttttgggcccagctttcctagtctcttcttttccttaccctcagccctgatcttgtgtttgtacggacagtgagctcaccctaggcctggacccaggcccagtttccaaagcaagcagcacacagcgcatgttcacataagcatgggggctggggggacactggggcttactgatctttttctaggggcctccagcccctggcaccacctagaggggaaagtgagtcacccaaaccattgcccctgggcttacgtcgctgtaagctcacactggccctgctgtgccctctttagtcacagacagcgtgtgagctgactctgtccctttaatgcccaggctgagcccagtgcctccttgagtatctgctccatcactggcgacgccacaggtaggtgtgaatggagtagccaggtgagattgtctccaggaagcccacagcaggatccttgatggtaagaggcacatccttagaggagctagggagcagggaggagaagctgagagtgtgatcctgccaaggcccccaacgctgtcttcagcccacttcccagacctcaccattgccctcaccggtttagcacgaccacaacagcagagccatcgggatgcatcagtgccactgcgtccaggtcgttcttctgactggcaaccagccccactctctgggagccctcaggaatgaacttgctgaatgtgggaacagatggtcagagtccctcggggtacctcccatgaaaccctcatctaagaagtcacccacccacggacccaccccataactcctgcagaggctctgccctggctctctaggcctggagccatgctgctgggcactgaccctgcttttctgcatcgcagtccagcctcaggcattggggttttctgttgctacctagtcacttcctgcctccatggtgcaaaaggggatgggtgtgcctcttccgaggttccaccctgaacaccttcctgctccctcgtggtgtagagtgatgtaagccatccgatgtaggagatgataggcctggtatggaatgggggtgcccgccctccactcacctgaagtggccaaggtggtagaacatgggctgtttgtaaaacgtgtccttggtgatgtctacaatgatgggactgtcgacaaagttacgcacccaattgggtcctccttcggggttcagggcaaggttccagtcggtccagccgaccacatggtacaggaggttctagggtaaggacaaaggcaaagagacaaaggcgcaacactgggggtccccagagagtgtaggtaagggtcacatgtgggagaggcagctgtgggtaggtcagccctgtgaggggcacattccttagtagctaaggagttgggggtgtgaagatccaggcatctcaaggggagctgagaagtctgaggcagctgcaagtgcctcagtagttgcaaaaggggcaatgaggtgtgcagacctgtgaaggaaagggaagatagggaatcatggttccccagagttgctcaaaagggcaggctagctggggaaagctggacaggaagggcttctgtcagtctttggtgaaactagtaagaggtctgaggtctgctttgcaggaagggagactggggtggcttaccgtgatgatgctgtggctgtactgcatccctcgatcccaggagcctagccgcacactctgctcccagaacttggagcccacacaggcctctgaggcaaagagcatggtgttggggaacaggcggtgtgtctcccctagggtggctttggctggagccagaaagtccaggtaccaatgtacagcaatgccatgaacatatttagctgcttctgggtctgtcagtacctgcaaaggaagagcaactgatcctggaccttgcacacaggcttctggaacttctagttcctgttgtaggaatcctggagttgggtgacgggaagaatgcaactagagaggtttggggagatttttttttgtttttgagacaggatgtcactctgtcactcgggctggagtacagtggcgcaatcacgactcactgcagccttgacttcctagggtcaactgatcctcccacattagcctcctgagtagctgggactacacgggtgccacacccagctaatttgtgtgtgtatgtgtgtgtatgtatgtgtgtgtgtgtatatatatatacatataaacacatatatatgtatatacacatacatacatgaaccaccacccccagcctagatagttttgttttgttttgtttcgagatggagtctcgctcagcctcccaggctggagtgcagtggcgcgatctcggctcaccgcaaccaccatctcccgggttcaactgattctcccttctcagcctcctgagtagctgggattacagacacccaccatcatgcccagataattttttttttttgtattttagtagacacagggtttcaacacgttggccagggtggtcttaaactcctgacctcaggtgatcacccgcctcggcttcccaaagtgctgggtttgcatgagtgagccacctcgcccagcccctagaaaggtttcaagcgacaactgtgggatccatggcaccctggaggtccaggggaatggtgctctaggaatccatagttgggtagagaaatcgctctaagtttgggagccagtcatttggatgctggatttgaaggtcactggagcaccatggaggtccaggccttaccacctttgcccagtggggcagcagcaagcgttggtcatccagcatgagtaggcggacattgtggtgagtactgttggcgagggtaggacctaggtcacgggcaatgaagtctcgctgatgttcaggggtgaagcccaggcactggaaggggtatccactcaacagcccagcagaaggctcattttcagctgtcactgcccagaactgtaacttgtgctcagcataggcatccaggaacctggcaagagaaaggtcatgaatgatccggccaagaaagtggaccagaccagctgggtgtggtggctcacacctgtaatcccagcactttgggaagccgaggcaggtggatcacttgagttcaggagttcgagaacagcctggcgaaaccccgtctctactaaaaatagaaaaatcagctgggcctggtggcaggcgcctataatcccagctacttggaaggctgaggcaggagaattgcttgaactcaggaggcagaggttacagtgagtgaagatggcgccacagcactccagcctgggtgacagagagagagactccttctcaaaaaaaaaaaaaagaagaaaaataaaaagaaagtgggccagaccgagagaacaggaagcctgatggagtgggcaagattgacaggcccaaggctgaaaggcccagaaggtagaaaggtgagctgaggacaggcagatctggaagtggaactaggttgagggttgggacacagatcagcatggctaaatgggaggccagtcctgatcccacatccttgctgatcccttacttcacaaagtatctggcccaggtctggtggtagatgtctccgggctgtcccttgagtgaccccttcccattcaccgctccattggtcttgagccaagtgggtgatgtccaggggctggcaaggagtgaaacgggacgctgggccaactgcagggctcggtgaatcaggggtatctagagacaaaggtagtgaagagagaagcacccagagttggaacacatactagcccaaccagtgcatccggttcagccattagcctccaccctcccacccccaggacaaaacagcaggggacaaaatgtctgtacaagcagacctaccctacagtttctcaacccccagacatcagggccctcagggcctgaaaaagctagaatgcctaccttgagcttggtatcttcctctgggaggctgaagttgtgcaactggaaatcatcaggggtgtctgcataggtgtaggtgcggatggagaagtcacagctggccatgggtacccggatgatgttatatccgattcctacagaaaaggatgatcaagatatggtagtccgagtcaataggagagtatgggactctgcttatcacttgccagtcctaatagtgtctgagtcagggccaaagggaacttgggctcctgggttggaacctgtggaggctggcacctgggtgaagcgcaggcctttctgagcctgagtccgtagcagttagcagatgataggcggtgaaatcttatttcacagggcattaaaacaggaaccaaatgtcagggatgggcagaagtcagggtccaaagaaagggcaaagaaaagtgtcagtggctcacgcatgtaatcccagcactttgggaggccgatgtgggcagatcacgaggtcaggagttcgcaatcatcctggccaacatagtgaaaccctgtctctactaaaaatacaaaaaattagccgggcgtggtggcaggcacctgtaatcccaggtactcgagaggctgagacaggagaatcacctgaacccgggaggtggaggttgcagtgagctgagattgtgccactgcactccagcctgggtgacagtgcgagactctgtctcaaaaaaaaaaaaagaaaaaagaaaagtgtctgctgggctcggtggctcacacttgtaattccagcacttcgggaggccaaggcaggtatatcatttgaggtcaggagtttaagactagcctggtcaacatgatgaaaccctgtctctactaaaaatacaaaaattagccaggtggtagtggcgcacgcctataattccagctactcaggaggctgaggcaggagaatcactatagcctgggaggcagaggttgctgtgaggggagatcacaccactgcactcctgtctcgccgacagaatgggcagagtgagattctgcctcaaaaaaattttaaaaaaagaaaagaaaaacgaaaagtttcaatggctctatgtcatcttgtccccttcctcctcaccttcttcagagaagtacgatttaagtagcaaattttgggcagggggtgacagggcaaggatgttgagagcagcagcatctgtcatggcccctccaaatcccttcactttctggaacttctgttctggctgcagggtcagtagcaggcctgaggacatccacagggaataagggtatcagtacccagcgggaaactccatggtgatcactgacaccatttacctctaggaggacccagcctggcccagggggtgaggggtgtaatggttacctgtgcccgtgtgattagcctggatgggccccatactcagctccatccgtcgcccactgcgtgtactctcatagcggctgaaggtaccaagggcaggaaaggtcggggggtcaaaggagtcacagtatgtggcattgcagacacacaccaccgagctgtagccgaagcttttagggatgcaggggcgggcacctgggagggagggagtacaagcagagtgaggtctgatgaagacatggagaatggacacatctgctaggagagactgaacacggtttcaaaattcctcaccccttggccgggcgcaggggctcacacctgtaatcctagcactttaggaggccgaggtgggcggatcacctgaggtgaggagtttgagacctgccttgccagcatggtgaaaccccgtctctactaaaaatacaaaaattagctgggcgtagtggtggccgcctgtaatcccagctacctgggaagctgaagcaagagaatcgcttgaatctgggaggcagaggttggaatgagccaaaattgcaccactgcactccagcccaggcaacagagtaagactctgtttcaaaagaaaaaaaacaaaatcctcaccccaaagttggtctcagtcactcaaaaggatttattgagcacctactaaaagtctaccaccagcttactggaaggctaccaaaggactatgaggcagaagggaggctctgtgctacctccccactgccttgactcactcacctgatgcccacgacactgcctgaagtagaagcaatcctgtgaggctgccagccatgatgcttaccctactcaaaggcttgggacattcctgaggacagaatgaggaatgactgaaaagcaagcccctctccacccctcaactactctcctgggcagggcttagctgcctttgggtgcccatggcccggtctcccacattcattaggacaaggcccacagaaacctgggtgcagctctccctgcaacccttctgatgacaactctctgcccaccccaaatcaggatgctccccaccactcttcccacccatttcaacttcgacccctccctccatctgtgccttgctcaaagagccatgatggccctggattcaaagagagtctgtcattcattaaattccagtgccaggattccagaagcactgtgacaatgctgattgggagctctctctcttacctctctggaaggacttgaaaactccatcccctcagggtcattagatgaagagaagaccacaggggttccagagtctctgaaggatagaggatccactaaacaaaaacaaggatgcaggtacctgccttagctataggcactaggttagccctgcaagtaattccggcttcccgatgtggatgggtcatgtgatgactaggagcgtcacatgacacaggaagtgaggcaatcacagccatatttctaaagggcaattggcttcctctcatctgttacagattatatgccctataaaactctggagggcatgtatgggtgacaactttaggaagagcctagaacacagatttgcatggagaatggggtagaggctcctaaatcccagaggatgggaaccacagcaggcacacttcttttttgatagaaatgtcaaaaaggtacaaataaagctgatataatttaaaaaaaaaaacttcactgtgcaaaatactgacactaccaagatgtttaagacagtctttgccctatagaggtgtgtgaggcatggaagtcagacacacagatatttacagataaagacagaaacggtaacaggtgtgcatggagagctctctgagatgaggagggaccattcgtgggtggggaattatccaggatggcttcacagaggaaaaagtgaggggggttattagccagtgaagtgcagggcacaagagggtgggacactggcagtgggatgacaggactggagggagggagtggtcagtgcggctcctctgcagcgtcccttgtttcatcatcagatgcagatggtaataactgttcttccttcctcacaagacagggaggtttgtaaagtcgttcaaaaaccaaagtgttgtacaaagccatatcctcagtggacacaaggaggaagctgtccatggtgtggcctcatgaaccacatcaaatgagatttagcgggagtggcacacacagtcatgacctgactaatcccagctctcagcccatttccttgcctggaaaatggaggcaatgccacaacctcaaagggtggttactgcagtcagtgaggtaagtgcagtgcctgaccacttggtaggcacctgggaactacttgtctcttgtttgtattttttgttgttgttgttttttgagacagagtctcactctgttgcccaggctggagtgcagtggcgtgatcttggctcactgcaacctccacctcctgggttcgagtgattctcctgcctcagcctcccaagtagctgggattacaggtgccctccaacatgctcagctaatttttttatttttagtagagatggggtttcactatgttggccaggctggtctgaaactctggacctcaggtgatctgctgccttggtatccgaaagtgctgggattacaggcatgagccactgcgctggaccccagccatcctttttgttctttaaatgatctcagtgaagtctttctagatacactccaagcaaaattgatcatttcttcttctaggttcctccagtaatttttttttttttggttttgagacagagtcttgccctgttgcccaggctggagtgcagtgtgatctcggctcaagcaattgccctgcctcagcctcctgagtagctgggattacaggagcccaccaccatgcccagctaatttttgtatttttagtagagatggggtttcaccatgttggccaggctggtctcgaacccctgacctcaggtgatccaccagcctcagcctcccaaggtgttgggattatagcaatgagccaccacacccggtccctccagtaattaagtacagtctcagtgagacagcaagtttggaatcctggctacaccatttactggctgtttgactttcaataaatcaatcactctaagcctctgtttcatctataaaaggggagtgataactcctacctcacagaattgttgtgaggtttgagtgtgataatgtgtctctagtacactgcttgacactaaacattgcaacatgtcaggcctctgttcctggagttccttgaaggaatgtcttatgcattctaagtatcctcgtagatagcctggcacaggggtagctgtcaagttgtagaattgaactagttgcttcactatgtcagtagccaccccttccagacttctcacttttcaaggaaacattgcacaggatttgttctgggtagggcaggtaatatctagtaccttacttccctcaagttcattcatctcacagatatttcctgagcacattctacatttgcctctcctgctctattgagtttagaagtccaaacatctccttccacttcccctctgcatagtgagcctcttcttttttggccaggtactgagcaattttttgtttgtttgtttgagacagggtctccgtatgttgccctggatggagtgtagtagcttgatttcggctcagtgcaacctctgcctccagaataagcaatcctcccacctcagcctccagaatagctgggactataggcgcacaccaccacacctggctaatttctgtattttttgtagagacggggtcttactgtgttgcgcaggttggtctcaaactcctaaactcaaaggatcctcccgccttggctttgcaaagtgctgggattacagggtgaggcactgcgcccagtccgcttgttttttgttttgtttttttttttgagacggagtatctctgtcacccaggctggagtgcagtggcacgatctcggttcactgcaacctccgcctcctaggttcaagcggagggtagggaccagtccatcctggcacccatctgcagctccagtgcgaatcccaaccccgacgctcgtcgccgggctccgtgaatgtttgtcacatgtctgaagaacgtatgaattacataaccttcttcccactccacccctcaaaaagcaagtggatataaagacttgaagattttataatctcttcttcattagtaaaatctgaccatccttaccgttaaaaataataatgatggttggccgggcgcggtggctcacgcctataatcacagctgtttggcaggccgaggcgggcggatcacgaggtcaggaattggagaccagcctgaccaacatggtgaaaccccgtctctactaaaagatacaaaaaattagccgtgcgtggtggcgggtgcctgtaatcccacgtacttgggaggctgaggtcggagaatcgcctgaacccgggaggcggaggttgcagtgagccaagaaggcgccattacactccagcctgggcgacagggcgagactccctcaaaaaataataataataacaataataataataatggtgaaaaaggttaagtgcgaacgcagggaggggacagctaagatccaaaggtcgaaatattcattacgcctaccgtcggcgaagagaaacagcagccccaaccggaggcaaaacgaaatcccaccgcagcctgcaaaggcgcctgggcgggactggagactggggccccgcgcagtaagactctgaaggcaggatgcagcccgaccacccgcagccgcgagaaaagcagccctggggagtcggggcgggacctggattggaaaagagacggtcactcatgcagaggcgggactcagagcccttcctcaagtctcattggtcaagttgaacgaacaagtgtcgctggcgagccggagagagagagagagcgagagcgagagagcgagcgagagagagcgagagagcgagagagcgagcgagagagagagagagagagagaggagccggcgcgagaactacgcatgcgtgtcggcgttttcccgccagcacactgttggtggatgggggcgattgaattcccacagtgagtccagcccaccgaagctcagaggattcctaaccttcctctttagagagcctcaggttagggaacgtccagtgcgcagaagctgccccgtgggaatcccattgtccatcgcctcctaatgtttcgtcctggccgtgccctatcccttcctgaggctgggttgttatgatgctgaattattcaagaagtcttgcagcctgacgccatctctgggcagtgctcctcccacctccctgtcttccttggagggcaccacgttgcccctacaagcacagggtcctgaagctgtttacagggccccaccctgccactttagtatcttaacgaatgtttgttgaggacctactgagtgttagacccactgcaatgagcaagtttctgatcttgggaccttaacattctatgggacgacacagatatataaacaaaaataataattttagatagtgatacatgctgaaaccatgggtatgtggccaggcgcggtgagtcacacctgtaatcccagcactttgggagaccgaggcaggcggattacttgaggtcaggagtttgagacccgcctggccaacatggtgaaaccctatctcttctaaaaatacaaaatttagctgggcgggcatggggacgggcgcctgtaatcccagctactcgggaggctgaggcaggagaattgcttgaacccaggaggtggaaggttgcagtgagcccaaatcacaccactgcactccagcctgggtaacaaagcaagaccctgtctcaaaaaaagacaaaatgaaacaaacaaacaaaaaaaaacatggctatgtgatagaatgactgcgatggggattgggtgctcactgaaatggtgagaatggagctaaaacctgatattaggtgatagcaccagatataggggccaagggtttcatgcttggaaaacagcaggtgcaaacgtccttgtgctgtgaggaaatttggctggaacagaaggtgggccagattgtagctgaccatagtctctgggagtttggagatgattctaaatacaatgggaagccattaaattgaggcagagccttgatgtgacgtgctttatcatcattttgctgagtggagaatggattgctgggaaaagtgaaagactgattcagaggttgtggagagaggatggctggagctaggtagctggggaggtggtgggatgtggatgaattcaggataggattggttgtgttggatatgggaaaagaagtcaccatggttcctgagcttttggaatgtgcaaatgggtaatggaggtgccattgagatgggaacagtggaaaaataaccggtgctggggtgggaatcaagagttcagttgtggccatgttgaatctgaaatgcccattagacatccaagcagacatgctgagttggatggcgctcat

SEQ ID NO: 3 - protein, 497 aa, Homo sapiens
  source 1..497: note = GBA1 (N370S mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITSLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 4 - protein, 497 aa, Homo sapiens
  source 1..497: note = GBA1 (L444P mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDPDAVALMHPDGSAVVVVLNRSSKDVPLTIKDPAVGFLETISPGYSIHTYLWRRQ

SEQ ID NO: 5 - protein, 497 aa, Homo sapiens
  source 1..497: note = GBA1 (E326K mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGKTHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 6 - protein, 497 aa, Homo sapiens
  source 1..497: note = GBA1 (T369M mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIIMNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 7 - protein, 497 aa, Homo sapiens
  source 1..497: note = GBA1 (G377S mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVSWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 8 - protein, 497 aa, Homo sapiens
  source 1..497: note = GBA1 (D409H mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKHTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 9 - protein, 497 aa, Homo sapiens
  source 1..497: note = GBA1 (R120W mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIWVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 10 - protein, 497 aa, Homo sapiens
  source 1..497: note = GBA1 (V394L mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWLRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 11 - protein, 497 aa, Homo sapiens
  source 1..497: note = GBA1 (R496H mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRHQ

SEQ ID NO: 12 - protein, 497 aa, Homo sapiens
  source 1..497: note = GBA1 (K178T mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASTWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 13 - protein, 497 aa, Homo sapiens
  source 1..497: note = GBA1 (R329C mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHCLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 14 - protein, 497 aa, Homo sapiens
  source 1..497: note = GBA1 (L444R mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDRDAVALMHPDGSAVVVVLNRSSKDVPLTIKDPAVGFLETISPGYSIHTYLWRRQ

SEQ ID NO: 15 - protein, 497 aa, Homo sapiens
  source 1..497: note = GBA1 (N188S mutant)
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTSGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

(19) United States
(12) Patent Application Publication
(10) Pub. No.: US 2023/0081881 A1
(43) Pub. Date: Mar. 16, 2023

(54) TRANSPOSON-BASED MODULATION OF GBA1 AND RELATED COMPOSITIONS AND USES THEREOF

(71) Applicant: Aspen Neuroscience, Inc., San Diego, CA (US)

(72) Inventors: Andres BRATT-LEAL, San Jose, CA (US); Ai ZHANG, San Diego, CA (US)

(73) Assignee: Aspen Neuroscience, Inc., San Diego, CA (US)

(21) Appl. No.: 17/813,915

(22) Filed: Jul. 20, 2022

Related U.S. Application Data
(60) Provisional application No. 63/224,395, filed on Jul. 21, 2021.
(60) Provisional application No. 63/272,625, filed on Oct. 27, 2021.

(51) Int. Cl. C12N 15/90; C12N 15/113; C12N 5/074

(52) U.S. Cl. CPC C12N 15/90; C12N 15/113; C12N 5/0696; C12Y 302/01045; C12N 2320/34; C12N 2510/00

(57) ABSTRACT

The present disclosure provides transposon-based methods of genetic editing in pluripotent stem cells, and methods of lineage specific differentiation of such edited pluripotent stem cells into floor plate midbrain progenitor cells, determined dopamine (DA) neuron progenitor cells, and/or DA neurons, or into glial cells, such as microglial cells, astrocytes, oligodendrocytes, or ependymocytes. Also provided are compositions and uses thereof, such as for treating neurodegenerative diseases and conditions, including Parkinson's disease.

### CROSS-REFERENCE TO RELATED APPLICATIONS

This application claims priority to U.S. provisional application 63/224,395, filed Jul. 21, 2021, entitled “TRANSPOSON-BASED MODULATION OF GBA1 AND RELATED COMPOSITIONS AND USES THEREOF,” and U.S. provisional application 63/272,625, filed Oct. 27, 2021, entitled “TRANSPOSON-BASED MODULATION OF GBA1 AND RELATED COMPOSITIONS AND USES THEREOF,” the contents of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 165622000900SeqList.xml, created Jul. 20, 2022, which is 37,209 bytes in size. The information in the electronic format of the Sequence Listing is incorporated by reference in its entirety.

### FIELD

The present disclosure relates to transposon-based methods of increasing expression of the glucosylceramidase beta (GBA1) gene in pluripotent stem cells, including induced pluripotent stem cells (iPSCs), and differentiation of such cells into floor plate midbrain progenitor cells, determined dopamine (DA) neuron progenitor cells, and/or dopamine (DA) neurons, or glial cells. Also provided are compositions of the cells having increased expression of GBA1 and therapeutic uses thereof, such as for treating neurodegenerative conditions and diseases, including Parkinson's disease.

### BACKGROUND

Reduced activity of certain proteins, including β-Glucocerebrosidase (GCase) (encoded by the glucosylceramidase beta (GBA1) gene), has been associated with an increased risk of developing certain neurodegenerative diseases or disorders, such as Parkinson's Disease (PD). Other diseases or disorders are also associated with reduced GCase activity, such as Gaucher's disease. In some cases, a variant in a gene encoding a protein may contribute to, or cause, reduced activity of the protein. Various methods for differentiating pluripotent stem cells into lineage specific cell populations and the resulting cellular compositions are contemplated to find use in cell replacement therapies for patients with diseases resulting in a loss of function of a defined cell population. However, in some cases, such methods are limited in their ability to produce cells with consistent physiological characteristics, and cells resulting from such methods may be limited in their ability to engraft and innervate other cells in vivo. Moreover, in some cases, such methods involve the use of cells having reduced activity of GCase, such as due to a gene variant, e.g., a SNP, in GBA1 that is associated with an increased risk of developing PD. Improved methods and cellular compositions thereof are needed, including to provide for improved methods for increasing GBA1 expression and/or increasing GCase activity in such differentiated cells.

### SUMMARY

Provided herein are methods of increasing expression of GBA1 in a cell, the methods including: (i) introducing, into a pluripotent stem cell, a deoxyribonucleic acid (DNA) sequence encoding GBA1 operably linked to a promoter, wherein the DNA sequence is positioned between inverted terminal repeats and is capable of integrating into DNA in the cell; and (ii) introducing, into the cell, a transposase or a nucleic acid sequence encoding a transposase, wherein the introducing in (i) and (ii) results in integration of the DNA sequence encoding GBA1 into the genome of the cell.

In some embodiments, the cell has reduced activity of GCase. In some embodiments, the cell endogenously contains a variant of GBA1. In some embodiments, the cell is heterozygous for the GBA1 variant. In some embodiments, the cell endogenously contains a variant of GBA1 associated with Parkinson's disease.

In some embodiments, the cell comprises biallelic variants in GBA1 or is homozygous for the GBA1 variant. In some embodiments, the cell comprises biallelic variants in GBA1. In some embodiments, the cell is homozygous for the GBA1 variant. In some embodiments, the cell endogenously contains one or more variant(s) of GBA1 associated with Gaucher's disease (GD).

Also provided herein are methods of increasing expression of GBA1 in a cell, the methods including: (i) introducing, into a pluripotent stem cell, a deoxyribonucleic acid (DNA) sequence encoding GBA1 operably linked to a promoter, wherein the DNA sequence is positioned between inverted terminal repeats and is capable of integrating into DNA in the cell; and (ii) introducing, into the cell, a transposase or a nucleic acid sequence encoding a transposase, wherein: the cell contains a variant of GBA1 associated with Parkinson's disease, and the introducing in (i) and (ii) results in integration of the DNA sequence encoding GBA1 into the genome of the cell. In some embodiments, the cell has reduced activity of GCase. In some embodiments, the cell is heterozygous for the GBA1 variant.

In some embodiments, GBA1 is the wild-type form of GBA1. In some embodiments, the wild-type form of GBA1 is encoded by the sequence set forth in SEQ ID NO:2. In some embodiments, the wild-type form of GBA1 is encoded by the sequence set forth in SEQ ID NO:2 or a sequence having at least 80%, at least 85%, at least 90%, at least 95%, or at least 99% sequence identity to the sequence set forth in SEQ ID NO:2. In some embodiments, the wild-type form of GBA1 encodes an amino acid sequence comprising the amino acid sequence set forth in SEQ ID NO: 1. In some embodiments, GBA1 is a functional GBA1 or a portion thereof. In some embodiments, a functional GBA1 is capable of being transcribed into GBA1 mRNA or a portion thereof. In some embodiments, a functional GBA1 is capable of being transcribed into GBA1 mRNA or a portion thereof, which is capable of being translated into a functional GCase enzyme or a portion thereof. In some embodiments, a functional GBA1 is capable of (i) being transcribed into GBA1 mRNA or a portion thereof; and (ii) being transcribed into GBA1 mRNA or a portion thereof, which is capable of being translated into a functional GCase enzyme or a portion thereof. In some embodiments, a functional GCase enzyme or a portion thereof has the enzymatic activity of a wild-type GCase enzyme. In some embodiments, the enzymatic activity of GCase is determined by any of the methods described herein.

In some embodiments, the DNA sequence encoding GBA1 is part of a plasmid.

In some embodiments, the promoter is selected from the group consisting of: ubiquitin C (UBC promoter) cytomegalovirus (CMV) promoter, phosphoglycerate kinase (PGK) promoter, CMV early enhancer/chicken b actin (CAG) promoter, glial fibrilary acidic protein (GFAP) promoter, synapsin-1 promoter, and Neuron Specific Enolase (NSE) promoter. In some embodiments, the promoter is a PGK or UBC promoter. In some embodiments, the promoter is a PGK promoter. In some embodiments, the promoter is a UBC promoter.

In some embodiments, the transposase is a Class II transposase. In some embodiments, the transposase is selected from the group consisting of: Sleeping Beauty, piggyBac, TcBuster, Frog Prince, Tol2, Tcl/mariner, or a derivative thereof having transposase activity. In some embodiments, the transposase is Sleeping Beauty, PiggyBac, or TcBuster. In some embodiments, the transposase is Sleeping Beauty. In some embodiments, the transposase is PiggyBac. In some embodiments, the transposase is TcBuster.

In some embodiments, the nucleic acid sequence encoding the transposase and/or the DNA sequence encoding GBA1 are introduced into the cell by electrotransfer; chemotransfer; or nanoparticles. In some embodiments, the nucleic acid sequence encoding the transposase is introduced into the cell by electrotransfer; chemotransfer; or nanoparticles. In some embodiments, the DNA sequence encoding GBA1 is introduced into the cell by electrotransfer; chemotransfer; or nanoparticles. In some embodiments, the nucleic acid sequence encoding the transposase and the DNA sequence encoding GBA1 are introduced into the cell by electrotransfer; chemotransfer; or nanoparticles. In some embodiments, the electrotransfer is by electroporation or nucleofection.

In some embodiments, the method includes introducing, into the cell, a nucleic acid encoding a transposase. In some embodiments, the nucleic acid encoding a transposase is part of a plasmid. In some embodiments, the nucleic acid encoding a transposase is ribonucleic acid (RNA). In some embodiments, the nucleic acid encoding a transposase is DNA.

In some embodiments, the plasmid containing the DNA sequence encoding GBA1 and the plasmid containing the nucleic acid sequence encoding the transposase are different plasmids. In some embodiments, the plasmid containing the DNA sequence encoding GBA1 and the plasmid containing the nucleic acid sequence encoding the transposase are the same plasmid.

In some embodiments, the method includes introducing, into the cell, a transposase.

In some embodiments, (i) the DNA sequence encoding GBA1 and (ii) the transposase or the nucleic acid sequence encoding the transposase are introduced into the cell at the same time. In some embodiments, the DNA sequence encoding GBA1 is not introduced into an exon. In some embodiments, the DNA sequence encoding GBA1 is introduced into an intron.

In some embodiments, the pluripotent stem cell exhibits decreased expression of GBA1 prior to being introduced with the DNA sequence encoding GBA1 and the transposase or the nucleic acid sequence encoding a transposase, as compared to a reference cell. In some embodiments, the pluripotent stem cell exhibits reduced activity of the β-Glucocerebrosidase (GCase) enzyme encoded by GBA1 prior to being introduced with the DNA sequence encoding GBA1 and the transposase or the nucleic acid sequence encoding a transposase, as compared to a reference cell. In some embodiments, the reference cell does not contain a GBA1 variant. In some embodiments, the reference cell does not exhibit decreased GCase activity. In some embodiments, the cell is from a subject who does not exhibit reduced GCase activity. In some embodiments, the reference cell is a cell from a subject without a Lewy body disease. In some embodiments, the reference cell is a cell from a subject without Parkinson's disease. In some embodiments, the reference cell is a cell from a subject without Gaucher's disease.

In some embodiments, GBA1 is human GBA1.

In some embodiments, the DNA sequence encoding GBA1 includes the sequence set forth in SEQ ID NO:2. In some embodiments, the DNA sequence encoding GBA1 includes a codon-optimized version of the sequence set forth in SEQ ID NO:2.

In some embodiments, the DNA sequence encoding GBA1 includes a coding region of the sequence set forth in SEQ ID NO:2. In some embodiments, the DNA sequence encoding GBA1 includes a codon-optimized version of a coding region of the sequence set forth in SEQ ID NO:2. In some embodiments, the DNA encoding GBA1 encodes an amino acid containing the amino acid sequence set forth in SEQ ID NO:1.

In some embodiments, the variant of GBA1 contains a single nucleotide polymorphism (SNP) that is associated with Parkinson's disease. In some embodiments, the cell is heterozygous for the GBA1 variant.

In some embodiments, the variant of GBA1 contains a single nucleotide polymorphism (SNP) that is associated with Gaucher's disease. In some embodiments, the cell is homozygous for the GBA1 variant. In some embodiments, the cell comprises biallelic variants in GBA1.

In some embodiments, the SNP is rs76763715. In some embodiments, the rs76763715 is a cytosine variant. In some embodiments, the variant of GBA1 containing a SNP encodes a serine, rather than an asparagine, at amino acid position 370 (N370S). In some embodiments, the wild-type form of GBA1 comprises a thymine instead of the cytosine variant.

In some embodiments, the SNP is rs421016. In some embodiments, the rs421016 is a guanine variant. In some embodiments, the variant of GBA1 comprising the SNP encodes a proline, rather than a leucine, at amino acid position 444 (L444P). In some embodiments, the wild-type form of GBA1 comprises an adenine instead of the guanine variant.

In some embodiments, the SNP is rs2230288. In some embodiments, the rs2230288 is a thymine variant. In some embodiments, the variant of GBA1 comprising the SNP encodes a lysine, rather than a glutamic acid, at position 326 (E326K). In some embodiments, the wild-type form of GBA1 comprises a cytosine instead of the thymine variant.

In some embodiments, the cell is an induced pluripotent stem cell (iPSC). In some embodiments, the iPSC is artificially derived from a non-pluripotent cell from a subject. In some embodiments, the non-pluripotent cell is a fibroblast. In some embodiments, the fibroblast has reduced GCase activity. In some embodiments, the subject has a a Lewy body disease (LBD). In some embodiments, the subject has Parkinson's disease. In some embodiments, the subject has Parkinson's disease dementia. In some embodiments, the subject has dementia with Lewy bodies (DLB). In some embodiments, the subject has Gaucher's disease.

In some embodiments, after the integration of the DNA sequence encoding GBA1 into the DNA of the cell, the method further includes determining the location of the integrated DNA sequence in the genome of the cell.

In some embodiments, after integration of the DNA sequence encoding GBA1 into the cell, the cell is differentiated into a hematopoietic stem cell (HSC), a dopaminergic (DA) neuron, a microglia, an astrocyte, an oligodendrocyte, or a macrophage. In some embodiments, after integration of the DNA sequence encoding GBA1 into the cell, the cell is differentiated into a dopaminergic (DA) neuron, a microglia, an astrocyte, or an oligodendrocyte. In some embodiments, the cell is differentiated into a DA neuron. In some embodiments, the cell is differentiated into a microglia. In some embodiments, the cell is differentiated into an astrocyte. In some embodiments, the cell is differentiated into an oligodendrocyte. In some embodiments, the cell is differentiated into a macrophage. In some embodiments, the cell is differentiated into an HSC.

Also provided herein are methods of of differentiating neural cells, the methods including: (a) performing a first incubation including culturing the cells produced by any of the methods provided herein in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and (b) performing a second incubation including culturing cells of the spheroid in a substrate-coated culture vessel under conditions to neurally differentiate the cells.

Also provided here are methods of differentiating neural cells, the methods including: (a) performing a first incubation including culturing a population of pluripotent stem cells that are modified by integration into the genome of the cells of an exogenous deoxyribonucleic acid (DNA) sequence encoding GBA1 operably linked to a promoter, wherein the culturing is in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and (b) performing a second incubation including culturing cells of the spheroid in a substrate-coated culture vessel under conditions to neurally differentiate the cells.

In some embodiments, prior to integration of the DNA sequence, the cells have reduced activity of GCase. In some embodiments, the cells endogenously contain a GBA1 variant. In some embodiments, the cells are heterozygous for the GBA1 variant. In some embodiments, the cells endogenously comprise a variant of GBA1 associated with Parkinson's Disease.

In some embodiments, the cells comprise biallelic variants in GBA1 or are homozygous for the GBA1 variant. In some embodiments, the cells comprise biallelic variants in GBA1. In some embodiments, the cells are homozygous for the GBA1 variant. In some embodiments, the cells endogenously contain one or more variant(s) of GBA1 associated with Gaucher's disease (GD).

In some embodiments, the cells are induced pluripotent stem cells.

Also provided here are methods of differentiating neural cells, the methods including: (a) performing a first incubation including culturing a population of pluripotent stem cells that are modified by integration into the genome of the cells of an exogenous deoxyribonucleic acid (DNA) sequence encoding GBA1 operably linked to a promoter, wherein the culturing is in a first culture vessel, wherein beginning at the initiation of the first incubation (day 0) the cells are exposed to (i) an inhibitor of TGF-β/activin-Nodal signaling; and (ii) an inhibitor of bone morphogenetic protein (BMP) signaling; and (b) performing a second incubation comprising culturing cells produced by the first incubation in a second culture vessel under conditions to neurally differentiate the cells.

In some embodiments, prior to integration of the DNA sequence, the cells have reduced activity of GCase. In some embodiments, the cells endogenously contain a GBA1 variant. In some embodiments, the cells are heterozygous for the GBA1 variant. In some embodiments, the cells endogenously comprise a variant of GBA1 associated with Parkinson's Disease.

In some embodiments, the cells comprise biallelic variants in GBA1 or are homozygous for the GBA1 variant. In some embodiments, the cells comprise biallelic variants in GBA1. In some embodiments, the cells are homozygous for the GBA1 variant. In some embodiments, the cells endogenously contain one or more variant(s) of GBA1 associated with Gaucher's disease (GD).

In some embodiments, the cells are induced pluripotent stem cells.

In some embodiments, the cells are exposed to the inhibitor of TGF-β/activin-Nodal signaling up to a day at or before day 7. In some embodiments, the cells are exposed to the inhibitor of TGF-β/activin-Nodal beginning at day 0 and through day 6, inclusive of each day.

In some embodiments, the cells are exposed to the at least one activator of SHH signaling up to a day at or before day 7. In some embodiments, the cells are exposed to the at least one activator of SHH signaling beginning at day 0 and through day 6, inclusive of each day.

In some embodiments, the cells are exposed to the inhibitor of BMP signaling up to a day at or before day 11. In some embodiments, the cells are exposed to the inhibitor of BMP signaling beginning at day 0 and through day 10, inclusive of each day.

In some embodiments, the cells are exposed to the inhibitor of GSK3β signaling up to a day at or before day 13. In some embodiments, the cells are exposed to the inhibitor of GSK3b signaling beginning at day 0 and through day 12, inclusive of each day.

In some embodiments, culturing the cells under conditions to neurally differentiate the cells includes exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch signaling.

In some embodiments, the cells are exposed to BAGCT and the inhibitor of Notch signaling beginning on day 11. In some embodiments, the cells are exposed to BAGCT and the inhibitor of Notch signaling beginning at day 11 and until harvest of the neurally differentiated cells.

In some embodiments, the inhibitor of TGF-β/activin-Nodal signaling is SB431542.

In some embodiments, the at least one activator of SHH signaling is SHH or purmorphamine. In some embodiments, the at least one activator of SHH signaling is SHH. In some embodiments, the at least one activator of SHH signaling is purmorphamine. In some embodiments, the at least one activator of SHH signaling is SHH and purmorphamine.

In some embodiments, the inhibitor of BMP signaling is LDN193189.

In some embodiments, the inhibitor of GSK3β signaling is CHIR99021.

In some embodiments, the neurally differentiated cells are harvested between about day 18 and about day 25. In some embodiments, the neurally differentiated cells are harvested between about day 18 and about day 20. In some embodiments, the neurally differentiated cells are harvested on about day 18. In some embodiments, the neurally differentiated cells are harvested on about day 20.

In some embodiments, the neurally differentiated cells are cryopreserved. In some embodiments, the method further includes cryopreserving the neurally differentiated cells. In some embodiments, the cryopreserving comprises formulating the neurally differentiated cell with a cryoprotectant.

Also provided herein is a cell produced by any of the methods provided herein.

Also provided herein is a pluripotent stem cell produced by any of the methods provided herein.

Also provided herein is a neurally differentiated cell produced by any of the methods provided herein.

Also provided herein is a microglial cell produced by any of the methods provided herein.

Also provided herein is a macrophage produced by any of the methods provided herein.

Also provided herein is a hematopoietic stem cell produced by any of the methods provided herein.

Also provided herein is a pluripotent stem cell that has been introduced with (i) a deoxyribonucleic acid (DNA) sequence encoding GBA1 operably linked to a promoter, wherein the DNA sequence is positioned between inverted terminal repeats and is capable of integrating into DNA in the cell; and (ii) a transposase or a nucleic acid sequence encoding a transposase, wherein the introducing in (i) and (ii) results in integration of the DNA sequence encoding GBA1 into the genome of the cell.

Also provided herein is a pluripotent stem cell comprising an exogenous deoxyribonucleic acid (DNA) sequence encoding GBA1 integrated into its genome. In some embodiments, the pluripotent stem cell is an induced pluripotent stem cell.

Also provided herein is a neurally differentiated cell comprising an exogenous deoxyribonucleic acid (DNA) sequence encoding GBA1 integrated into its genome. In some embodiments, the neurally differentiated cell expresses EN1 and CORIN. In some embodiments, the neurally differentiated cell is a committed dopaminergic precursor cells.

Also provided herein is a microglial cell comprising an exogenous deoxyribonucleic acid (DNA) sequence encoding GBA1 integrated into its genome.

Also provided herein is a macrophage comprising an exogenous deoxyribonucleic acid (DNA) sequence encoding GBA1 integrated into its genome.

Also provided herein is a hematopoietic stem cell comprising an exogenous deoxyribonucleic acid (DNA) sequence encoding GBA1 integrated into its genome.

In some embodiments, the cell is formulated with a cryoprotectant.

In some embodiments, the DNA sequence is operably linked to a promoter. In some embodiments, the DNA sequence was integrated into the genome of the cell by a transposon-based system.

In some embodiments, prior to being introduced with the DNA sequence and the transposase or nucleic acid sequencing encoding the transposase, the cell has reduced GCase activity. In some embodiments, the cell endogenously comprises a GBA1 variant. In some embodiments, the cell is heterozygous for the GBA1 variant. In some embodiments, the cell contains a variant of GBA1 associated with Parkinson's disease.

In some embodiments, the cell is homozygous for the GBA1 variant or comprises biallelic variants in GBA1. In some embodiments, the cell is homozygous for the GBA1 variant. In some embodiments, the cell comprises biallelic variants in GBA1. In some embodiments, the cell endogenously comprises a variant of GBA1 associated with Gaucher's disease.

In some embodiments, GBA1 is the wild-type form of GBA1. In some embodiments, the wild-type form of GBA1 is encoded by the sequence set forth in SEQ ID NO:2. In some embodiments, the wild-type form of GBA1 is encoded by the sequence set forth in SEQ ID NO:2 or a sequence having at least 80%, at least 85%, at least 90%, at least 95%, or at least 99% sequence identity to the sequence set forth in SEQ ID NO:2. In some embodiments, the wild-type form of GBA1 encodes an amino acid sequence comprising the amino acid sequence set forth in SEQ ID NO: 1. In some embodiments, GBA1 is a functional GBA1 or a portion thereof. In some embodiments, a functional GBA1 is capable of being transcribed into GBA1 mRNA or a portion thereof. In some embodiments, a functional GBA1 is capable of being transcribed into GBA1 mRNA or a portion thereof, which is capable of being translated into a functional GCase enzyme or a portion thereof. In some embodiments, a functional GBA1 is capable of (i) being transcribed into GBA1 mRNA or a portion thereof; and (ii) being transcribed into GBA1 mRNA or a portion thereof, which is capable of being translated into a functional GCase enzyme or a portion thereof. In some embodiments, a functional GCase enzyme or a portion thereof has the enzymatic activity of a wild-type GCase enzyme. In some embodiments, the enzymatic activity of GCase is determined by any of the methods provided herein.

In some embodiments, the DNA sequence encoding GBA1 is part of a plasmid.

In some embodiments, the promoter is selected from the group consisting of: ubiquitin C (UBC promoter) cytomegalovirus (CMV) promoter, phosphoglycerate kinase (PGK) promoter, CMV early enhancer/chicken b actin (CAG) promoter, glial fibrilary acidic protein (GFAP) promoter, synapsin-1 promoter, and Neuron Specific Enolase (NSE) promoter. In some embodiments, the promoter is a PGK or UBC promoter. In some embodiments, the promoter is a PGK promoter. In some embodiments, the promoter is a UBC promoter.

In some embodiments, the transposase is a Class II transposase. In some embodiments, the transposase is selected from the group consisting of: Sleeping Beauty, piggyBac, TcBuster, Frog Prince, Tol2, Tcl/mariner, or a derivative thereof having transposase activity. In some embodiments, the transposase is Sleeping Beauty, PiggyBac, or TcBuster. In some embodiments, the transposase is Sleeping Beauty. In some embodiments, the transposase is PiggyBac. In some embodiments, the transposase is TcBuster.

In some embodiments, the nucleic acid sequence encoding the transposase and/or the DNA sequence encoding GBA1 are introduced into the cell by electrotransfer; chemotransfer; or nanoparticles. In some embodiments, the nucleic acid sequence encoding the transposase is introduced into the cell by electrotransfer; chemotransfer; or nanoparticles. In some embodiments, the DNA sequence encoding GBA1 is introduced into the cell by electrotransfer; chemotransfer; or nanoparticles. In some embodiments, the nucleic acid sequence encoding the transposase and the DNA sequence encoding GBA1 are introduced into the cell by electrotransfer; chemotransfer; or nanoparticles. In some embodiments, the electrotransfer is by electroporation or nucleofection.

In some embodiments, the cell is introduced with a nucleic acid encoding a transposase. In some embodiments, the nucleic acid encoding a transposase is part of a plasmid. In some embodiments, the nucleic acid encoding a transposase is ribonucleic acid (RNA). In some embodiments, the nucleic acid encoding a transposase is DNA.

In some embodiments, the plasmid containing the DNA sequence encoding GBA1 and the plasmid containing the nucleic acid sequence encoding the transposase are different plasmids. In some embodiments, the plasmid containing the DNA sequence encoding GBA1 and the plasmid containing the nucleic acid sequence encoding the transposase are the same plasmid.

In some embodiments, the cell is introduced with a transposase.

In some embodiments, (i) the DNA sequence encoding GBA1 and the (ii) the transposase or the nucleic acid sequence encoding the transposase are introduced into the cell at the same time.

In some embodiments, the pluripotent stem cell exhibits decreased expression of GBA1 prior to being introduced with the DNA sequence encoding GBA1 and the transposase or the nucleic acid sequence encoding a transposase, as compared to a reference cell. In some embodiments, the pluripotent stem cell exhibits reduced activity of the β-Glucocerebrosidase (GCase) enzyme encoded by GBA1 prior to being introduced with the DNA sequence encoding GBA1 and the transposase or the nucleic acid sequence encoding a transposase, as compared to a reference cell. In some embodiments, the reference cell does not contain a GBA1 variant. In some embodiments, the reference cell does not exhibit decreased GCase activity. In some embodiments, the cell is from a subject who does not exhibit reduced GCase activity. In some embodiments, the reference cell is a cell from a subject without an LBD. In some embodiments, the reference cell is a cell from a subject without Parkinson's disease. In some embodiments, the reference cell is a cell from a subject without Parkinson's disease dementia. In some embodiments, the reference cell is a cell from a subject without DLB. In some embodiments, the reference cell is a cell from a subject without Gaucher's disease.

In some embodiments, GBA1 is human GBA1.

In some embodiments, the DNA sequence encoding GBA1 includes the sequence set forth in SEQ ID NO:2. In some embodiments, the DNA sequence encoding GBA1 includes a codon-optimized version of the sequence set forth in SEQ ID NO:2.

In some embodiments, the DNA sequence encoding GBA1 includes a coding region of the sequence set forth in SEQ ID NO:2. In some embodiments, the DNA sequence encoding GBA1 includes a codon-optimized version of a coding region of the sequence set forth in SEQ ID NO:2. In some embodiments, the DNA encoding GBA1 encodes an amino acid containing the amino acid sequence set forth in SEQ ID NO:1.

In some embodiments, the variant of GBA1 contains a single nucleotide polymorphism (SNP) that is associated with Parkinson's disease. In some embodiments, the cell is heterozygous for the GBA1 variant.

In some embodiments, the variant of GBA1 contains a single nucleotide polymorphism (SNP) that is associated with Gaucher's disease. In some embodiments, the cell is homozygous for the GBA1 variant. In some embodiments, the cell comprises biallelic variants of GBA1.

In some embodiments, the SNP is rs76763715. In some embodiments, the rs76763715 is a cytosine variant. In some embodiments, the variant of GBA1 containing a SNP encodes a serine, rather than an asparagine, at amino acid position 370 (N370S). In some embodiments, GBA1 comprises a thymine instead of the cytosine variant.

In some embodiments, the SNP is rs421016. In some embodiments, the rs421016 is a guanine variant. In some embodiments, the variant of GBA1 comprising the SNP encodes a proline, rather than a leucine, at amino acid position 444 (L444P). In some embodiments, GBA1 comprises an adenine instead of the guanine variant.

In some embodiments, the SNP is rs2230288. In some embodiments, the rs2230288 is a thymine variant. In some embodiments, the variant of GBA1 comprising the SNP encodes a lysine, rather than a glutamic acid, at position 326 (E326K). In some embodiments, GBA1 comprises a cytosine instead of the thymine variant.

In some embodiments, the cell is an induced pluripotent stem cell (iPSC). In some embodiments, the iPSC is artificially derived from a non-pluripotent cell from a subject. In some embodiments, the non-pluripotent cell is a fibroblast. In some embodiments, the fibroblast exhibits reduced GCase activity. In some embodiments, the subject has an LBD. In some embodiments, the subject has Parkinson's disease. In some embodiments, the subject has Parkinson's disease dementia. In some embodiments, the subject has DLB. In some embodiments, the subject has Gaucher's disease.

In some embodiments, after the integration of the DNA sequence encoding GBA1 into the DNA of the cell, the location of the integrated DNA sequence in the genome of the cell is determined.

Also provided herein are therapeutic compositions of cells produced by any of the methods provided herein.

In some embodiments, cells of the composition express EN1 and CORIN and less than 10% of the total cells in the composition express TH. In some embodiments, less than 5% of the total cells in the composition express TH. In some embodiments, the therapeutic composition comprises a cryoprotectant.

In some embodiments, the therapeutic composition is for use in a method of treating a subject with reduced GCase activity. In some embodiments, the therapeutic composition is for use in treating a subject with reduced GCase activity. In some embodiments, the therapeutic composition is for use in the manufacture of a medicament for treatment of reduced GCase activity.

In some embodiments, the therapeutic composition is for use in a method of treating a Lewy body disease (LBD). In some embodiments, the therapeutic composition is for use in treating a subject with an LBD. In some embodiments, the therapeutic composition is for use in the manufacture of a medicament for treatment of an LBD.

In some embodiments, the therapeutic composition is for use in a method of treating Parkinson's disease. In some embodiments, the therapeutic composition is for use in treating a subject with Parkinson's disease. In some embodiments, the therapeutic composition is for use in the manufacture of a medicament for treatment of Parkinson's disease.

In some embodiments, the therapeutic composition is for use in a method of treating Parkinson's disease dementia. In some embodiments, the therapeutic composition is for use in treating a subject with Parkinson's disease dementia. In some embodiments, the therapeutic composition is for use in the manufacture of a medicament for treatment of Parkinson's disease dementia.

In some embodiments, the therapeutic composition is for use in a method of treating dementia with Lewy bodies (DLB). In some embodiments, the therapeutic composition is for use in treating a subject with DLB. In some embodiments, the therapeutic composition is for use in the manufacture of a medicament for treatment of DLB.

In some embodiments, the therapeutic composition is for use in a method of treating a subject with a heterozygous variant of GBA1. In some embodiments, the therapeutic composition is for use in treating a subject with a heterozygous variant of GBA1. In some embodiments, the therapeutic composition is for use in the manufacture of a medicament for treatment of a heterozygous variant of GBA.

In some embodiments, the therapeutic composition is for use in a method of treating Gaucher's disease. In some embodiments, the therapeutic composition is for use in treating a subject with Gaucher's disease. In some embodiments, the therapeutic composition is for use in the manufacture of a medicament for treatment of Gaucher's disease.

In some embodiments, the therapeutic composition is for use in a method of treating a subject with biallelic variants of GBA1. In some embodiments, the therapeutic composition is for use in treating a subject with biallelic variants of GBA1. In some embodiments, the therapeutic composition is for use in the manufacture of a medicament for treatment of biallelic variants of GBA1.

In some embodiments, the therapeutic composition is for use in a method of treating a subject with a homozygous variant of GBA1. In some embodiments, the therapeutic composition is for use in treating a subject with a homozygous variant of GBA1. In some embodiments, the therapeutic composition is for use in the manufacture of a medicament for treatment of a homozygous variant of GBA1.

Also provided herein are methods of treatment including administering to a subject a therapeutically effective amount of any of the therapeutic compositions provided herein.

In some embodiments, the cells of the therapeutic composition are autologous to the subject. In some embodiments, prior to be administered the therapeutic composition, the subject has reduced GCase activity. In some embodiments, the subject has a heterozygous variant of GBA1.

In some embodiments, the subject has a disease or disorder associated with reduced GCase activity. In some embodiments, the subject has Parkinson's disease. In some embodiments, the subject has a homozygous variant of GBA1 or biallelic variants of GBA1. In some embodiments, the subject has a homozygous variant of GBA1. In some embodiments, the subject has biallelic variants of GBA1. In some embodiments, the subject has Gaucher's disease.

In some embodiments, the administering comprises delivering cells of a composition by stereotactic injection. In some embodiments, the administering comprises delivering cells of a composition through a catheter. In some embodiments, the cells are delivered to the striatum of the subject.

Also provided herein are uses of any of the compositions provided herein for the treatment of a Lewy body disease (LBD). In some embodiments the LBD is Parkinson's disease. In some embodiments, the LBD is Parkinson's disease with dementia. In some embodiments, the LBD is dementia with Lewy bodies.

Also provided herein are uses of any of the compositions provided herein for the treatment of Parkinson's disease.

Also provided herein are uses of any of the compositions provided herein for the treatment of reduced GCase activity.

Also provided herein are uses of any of the compositions provided herein for the treatment of Gaucher's disease.

Also provided herein are uses of any of the compositions provided herein for the treatment of a subject with a Lewy body disease (LBD). In some embodiments the LBD is Parkinson's disease. In some embodiments, the LBD is Parkinson's disease with dementia. In some embodiments, the LBD is dementia with Lewy bodies.

Also provided herein are uses of any of the compositions provided herein for the treatment of a subject with Parkinson's disease.

Also provided herein are uses of any of the compositions provided herein for the treatment of a subject with reduced GCase activity.

Also provided herein are uses of any of the compositions provided herein for the treatment of a subject with Gaucher's disease.

Also provided herein are uses of any of the compositions provided herein in the manufacture of a medicament for the treatment of a Lewy body disease (LBD). In some embodiments the LBD is Parkinson's disease. In some embodiments, the LBD is Parkinson's disease with dementia. In some embodiments, the LBD is dementia with Lewy bodies.

Also provided herein are uses of any of the compositions provided herein in the manufacture of a medicament for the treatment of Parkinson's Disease.

Also provided herein are uses of any of the compositions provided herein in the manufacture of a medicament for the treatment of reduced GCase activity.

Also provided herein are uses of any of the compositions provided herein in the manufacture of a medicament for the treatment of Gaucher's Disease.

Also provided herein is a transposon-based system for increasing expression of GBA1 in a cell, the system including: (i) a deoxyribonucleic acid (DNA) sequence encoding GBA1, wherein the DNA sequence is positioned between at least two inverted terminal repeats and is capable of integrating into DNA in a cell; and (ii) a transposase or a nucleic acid sequence encoding a transposase, wherein the cell exhibits (i) reduced activity of the β-Glucocerebrosidase (GCase) enzyme encoded by GBA1 and/or (ii) reduced expression of GBA1, prior to being introduced with the DNA sequence encoding GBA1 and the transposase or the nucleic acid sequence encoding a transposase, compared to a reference cell. In some embodiments, the reference cell does not contain a GBA1 variant. In some embodiments, the reference cell does not exhibit decreased GCase activity. In some embodiments, the reference cell is a cell from a subject without an LBD. In some embodiments, the reference cell is a cell from a subject without Parkinson's disease. In some embodiments, the reference cell is a cell from a subject without Parkinson's disease dementia. In some embodiments, the reference cell is a cell from a subject without DLB. In some embodiments, the reference cell is a cell from a subject without Gaucher's disease.

In some embodiments, GBA1 is the wild-type form of GBA1. In some embodiments, the wild-type form of GBA1 is encoded by the sequence set forth in SEQ ID NO:2. In some embodiments, the wild-type form of GBA1 is encoded by the sequence set forth in SEQ ID NO:2 or a sequence having at least 80%, at least 85%, at least 90%, at least 95%, or at least 99% sequence identity to the sequence set forth in SEQ ID NO:2. In some embodiments, the wild-type form of GBA1 encodes an amino acid sequence comprising the amino acid sequence set forth in SEQ ID NO: 1. In some embodiments, GBA1 is a functional GBA1 or a portion thereof. In some embodiments, a functional GBA1 is capable of being transcribed into GBA1 mRNA or a portion thereof. In some embodiments, a functional GBA1 is capable of being transcribed into GBA1 mRNA or a portion thereof, which is capable of being translated into a functional GCase enzyme or a portion thereof. In some embodiments, a functional GBA1 is capable of (i) being transcribed into GBA1 mRNA or a portion thereof; and (ii) being transcribed into GBA1 mRNA or a portion thereof, which is capable of being translated into a functional GCase enzyme or a portion thereof. In some embodiments, a functional GCase enzyme or a portion thereof has the enzymatic activity of a wild-type GCase enzyme. In some embodiments, the enzymatic activity of GCase is determined by any of the methods described in Section II.D.

In some embodiments, the cell endogenously contains a variant of GBA1 associated with Parkinson's Disease. In some embodiments, the cell is heterozygous for the GBA1 variant. In some embodiments, the variant of GBA1 is a single nucleotide polymorphism (SNP) that is associated with Parkinson's disease.

In some embodiments, the cell is homozygous for the GBA1 variant or comprises biallelic GBA1 variants. In some embodiments, the cell is homozygous for the GBA1 variant. In some embodiments, the cell comprises biallelic GBA1 variants. In some embodiments, the variant of GBA1 contains one or more single nucleotide polymorphism(s) (SNP) that is associated with Gaucher's disease.

In some embodiments, the SNP is rs76763715. In some embodiments, the rs76763715 is a cytosine variant. In some embodiments, the variant of GBA1 containing a SNP encodes a serine, rather than an asparagine, at amino acid position 370 (N370S). In some embodiments, the wild-type form of GBA1 contains a thymine instead of the cytosine variant.

In some embodiments, the SNP is rs421016. In some embodiments, the rs421016 is a guanine variant. In some embodiments, the variant of GBA1 containing the SNP encodes a proline, rather than a leucine, at amino acid position 444 (L444P). In some embodiments, wherein the wild-type form of GBA1 contains an adenine instead of the guanine variant.

In some embodiments, the SNP is rs2230288. In some embodiments, the rs2230288 is a thymine variant. In some embodiments, the variant of GBA1 containing the SNP encodes a lysine, rather than a glutamic acid, at position 326 (E326K). In some embodiments, the wild-type form of GBA1 contains a cytosine instead of the thymine variant.

In some embodiments, the cell is a pluripotent stem cell (PSC). In some embodiments, the cell is an induced pluripotent stem cell (iPSC).

In some embodiments, a plurality of the PSCs are neurally differentiated by a method including: (a) performing a first incubation including culturing the PSCs in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and (b) performing a second incubation including culturing cells of the spheroid in a substrate-coated culture vessel under conditions to neurally differentiate the cells. In some embodiments, the PSC is an induced pluripotent stem cell (iPSC).

In some embodiments, the PSCs are exposed to the inhibitor of TGF-β/activin-Nodal signaling and the at least one activator of SHH signaling up to a day at or before day 7.

In some embodiments, the PSCs are exposed to the inhibitor of BMP signaling up to a day at or before day 11.

In some embodiments, the PSCs are exposed to the inhibitor of GSK3β signaling up to a day at or before day 13.

In some embodiments, culturing the cells under conditions to neurally differentiate the cells includes exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch signaling.

### BRIEF DESCRIPTION OF THE DRAWINGS

FIG. 1 shows an exemplary non-adherent protocol for the differentiation of pluripotent stem cells into determined dopamine (DA) neuron progenitor cells or DA neurons.

FIG. 2 shows an exemplary adherent protocol for the differentiation of pluripotent stem cells into determined dopamine (DA) neuron progenitor cells or DA neurons.

FIG. 3A shows GFP expression in day 0 iPSCs that are non-transfected, transfected with a UBC-GBA-T2A-GFP construct, or transfected with a PGK-GBA-T2A-GFP construct (left to right, respectively).

FIG. 3B shows GFP expression in day 25 differentiated cells that are non-transfected, transfected with the UBC-GBA-T2A-GFP construct, or transfected with the PGK-GBA-T2A-GFP construct (left to right, respectively).

FIG. 4 shows the GCase activity in day 0 iPSCs or day 25 differentiated cells from Donor 1 that were transfected (transposon), as compared to non-transfected cells from the Donor 1 parental cell line (N370S), healthy control cells (Ctrl), cells from a donor having idiopathic Parkinson's disease (ID-PD), and non-transfected clones derived from Donor 1's parental cell line (N370S clones).

FIG. 5 shows in vitro GCase activity in day 60 differentiated cells from three different unaffected donors (each dot represents a different donor) or from two different isogenic cell lines.

FIG. 6 shows the number of wild-type GBA1 transgene copies integrated into cells transfected with the indicated UBC-GBA-T2A-GFP or PGK-GBA-T2A-GFP transposon constructs.

FIG. 7 shows the number of wild-type GBA1 transgene copies integrated into cells transfected with the a PGK-GBA-T2A-GFP transposon construct from two different donors.

FIG. 8 shows the integration site of the wild-type GBA1 transgene in iPSC clones transfected with the indicated UBC-GBA-T2A-GFP or PGK-GBA-T2A-GFP transposon constructs.

FIGS. 9A and 9B show gene expression analyses of 14 and 25 different genes in cells of clones 16 and 18, respectively, from FIG. 8, as compared to unmodified (“unperturbed”) cells.

FIG. 10 shows genome-wide gene expression analysis among differentiated cells derived from iPSCs transfected with a low PGK-GBA-T2A-GFP transposon construct and differentiated cells derived from non-transfected iPSCs (day of harvest is indicated). Scale shows the Euclidian distance between each sample pair.

FIG. 11 shows the gene expression levels of FOXA2, LMX1A, and PAX6 in differentiated cells harvested on day 20 and derived from iPSCs transfected with a PGK-GBA-T2A-GFP transposon construct or non-transfected iPSCs.

FIG. 12 shows the percentage of day 35 differentiated cells surface positive for FOXA2 and FOXA2/TH expression, following differentiation from iPSCs transfected with a PGK-GBA-T2A-GFP transposon construct and having different copy numbers of the wild-type GBA1 transgene integrated.

FIGS. 13A and 13B show GCase protein expression and activity, respectively, in iPSCs transfected with a PGK-GBA-T2A-GFP transposon construct (day 0) or cells differentiated therefrom (day 35). The copy number of the wild-type GBA1 transgene is indicated on the left of each graph.

FIG. 14 shows GCase activity in iPSCs transfected with a PGK-GBA-T2A-GFP transposon construct (day 0) or cells differentiated therefrom (day 35) among clones having different copy numbers of the wild-type GBA1 transgene integrated. The copy number of the wild-type GBA1 transgene is indicated on the left of the graph.

FIG. 15 shows GCase activity in differentiated cells at day 40 following modulation of GBA1 expression by transfection with a PGK-GBA-T2A-GFP transposon construct (“transposon clones”), overexpression of GBA1 by an AAV-based method (“AAV treated”), or correction of the N370S mutation by a CRISPR/Cas-based method (“CRISPR corrected”).

FIG. 16 shows GCase protein levels in differentiated cells on days 35, 50, and 65 among cells modified by transposon-, AAV-, and CRISPR-based methods, as compared to cells from a donor having idiopathic Parkinson's disease (“idiopathic”), cells having a GBA N370S mutation, and cells completely knocked out for GBA1.

FIGS. 17A and 17B show the relationship between GCase activity (“substrate converted”) and the number of GBA1 copies in day 0 iPSCs and day 35 differentiated cells, respectively, transfected with a PGK-GBA-T2A-GFP transposon construct.

FIGS. 17C and 17D show the relationship between GCase activity (“substrate converted”) and the number of copies of GBA1 integrated into mRNA in day 0 iPSCs and day 35 differentiated cells, respectively, transfected with a PGK-GBA-T2A-GFP transposon construct.

FIGS. 17E and 17F show the relationship between GCase activity (“substrate converted”) and the number of copies of GBA1 integrated into intergenic regions in day 0 iPSCs and day 35 differentiated cells, respectively, transfected with a PGK-GBA-T2A-GFP transposon construct.

FIG. 17G shows the relationship between GCase activity (“substrate converted”) between day 0 iPSCs and day 35 differentiated cells transfected with a PGK-GBA-T2A-GFP transposon construct.

### DETAILED DESCRIPTION

The present disclosure relates to methods of increasing expression and/or activity of 3-Glucocerebrosidase (GCase), such as in a subject having reduced GCase activity and/or a variant of the GBA1 gene encoding GCase, e.g., a gene variant associated with Parkinson's Disease (PD) and/or reduced GCase activity. In some embodiments, the subject has reduced GCase activity. In some embodiments, the subject has a variant of GBA1. In some embodiments, the subject is heterozygous for a variant of GBA1. In some cases, subjects having a genetic variation of GBA1, e.g., a single nucleotide polymorphism (SNP), associated with Parkinson's Disease (PD) exhibit decreased activity of GCase. In some cases, subjects having a genetic variation of GBA1, e.g., one or more single nucleotide polymorphism (SNP), associated with Gaucher's disease (GD) exhibit decreased activity of GCase. In particular, the present disclosure relates to methods of stably overexpressing the GBA1 gene by transposon-based methods, including in subjects having reduced activity of GCase to increase expression and/or activity of GCase.

In particular, the present disclosure also relates to methods of stably overexpressing the GBA1 gene by transposon-based methods, including in subjects having a SNP in the GBA1 gene, to increase expression and/or activity of GCase. The provided methods include transposon-based vectors to increase expression of GBA1 and/or GCase activity in cells from a subject with PD, and use of such cells or descendants of such cells in replacement cell therapy for treating PD. In particular embodiments, the cell is a pluripotent stem cell, and, in some embodiments, the present disclosure further includes methods of lineage specific differentiation of such pluripotent stem cells, having stable overexpression of GBA1.

The overexpressing cells made using the methods provided herein are further contemplated for various uses including, but not limited to, use as a therapeutic to reverse disease of, damage to, or a lack of, a certain cell type, such as dopaminergic (DA) neurons, microglia, astrocytes, or oligodendrocytes, in a patient. In some embodiments, the patient has an LBD, such as Parkinson's disease, Parkinson's disease dementia, or dementia with Lewy bodies. The overexpressing cells made using the methods provided herein are contemplated for various uses including, but not limited to, use as a therapeutic to provide, a certain cell type, such hematopoietic stem cells (HSCs), to a patient. In some embodiments, overexpressing cells such as HSCs or microglia are contemplated for use in treating Gaucher's disease.

Specifically described are methods of overexpressing GBA1 in pluripotent stem (PS) cells, such as in subjects with decreased activity of GCase and/or a gene variant, e.g., a SNP, in GBA1 that is associated with PD, and methods for differentiating the PS cells into one or more neural cell types. In some embodiments, the subject has decreased GCase activity. In some embodiments, the subject has a variant in GBA1. In some embodiments, the variant is associated with Parkinson's disease. In some embodiments, the variant is associated with Gaucher's disease. In some embodiments, the GBA1 gene is the wild-type form thereof. In some embodiments, the GBA1 gene is a functional GBA1 or a portion thereof.

Parkinson's disease (PD) is a progressive neurodegenerative disorder that primarily affects dopaminergic neurons of the substantia nigra. It is currently the second most common neurodegenerative, estimated to affect 4-5 million patients worldwide. This number is predicted to more than double by 2030. PD is the second most common neurodegenerative disorder after Alzheimer's disease, affecting approximately 1 million patients in the US with 60,000 new patients diagnosed each year. Currently there is no cure for PD, which is characterized pathologically by a selective loss of midbrain DA neurons in the substantia nigra. A fundamental characteristic of PD is therefore progressive, severe and irreversible loss of midbrain dopamine (DA) neurons resulting in ultimately disabling motor dysfunction.

Mutations in certain genes can increase the risk of developing neurodegenerative diseases, such as PD or Parkinsonism. For instance, certain mutations in the GBA1 gene have been associated with the development of PD and Parkinsonism. Hundreds of mutations have been found throughout the GBA1 gene, including common and rare variants. See Sidransky et al., New England J. Med. (2009) 361(17): 1651-61. Mutations in GBA1 are hypothesized to lead to degradation of the expressed protein product, glucocerebrosidase (GBA), an enzyme in the lysosome, as well as to disruptions in its lysosomal targeting and performance therein. See Do et al., Mol. Neurodegeneration (2019) 14:36. It has been estimated that at least 7-10% of PD patients have a GBA1 mutation, that GBA1 mutations increase risk for developing PD by 20- to 30-fold, and that 30% of carriers of a GBA1 mutation will develop PD by 80 years of age. See Migdalska-Richards and Schapira, J. Neurochem. (2016); 139 (Suppl 1): 77-90. Further, biallelic or homozygous mutations in GBA1 result in the autosomal recessive lysosomal storage disorder, Gaucher's disease (GD), which can have neurodegenerative features. See Stoker et al., “Pathological Mechanisms and Clinical Aspects of GBA1 Mutation-Associated Parkinson's Disease,” Parkinson's Disease: Pathogenesis and Clinical Aspects, Codon Publications (2018) Chapter 3.

GBA1 mutations that are associated with the development of PD and Parkinsonism include mutations in the GBA1 gene that result in an N370S amino acid change due to the presence of a serine, rather than an asparagine, at amino acid position 370 in the expressed Glucocerebrosidase (GCase) enzyme (e.g., with reference to SEQ ID NO:1). Other mutations in the GBA1 gene that are associated with the development of PD and Parkinsonism include mutations that result in an L444P amino acid change due to the presence of a proline, rather than a leucine, at position 444 in the expressed GCase enzyme, and mutations that result in an E326K amino acid change due to the presence of a lysine, rather than a glutamic acid, at position 326 in the expressed GCase enzyme (e.g., with reference to SEQ ID NO:1). Additional GBA1 mutations that have been identified as associated with the development of PD and Parkinsonism include any of those as described in Han et al., Int J Neurosci (2016) 126(5):415-21 and Sidranksy et al., NEJM (2009) 361:1651-61, such as T369M, G377S, D409H, R496H, R120W, V394L, K178T, R329C, L444R, and N188S.

In some cases, it is contemplated that a subject having a Lewy body disease (LBD) other than PD, such as Parkinson's disease dementia or dementia with Lewy bodies (DLB) may benefit from cells overexpressing GBA1. Accumulation of the protein α-synuclein into insoluble intracellular deposits termed Lewy bodies (LBs) is the characteristic neuropathological feature of LBDs. The influence of lipidosis-causing genetic mutations such as in GBA1 is thought to be two-fold, with both reduced clearance of α-synuclein due to autophagic impairments leading to a state of increased abundance of α-synuclein within cells, and the accumulation of lipids known to promote α-synuclein aggregation. Erskine et al., Acta Neuropathologica (2021) 121:511-26; and Stojkovska et al., Cell Tissue Res (2018) 373:51-60.

It is also contemplated that a subject may have reduced GCase activity without having a known or identified mutation in GBA1.

The provided embodiments also address problems related to the use of iPSCs derived from a subject, such as a subject having PD, that exhibited decreased activity of GCase and/or contain a variant in GBA1 that increases the risk of developing PD. The provided embodiments also contemplate that the iPSCs may be derived from any subject exhibiting decreased activity of GCase, such as in the iPSCs. In some embodiments, the subject has reduced GCase activity. In some embodiments, the subject has PD. In some embodiments, the subject has GD.

For instance, a strategy for the treatment of PD includes the differentiation of iPSCs derived from a patient with PD into certain cells, such as dopaminergic (DA) neurons, for autologous transplantation into the patient. However, if the patient's cells exhibit low activity of GCase and/or a variant in GBA1 associated with the development of PD, which may have contributed to the patient's development of PD and need for such cell transplantation, then the transplanted cells, e.g., DA neurons, would contribute to an increased risk of recurrence of PD by containing GCase with lower activity and/or the GBA1 gene variant. Thus, stably overexpressing the wildtype form of a GBA1 having reduced expression and/or a variant associated with PD in cells differentiated from iPSCs derived from a patient would allow for the benefits of autologous transplantation (e.g., avoiding ethical concerns, and avoiding risks of immune rejection) while reducing the risk of disease recurrence by providing the wildtype gene product capable of carrying out its normal functions.

Moroever, the human GBA1 gene has a pseudogene known as glucosylceramidase beta pseudogene 1 (GBAP1) that is approximately 96% homologous to GBA1. Horowitz et al., Genomics (1989), Vol. 4(1): 87-96. Specifically, the GBA1 gene, located on 1q21-22, includes 11 exons and is 16 kb upstream from GBAP1. The 85-kb region surrounding GBA is particularly gene-rich, encompassing seven genes and two pseudogenes. Recombination within and around the GBA locus occurs relatively frequently, complicating genotype analyses. Sidransky et al., New England J. Med. (2009) 361(17): 1651-61. Further, strategies for correcting gene variants in the GBA1 gene through gene editing run the risk of adversely affecting the GBAP1 pseudogene by also targeting its gene sequence due to the homology between GBA1 and GBAP1. Thus, alternative strategies are needed to compensate for GBA1 gene variants that do not adversely affect the GBAP1 pseudogene. The provided embodiments include such strategies.

Further, an advantage of the provided strategies is that they do not require that a GBA1 variant be known or identified in a subject, such as is required by other methods (e.g., CRISPR-based methods) that directly target a known variant. Rather, the provided methods can be used to increase expression of GBA1 in cells, without the need to determine whether the cells contain one or more GBA1 variants.

Thus, the provided methods may be useful for increasing GCase expression in any cells having or suspected of having reduced GCase activity. Such cells may be from a subject having or suspected of having an LBD. In some embodiments, the LDB is PD. In some embodiments, the LBD is Parkinson's disease dementia. In some embodiments, the LBD is DLB. Such cells may be from a subject having or suspected of having Parkinson's disease (PD) or Gaucher's disease. Such cells may be from a subject having or suspected of having PD. Such cells may be from a subject having or suspected of having Gaucher's disease.

The present disclosure also relates to methods of lineage specific differentiation of pluripotent stem cells (PSCs), such as embryonic stem (ES) cells or induced pluripotent stem cells (iPSCs), as well as stable overexpression of GBA1 in such cells, such as to increase GCase activity. In some embodiments, GBA1 is the wildtype form or a functional form or portion thereof. Specifically described are methods of directing lineage specific differentiation of PSCs or iPSCs into floor plate midbrain progenitor cells, determined dopamine (DA) neuron progenitor cells (DDPCs), dopamine (DA) neurons, or glial cells, such as microglia, astrocytes, oligodendrocytes, or ependymocytes. The differentiated cells made using the methods provided herein are further contemplated for various uses including, but not limited to, use as a therapeutic to reverse disease of, or damage to, a lack of dopamine neurons in a patient. For example, the pluripotent stem cells produced by any of the methods described herein may be differentiated into one or more types of cells, such as for cell therapy. In some embodiments, the pluripotent stem cells produced by any of the methods described herein are differentiated into hematopoietic stem cells (HSCs), macrophages, neurons, microglia, astrocytes, and/or oligodendrocytes. In some embodiments, the pluripotent stem cells produced by any of the methods described herein are differentiated into neurons, microglia, astrocytes, and/or oligodendrocytes. In some embodiments, the pluripotent stem cells are differentiated into neurons, e.g., DA neurons. In some embodiments, the pluripotent stem cells are differentiated into microglia. In some embodiments, the the pluripotent stem cells are differentiated into macrophages. In some embodiments, the the pluripotent stem cells are differentiated into HSCs.

Provided herein are methods for lineage specific differentiation of pluripotent stem cells (PSCs), such as embryonic stem (ES) cells or induced pluripotent stem cells (iPSCs) into floor plate midbrain progenitor cells, determined dopamine (DA) neuron progenitor cells, and/or dopamine (DA) neurons; or into glial cells, such as microglia, astrocytes, oligodendrocytes, or ependymocytes. In some aspects, PSCs are differentiated into floor plate midbrain progenitor cells. In some aspects, such floor plate midbrain progenitor cells are further differentiated into determined dopamine (DA) neuron progenitor cells. In some aspects, such determined dopamine (DA) neuron progenitor cells are further differentiated into dopamine (DA) neurons. In some aspects, PSCs are differentiated into floor plate midbrain progenitor cells, then into determined dopamine (DA) neuron progenitor cells, and finally, into dopamine (DA) neurons.

The provided embodiments address problems related to characteristics of Parkinson's disease (PD) including the selective degeneration of midbrain dopamine (mDA) neurons in patients' brains. Because PD symptoms are primarily due to the selective loss of DA neurons in the substantia nigra of the ventral midbrain, PD is considered suitable for cell replacement therapeutic strategies.

A challenge in developing a cell based therapy for PD has been the identification of an appropriate cell source for use in neuronal replacement. The search for an appropriate cell source is decades-long, and many potential sources for DA neuron replacement have been proposed. Kriks, Protocols for generating ES cell-derived dopamine neurons in Development and engineering of dopamine neurons (eds. Pasterkamp, R. J., Smidt, & Burbach) Landes Biosciences (2008); Fitzpatrick, et al., Antioxid. Redox. Signal. (2009) 11:2189-2208. Several of these sources progressed to early stage clinical trials including catecholaminergic cells from the adrenal medulla, carotid body transplants, or encapsulated retinal pigment epithelial cells. Madrazo, et al., N. Engl. J. Med. (1987) 316: 831-34; Arjona, et al., Neurosurgery (2003) 53: 321-28; Spheramine trial Bakay, et al., Front Biosci. (2004) 9:592-602. However, those trials largely failed to show clinical efficacy and resulted in poor long-term survival and low DA release from the grafted cells.

Another approach was the transplantation of fetal midbrain DA neurons, such as was performed in over 300 patients worldwide. Brundin, et al., Prog. Brain Res. (2010) 184:265-94; Lindvall, & Kokaia, J. Clin. Invest (2010) 120:29-40. Therapy using human fetal tissue in these patients demonstrated evidence of DA neuron survival and in vivo DA release up to 10 or 20 years after transplantation in some patients. In many patients, though, fetal tissue transplantation fails to replace DA neuronal function. Further, fetal tissue transplantation is plagued by challenges including low quantity and quality of donor tissue, ethical and practical issues surrounding tissue acquisition, and the poorly defined heterogeneous nature of transplanted cells, which are some of the factors contributing to the variable clinical outcomes. Mendez, et al. Nature Med. (2008); Kordower, et al. N. Engl. J. Med. (1995) 332:1118-24; and Piccini, et al. Nature Neuroscience (1999) 2:1137-40. Hypotheses as to the limited efficacy observed in the human fetal grafting trials include that fetal grafting may not provide a sufficient number of cells at the correct developmental stage and that fetal tissue is quite poorly defined by cell type and variable with regard to the stage and quality of each tissue sample. Bjorklund, et al. Lancet Neurol. (2003) 2:437-45. A further contributing factor may be inflammatory host response to the graft. Id.

Stem cell-derived cells, such as pluripotent stem cells (PSCs), are contemplated as a source of cells for applications in regenerative medicine. Pluripotent stem cells have the ability to undergo self-renewal and give rise to all cells of the tissues of the body. PSCs include two broad categories of cells: embryonic stem (ES) cells and induced pluripotent stem cells (iPSCs). ES cells are derived from the inner cell mass of preimplantation embryos and can be maintained indefinitely and expanded in their pluripotent state in vitro. Romito and Cobellis, Stem Cells Int. (2016) 2016:9451492. iPSCs can be obtained by reprogramming (“dedifferentiating”) adult somatic cells to become more ES cell-like, including having the ability to expand indefinitely and differentiate into all three germ layers. Id.

Pluripotent stem cells such as ES cells have been tested as sources for generating engraftable cells. Early studies in the 1990s using mouse ES cells demonstrated the feasibility of deriving specific lineages from pluripotent cells in vitro, including neurons. Okabe, et al., Mech. Dev. (1996) 59:89-102; Bain, et al., Dev. Biol. (1995) 168v342-357. Midbrain DA neurons were generated using a directed differentiation strategy based on developmental insights from early explants studies. Lee, et al., Nat. Biotechnol. (2000) 18v675-679; Ye, et al., Cell (1998) 93:755-66. However, these efforts did not result in cell populations containing high percentages of midbrain DA neurons or cells capable of restoring neuronal function in vivo. Additionally, the resulting populations contained a mixture of cell types in addition to midbrain DA neurons.

Existing strategies for using human PSCs (hPSCs) for cell therapy have not been entirely satisfactory. DA neurons derived from human PSCs generally have displayed poor in vivo performance, failing to compensate for the endogenous loss of neuronal function. Tabar, et al. Nature Med. (2008) 14:379-81; Lindvall and Kokaia, J. Clin. Invest (2010) 120: 29-40.

More recently, preclinical studies in which human ES cells were first differentiated into midbrain floor intermediates, and then further into DA neurons, exhibited in vivo survival and led to motor deficit recovery in animal models. Krik et al., Nature (2011) 480:547-51; Kirkeby et al., Cell Rep. (2012) 1:703-14. Despite these advances, the use of embryonic stem cells is plagued by ethical concerns, as well as the possibility that such cells may form tumors in patients. Finally, ES cell-derived transplants may cause immune reactions in patients in the context of allogeneic stem cell transplant.

The use of induced pluripotent stem cells (iPSCs), rather than ES-derived cells, has the advantages of avoiding ethical concerns. Further, derivation of iPSCs from a patient to be treated (i.e., the patient receives an autologous cell transplant) avoids risks of immune rejection inherent in the use of embryonic stem cells. As previous studies revealed that poor standardization of transplanted cell material contributes to high variability, new methods of producing substantial numbers of standardized cells, such as for autologous stem cell transplant, are needed. Lindvall and Kokaia, J. Clin. Invest (2010) 120: 29-40.

Thus, existing strategies have not yet proved to be successful in producing a population of differentiated cells for use in engraftment procedures for restoring neuronal function in vivo. Provided herein are methods of differentiating PSCs into determined dopaminergic neuron progenitor cells (DDPCs) and/or DA neurons cells.

Unlike previously reported methods, the differentiated cells produced by the methods described herein demonstrate physiological consistency. Importantly, this physiological consistency is maintained across cells differentiated from different subjects. This method therefore reduces variability both within and among subjects, and allows for better predictability of cell behavior in vivo. These benefits are associated with a successful therapeutic strategy, especially in the setting of autologous stem cell transplant, where cells are generated separately for each patient. Such reproducibility benefits among different subjects may also enable scaling in manufacturing and production processes.

Collectively, the methods described herein, including those for stably overexpressing GBA1 and those for differentiating cells containing the overexpressed GBA1, can be used in combination to provide the benefits described above.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

The term “expression” or “expressed” as used herein in reference to a gene refers to the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989, Molecular Cloning: A Laboratory Manual, 18.1-18.88).

The term “gene” can refer to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, guide RNA (e.g., a small guide RNA), or micro RNA.

The term “gene variant associated with Parkinson's Disease,” or “gene variant associated with PD,” or the like, refers to a variant of a gene, such as a single nucleotide polymorphism (SNP) or a mutation, where the presence of that variant in subjects, in either heterozygous or homozygous form, has been associated with an increased risk of developing Parkinson's Disease for those subjects, as compared to the risk of developing Parkinson's Disease for the general population. The term “SNP associated with Parkinson's Disease,” or “SNP associated with PD,” or “SNP that is associated with PD,” or the like, refers to a single nucleotide polymorphism (SNP), where the presence of that particular SNP in subjects, in either heterozygous or homozygous form, has been associated with an increased risk of developing Parkinson's Disease for those subjects, as compared to the risk of developing Parkinson's Disease for the general population. The increased risk of developing Parkinson's Disease can be an increased risk of developing Parkinson's Disease over the course of a lifetime or by a certain age, such as by, e.g., 40 years of age, 45 years of age, 50 years of age, 55 years of age, 60 years of age, 65 years of age, 70 years of age, 75 years of age, or 80 years of age. The general population can either be the general population worldwide, or the general population in one or more countries, continents, or regions, such as the United States. The extent of the increased risk is not particularly limited and can be, e.g., a risk that is or is at least 0.5-fold, 1-fold, 2-fold, 3-fold, 4-fold, 5-fold, 10-fold, 15-fold, 20-fold, 25-fold, or 30-fold higher than the risk for the general population.

As used herein, the term “stem cell” refers to a cell characterized by the ability of self-renewal through mitotic cell division and the potential to differentiate into a tissue or an organ. Among mammalian stem cells, embryonic and somatic stem cells can be distinguished. Embryonic stem cells reside in the blastocyst and give rise to embryonic tissues, whereas somatic stem cells reside in adult tissues for the purpose of tissue regeneration and repair.

As used herein, the term “adult stem cell” refers to an undifferentiated cell found in an individual after embryonic development. Adult stem cells multiply by cell division to replenish dying cells and regenerate damaged tissue. An adult stem cell has the ability to divide and create another cell like itself or to create a more differentiated cell. Even though adult stem cells are associated with the expression of pluripotency markers such as Rex1, Nanog, Oct4 or Sox2, they do not have the ability of pluripotent stem cells to differentiate into the cell types of all three germ layers.

As used herein, the terms “induced pluripotent stem cell,” “iPS” and “iPSC” refer to a pluripotent stem cell artificially derived (e.g., through man-made manipulation) from a non-pluripotent cell. A “non-pluripotent cell” can be a cell of lesser potency to self-renew and differentiate than a pluripotent stem cell. Cells of lesser potency can be, but are not limited to adult stem cells, tissue specific progenitor cells, primary or secondary cells.

As used herein, the term “pluripotent” or “pluripotency” refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism.

As used herein, the term “pluripotent stem cell characteristics” refer to characteristics of a cell that distinguish pluripotent stem cells from other cells. Expression or non-expression of certain combinations of molecular markers are examples of characteristics of pluripotent stem cells. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. Cell morphologies associated with pluripotent stem cells are also pluripotent stem cell characteristics.

As used herein, the term “reprogramming” refers to the process of dedifferentiating a non-pluripotent cell into a cell exhibiting pluripotent stem cell characteristics.

As used herein, the term “adherent culture vessel” refers to a culture vessel to which a cell may attach via extracellular matrix molecules and the like, and requires the use of an enzyme (e.g., trypsin, dispase, etc.) for detaching cells from the culture vessel. An “adherent culture vessel” is opposed to a culture vessel to which cell attachment is reduced and does not require the use of an enzyme for removing cells from the culture vessel.

As used herein, the term “non-adherent culture vessel” refers to a culture vessel to which cell attachment is reduced or limited, such as for a period of time. A non-adherent culture vessel may contain a low attachment or ultra-low attachment surface, such as may be accomplished by treating the surface with a substance to prevent cell attachment, such as a hydrogel (e.g. a neutrally charged and/or hydrophilic hydrogel) and/or a surfactant (e.g., pluronic acid). A non-adherent culture vessel may contain rounded or concave wells, and/or microwells (e.g., Aggrewells™). In some embodiments, a non-adherent culture vessel is an Aggrewell™ plate. For non-adherent culture vessels, use of an enzyme to remove cells from the culture vessel may not be required.

As used herein, the term “cell culture” may refer to an in vitro population of cells residing outside of an organism. The cell culture can be established from primary cells isolated from a cell bank or animal, or secondary cells that are derived from one of these sources and immortalized for long-term in vitro cultures.

As used herein, the terms “culture,” “culturing,” “grow,” “growing,” “maintain,” “maintaining,” “expand,” “expanding,” etc., when referring to cell culture itself or the process of culturing, can be used interchangeably to mean that a cell is maintained outside the body (e.g., ex vivo) under conditions suitable for survival. Cultured cells are allowed to survive, and culturing can result in cell growth, differentiation, or division.

As used herein, a composition refers to any mixture of two or more products, substances, or compounds, including cells. It may be a solution, a suspension, liquid, powder, a paste, aqueous, non-aqueous or any combination thereof.

The term “pharmaceutical composition” refers to a composition suitable for pharmaceutical use, such as in a mammalian subject (e.g., a human). A pharmaceutical composition typically comprises an effective amount of an active agent (e.g., cells) and a carrier, excipient, or diluent. The carrier, excipient, or diluent is typically a pharmaceutically acceptable carrier, excipient or diluent, respectively.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, a “subject” is a mammal, such as a human or other animal, and typically is human.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

A “vector,” as used herein, refers to a recombinant plasmid or virus that comprises a nucleic acid to be delivered into a host cell, either in vitro or in vivo.

The term “vector” or “gene transfer vector” is used interchangeably with the terms “construct”, “DNA construct”, “genetic construct”, and “polynucleotide cassette” and refers to a polynucleotide sequence that is used to perform a “carrying” function for another polynucleotide. It is understood by one skilled in the art that vectors may contain synthetic DNA sequences, naturally occurring DNA sequences, or both. For example vectors may be used to allow a polynucleotide to be propagated within a living cell, to allow a polynucleotide to be packaged for delivery into a cell, or to allow a polynucleotide to be integrated into the genomic DNA of a cell. A vector may further comprise additional functional elements, for example it may comprise a transposon.

A “promoter” is a nucleotide sequence that directs the transcription of a structural gene. In some embodiments, a promoter is located in the 5′ non-coding region of a gene, proximal to the transcriptional start site of a structural gene. Sequence elements within promoters that function in the initiation of transcription are often characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993); incorporated by reference in its entirety), cyclic AMP response elements (CREs), serum response elements (SREs; Treisman, Seminars in Cancer Biol. 1:47 (1990)), glucocorticoid response elements (GREs), and binding sites for other transcription factors, such as CRE/ATF (O'Reilly et al, J. Biol. Chem. 267: 19938 (1992)), AP2 (Ye et al., J. Biol. Chem. 269:25728 (1994)), SP1, cAMP response element binding protein (CREB; Loeken, Gene Expr. 3:253 (1993)) and octamer factors (see, in general, Watson et al, eds., Molecular Biology of the Gene, 4th ed. (The Benjamin/Cummings Publishing Company, Inc. 1987)), and Lemaigre and Rousseau, Biochem. J. 303: 1 (1994)). As used herein, a promoter can be constitutively active or inducible. If a promoter is an inducible promoter, then the rate of transcription increases in response to an inducing agent. In contrast, the rate of transcription is not regulated by an inducing agent if the promoter is a constitutive promoter.

An “inverted repeat”, “terminal inverted repeat,” or “inverted terminal repeat” is a nucleotide sequence that has a reverse complementary sequence downstream. An inverted repeat can refer to short sequence repeats flanking the transposase gene in a natural transposon or a cassette cargo in an artificially engineered transposon. This inverted repeat sequence determines the boundaries of the transposon and indicates a region where a self-complementary base pair can be formed (a plurality of regions capable of forming a base pair within a single sequence). The two inverted repeats are generally required for the mobilization of the transposon in the presence of a corresponding transposase. In some embodiments, transposon-based vectors are provided. In some embodiments, the transposon-based vector comprises a first inverted terminal repeat gene sequence and a second inverted terminal repeat gene sequence. In some embodiments, the transposon-based vector comprises a transposon disposed between two inverted repeats.

As used herein, the term “transposon” or “transposable element” refers to a polynucleotide that can be excised from a first polynucleotide, for instance, a vector, and be integrated into a second position in the same polynucleotide, or into a second polynucleotide, for instance, the genomic or extrachromosomal DNA of a cell, by the action of a trans-acting transposase. A transposon comprises a first transposon end and a second transposon end which are polynucleotide sequences recognized by and transposed by a transposase. A transposon usually further comprises a first polynucleotide sequence between the two transposon ends, such that the first polynucleotide sequence is transposed along with the two transposon ends by the action of the transposase.

As used herein, the term “transposase” refers to a polypeptide that catalyzes the excision of a transposon from a donor polynucleotide, for example a vector, and the subsequent integration of the transposon into the genomic or extrachromosomal DNA of a target cell. The transposase binds a transposon end. The transposase may be present as a polypeptide or as a polynucleotide that includes a coding sequence encoding a transposase. The polynucleotide can be RNA, for instance an mRNA encoding the transposase, or DNA, for instance a coding sequence encoding the transposase. When the transposase is present as a coding sequence encoding the transposase, in some aspects of the invention the coding sequence may be present on the same vector that includes the transposon, that is, in cis. In other aspects of the invention, the transposase coding sequence may be present on a second vector, that is, in trans.

### II. Methods of Modulating GBA1 Expression

Provided herein are methods of increasing expression of GBA1 in a pluripotent stem cell. In some embodiments, GBA1 is the wildtype form and/or a functional GBA1 or portion thereof. Also provided herein are methods of increasing expression of the wild-type form of GBA1 in a pluripotent stem cell.

Provided herein are methods of increasing expression of GBA1 in a cell, the methods including: (i) introducing, into a pluripotent stem cell, a deoxyribonucleic acid (DNA) sequence encoding GBA1 operably linked to a promoter, wherein the nucleic acid sequence is positioned between inverted terminal repeats and is capable of integrating into DNA in the cell; and (ii) introducing, into the cell, a transposase or a nucleic acid sequence encoding a transposase, wherein the introducing in (i) and (ii) results in integration of the DNA sequence encoding GBA1 into the genome of the cell. In some embodiments, GBA1 is the wildtype form thereof. In some embodiments, GBA1 is a functional GBA1 or a portion thereof. In some embodiments, GBA1 is the wildtype form thereof. In some embodiments, GBA1 is a functional GBA1.

Provided herein are methods of increasing expression of the wild-type form of GBA1 in a cell, the methods including: (i) introducing, into a pluripotent stem cell, a deoxyribonucleic acid (DNA) sequence encoding the wild-type form of GBA1 operably linked to a promoter, wherein the nucleic acid sequence is positioned between inverted terminal repeats and is capable of integrating into DNA in the cell; and (ii) introducing, into the cell, a transposase or a nucleic acid sequence encoding a transposase, wherein the introducing in (i) and (ii) results in integration of the nucleic acid sequence encoding the wild-type form of GBA1 into the genome of the cell.

In some embodiments, prior to the introducing, the cell has reduced activity of GCase. In some embodiments, the cell endogenously contains a variant of GBA1. In some embodiments, the cell is heterozygous for the GBA1 variant. In some embodiments, the cell endogenously comprises a variant of GBA1 associated with Parkinson's Disease.

In some embodiments, the cell comprises biallelic variants in GBA1 or is homozygous for the GBA1 variant. In some embodiments, the cell comprises biallelic variants in GBA1. In some embodiments, the cell is homozygous for the GBA1 variant. In some embodiments, the cell endogenously contains a variant of GBA1 associated with Gaucher's disease (GD).

Also provided here are methods of increasing expression of GBA1 in a cell, the methods including: (i) introducing, into a pluripotent stem cell, a deoxyribonucleic acid (DNA) sequence encoding GBA1 operably linked to a promoter, wherein the nucleic acid sequence is positioned between inverted terminal repeats and is capable of integrating into DNA in the cell; and (ii) introducing, into the cell, a transposase or a nucleic acid sequence encoding a transposase, wherein the cell comprises a variant of GBA1 associated with Parkinson's Disease, and the introducing in (i) and (ii) results in integration of the DNA sequence encoding GBA into the genome of the cell. In some embodiments, GBA1 is the wildtype form thereof. In some embodiments, GBA1 is a functional form thereof.

Also provided here are methods of increasing expression of the wild-type form of GBA1 in a cell, the methods including: (i) introducing, into a pluripotent stem cell, a deoxyribonucleic acid (DNA) sequence encoding the wild-type form of GBA1 operably linked to a promoter, wherein the nucleic acid sequence is positioned between inverted terminal repeats and is capable of integrating into DNA in the cell; and (ii) introducing, into the cell, a transposase or a nucleic acid sequence encoding a transposase, wherein the cell comprises a variant of GBA1 associated with Parkinson's Disease, and the introducing in (i) and (ii) results in integration of the nucleic acid sequence encoding the wild-type form of GBA1 into the genome of the cell.

Also provided here are methods of differentiating neural cells, the methods including: (a) performing a first incubation comprising culturing the cells produced by any of the methods provided herein in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and (b) performing a second incubation comprising culturing cells of the spheroid in a substrate-coated culture vessel under conditions to neurally differentiate the cells. In some embodiments, the cells comprise a variant of GBA1 associated with Parkinson's Disease.

Also provided here are methods of differentiating neural cells, the methods including: (a) performing a first incubation including culturing a population of pluripotent stem cells that are modified by integration into the genome of the cells of an exogenous deoxyribonucleic acid (DNA) sequence encoding GBA1 operably linked to a promoter, wherein the culturing is in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and (b) performing a second incubation including culturing cells of the spheroid in a substrate-coated culture vessel under conditions to neurally differentiate the cells.

In some embodiments, the cells exhibit reduced activity of GCase. In some embodiments, the cells endogenously comprise a variant of GBA1. In some embodiments, the cells are heterozygous for the GBA1 variant. In some embodiments, the cells endogenously comprise a variant of GBA1 associated with Parkinson's Disease.

In some embodiments, the cells comprise biallelic variants in GBA1 or are homozygous for the GBA1 variant. In some embodiments, the cells comprise biallelic variants in GBA1. In some embodiments, the cells are homozygous for the GBA1 variant. In some embodiments, the cells endogenously contain one or more variant(s) of GBA1 associated with Gaucher's disease (GD).

In some embodiments, the cells are induced pluripotent stem cells.

In some embodiments, the method includes introducing, into the cell, a transposase. In some embodiments, the method includes introducing, into the cell, a a nucleic acid sequence encoding a transposase.

In some embodiments, the pluripotent stem cell exhibits decreased expression of GBA1 prior to being introduced with the DNA sequence encoding GBA1 and the transposase or the nucleic acid sequence encoding a transposase, as compared to a reference cell. In some embodiments, the pluripotent stem cell exhibits reduced activity of the β-Glucocerebrosidase (GCase) enzyme encoded by GBA1 prior to being introduced with the DNA sequence encoding GBA1 and the transposase or the nucleic acid sequence encoding a transposase, as compared to a reference cell. In some embodiments, the reference cell does not exhibit reduced GCase activity. In some embodiments, the reference cell is a cell from a subject without an LBD. In some embodiments, the LBD is PD. In some embodiments, the LBD is Parkinson's disease dementia. In some embodiments, the LBD is DLB. In some embodiments, the reference cell is a cell from a subject without Parkinson's disease (PD). In some embodiments, the reference cell is a cell from a subject without Gaucher's Disease.

The provided methods, in some embodiments, result in increased expression of GBA1 in a cell, increased activity of the GCase enzyme encoded by GBA1 in the cell, or both, by introducing a deoxyribonucleic acid (DNA) sequence encoding GBA1 into into the cell, thereby resulting in overexpression of GBA1 in the cell. In some embodiments, the method results in increased activity of the GCase enzyme.

In any of the provided embodiments, a DNA sequence encoding GBA1 is introduced into a cell by non-targeted integration, such as using a transposon-based system. In any of the provided embodiments, a DNA sequence encoding GBA1 is introduced into a cell by targeted integration, such as using a transposon-based system. Such methods of targeted integration using a transposon-based system are known in the art and include any of those as described in Yant et al., Nucleic Acids Res (2007) 35(7):e50; Demattei et al., Genetica (2010) 138:531-40; Klompe et al., Nature (2019) 571:219-25; Bazaz et al., Scientific Reports (2022) 12:3390; and Bhatt and Chalmers, Nucleic Acids Res (2019) 47(15):8126-35.

In any of the provided embodiments, a DNA sequence encoding GBA1 is introduced into a cell by targeted integration. Promising sites for targeted integration include, but are not limited to, safe harbor loci, or genomic safe harbor (GSH), which are intragenic or extragenic regions of the human genome that, theoretically, are able to accommodate predictable expression of newly integrated DNA without adverse effects on the host cell or organism. A useful safe harbor must permit sufficient transgene expression to yield desired levels of the vector-encoded protein or non-coding RNA. A safe harbor also must not predispose cells to malignant transformation nor alter cellular functions. For an integration site to be a potential safe harbor locus, it ideally needs to meet criteria including, but not limited to: absence of disruption of regulatory elements or genes, as judged by sequence annotation; is an intergenic region in a gene dense area, or a location at the convergence between two genes transcribed in opposite directions; keep distance to minimize the possibility of long-range interactions between vector-encoded transcriptional activators and the promoters of adjacent genes, particularly cancer-related and microRNA genes; and has apparently ubiquitous transcriptional activity, as reflected by broad spatial and temporal expressed sequence tag (EST) expression patterns, indicating ubiquitous transcriptional activity. This latter feature is especially important in stem cells, where during differentiation, chromatin remodeling typically leads to silencing of some loci and potential activation of others. Within the region suitable for exogenous insertion, a precise locus chosen for insertion should be devoid of repetitive elements and conserved sequences and to which primers for amplification of homology arms could easily be designed.

Safe harbor loci include any of those known in the art, including those described in U.S. Pat. No. 11,072,781, which is incorporated by reference herein in its entirety. Suitable sites for human genome editing, or specifically, targeted integration, include, but are not limited to the adeno-associated virus site 1 (AAVS1), the chemokine (CC motif) receptor 5 (CCR5) gene locus, the mitochondrial citramalyl-CoA lyase (CLYBL) gene locus, the proprotein convertase subtilisin/kexin type 9 (PCSK9) gene locus, and the human orthologue of the mouse ROSA26 locus. Additionally, the human orthologue of the mouse H11 locus may also be a suitable site for insertion using the composition and method of targeted integration disclosed herein. Further, collagen and HTRP gene loci may also be used as safe harbor for targeted integration. However, validation of each selected site has been shown to be necessary especially in stem cells for specific integration events, and optimization of insertion strategy including promoter election, exogenous gene sequence and arrangement, and construct design is often needed.

For targeted in/dels, the editing site is often comprised in an endogenous gene whose expression and/or function is intended to be disrupted. In one embodiment, the endogenous gene comprising a targeted in/del is associated with immune response regulation and modulation. In some other embodiments, the endogenous gene comprising a targeted in/del is associated with targeting modality, receptors, signaling molecules, transcription factors, drug target candidates, immune response regulation and modulation, or proteins suppressing engraftment, trafficking, homing, viability, self-renewal, persistence, and/or survival of stem cells and/or progenitor cells, and the derived cells therefrom.

As such, one aspect of the present invention provides a method of targeted integration in a selected locus including genome safe harbor or a preselected locus known or proven to be safe and well-regulated for continuous or temporal gene expression such as the B2M, TAP1, TAP2 or tapasin locus as provided herein. In one embodiment, the genome safe harbor for the method of targeted integration comprises one or more desired integration site comprising AAVS1, CCR5, CLYBL, PCSK9, ROSA26, collagen, HTRP, H11, beta-2 microglobulin, GAPDH, TCR or RUNX1, or other loci meeting the criteria of a genome safe harbor. In one embodiment, the method of targeted integration in a cell comprising introducing a construct comprising one or more exogenous polynucleotides to the cell, and introducing a construct comprising a pair of homologous arms specific to a desired integration site and one or more exogenous sequence, to enable site specific homologous recombination by the cell host enzymatic machinery, wherein the desired integration site comprises AAVS1, CCR5, CLYBL, PCSK9, ROSA26, collagen, HTRP, H11, beta-2 microglobulin, GAPDH, TCR or RUNX1, or other loci meeting the criteria of a genome safe harbor.

In another embodiment, the method of targeted integration in a cell comprises introducing a construct comprising one or more exogenous polynucleotides to the cell, and introducing a ZFN expression cassette comprising a DNA-binding domain specific to a desired integration site to the cell to enable a ZFN-mediated insertion, wherein the desired integration site comprises AAVS1, CCR5, CLYBL, PCSK9, ROSA26, collagen, HTRP, H11, beta-2 microglobulin, GAPDH, TCR or RUNX1, or other loci meeting the criteria of a genome safe harbor. In yet another embodiment, the method of targeted integration in a cell comprises introducing a construct comprising one or more exogenous polynucleotides to the cell, and introducing a TALEN expression cassette comprising a DNA-binding domain specific to a desired integration site to the cell to enable a TALEN-mediated insertion, wherein the desired integration site comprises AAVS1, CCR5, CLYBL, PCSK9, ROSA26, collagen, HTRP, H11, beta-2 microglobulin, GAPDH, TCR or RUNX1, or other loci meeting the criteria of a genome safe harbor. In another embodiment, the method of targeted integration in a cell comprises introducing a construct comprising one or more exogenous polynucleotides to the cell, introducing a Cas (e.g., Cas9) expression cassette, and a gRNA comprising a guide sequence specific to a desired integration site to the cell to enable a Cas (e.g., Cas9)-mediated insertion, wherein the desired integration site comprises AAVS1, CCR5, CLYBL, PCSK9, ROSA26, collagen, HTRP, H11, beta-2 microglobulin, GAPDH, TCR or RUNX1, or other loci meeting the criteria of a genome safe harbor. In still another embodiment, the method of targeted integration in a cell comprises introducing a construct comprising one or more att sites of a pair of DICE recombinases to a desired integration site in the cell, introducing a construct comprising one or more exogenous polynucleotides to the cell, and introducing an expression cassette for DICE recombinases, to enable DICE-mediated targeted integration, wherein the desired integration site comprises AAVS1, CCR5, CLYBL, PCSK9, ROSA26, collagen, HTRP, H11, beta-2 microglobulin, GAPDH, TCR or RUNX1, or other loci meeting the criteria of a genome safe harbor.

In some embodiments, the expression of GBA1 is increased in the cell. In some embodiments, the expression of GBA1 is increased in the cell by about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, or about 1,000%. In some embodiments, the expression of GBA1 is increased in the cell by about 10%. In some embodiments, the expression of GBA1 is increased in the cell by about 20%. In some embodiments, the expression of GBA1 is increased in the cell by about 30%. In some embodiments, the expression of GBA1 is increased in the cell by about 40%. In some embodiments, the expression of GBA1 is increased in the cell by about 50%. In some embodiments, the expression of GBA1 is increased in the cell by about 60%. In some embodiments, the expression of GBA1 is increased in the cell by about 70%. In some embodiments, the expression of GBA1 is increased in the cell by about 80%. In some embodiments, the expression of GBA1 is increased in the cell by about 90%. In some embodiments, the expression of GBA1 is increased in the cell by about 100%. In some embodiments, the expression of GBA1 is increased in the cell by about 200%. In some embodiments, the expression of GBA1 is increased in the cell by about 300%. In some embodiments, the expression of GBA1 is increased in the cell by about 400%. In some embodiments, the expression of GBA1 is increased in the cell by about 500%. In some embodiments, the expression of GBA1 is increased in the cell by about 600%. In some embodiments, the expression of GBA1 is increased in the cell by about 700%. In some embodiments, the expression of GBA1 is increased in the cell by about 800%. In some embodiments, the expression of GBA1 is increased in the cell by about 900%. In some embodiments, the expression of GBA1 is increased in the cell by about 1,000%.

In some embodiments, the activity of GCase is increased in the cell. In some embodiments, the activity of GCase is increased in the cell by about 10%, about 20%, about 30%, about 40%, about 50%, about 60%, about 70%, about 80%, about 90%, about 100%, about 200%, about 300%, about 400%, about 500%, about 600%, about 700%, about 800%, about 900%, or about 1,000%. In some embodiments, the activity of GCase is increased in the cell by about 10%. In some embodiments, the activity of GCase is increased in the cell by about 20%. In some embodiments, the activity of GCase is increased in the cell by about 30%. In some embodiments, the activity of GCase is increased in the cell by about 40%. In some embodiments, the activity of GCase is increased in the cell by about 50%. In some embodiments, the activity of GCase is increased in the cell by about 60%. In some embodiments, the activity of GCase is increased in the cell by about 70%. In some embodiments, the activity of GCase is increased in the cell by about 80%. In some embodiments, the activity of GCase is increased in the cell by about 90%. In some embodiments, the activity of GCase is increased in the cell by about 100%. In some embodiments, the activity of GCase is increased in the cell by about 200%. In some embodiments, the activity of GCase is increased in the cell by about 300%. In some embodiments, the activity of GCase is increased in the cell by about 400%. In some embodiments, the activity of GCase is increased in the cell by about 500%. In some embodiments, the activity of GCase is increased in the cell by about 600%. In some embodiments, the activity of GCase is increased in the cell by about 700%. In some embodiments, the activity of GCase is increased in the cell by about 800%. In some embodiments, the activity of GCase is increased in the cell by about 900%. In some embodiments, the activity of GCase is increased in the cell by about 1,000%.

A. Samples, Cells, and Cell Preparations

In embodiments of the provided methods, cells (e.g., pluripotent stem cells) are introduced with (i) a deoxyribonucleic acid (DNA) sequence encoding GBA1 operably linked to a promoter, wherein the nucleic acid sequence is positioned between inverted terminal repeats and is capable of integrating into DNA in the cell; and (ii) a transposase or a nucleic acid sequence encoding a transposase. In some embodiments, cells (e.g., pluripotent stem cells) are introduced with (i) a deoxyribonucleic acid (DNA) sequence encoding GBA1 operably linked to a promoter, wherein the nucleic acid sequence is positioned between inverted terminal repeats and is capable of integrating into DNA in the cell; and (ii) a transposase. In some embodiments, cells (e.g., pluripotent stem cells) are introduced with (i) a deoxyribonucleic acid (DNA) sequence encoding GBA1 operably linked to a promoter, wherein the nucleic acid sequence is positioned between inverted terminal repeats and is capable of integrating into DNA in the cell; and (ii) a nucleic acid sequence encoding a transposase.

In some embodiments, prior to the introducing, the cell exhibits reduced activity of GCase. In some embodiments, the cell endogenously contains a variant of GBA1. In some embodiments, the cell is heterozygous for the GBA1 variant. In some embodiments, the cell endogenously comprises a variant of GBA1 associated with Parkinson's disease.

In some embodiments, the cell comprises biallelic variants in GBA1 or is homozygous for the GBA1 variant. In some embodiments, the cell comprises biallelic variants in GBA1. In some embodiments, the cell is homozygous for the GBA1 variant. In some embodiments, the cell endogenously contains a variant of GBA1 associated with Gaucher's disease (GD).

In some embodiments, the cell is a pluripotent stem cell. Various sources of pluripotent stem cells can be used in the method, including embryonic stem (ES) cells and induced pluripotent stem cells (iPSCs). In some embodiments, the cell is an iPSC. In some embodiments, the pluripotent stem cell is an iPSC. In some embodiments, the pluripotent stem cell is an iPSC, artificially derived from a non-pluripotent cell. In some aspects, a non-pluripotent cell is a cell of lesser potency to self-renew and differentiate than a pluripotent stem cell. iPSCs may be generated by a process known as reprogramming, wherein non-pluripotent cells are effectively “dedifferentiated” to an embryonic stem cell-like state by engineering them to express genes such as OCT4, SOX2, and KLF4. Takahashi and Yamanaka, Cell (2006) 126: 663-76.

In some embodiments, the cell is a pluripotent stem cell. In some embodiments, the cell is a pluripotent stem cell that was artificially derived from a non-pluripotent cell of a subject. In some embodiments, the non-pluripotent cell is a fibroblast. In some embodiments, the fibroblast exhibits reduced GCase activity. In some embodiments, the subject is a human. In some embodiments, the subject is a human with an LBD. In some embodiments, the LBD is PD. In some embodiments, the LBD is Parkinson's disease dementia. In some embodiments, the LBD is DLB. In some embodiments, the subject is a human with Parkinson's disease (PD). In some embodiments, the subject is a human with Gaucher's disease. In some embodiments, the pluripotent stem cell is an iPSC.

In some aspects, pluripotency refers to cells with the ability to give rise to progeny that can undergo differentiation, under appropriate conditions, into cell types that collectively exhibit characteristics associated with cell lineages from the three germ layers (endoderm, mesoderm, and ectoderm). Pluripotent stem cells can contribute to tissues of a prenatal, postnatal or adult organism. A standard art-accepted test, such as the ability to form a teratoma in 8-12 week old SCID mice, can be used to establish the pluripotency of a cell population. However, identification of various pluripotent stem cell characteristics can also be used to identify pluripotent cells. In some aspects, pluripotent stem cells can be distinguished from other cells by particular characteristics, including by expression or non-expression of certain combinations of molecular markers. More specifically, human pluripotent stem cells may express at least some, and optionally all, of the markers from the following non-limiting list: SSEA-3, SSEA-4, TRA-1-60, TRA-1-81, TRA-2-49/6E, ALP, Sox2, E-cadherin, UTF-1, Oct4, Lin28, Rex1, and Nanog. In some aspects, a pluripotent stem cell characteristic is a cell morphology associated with pluripotent stem cells.

Methods for generating iPSCs are known. For example, mouse iPSCs were reported in 2006 (Takahashi and Yamanaka), and human iPSCs were reported in late 2007 (Takahashi et al. and Yu et al.). Mouse iPSCs demonstrate important characteristics of pluripotent stem cells, including the expression of stem cell markers, the formation of tumors containing cells from all three germ layers, and the ability to contribute to many different tissues when injected into mouse embryos at a very early stage in development. Human iPSCs also express stem cell markers and are capable of generating cells characteristic of all three germ layers.

In some embodiments, the PSCs (e.g., iPSCs) are from a subject having reduced activity of GCase and/or a variant in GBA1, wherein reduced activity of GCase and/or the variant in GBA1 is associated with PD. In some embodiments, the PSCs have reduced activity of GCase (e.g., as compared to cells from a subject not having Parkinson's disease). In some embodiments, the PSCs have a variant in GBA1. In some embodiments, the PSCs have reduced activity of GCase and a variant in GBA1.

In some embodiments, the subject is homozygous for a GBA1 variant or has biallelic GBA1 variants. In some embodiments, the subject is homozygous for a GBA1 variant. In some embodiments, the subject has biallelic GBA1 variants. In some embodiments, the PSCs (e.g., iPSCs) are from a subject having one or more variant(s) in GBA1 that is associated with GD. The gene variant in GBA1 that is associated with GD is not limited and can be any gene variant, e.g., SNP, in GBA1 that is associated with GD.

In some embodiments, the PSCs (e.g., iPSCs) are from a subject having a gene variant, e.g., SNP, in GBA1 that is associated with PD. The gene variant in GBA1 that is associated with PD is not limited and can be any gene variant, e.g., SNP, in GBA1 that is associated with PD, e.g., is associated with an increased risk of developing PD. The gene variant in GBA1 that is associated with PD can be any gene variant, e.g., SNP, in GBA1 that is associated with reduced activity of GCase. In some embodiments, the gene variant is a mutation in the GBA1 gene that results in an N370S amino acid change due to the presence of a serine, rather than an asparagine, at amino acid position 370 in the expressed GCase enzyme; or is a mutation in the GBA1 gene that results in an L444P amino acid change due to the presence of a proline, rather than a leucine, at position 444 in the expressed GCase enzyme; or is a mutation that results in an E326K amino acid change due to the presence of a lysine, rather than a glutamic acid, at position 326 in the expressed GCase enzyme (e.g., with reference to SEQ ID NO:1). In some embodiments, the gene variant is a SNP in the GBA1 gene selected from the group consisting of rs76763715, rs421016, and rs2230288 (e.g., with reference to SEQ ID NO:2).

In some embodiments, the PSCs (e.g., iPSCs) are autologous to the subject to be treated, i.e. the PSCs are derived from the same subject to whom the differentiated cells that were previously engineered to stably express one or more GBA1-containing transgene(s) are administered.

In some embodiments, non-pluripotent cells (e.g., fibroblasts) having reduced GCase activity are reprogrammed to become iPSCs before integration of one or more GBA1-containing transgene(s) and/or differentiation into neural and/or neuronal cells. In some embodiments, non-pluripotent cells (e.g., fibroblasts) derived from patients having a Lewy body disease (LBD) are reprogrammed to become iPSCs before integration of one or more GBA1-containing transgene(s) and/or differentiation into neural and/or neuronal cells. In some embodiments, the LBD is Parkinson's disease (PD). In some embodiments, the LBD is Parkinson's disease dementia. In some embodiments, the LBD is dementia with Lewy bodies (DLB). In some embodiments, non-pluripotent cells (e.g., fibroblasts) derived from patients having Parkinson's disease (PD) are reprogrammed to become iPSCs before integration of one or more GBA1-containing transgene(s) and/or differentiation into neural and/or neuronal cells. In some embodiments, non-pluripotent cells (e.g., fibroblasts) derived from patients having Gaucher's disease (PD) are reprogrammed to become iPSCs before integration of one or more GBA1-containing transgene(s) and/or differentiation into neural and/or neuronal cells. In some embodiments, fibroblasts may be reprogrammed to iPSCs by transforming fibroblasts with genes (OCT4, SOX2, NANOG, LIN28, and KLF4) cloned into a plasmid (for example, see, Yu, et al., Science DOI: 10.1126/science.1172482). In some embodiments, non-pluripotent fibroblasts derived from patients having PD are reprogrammed to become iPSCs before integration of one or more GBA1-containing transgene(s) and/or differentiation into determined DA neuron progenitors cells and/or DA neurons, such as by use of the non-integrating Sendai virus to reprogram the cells (e.g., use of CTS™ CytoTune™-iPS 2.1 Sendai Reprogramming Kit). In some embodiments, the resulting overexpressing and differentiated cells are then administered to the patient from whom they are derived in an autologous stem cell transplant. In some embodiments, the PSCs (e.g., iPSCs) are allogeneic to the subject to be treated, i.e., the PSCs are derived from a different individual than the subject to whom the overexpressing and differentiated cells will be administered. In some embodiments, non-pluripotent cells (e.g., fibroblasts) derived from another individual (e.g., an individual not having a neurodegenerative disorder, such as Parkinson's disease) are reprogrammed to become iPSCs before integration one or more GBA1-containing transgene(s) and/or differentiation into determined DA neuron progenitor cells and/or DA neurons. In some embodiments, reprogramming is accomplished, at least in part, by use of the non-integrating Sendai virus to reprogram the cells (e.g., use of CTS™ CytoTune™-iPS 2.1 Sendai Reprogramming Kit). In some embodiments, the resulting overexpressing and differentiated cells are then administered to an individual who is not the same individual from whom the overexpressing and differentiated cells are derived (e.g., allogeneic cell therapy or allogeneic cell transplantation).

In any of the provided embodiments, the PSCs described herein (e.g., allogeneic cells) may be genetically engineered to be hypoimmunogenic. In some embodiments, methods for reducing the immunogenicity generally include ablating expression of HLA molecules and/or introducing immunomodulatory factors into a safe harbor locus. Newly integrated genes may affect the surrounding endogenous genes and chromatin, potentially altering cell behavior or favoring cellular transformation. Thus, inserting exogenous DNA (e.g., encoding immunomodulatory factors) in a pre-selected locus such as a safe harbor locus, or genomic safe harbor (GSH) is important for safety, efficiency, copy number control, and for reliable gene response control. Safe harbor loci include any of those known in the art, including those described in U.S. Pat. No. 11,072,781, which is incorporated by reference herein in its entirety. In some embodiments, the safe harbor locus may be AAVS1, CCR5, CLYBL, ROSA26, collagen, HTRP, H11, beta-2 microglobulin, GAPDH, PCSK9, TCR or RUNX1. Particular methods for reducing the immunogenicity are known, and include ablating polymorphic HLA-A/-B/-C and HLA class II molecule expression and introducing the immunomodulatory factors PD-L1, HLA-G, and CD47 into the AAVS1 safe harbor locus in differentiated cells. Han et al., PNAS (2019) 116(21):10441-46. Thus, in some embodiments, the PSCs described herein are engineered to delete highly polymorphic HLA-A/-B/-C genes and to introduce immunomodulatory factors, such as PD-L1, HLA-G, and/or CD47, into the AAVS1 safe harbor locus.

In some embodiments, following the introducing of (i) the DNA sequence encoding GBA1 and (ii) the transposase or the nucleic acid sequence encoding a transposase into the cells, the cells (e.g., PSCs, such as iPSCs) are cultured in the absence of feeder cells, until they reach 80-90% confluency, at which point they are harvested and further cultured for differentiation (day 0). In one aspect of the method described herein, once iPSCs reach 80-90% confluence, they are washed in phosphate buffered saline (PBS) and subjected to enzymatic dissociation, such as with Accutase™, until the cells are easily dislodged from the surface of a culture vessel. The dissociated iPSCs are then re-suspended in media for downstream differentiation into the desired cell type(s), such as determined DA neuron progenitor cells and/or DA neurons. Section III, below, provides exemplary methods for differentiation of PSCs, e.g., iPSCs, that have been engineered to contain one or more GBA1-containing transgene(s) by the provided methods.

In some embodiments, following the introducing of (i) the DNA sequence encoding the GBA1 and (ii) the transposase or the nucleic acid sequence encoding a transposase into the cells (e.g., PSCs, such as iPSCs), the PSCs are resuspended in a basal induction media. In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, β-mercaptoethanol, and insulin. In some embodiments, the basal induction media is further supplemented with serum replacement, a Rho-associated protein kinase (ROCK) inhibitor, and various small molecules, for differentiation. In some embodiments, the PSCs are resuspended in the same media they will be cultured in for at least a portion of the first incubation.

In some embodiments, GBA1 is the wild-type form of GBA1. In some embodiments, the wild-type form of GBA1 is encoded by the sequence set forth in SEQ ID NO:2. In some embodiments, the wild-type form of GBA1 is encoded by the sequence set forth in SEQ ID NO:2 or a sequence having at least 80%, at least 85%, at least 90%, at least 95%, or at least 99% sequence identity to the sequence set forth in SEQ ID NO:2. In some embodiments, the wild-type form of GBA1 encodes an amino acid sequence comprising the amino acid sequence set forth in SEQ ID NO: 1. In some embodiments, GBA1 is a functional GBA1 or a portion thereof. In some embodiments, a functional GBA1 is capable of being transcribed into GBA1 mRNA or a portion thereof. In some embodiments, a functional GBA1 is capable of being transcribed into GBA1 mRNA or a portion thereof, which is capable of being translated into a functional GCase enzyme or a portion thereof. In some embodiments, a functional GBA1 is capable of (i) being transcribed into GBA1 mRNA or a portion thereof; and (ii) being transcribed into GBA1 mRNA or a portion thereof, which is capable of being translated into a functional GCase enzyme or a portion thereof. In some embodiments, a functional GCase enzyme or a portion thereof has the enzymatic activity of a wild-type GCase enzyme. In some embodiments, the enzymatic activity of GCase is determined by any of the methods described in Section II.D.

B. Transposon-Based Modulation of GBA1 Expression

The provided methods involve, in some embodiments, introducing into a pluripotent stem cell (i) a deoxyribonucleic acid (DNA) sequence encoding GBA1 operably linked to a promoter; and (ii) a transposase or a nucleic acid sequence encoding a transposase, such as any cell as described in Section II.A.

1. Transposon Systems

Provided herein are transposon-based systems for increasing expression of GBA1 in a cell, the systems including: (i) a deoxyribonucleic acid (DNA) sequence encoding GBA1, wherein the DNA sequence is positioned between at least two inverted terminal repeats and is capable of integrating into DNA in a cell; and (ii) a transposase or a nucleic acid sequence encoding a transposase).

Also provided herein are methods of increasing expression of GBA1 in a cell, the methods including: (i) introducing, into a pluripotent stem cell, a deoxyribonucleic acid (DNA) sequence encoding GBA1 operably linked to a promoter, wherein the nucleic acid sequence is positioned between inverted terminal repeats and is capable of integrating into DNA in the cell; and (ii) introducing, into the cell, a transposase or a nucleic acid sequence encoding a transposase, wherein the introducing in (i) and (ii) results in integration of the DNA sequence encoding GBA1 into the genome of the cell.

Thus, in some aspects, the disclosure relates transposon-based systems including a transposable element comprising a transgene that encodes GBA1 and a transposase or a nucleic acid sequence encoding the same.

Transposable genetic elements, also called transposons, are segments of DNA that can be mobilized from one genomic location to another within a single cell. Transposons can be divided into two major groups according to their mechanism of transposition: transposition can occur (1) via reverse transcription of an RNA intermediate for elements termed retrotransposons, and (2) via direct transposition of DNA flanked by terminal inverted repeats (TIRs) for DNA transposons. Active transposons encode one or more proteins that are required for transposition. The natural active DNA transposons harbor a transposase enzyme gene.

DNA transposons (e.g., Class II transposons) can translocate via a non-replicative, ‘cut-and-paste’ mechanism. This requires recognition of the two terminal inverted repeats by a catalytic enzyme, i.e., transposase, which can cleave its target and consequently release the DNA transposon from its donor template. Upon excision, the DNA transposons may subsequently integrate into the acceptor DNA that is cleaved by the same transposase. In some of their natural configurations, DNA transposons are flanked by two inverted repeats and may contain a gene encoding a transposase that catalyzes transposition.

The provided methods including introducing a DNA sequence encoding GBA1 into a cell. In some embodiments, the DNA sequence encoding GBA1 is part of a plasmid

In some embodiments, the methods include introducing a transposase into the cell.

In some embodiments, the methods include introducing a nucleic acid sequence encoding a transposase into the cell. In some embodiments, the nucleic acid sequence encoding a transposase is part of a plasmid. In some embodiments, the nucleic acid sequence encoding a transposase is RNA. In some embodiments, the nucleic acid sequence encoding a transposase is DNA.

In some embodiments, the plasmid containing the DNA sequence encoding GBA1 and the plasmid containing the nucleic acid sequence encoding the transposase are the same plasmid.

In some embodiments, it is desirable to design a transposon to develop a binary system based on two distinct plasmids whereby the transposase is physically separated from the transposon DNA containing the gene of interest flanked by the inverted repeats (i.e., the transposon DNA containing the gene of interest is positioned between the inverted repeats). Co-delivery of the transposon and transposase plasmids into the target cells enables transposition via a conventional cut-and-paste mechanism. Thus, in some embodiments, the plasmid containing the DNA sequence encoding GBA1 and the plasmid containing the nucleic acid sequence encoding the transposase are different plasmids

DNA transposons in the hAT family are widespread in plants and animals. A number of active hAT transposon systems have been identified and found to be functional, including but not limited to, the Hermes transposon, Ac transposon, hobo transposon, and the Tol2 transposon. The hAT family is composed of two families that have been classified as the AC subfamily and the Buster subfamily, based on the primary sequence of their transposases. Members of the hAT family belong to Class II transposable elements. Class II mobile elements use a cut and paste mechanism of transposition. hAT elements share similar transposases, short terminal inverted repeats, and an eight base-pairs duplication of genomic target.

TcBuster™ is a member of the hAT family of DNA transposons. Arensburger et al., Genetics (2011) 188(1):45-57. Other members of the family include Sleeping Beauty™ and PiggBac®. Ivics et al., Cell (1997) 91(4):P501-10; Miskey et al., Nucleic Acids Res (2003) 31:6873-811; Ding et al., Cell (2005) 122(3):473-83; Wilson et al., Mol Ther (2007) 12:139-45; Kawakami et al., Genome Biol (2007) 9(Suppl. 1):S7. Discussed herein are various systems and methods relating to approaches to increase expression of the wildtype form of a GBA1 in a cell (e.g., a pluripotent stem cell, such as an iPSC) using hAT family transposon components. In some embodiments, increased expression of the wildtype form of the target gene is achieved by stable integration of the DNA sequence encoding the wildtype form of GBA1 into the genome of the cell. The present disclosure relates to transposon-based delivery of GBA1, including into cells having a variant form of GBA1 associated with Parkinson's Disease.

In some embodiments, the transposase is a Class II transposase. In some embodiments, the transposase is selected from the group consisting of: Sleeping Beauty™, PiggyBac®, TcBuster™, Frog Prince, Tol2, Tcl/mariner, or a derivative thereof having transposase activity. In some embodiments, the transposase is Sleeping Beauty™, PiggyBac®, or TcBuster™. In some embodiments, the transposase is Sleeping Beauty™. In some embodiments, the DNA sequence encoding the wild-type form of GBA1 is part of a Sleeping Beauty™ transposon. In some embodiments, the transposase is Sleeping Beauty™, and the DNA sequence encoding GBA1 is part of a Sleeping Beauty™ transposon. In some embodiments, the transposase is PiggyBa®. In some embodiments, the DNA sequence encoding GBA1 is part of a PiggyBac® transposon. In some embodiments, the transposase is PiggyBac®, and the DNA sequence encoding GBA1 is part of a PiggyBac® transposon. In some embodiments, the transposase is TcBuster™ In some embodiments, the DNA sequence encoding GBA1 is part of a TcBuster™ transposon. In some embodiments, the transposase is TcBuster™, and the DNA sequence encoding GBA1 is part of a TcBuster™ transposon. In some embodiments, the transposon and/or tranposase is any of those as described in WO2018112415, WO2019246486, US20200323902.

2. GBA1-Containing Transgene

Provided herein are deoxyribonucleic acid (DNA) sequences encoding GBA1 operably linked to a promoter. In some embodiments, the DNA sequence is capable of integrating into DNA in the cell (e.g., the pluripotent stem cell). In some embodiments, the DNA sequence encoding GBA1 is part of a plasmid.

In some embodiments, GBA1 is the wild-type form of GBA1. In some embodiments, the wild-type form of GBA1 is encoded by the sequence set forth in SEQ ID NO:2. In some embodiments, the wild-type form of GBA1 is encoded by the sequence set forth in SEQ ID NO:2 or a sequence having at least 80%, at least 85%, at least 90%, at least 95%, or at least 99% sequence identity to the sequence set forth in SEQ ID NO:2. In some embodiments, the wild-type form of GBA1 encodes an amino acid sequence comprising the amino acid sequence set forth in SEQ ID NO: 1. In some embodiments, GBA1 is a functional GBA1 or a portion thereof. In some embodiments, a functional GBA1 is capable of being transcribed into GBA1 mRNA or a portion thereof. In some embodiments, a functional GBA1 is capable of being transcribed into GBA1 mRNA or a portion thereof, which is capable of being translated into a functional GCase enzyme or a portion thereof. In some embodiments, a functional GBA1 is capable of (i) being transcribed into GBA1 mRNA or a portion thereof; and (ii) being transcribed into GBA1 mRNA or a portion thereof, which is capable of being translated into a functional GCase enzyme or a portion thereof. In some embodiments, a functional GCase enzyme or a portion thereof has the enzymatic activity of a wild-type GCase enzyme. In some embodiments, the enzymatic activity of GCase is determined by any of the methods described in Section II.D.

In some embodiments, the DNA sequence encoding GBA1 encodes the wild-type form of human GBA1. In some embodiments, the DNA sequence encoding GBA1 encodes an amino acid comprising the amino acid sequence set forth in SEQ ID NO:1 (i.e., GCase).

In some embodiments, the DNA sequence encoding GBA1 is codon optimized. In some embodiments, the DNA sequence encoding GBA1 is modified by optimization of the codons for expression in humans. Codon optimization generally involves balancing the percentages of codons selected with the abundance, e.g., published abundance, of human transfer RNAs, for example, so that none is overloaded or limiting. In some cases, such balancing is necessary or useful because most amino acids are encoded by more than one codon, and codon usage generally varies from organism to organism. Differences in codon usage between transfected or transduced genes or nucleic acids and host cells can have effects on protein expression from the nucleic acid molecule. Table 1 below sets forth an exemplary human codon usage frequency table. In some embodiments, to generate codon-optimized nucleic acid sequences, codons are chosen to select for those codons that are in balance with human usage frequency. The redundancy of the codons for amino acids is such that different codons code for one amino acid, such as depicted in Table 1. In selecting a codon for replacement, it is desired that the resulting mutation is a silent mutation such that the codon change does not affect the amino acid sequence. Generally, the last nucleotide of the codon (e.g., at the third position) can remain unchanged without affecting the amino acid sequence.

TABLE 1
Human Codon Usage Frequency

| Hu- | ami- | | | Hu- | ami- | | |
|---|---|---|---|---|---|---|---|
| man | no | freq./ | | man | no | freq./ | |
| codon | acid | 1000 | number | codon | acid | 1000 | number |
| TTT | F | 17.6 | 714298 | TCT | S | 15.2 | 618711 |
| TTC | F | 20.3 | 824692 | TCC | S | 17.7 | 718892 |
| TTA | L | 7.7 | 311881 | TCA | S | 12.2 | 496448 |
| TTG | L | 12.9 | 525688 | TCG | S | 4.4 | 179419 |
| CTT | L | 13.2 | 536515 | CCT | P | 17.5 | 713233 |
| CTC | L | 19.6 | 796638 | CCC | P | 19.8 | 804620 |
| CTA | L | 7.2 | 290751 | CCA | P | 16.9 | 688038 |
| CTG | L | 39.6 | 1611801 | CCG | P | 6.9 | 281570 |
| ATT | I | 16 | 650473 | ACT | T | 13.1 | 533609 |
| ATC | I | 20.8 | 846466 | ACC | T | 18.9 | 768147 |
| ATA | I | 7.5 | 304565 | ACA | T | 15.1 | 614523 |
| ATG | M | 22 | 896005 | ACG | T | 6.1 | 246105 |
| GTT | V | 11 | 448607 | GCT | A | 18.4 | 750096 |
| GTC | V | 14.5 | 588138 | GCC | A | 27.7 | 1127679 |
| GTA | V | 7.1 | 287712 | GCA | A | 15.8 | 643471 |
| GTG | V | 28.1 | 1143534 | GCG | A | 7.4 | 299495 |
| TAT | Y | 12.2 | 495699 | TGT | C | 10.6 | 430311 |
| TAC | Y | 15.3 | 622407 | TGC | C | 12.6 | 513028 |
| TAA | * | 1 | 40285 | TGA | * | 1.6 | 63237 |
| TAG | * | 0.8 | 32109 | TGG | W | 13.2 | 535595 |
| CAT | H | 10.9 | 441711 | CGT | R | 4.5 | 184609 |
| CAC | H | 15.1 | 613713 | CGC | R | 10.4 | 423516 |
| CAA | Q | 12.3 | 501911 | CGA | R | 6.2 | 250760 |
| CAG | Q | 34.2 | 1391973 | CGG | R | 11.4 | 464485 |
| AAT | N | 17 | 689701 | AGT | S | 12.1 | 493429 |
| AAC | N | 19.1 | 776603 | AGC | S | 19.5 | 791383 |
| AAA | K | 24.4 | 993621 | AGA | R | 12.2 | 494682 |
| AAG | K | 31.9 | 1295568 | AGG | R | 12 | 486463 |
| GAT | D | 21.8 | 885429 | GGT | G | 10.8 | 437126 |
| GAC | D | 25.1 | 1020595 | GGC | G | 22.2 | 903565 |
| GAA | E | 29 | 1177632 | GGA | G | 16.5 | 669873 |
| GAG | E | 39.6 | 1609975 | GGG | G | 16.5 | 669768 |

For example, the codons TCT, TCC, TCA, TCG, AGT and AGC all code for Serine (note that T in the DNA equivalent to the U in RNA). From a human codon usage frequency, such as set forth in Table 1 above, the corresponding usage frequencies for these codons are 15.2, 17.7, 12.2, 4.4, 12.1, and 19.5, respectively. Since TCG corresponds to 4.4%, if this codon were commonly used in a gene synthesis, the tRNA for this codon would be limiting. In codon optimization, the goal is to balance the usage of each codon with the normal frequency of usage in the species of animal in which the transgene is intended to be expressed.

In some embodiments, the DNA sequence encoding GBA1 (i.e., GCase) comprises a codon-optimized version of the sequence set forth in SEQ ID NO:2. In some embodiments, the DNA sequence encoding GBA1 (i.e., GCase) comprises a codon-optimized version of a coding region of the sequence set forth in SEQ ID NO:2.

In some embodiments, the DNA sequence is positioned between inverted terminal repeats (ITRs). In some embodiments, a nucleic acid sequence encoding an amino acid comprising the amino acid sequence set forth in SEQ ID NO:1 is positioned between ITRs. In some embodiments, the nucleic acid sequence set forth in SEQ ID NO:2 is positioned between ITRs. In some embodiments, the nucleic acid sequence set forth in SEQ ID NO:2 or a codon-optimized version of the nucleic acid sequence set forth in SEQ ID NO:2 is positioned between ITRs. In some embodiments, a nucleic acid sequence comprising a coding region of the nucleic acid sequence set forth in SEQ ID NO:2 is positioned between ITRs. In some embodiments, a codon-optimized version of the nucleic acid sequence set forth in SEQ ID NO:2 is positioned between ITRs. In some embodiments, a nucleic acid sequence comprising a coding region of the nucleic acid sequence set forth in SEQ ID NO:2 or a codon-optimized version thereof is positioned between ITRs. In some embodiments, a codon-optimized version of a nucleic acid sequence comprising a coding region of the nucleic acid sequence set forth in SEQ ID NO:2 is positioned between ITRs.

In some embodiments, the DNA sequence encoding GBA1 is operably linked to a promoter (i.e., the DNA sequence is under the control of the promoter). In some embodiments, the promoter is selected from the group consisting of: ubiquitin C (UBC promoter) cytomegalovirus (CMV) promoter, phosphoglycerate kinase (PGK) promoter, CMV early enhancer/chicken beta actin (CAG) promoter, glial fibrilary acidic protein (GFAP) promoter, synapsin-1 promoter, and Neuron Specific Enolase (NSE) promoter. In some embodiments, the promoter is PGK or UBC. In some embodiments, the promoter is PGK. In some embodiments, the promoter is UBC.

C. Delivery of Transposon and Transposase

Provided herein are methods including introducing into a cell (e.g., a pluripotent stem cell) (i) a deoxyribonucleic acid (DNA) sequence encoding GBA1 operably linked to a promoter; and (ii) a transposase or a nucleic acid sequence encoding a transposase.

In some embodiments, the nucleic acid sequence encoding the transposase and/or the DNA sequence encoding GBA1 are introduced into the cell by electrotransfer, optionally electroporation or nucleofection; chemotransfer; or nanoparticles. In some embodiments, the nucleic acid sequence encoding the transposase is introduced into the cell by electrotransfer, optionally electroporation or nucleofection; chemotransfer; or nanoparticles. In some embodiments, the DNA sequence encoding GBA1 is introduced into the cell by electrotransfer, optionally electroporation or nucleofection; chemotransfer; or nanoparticles. In some embodiments, the nucleic acid sequence encoding the transposase and the DNA sequence encoding GBA1 are introduced into the cell by electrotransfer, optionally electroporation or nucleofection; chemotransfer; or nanoparticles. In some embodiments, the nucleic acid sequence encoding the transposase and/or the DNA sequence encoding GBA1 are introduced into the cell by electroporation or nucleofection. In some embodiments, the nucleic acid sequence encoding the transposase is introduced into the cell by electroporation or nucleofection. In some embodiments, the DNA sequence encoding GBA1 are introduced into the cell by electroporation or nucleofection. In some embodiments, the nucleic acid sequence encoding the transposase and the DNA sequence encoding GBA1 are introduced into the cell by electroporation or nucleofection.

In some embodiments, (i) the DNA sequence encoding GBA1 and the (ii) the transposase or the nucleic acid sequence encoding the transposase are introduced into the cell at the same time. In some embodiments, (i) the DNA sequence encoding of GBA1 and the (ii) the transposase are introduced into the cell at the same time. (i) the DNA sequence encoding GBA1 and the (ii) the nucleic acid sequence encoding the transposase are introduced into the cell at the same time.

D. Selection of Cells Having Stably Integrated GBA1-Containing Transgene(s)

In some embodiments, the cells (e.g. pluripotent stem cells) introduced with (i) a DNA sequence encoding GBA1 (i.e., a GBA1-containing transgene) and (ii) a transposase or a nucleic acid sequence encoding a transposase, in accordance with the methods herein, e.g., as described in Section II.B, are screened and/or selected for cells where the GBA1 expression is increased as compared to prior to the introducing. In some embodiments, the cells introduced with with (i) a DNA sequence encoding GBA1 (i.e., a GBA1-containing transgene) and (ii) a transposase or a nucleic acid sequence encoding a transposase in accordance with the methods herein, e.g., as described in Section II.B, are screened and/or selected for cells where the GCase activity is increased as compared to prior to the introducing. In some embodiments, the cells are assessed to identify changes attributable to the methods described herein, e.g, as described in Section II.B, such as stable integration of the DNA sequence encoding GBA1 into the genome of the cells.

In some embodiments, the assessment includes determining the expression of GBA1 in the cells introduced by stable integration of the DNA sequence encoding GBA1, such as by any methods known in the art. In particular embodiments, assessing, measuring, and/or determining gene expression is or includes determining or measuring the level, amount, or concentration of the gene product. In certain embodiments, the level, amount, or concentration of the gene product may be transformed (e.g., normalized) or directly analyzed (e.g., raw). In some embodiments, the gene product is a protein that is encoded by the gene. In certain embodiments, the gene product is a polynucleotide, e.g., an mRNA or a protein, that is encoded by the gene.

In particular embodiments, the amount or level of a polynucleotide in a sample may be assessed, measured, determined, and/or quantified by any suitable means known in the art. For example, in some embodiments, the amount or level of a polynucleotide gene product can be assessed, measured, determined, and/or quantified by polymerase chain reaction (PCR), including reverse transcriptase (rt) PCR, droplet digital PCR, real-time and quantitative PCR (qPCR) methods; northern blotting; Southern blotting, e.g., of reverse transcription products and derivatives; array based methods, including blotted arrays, microarrays, or in situ-synthesized arrays; and sequencing, e.g., sequencing by synthesis, pyrosequencing, dideoxy sequencing, or sequencing by ligation, or any other methods known in the art.

In some embodiments, the assessment includes determining the protein level of the GCase enzyme, such as by any methods known in the art. Qualifying the level of the Gcase enzyme protein may be carried out by any suitable means known in the art. Suitable methods for assessing, measuring, determining, and/or quantifying the level, amount, or concentration or more or more protein gene products include, but are not limited to detection with immunoassays, nucleic acid-based or protein-based aptamer techniques, HPLC (high precision liquid chromatography), peptide sequencing (such as Edman degradation sequencing or mass spectrometry (such as MS/MS), optionally coupled to HPLC), and microarray adaptations of any of the foregoing (including nucleic acid, antibody or protein-protein (i.e., non-antibody) arrays). In some embodiments, the immunoassay is or includes methods or assays that detect proteins based on an immunological reaction, e.g., by detecting the binding of an antibody or antigen binding antibody fragment to a gene product. Immunoassays include, but are not limited to, quantitative immunocytochemisty or immunohistochemisty, ELISA (including direct, indirect, sandwich, competitive, multiple and portable ELISAs (see, e.g., U.S. Pat. No. 7,510,687), western blotting (including one, two or higher dimensional blotting or other chromatographic means, optionally including peptide sequencing), enzyme immunoassay (EIA), RIA (radioimmunoassay), and SPR (surface plasmon resonance).

In some embodiments, the assessment includes determining the activity level of the GCase enzyme, such as by any methods known in the art. For example, in some embodiments, the activity level of the GCase enzyme is assessed by an enzymatic activity reaction wherein protein isolated from cells is combined with 4-methylumbelliferyl beta-D-glucopyranosidase (4-MBDG) substrate, and cleavage of the substrate by GCase yields 4-methylumbelliferone (4-MU), the concentration of which may be measured, such as by reference to standard or known value(s).

In some embodiments, it is desirable to increase the GCase activity in one or more cells (e.g., a clone) into which a DNA sequence encoding GBA1 is introduced by between about 100% and about 200%, as compared to the GCase activity in the one or more cells prior to introduction of the DNA sequence encoding GBA1. Thus, in some embodiments, one or more cells (e.g., a clone) is selected in which the introduction of a DNA sequence encoding GBA1 has increased the GCase activity by between about 100% and about 200%. In some embodiments, one or more cells (e.g., a clone) is selected in which the introduction of GBA1 has increased its GCase activity by about 100%. In some embodiments, one or more cells (e.g., a clone) is selected in which the introduction of GBA1 has increased its GCase activity by about 120%. In some embodiments, one or more cells (e.g., a clone) is selected in which the introduction of GBA1 has increased its GCase activity by about 140%. In some embodiments, one or more cells (e.g., a clone) is selected in which the introduction of GBA1 has increased its GCase activity by about 160%. In some embodiments, one or more cells (e.g., a clone) is selected in which the introduction of GBA1 has increased its GCase activity by about 180%. In some embodiments, one or more cells (e.g., a clone) is selected in which the introduction of GBA1 has increased its GCase activity by about 200%. In some embodiments, the one or more cells (e.g., a clone) is selected for differentiation. In some embodiments, the one or more cells (e.g., a clone) is selected for use in treating a disease or condition.

In some embodiments, the assessment includes determining the integration site of the DNA sequence encoding GBA1 into the genome of the cell, such as by any methods known in the art. In some embodiments, the cells introduced with (i) a DNA sequence encoding GBA1 (i.e. a GBA1-containing transgene) and (ii) a transposase or a nucleic acid sequence encoding a transposase, in accordance with the methods herein, e.g., as described in Section II.B, are subjected to integration site analysis. Integration site of the SNA sequence encoding GBA1 may be determined by any method known in the art, including inverse PCR (iPCR), whole genome sequencing, sequence capture followed by next generation sequencing (NGS), and/or targeted locus amplification (TLA). Uemura et al., Neurosci Res. (2014) 80:91-4; Liang et al., Transgenic Res (2008) 17(5):979-83; Srivastava et al., BMC Genomics (2014) 15:367; Ji et al., PLoS One (2014) 9(5):e96650; Dubose et al. Nucleic Acids Res (2013) 41(6):e70; deVree et al., Nat Biotechnol (2014) 32(10):1019-25.

In some embodiments, one or more cells (e.g., a clone) wherein the DNA sequence encoding GBA1 is integrated into a non-coding region of DNA is selected for differentiation, such as by any of the methods described in Section III. Thus, in some embodiments, one or more cells (e.g., a clone) wherein the sequence encoding GBA1 is integrated into an intron is selected for differentiation. In some embodiments, one or more cells (e.g., a clone) wherein the DNA sequence encoding GBA1 is integrated into a coding region of DNA (i.e., the DNA sequence disrupted a gene body) is not selected for differentiation, such as by any of the methods described in Section III. Thus, in some embodiments, one or more cells (e.g., a clone) wherein the sequence encoding GBA1 is integrated into an exon is not selected for differentiation.

In some embodiments, the assessment includes determining the number of copies of the DNA sequence encoding GBA1 introduced into the genome of a cell, such as by any methods known in the art. In some embodiments, it is desirable to introduce between one and five copies of GBA1 into a cell. Thus, in some embodiments, one or more cells (e.g., a clone) is selected that has between one and five integrated copies of GBA1. In some embodiments, a clone is selected that has one copy of GBA1. In some embodiments, a clone is selected that has two copies of GBA1. In some embodiments, a clone is selected that has one copy of GBA1. In some embodiments, a clone is selected that has three copies of GBA1. In some embodiments, a clone is selected that has four copies of GBA1. In some embodiments, a clone is selected that has five copies of GBA1. In some embodiments, the one or more cells (e.g., a clone) is selected for differentiation. In some embodiments, the one or more cells (e.g., a clone) is selected for use in treating a disease or condition.

In some embodiments, the cells that have undergone stable integration of the DNA sequence encoding GBA1 are referred to as “overexpressing cells.”

### III. Methods for Differentiating Cells

Provided herein are methods of differentiating neural cells, such as pluripotent stem cells (e.g., iPSCs), in which a DNA sequence encoding GBA1 (i.e., a GBA1-containing transgene) is stably integrated into the genome of the cells, such as by any method described herein in Section II. Unless otherwise indicated, the methods of differentiation provided herein involve the cells, e.g., the pluripotent stem cells, such as iPSCs, that underwent stable integration of one or more GBA1-containing transgene(s) using any of the methods as described herein in Section II.

In some embodiments, the methods of differentiating neural cells can be methods that differentiate neural cells, e.g., the iPSCs, that underwent integration of one or more GBA1-containing transgene(s), as described herein in Section II, into any neural cell type using any available or known method for inducing the differentiation of cells, e.g., pluripotent stem cells. In some embodiments, the method induces differentiation of the cells, e.g., pluripotent stem cells, into floor plate midbrain progenitor cells, determined dopaminergic (DA) neuron progenitor cells, and/or dopaminergic (DA) neurons. Any available and known method for inducing differentiation of the cells, e.g., pluripotent stem cells, into floor plate midbrain progenitor cells, determined dopaminergic (DA) neuron progenitor cells, and/or dopaminergic (DA) neurons can be used, including any of those described, e.g., in Section III.A.

In some embodiments, the method induces differentiation of the cells, e.g., pluripotent stem cells, into glial cells. In some embodiments, the glial cells are selected from the group consisting of microglia, astrocytes, oligodendrocytes, and ependymocytes.

In some embodiments, the method induces differentiation of the cells, e.g., pluripotent stem cells, into microglia or microglial-like cells. Any available and known method for inducing differentiation of the cells, e.g., pluripotent stem cells, into microglia or microglial-like cells can be used. Exemplary methods of inducing differentiation of pluripotent stem cells into microglia or microglial-like cells can be found in, e.g., Abud et al., Neuron (2017), Vol. 94: 278-293; Douvaras et al., Stem Cell Reports (2017), Vol. 8: 1516-1524; Muffat et al., Nature Medicine (2016), Vol. 22(11): 1358-1367; and Pandya et al., Nature Neuroscience (2017), Vol. 20(5): 753-759, the contents of which are hereby incorporated by reference in their entirety. Exemplary methods of inducing differentiation of pluripotent stem cells into microglia can also include, in some embodiments, the use of commercially available kits, and provided methods for use of such kits, including, e.g., STEMdiff™ Microglia Differentiation Kit, Catalog #100-0019 (STEMCELL Technologies, Cambridge, Mass.).

In some embodiments, the method induces differentiation of the cells, e.g., pluripotent stem cells, into macrophages. Any available and known method for inducing differentiation of the cells, e.g., pluripotent stem cells, into macrophages can be used. Exemplary methods of differentiation of pluripotent stem cells into macrophages can be found in, e.g., Lyadova et al., Front. Cell Dev. Biol., (2021) 9:640703; Mukherjee et al., Methods Mol Biol (2018) 1784:13-28; and Vaughan-Jackson et al., Stem Cell Reports (2021) 16(7):1735-48. Exemplary methods of inducing differentiation of pluripotent stem cells into macrophages can also include, in some embodiments, the use of commercially available kits and products, and provided methods for use of such kits and products, including, e.g., ImmunoCult™-SF Macrophage Medium, Catalog #10961 (STEMCELL Technologies, Cambridge, Mass.); CellXVivo Human M1 Macrophage Differentiation Kit, Cataolog #CDKO12 (R&D Systems, Minneapolis, Minn.); and CellXVivo Human M2 Macrophage Differentiation Kit, Catalog #CDK013 ((R&D Systems, Minneapolis, Minn.).

In some embodiments, the method induces differentiation of the cells, e.g., pluripotent stem cells, into hematopoietic stem cells (HSCs). Any available and known method for inducing differentiation of the cells, e.g., pluripotent stem cells, into HSCs can be used. Exemplary methods of differentiation of pluripotent stem cells into HSCs can be found in, e.g., Demirci et al., Stem Cells Transl Med. (2020) 9(12):1549-57; Alsayegh et al., Curr Genomics. (2019) 20(6):438-52; Tan et al., PNAS (2018) 115(9):2180-85; and Suzuki et al., Mol Ther (2013) 21(7): 1424-31. Exemplary methods of inducing differentiation of pluripotent stem cells into HSCs can also include, in some embodiments, the use of commercially available kits and products, and provided methods for use of such kits and products, including, e.g., STEMdiff™ Hematopoietic Kit, Catalog #05310 (STEMCELL Technologies, Cambridge, Mass.).

In some embodiments, the method induces differentiation of the cells, e.g., pluripotent stem cells, into astrocytes. Any available and known method for inducing differentiation of the cells, e.g., pluripotent stem cells, into astrocytes can be used. Exemplary methods of inducing differentiation of the cells, e.g., pluripotent stem cells, into astrocytes can be found in, e.g., TCW et al., Stem Cell Reports (2017), Vol. 9: 600-614, including the methods described in the references cited therein, e.g., in Table 1, the contents of which are hereby incorporated by reference in their entirety. Exemplary methods of inducing differentiation of pluripotent stem cells into astrocytes can include, in some embodiments, the use of commercially available kits, and provided methods for use of such kits, including, e.g., Astrocyte Medium, Catalog #1801 (ScienCell Research Laboratories, Carlsbad, Calif.); Astrocyte Medium, Catalog #A1261301 (ThermoFisher Scientific Inc, Waltham, Mass.); and AGM Astrocyte Growth Medium BulletKit, Catalog #CC-3186 (Lonza, Basel, Switzerland), the contents of which are hereby incorporated by reference in their entirety.

In some embodiments, the method induces differentiation of the cells, e.g., pluripotent stem cells, into oligodendrocytes. Any available and known method for inducing differentiation of the cells, e.g., pluripotent stem cells, into oligodendrocytes can be used. Exemplary methods of inducing differentiation of pluripotent stem cells into oligodendrocytes can be found in, e.g., Ehrlich et al., PNAS (2017), Vol. 114(11): E2243-E2252; Douvaras et al., Stem Cell Reports (2014), Vol. 3(2): 250-259; Stacpoole et al., Stem Cell Reports (2013), Vol. 1(5): 437-450; Wang et al., Cell Stem Cell (2013), Vol. 12(2): 252-264; and Piao et al., Cell Stem Cell (2015), Vol. 16(2): 198-210, the contents of which are hereby incorporated by reference in their entirety.

A. Differentiation of Neural Cells

Provided herein are methods of differentiating neural cells that comprise differentiating pluripotent stem cells, such as any of the cells produced by the methods as described, e.g., in Section II. The methods of differentiating neural cells are not limited and can be any available or known method for inducing the differentiation of pluripotent stem cells into floor plate midbrain progenitor cells, determined dopaminergic (DA) neuron progenitor cells, and/or dopaminergic (DA) neurons. Exemplary methods of differentiating neural cells can be found, e.g., in WO2013104752, WO2010096496, WO2013067362, WO2014176606, WO2016196661, WO2015143342, US20160348070, the contents of which are hereby incorporated by reference in their entirety.

Provided herein are methods of differentiating neural cells, involving (1) performing a first incubation including culturing pluripotent stem cells in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and (b) performing a second incubation including culturing cells of the spheroid in a substrate-coated culture vessel under conditions to neurally differentiate the cells.

The provided methods of differentiating neural cells, such as by subjecting iPSCs to cell culture methods that induce their differentiation into floor plate midbrain progenitor cells, determined dopaminergic (DA) neuron progenitor cells, and/or, dopaminergic (DA) neurons.

As described herein, iPSCs were generated from fibroblasts of human patients with Parkinson's disease. In a first incubation, the iPSCs were then differentiated to midbrain floor plate precursors and grown as spheroids in a non-adherent culture by exposure to small molecules, such as LDN, SB, PUR, SHH, CHIR, and combinations thereof, beginning on day 0. The resulting spheroids were then transferred to an adherent culture as part of a second incubation, optionally following dissociation of the spheroid, before being exposed to additional small molecules (e.g., LDN, CHIR, BDNF, GDNF, ascorbic acid, dbcAMP, TGFβ3, DAPT, and combinations thereof) to induce further differentiation into engraftable determined DA neuron progenitor cells or DA neurons. The provided methods may include any of those described in PCT/US2021/013324, which is incorporated herein by reference in its entirety.

Also provided herein are methods of differentiating neural cells, comprising differentiating pluripotent stem cells, such as any of the cells produced by the methods as described, e.g., in Section II, using any of the methods disclosed in any one of WO2013104752, WO2010096496, WO2013067362, WO2014176606, WO2016196661, WO2015143342, and US20160348070.

Also provided are methods of differentiating neural cells, involving: exposing pluripotent stem cells to (a) an inhibitor of bone morphogenetic protein (BMP) signaling; (b) an inhibitor of TGF-β/activin-Nodal signaling; and (c) at least one activator of Sonic Hedgehog (SHH) signaling. In some embodiments, the method further comprising exposing the pluripotent stem cells to at least one inhibitor of GSK3β signaling. In some embodiments, the exposing to an inhibitor of BMP signaling and the inhibitor of TGF-β/activin-Nodal signaling occurs while the pluripotent stem cells are attached to a substrate. In some embodiments, the inhibitor of BMP signaling is any inhibitor of BMP signaling described herein, the inhibitor of TGF-β/activin-Nodal signaling is any inhibitor of TGF-β/activin-Nodal signaling described herein, and the at least one activator of SHH signaling is any activator of SHH signaling described herein. In some embodiments, during the exposing to the inhibitor of BMP signaling, the inhibitor of TGF-β/activin-Nodal signaling, and the at least one activator of SHH signaling, the pluripotent stem cells are attached to a substrate. In some embodiments, during the exposing to the at least one inhibitor of GSK3β signaling, the pluripotent stem cells are attached to a substrate. In some embodiments, during the exposing to the inhibitor of BMP signaling, the inhibitor of TGF-β/activin-Nodal signaling, and the at least one activator of SHH signaling, the pluripotent stem cells are in a non-adherent culture vessel under conditions to produce a cellular spheroid. In some embodiments, during the exposing to the at least one inhibitor of GSK3β signaling, the pluripotent stem cells are in a non-adherent culture vessel under conditions to produce a cellular spheroid.

1. Cells Selected for Differentiation

In some embodiments, the cells selected to undergo differentiation are pluripotent stem cells (PSCs), e.g., iPSCs, that underwent stable integration of one or more GBA1-containing transgene(s) as described in Section II. In some embodiments, the cells selected to undergo differentiation are any cells stably expressing one or more GBA1-containing transgene(s) in accordance with the methods provided herein, e.g., in Section II. In some embodiments, the cells selected to undergo differentiation are any cells produced by the methods described herein, e.g., in Section II. In some embodiments, the cells selected to undergo differentiation are any cells selected by the methods described herein, e.g., in Section II.D.

2. Non-Adherent Culture

The provided methods include culturing PSCs (e.g., iPSCs) by incubation with certain molecules (e.g., small molecules) to induce their differentiation into floor plate midbrain progenitor cells, determined dopamine (DA) neuron progenitor cells, and/or, dopamine (DA) neurons. In particular, in some embodiments, the provided embodiments include a first incubation of PSCs under non-adherent conditions to produce spheroids, in the presence of certain molecules (e.g., small molecules), which can, in some aspects, improve the consistency of producing physiologically relevant cells for implantation. In some embodiments, the methods include performing a first incubation involving culturing pluripotent stem cells in a non-adherent culture vessel under conditions to produce a cell spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling.

In some embodiments, a non-adherent culture vessel is a culture vessel with a low or ultra-low attachment surface, such as to inhibit or reduce cell attachment. In some embodiments, culturing cells in a non-adherent culture vessel does not prevent all cells of the culture from attaching the surface of the culture vessel.

In some embodiments, a non-adherent culture vessel is a culture vessel with an ultra-low attachment surface. In some aspects, an ultra-low attachment surface may inhibit cell attachment for a period of time. In some embodiments, an ultra-low attachment surface may inhibit cell attachment for the period of time necessary to obtain confluent growth of the same cell type on an adherent surface. In some embodiments, the ultra-low attachment surface is coated or treated with a substance to prevent cell attachment, such as a hydrogel layer (e.g., a neutrally charged and/or hydrophilic hydrogel layer). In some embodiments, a non-adherent culture vessel is coated or treated with a surfactant prior to the first incubation. In some embodiments, the surfactant is pluronic acid.

In some embodiments, the non-adherent culture vessel is a plate, a dish, a flask, or a bioreactor. In some embodiments, the non-adherent culture vessel is a plate, such as a multi-well plate. In some embodiments, the non-adherent culture vessel is a 6-well or 24-well plate. In some embodiments, the wells of the multi-well plate further include micro-wells. In some any of the provided embodiments, a non-adherent culture vessel, such as a multi-well plate, has round or concave wells and/or microwells. In any of the provided embodiments, a non-adherent culture vessel, such as a multi-well plate, does not have corners or seams.

In some embodiments, a non-adherent culture vessel allows for three-dimensional formation of cell aggregates. In some embodiments, iPSCs are cultured in a non-adherent culture vessel, such as a multi-well plate, to produce cell aggregates (e.g., spheroids). In some embodiments, iPSCs are cultured in a non-adherent culture vessel, such as a multi-well plate, to produce cell aggregates (e.g., spheroids) on about day 7 of the method. In some embodiments, the cell aggregate (e.g., spheroid) expresses at least one of PAX6 and OTX2 on or by about day 7 of the method.

In some embodiments, the first incubation includes culturing pluripotent stem cells in a non-adherent culture vessel under conditions to produce a cellular spheroid.

In some embodiments, the number of PSCs plated on day 0 of the method is between about between about 0.1×10⁶ cells/cm² and about 2×10⁶ cells/cm², between about 0.1×10⁶ cells/cm² and about 1×10⁶ cells/cm², between about 0.1×10⁶ cells/cm² and about 0.8×10⁶ cells/cm², between about 0.1×10⁶ cells/cm² and about 0.6×10⁶ cells/cm², between about 0.1×10⁶ cells/cm² and about 0.4×10⁶ cells/cm², between about 0.1×10⁶ cells/cm² and about 0.2×10⁶ cells/cm², between about 0.2×10⁶ cells/cm² and about 2×10⁶ cells/cm², between about 0.2×10⁶ cells/cm² and about 1×10⁶ cells/cm², between about 0.2×10⁶ cells/cm² and about 0.8×10⁶ cells/cm², between about 0.2×10⁶ cells/cm² and about 0.6×10⁶ cells/cm², between about 0.2×10⁶ cells/cm² and about 0.4×10⁶ cells/cm², between about 0.4×10⁶ cells/cm² and about 2×10⁶ cells/cm², between about 0.4×10⁶ cells/cm² and about 1×10⁶ cells/cm², between about 0.4×10⁶ cells/cm² and about 0.8×10⁶ cells/cm², between about 0.4×10⁶ cells/cm² and about 0.6×10⁶ cells/cm², between about 0.6×10⁶ cells/cm² and about 2×10⁶ cells/cm², between about 0.6×10⁶ cells/cm² and about 1×10⁶ cells/cm², between about 0.6×10⁶ cells/cm² and about 0.8×10⁶ cells/cm², between about 0.8×10⁶ cells/cm² and about 2×10⁶ cells/cm², between about 0.8×10⁶ cells/cm² and about 1×10⁶ cells/cm², or between about 1.0×10⁶ cells/cm² and about 2×10⁶ cells/cm². In some embodiments, the number of cells plated on the substrate-coated culture vessel is between about 0.4×10⁶ cells/cm² and about 0.8×10⁶ cells/cm².

In some embodiments, the number of PSCs plated on day 0 of the method is between about 1×10⁵ pluripotent stem cells per well and about 20×10⁶ pluripotent stem cells per well, between about 1×10⁵ pluripotent stem cells per well and about 15×10⁶ pluripotent stem cells per well, between about 1×10⁵ pluripotent stem cells per well and about 10×10⁶ pluripotent stem cells per well, between about 1×10⁵ pluripotent stem cells per well and about 5×10⁶ pluripotent stem cells per well, between about 1×10⁵ pluripotent stem cells per well and about 1×10⁶ pluripotent stem cells per well, between about 1×10⁵ pluripotent stem cells per well and about 5×10⁵ pluripotent stem cells per well, between about 5×10⁵ pluripotent stem cells per well and about 20×10⁶ pluripotent stem cells per well, between about 5×10⁵ pluripotent stem cells per well and about 15×10⁶ pluripotent stem cells per well, between about 5×10⁵ pluripotent stem cells per well and about 10×10⁶ pluripotent stem cells per well, between about 5×10⁵ pluripotent stem cells per well and about 5×10⁶ pluripotent stem cells per well, between about 5×10⁵ pluripotent stem cells per well and about 1×10⁶ pluripotent stem cells per well, between about 1×10⁶ pluripotent stem cells per well and about 20×10⁶ pluripotent stem cells per well, between about 1×10⁶ pluripotent stem cells per well and about 15×10⁶ pluripotent stem cells per well, between about 1×10⁶ pluripotent stem cells per well and about 10×10⁶ pluripotent stem cells per well, between about 1×10⁶ pluripotent stem cells per well and about 5×10⁶ pluripotent stem cells per well, between about 5×10⁶ pluripotent stem cells per well and about 20×10⁶ pluripotent stem cells per well, between about 5×10⁶ pluripotent stem cells per well and about 15×10⁶ pluripotent stem cells per well, between about 5×10⁶ pluripotent stem cells per well and about 10×10⁶ pluripotent stem cells per well, between about 10×10⁶ pluripotent stem cells per well and about 20×10⁶ pluripotent stem cells per well, between about 10×10⁶ pluripotent stem cells per well and about 15×10⁶ pluripotent stem cells per well, or between about 15×10⁶ pluripotent stem cells per well and about 20×10⁶ pluripotent stem cells per well.

In some embodiments, the number of PSCs plated in a 6-well plate on day 0 of the method is between about 1×10⁶ pluripotent stem cells per well and about 20×10⁶ pluripotent stem cells per well, between about 1×10⁶ pluripotent stem cells per well and about 15×10⁶ pluripotent stem cells per well, between about 1×10⁶ pluripotent stem cells per well and about 10×10⁶ pluripotent stem cells per well, between about 1×10⁶ pluripotent stem cells per well and about 5×10⁶ pluripotent stem cells per well, between about 5×10⁶ pluripotent stem cells per well and about 20×10⁶ pluripotent stem cells per well, between about 5×10⁶ pluripotent stem cells per well and about 15×10⁶ pluripotent stem cells per well, between about 5×10⁶ pluripotent stem cells per well and about 10×10⁶ pluripotent stem cells per well, between about 10×10⁶ pluripotent stem cells per well and about 20×10⁶ pluripotent stem cells per well, between about 10×10⁶ pluripotent stem cells per well and about 15×10⁶ pluripotent stem cells per well, or between about 15×10⁶ pluripotent stem cells per well and about 20×10⁶ pluripotent stem cells per well.

In some embodiments, the number of PSCs plated in a 24-well plate on day 0 of the method is between about 1×10⁵ pluripotent stem cells per well and about 5×10⁶ pluripotent stem cells per well, between about 1×10⁵ pluripotent stem cells per well and about 1×10⁶ pluripotent stem cells per well, between about 1×10⁵ pluripotent stem cells per well and about 5×10⁵ pluripotent stem cells per well, between about 5×10⁵ pluripotent stem cells per well and about 5×10⁶ pluripotent stem cells per well, between about 5×10⁵ pluripotent stem cells per well and about 1×10⁶ pluripotent stem cells per well, or between about 1×10⁶ pluripotent stem cells per well and about 5×10⁶ pluripotent stem cells per well.

In some days, the number of PSCs plated on day 0 of the method is a number of cells sufficient to produce a cellular spheroid containing between about 1,000 cells and about 5,000 cells, or between about 2,000 cells and about 3,000 cells. In some days, the number of PSCs plated on day 0 of the method is a number of cells sufficient to produce a cellular spheroid containing between about 1,000 cells and about 5,000 cells. In some days, the number of PSCs plated on day 0 of the method is a number of cells sufficient to produce a cellular spheroid containing between about 2,000 cells and about 3,000 cells. In some days, the number of PSCs plated on day 0 of the method is a number of cells sufficient to produce a cellular spheroid containing about 2,000 cells. In some days, the number of PSCs plated on day 0 of the method is a number of cells sufficient to produce a cellular spheroid containing about 3,000 cells. In some embodiments, the spheroids containing the desired number is produced by the method on or by about day 7.

In some embodiments of the method provided herein, the first incubation includes culturing pluripotent stem cells in a non-adherent culture vessel under conditions to produce a cellular spheroid. In some embodiments, the first incubation is from about day 0 through about day 6. In some embodiments, the first incubation comprises culturing pluripotent stem cells in a culture media (“media”). In some embodiments, the first incubation comprises culturing pluripotent stem cells in the media from about day 0 through about day 6. In some embodiments, the first incubation comprises culturing pluripotent stem cells in the media to induce differentiation of the PSCs into floor plate midbrain progenitor cells.

In some embodiments, the media is also supplemented with a serum replacement containing minimal non-human-derived components (e.g., KnockOut™ serum replacement). In some embodiments, the serum replacement is provided in the media at 5% (v/v) for at least a portion of the first incubation. In some embodiments, the serum replacement is provided in the media at 5% (v/v) on day 0 and day 1. In some embodiments, the serum replacement is provided in the media at 2% (v/v) for at least a portion of the first incubation. In some embodiments, the serum replacement is provided in the media at 2% (v/v) from day 2 through day 6. In some embodiments, the serum replacement is provided in the media at 5% (v/v) on day 0 and day 1, and at 2% (v/v) from day 2 through day 6.

In some embodiments, the media is further supplemented with small molecules, such as any described above. In some embodiments, the small molecules are selected from among the group consisting of: a Rho-associated protein kinase (ROCK) inhibitor, an inhibitor of TGF-β/activin-Nodal signaling, at least one activator of Sonic Hedgehog (SHH) signaling, an inhibitor of bone morphogenetic protein (BMP) signaling, an inhibitor of glycogen synthase kinase 3β (GSK3β) signaling, and combinations thereof.

In some embodiments the media is supplemented with a Rho-associated protein kinase (ROCK) inhibitor on one or more days when cells are passaged. In some embodiments the media is supplemented with a ROCK inhibitor each day that cells are passaged. In some embodiments the media is supplemented with a ROCK inhibitor on day 0.

In some embodiments, cells are exposed to the ROCK inhibitor at a concentration of between about 1 μM and about 20 μM, between about 5 μM and about 15 μM, or between about 8 μM and about 12 μM. In some embodiments, cells are exposed to the ROCK inhibitor at a concentration of between about 1 μM and about 20 μM. In some embodiments, cells are exposed to the ROCK inhibitor at a concentration of between about 5 μM and about 15 μM. In some embodiments, cells are exposed to the ROCK inhibitor at a concentration of between about 8 μM and about 12 μM. In some embodiments, cells are exposed to the ROCK inhibitor at a concentration of about 10 μM.

In some embodiments, the ROCK inhibitor is selected from among the group consisting of: Fasudil, Ripasudil, Netarsudil, RKI-1447, Y-27632, GSK429286A, Y-30141, and combinations thereof. In some embodiments, the ROCK inhibitor is a small molecule. In some embodiments, the ROCK inhibitor selectively inhibits p160ROCK. In some embodiments, the ROCK inhibitor is Y-27632, having the formula:

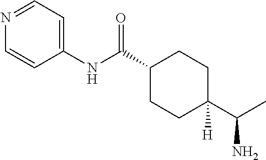 [SMILES: C[C@@H](N)[C@H]1CC[C@H](C(=O)Nc2ccncc2)CC1]

In some embodiments, cells are exposed to Y-27632 at a concentration of about 10 μM. In some embodiments, cells are exposed to Y-27632 at a concentration of about 10 μM on day 0.

In some embodiments the media is supplemented with an inhibitor of TGF-β/activin-Nodal signaling. In some embodiments the media is supplemented with an inhibitor of TGF-β/activin-Nodal signaling up to about day 7 (e.g., day 6 or day 7). In some embodiments the media is supplemented with an inhibitor of TGF-β/activin-Nodal signaling from about day 0 through day 6, each day inclusive.

In some embodiments, cells are exposed to the inhibitor of TGF-β/activin-Nodal signaling at a concentration of between about 1 μM and about 20 μM, between about 5 μM and about 15 μM, or between about 8 μM and about 12 μM. In some embodiments, cells are exposed to the inhibitor of TGF-β/activin-Nodal signaling at a concentration of between about 1 μM and about 20 μM. In some embodiments, cells are exposed to the inhibitor of TGF-β/activin-Nodal signaling at a concentration of between about 5 μM and about 15 μM. In some embodiments, cells are exposed to the inhibitor of TGF-β/activin-Nodal signaling at a concentration of between about 8 μM and about 12 μM. In some embodiments, cells are exposed to the inhibitor of TGF-β/activin-Nodal signaling at a concentration of about 10 μM.

In some embodiments, the inhibitor of TGF-β/activin-Nodal signaling is a small molecule. In some embodiments, the inhibitor of TGF-β/activin-Nodal signaling is capable of lowering or blocking transforming growth factor beta (TGFβ)/activin-Nodal signaling. In some embodiments, the inhibitor of TGF-β/activin-Nodal signaling inhibits ALK4, ALK5, ALK7, or combinations thereof. In some embodiments, the inhibitor of TGF-β/activin-Nodal signaling inhibits ALK4, ALK5, and ALK7. In some embodiments, the inhibitor of TGF-β/activin-Nodal signaling does not inhibit ALK2, ALK3, ALK6, or combinations thereof. In some embodiments, the inhibitor does not inhibit ALK2, ALK3, or ALK6. In some embodiments, the inhibitor of TGF-β/activin-Nodal signaling is SB431542 (e.g., CAS 301836-41-9, molecular formula of C22H18N4O3, and name of 4-[4-(1,3-benzodioxol-5-yl)-5-(2-pyridinyl)-1H-imidazol-2-yl]-benzamide), having the formula:

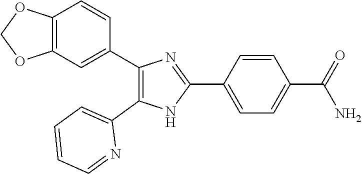 [SMILES: NC(=O)c1ccc(-c2nc(-c3ccc4c(c3)OCO4)c(-c3ccccn3)[nH]2)cc1]

In some embodiments, cells are exposed to SB431542 at a concentration of about 10 M. In some embodiments, cells are exposed to SB431542 at a concentration of about 10 μM until about day 7. In some embodiments, cells are exposed to SB431542 at a concentration of about 10 μM from about day 0 through about day 6, inclusive of each day.

In some embodiments the media is supplemented with at least one activator of sonic hedghehog (SHH) signaling. SHH refers to a protein that is one of at least three proteins in the mammalian signaling pathway family called hedgehog, another is desert hedgehog (DHH) while a third is Indian hedgehog (IHH). Shh interacts with at least two transmembrane proteins by interacting with transmembrane molecules Patched (PTC) and Smoothened (SMO). In some embodiments the media is supplemented with the at least one activator of SHH signaling up to about day 7 (e.g., day 6 or day 7). In some embodiments the media is supplemented with the at least one activator of SHH signaling from about day 0 through day 6, each day inclusive.

In some embodiments, the at least one activator of SHH signaling is SHH protein. In some embodiments, the at least one activator of SHH signaling is recombinant SHH protein. In some embodiments, the at least one activator of SHH signaling is recombinant mouse SHH protein. In some embodiments, the at least one activator of SHH signaling is recombinant human SHH protein. In some embodiments, the least one activator of SHH signaling is a recombinant N-Terminal fragment of a full-length murine sonic hedgehog protein capable of binding to the SHH receptor for activating SHH. In some embodiments, the at least one activator of SHH signaling is C25II SHH protein.

In some embodiments, cells are exposed to the at least one activator of SHH signaling at a concentration of between about 10 ng/mL and about 500 ng/mL, between about 20 ng/mL and 400 μg/mL, between about 30 ng/mL and about 300 ng/mL, between about about 40 ng/mL and about 200 ng/mL, or between about 50 ng/mL and about 100 ng/mL, each inclusive. In some embodiments, cells are exposed to the at least one activator of SHH signaling at a concentration of between about 50 ng/mL and about 100 ng/mL, each inclusive. In some embodiments, cells are exposed to the at least one activator of SHH signaling at a concentration of about 100 ng/mL. In some embodiments, the cells are exposed to SHH protein at about 100 ng/mL. In some embodiments, the cells are exposed to recombinant SHH protein at about 100 ng/mL. In some embodiments, the cells are exposed to recombinant mouse SHH protein at about 100 ng/mL. In some embodiments, the cells are exposed to C25II SHH protein at about 100 ng/mL.

In some embodiments, cells are exposed to recombinant SHH protein at a concentration of about 10 ng/mL. In some embodiments, cells are exposed to recombinant SHH protein at a concentration of about 10 ng/mL up to about day 7 (e.g., day 6 or day 7). In some embodiments, cells are exposed to recombinant SHH protein at a concentration of about 10 ng/mL from about day 0 through about day 6, inclusive of each day.

In some embodiments, cells are exposed to the at least one activator of SHH signaling at a concentration of between about 1 μM and about 20 μM, between about 5 μM and about 15 μM, or between about 8 μM and about 12 μM. In some embodiments, cells are exposed to the at least one activator of SHH signaling at a concentration of between about 1 μM and about 20 μM. In some embodiments, cells are exposed to the at least one activator of SHH signaling at a concentration of between about 5 μM and about 15 μM. In some embodiments, cells are exposed to the at least one activator of SHH signaling at a concentration of between about 8 μM and about 12 μM. In some embodiments, cells are exposed to the at least one activator of SHH signaling at a concentration of about 10 μM.

In some embodiments, the at least one activator of SHH signaling is an activator of the Hedgehog receptor Smoothened. It some embodiments, the at least one activator of SHH signaling is a small molecule. In some embodiments, the least one activator of SHH signaling is purmorphamine (e.g., CAS 483367-10-8), having the formula below:

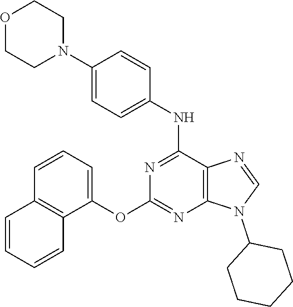 [SMILES: c1ccc2c(Oc3nc(Nc4ccc(N5CCOCC5)cc4)c4ncn(C5CCCCC5)c4n3)cccc2c1]

In some embodiments, cells are exposed to purmorphamine at a concentration of about 10 μM. In some embodiments, cells are exposed to purmorphamine at a concentration of about 10 μM up to day 7 (e.g., day 6 or day 7). In some embodiments, cells are exposed to purmorphamine at a concentration of about 10 μM from about day 0 through about day 6, inclusive of each day.

In some embodiments, the at least one activator of SHH signaling is SHH protein and purmorphamine. In some embodiments, cells are exposed to SHH protein and purmorphamine at a concentration up to about day 7 (e.g., day 6 or day 7). In some embodiments, cells are exposed to SHH protein and purpomorphamine from about day 0 through about day 6, inclusive of each day. In some embodiments, cells are exposed to 100 ng/mL SHH protein and 10 μM purmorphamine at a concentration up to about day 7 (e.g., day 6 or day 7). In some embodiments, cells are exposed to 100 ng/mL SHH protein and 10 μM purpomorphamine from about day 0 through about day 6, inclusive of each day.

In some embodiments the media is supplemented with an inhibitor of BMP signaling. In some embodiments the media is supplemented with an inhibitor of BMP signaling up to about day 7 (e.g., day 6 or day 7). In some embodiments the media is supplemented with an inhibitor of BMP signaling from about day 0 through day 6, each day inclusive.

In some embodiments, cells are exposed to the inhibitor of BMP signaling at a concentration of between about 0.01 μM and about 5 μM, between about 0.05 μM and about 1 μM, or between about 0.1 μM and about 0.5 μM, each inclusive. In some embodiments, cells are exposed to the inhibitor of BMP signaling at a concentration of between about 0.01 μM and about 5 μM. In some embodiments, cells are exposed to the inhibitor of BMP signaling at a concentration of between about 0.05 μM and about 1 μM. In some embodiments, cells are exposed to the inhibitor of BMP signaling at a concentration of between about 0.1 μM and about 0.5 μM. In some embodiments, cells are exposed to the inhibitor of BMP signaling at a concentration of about 0.1 μM.

In some embodiments, the inhibitor of BMP signaling is a small molecule. In some embodiments, the inhibitor of BMP signaling is selected from LDN193189 or K02288. In some embodiments, the inhibitor of BMP signaling is capable of inhibiting “Small Mothers Against Decapentaplegic” SMAD signaling. In some embodiments, the inhibitor of BMP signaling inhibits ALK1, ALK2, ALK3, ALK6, or combinations thereof. In some embodiments, the inhibitor of BMP signaling inhibits ALK1, ALK2, ALK3, and ALK6. In some embodiments, the inhibitor of BMP signaling inhibits BMP2, BMP4, BMP6, BMP7, and Activin cytokine signals and subsequently SMAD phosphorylation of Smad1, Smad5, and Smad8. In some embodiments, the inhibitor of BMP signaling is LDN193189. In some embodiments, the inhibitor of BMP signaling is LDN193189 (e.g., IUPAC name 4-(6-(4-(piperazin-1-yl)phenyl)pyrazolo[1,5-a]pyrimidin-3-yl)quinoline, with a chemical formula of C25H22N6), having the formula:

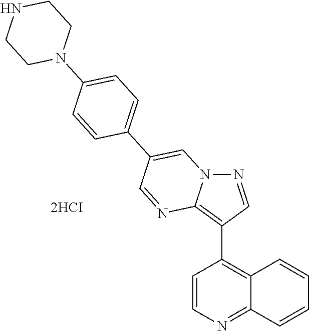 [SMILES: c1ccc2c(-c3cnn4cc(-c5ccc(N6CCNCC6)cc5)cnc34)ccnc2c1]

In some embodiments, cells are exposed to LDN193189 at a concentration of about 0.1 μM. In some embodiments, cells are exposed to LDN193189 at a concentration of about 0.1 μM up to about day 7 (e.g., day 6 or day 7). In some embodiments, cells are exposed to LDN193189 at a concentration of about 0.1 μM from about day 0 through about day 6, inclusive of each day.

In some embodiments the media is supplemented with an inhibitor of GSK3β signaling. In some embodiments the media is supplemented with an inhibitor of GSK3β signaling up to about day 7 (e.g., day 6 or day 7). In some embodiments the media is supplemented with an inhibitor of GSK3β signaling from about day 0 through day 6, each day inclusive.

In some embodiments, cells are exposed to the inhibitor of GSK3β signaling at a concentration of between about 0.1 μM and about 10 μM, between about 0.5 μM and about 8 μM, or between about 1 μM and about 4 μM, or between about 2 μM and about 3 μM, each inclusive. In some embodiments, cells are exposed to the inhibitor of GSK3β signaling at a concentration of between about 0.1 μM and about 10 μM. In some embodiments, cells are exposed to the inhibitor of GSK3β signaling at a concentration of between about 0.5 μM and about 8 μM. In some embodiments, cells are exposed to the inhibitor of BMP signaling at a concentration of between about 1 μM and about 4 μM. In some embodiments, cells are exposed to the inhibitor of BMP signaling at a concentration of between about 2 μM and about 3 μM. In some embodiments, cells are exposed to the inhibitor of GSK3β signaling at a concentration of about 2 μM.

In some embodiments, the inhibitor of GSK3β signaling is selected from among the group consisting of: lithium ion, valproic acid, iodotubercidin, naproxen, famotidine, curcumin, olanzapine, CHIR99012, and combinations thereof. In some embodiments, the inhibitor of GSK3β signaling is a small molecule. In some embodiments, the inhibitor of GSK3β signaling inhibits a glycogen synthase kinase 3β enzyme. In some embodiments, the inhibitor of GSK3β signaling inhibits GSK3α. In some embodiments, the inhibitor of GSK3β signaling modulates TGF-β and MAPK signaling. In some embodiments, the inhibitor of GSK3β signaling is an agonist of wingless/integrated (Wnt) signaling. In some embodiments, the inhibitor of GSK3β signaling has an IC50=6.7 nM against human GSK3β. In some embodiments, the inhibitor of GSK3β signaling is CHIR99021 (e.g., “3-[3-(2-Carboxyethyl)-4-methylpyrrol-2-methylidenyl]-2-indolinone” or IUPAC name 6-(2-(4-(2,4-dichlorophenyl)-5-(4-methyl-1H-imidazol-2-yl)pyrimidin-2-ylamino)ethylamino)nicotinonitrile), having the formula:

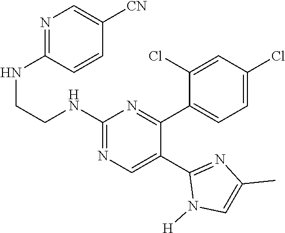 [SMILES: Cc1c[nH]c(-c2cnc(NCCNc3ccc(C#N)cn3)nc2-c2ccc(Cl)cc2Cl)n1]

In some embodiments, cells are exposed to CHIR99021 at a concentration of about 2.0 μM. In some embodiments, cells are exposed to CHIR99021 at a concentration of about 2.0 μM up to about day 7 (e.g., day 6 or day 7). In some embodiments, cells are exposed to CHIR99021 at a concentration of about 2.0 μM from about day 0 through about day 6, inclusive of each day.

In some embodiments, from day about 2 to about day 6, at least about 50% of the media is replaced daily. In some embodiments, from about day 2 to about day 6, about 50% of the media is replaced daily, every other day, or every third day. In some embodiments, from about day 2 to about day 6, about 50% of the media is replaced daily. In some embodiments, at least about 75% of the media is replaced on day 1. In some embodiments, about 100% of the media is replaced on day 1. In some embodiments, the replacement media contains small molecules about twice as concentrated as compared to the concentration of the small molecules in the media on day 0.

In some embodiments, the first incubation comprises culturing pluripotent stem cells in a “basal induction media.” In some embodiments, the first incubation comprises culturing pluripotent stem cells in the basal induction media from about day 0 through about day 6. In some embodiments, the first incubation comprises culturing pluripotent stem cells in the basal induction media to induce differentiation of the PSCs into floor plate midbrain progenitor cells.

In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, β-mercaptoethanol, and insulin. In some embodiments, the basal induction media is further supplemented with any of the small molecules as described above.

3. Transfer or Dissociation of Spheroids

In some embodiments, cell aggregates (e.g., spheroids) that are produced following the first incubation of culturing pluripotent stem cells in a non-adherent culture vessel are transferred or dissociated, prior to carrying out a second incubation of the cells on a substrate (adherent culture).

In some embodiments, the first incubation is carried out to produce a cell aggregate (e.g., a spheroid) that expresses at least one of PAX6 and OTX2. In some embodiments, the first incubation produces a cell aggregate (e.g., a spheroid) that expresses PAX6 and OTX2. In some embodiments, the first incubation produces a cell aggregate (e.g., a spheroid) on or by about day 7 of the methods provided herein. In some embodiments, the first incubation produces a cell aggregate (e.g., a spheroid) that expresses at least one of PAX6 and OTX2 on or by about day 7 of the methods provided herein. In some embodiments, the first incubation produces a cell aggregate (e.g., a spheroid) that expresses PAX6 and OTX2 on or by about day 7 of the methods provided herein.

In some embodiments, the cell aggregate (e.g., spheroid) produced by the first incubation is dissociated prior to the second incubation of the cells on a substrate. In some embodiments, the cell aggregate (e.g., spheroid) produced by the first incubation is dissociated to produce a cell suspension. In some embodiments, the cell suspension produced by the dissociation is a single cell suspension. In some embodiments, the dissociation is carried out at a time when the spheroid cells express at least one of PAX6 and OTX2. In some embodiments, the dissociation is carried out at a time when the spheroid cells express PAX6 and OTX2. In some embodiments, the dissociation is carried out on about day 7. In some embodiments, the cell aggregate (e.g., spheroid) is dissociated by enzymatic dissociation. In some embodiments, the enzyme is selected from among the group consisting of: accutase, dispase, collagenase, and combinations thereof. In some embodiments, the enzyme comprises accutase. In some embodiments, the enzyme is accutase. In some embodiments, the enzyme is dispase. In some embodiments, the enzyme is collagenase.

In some embodiments, the cell aggregate or cell suspension produced therefrom is transferred to a substrate-coated culture vessel for a second incubation. In some embodiments, the cell aggregate (e.g., spheroid) or cell suspension produced therefrom is transferred to a substrate-coated culture vessel following dissociation of the cell aggregate (e.g., spheroid). In some embodiments, the transferring is carried out immediately after the dissociating. In some embodiments, the transferring is carried out on about day 7.

In some embodiments, the cell aggregate (e.g., spheroid) is not dissociated prior to a second incubation. In some embodiments, a cell aggregate (e.g., spheroid) is transferred in its entirety to a substrate-coated culture vessel for a second incubation. In some embodiments, the transferring is carried out at a time when the spheroid cells express at least one of PAX6 and OTX2. In some embodiments, the transferring is carried out at a time when the spheroid cells express PAX6 and OTX2. In some embodiments, the transferring is carried out on about day 7.

In some embodiments, the transferring is to an adherent culture vessel. In some embodiments, the culture vessel is a plate, a dish, a flask, or a bioreactor. In some embodiments, the culture vessel is substrate-coated. In some embodiments, the substrate is a basement membrane protein. In some embodiments, the substrate is selected from laminin, collagen, entactin, heparin sulfate proteoglycans, and combinations thereof. In some embodiments, the substrate is laminin. In some embodiments, the substrate is recombinant. In some embodiments, the substrate is recombinant laminin. In some embodiments, the substrate is xeno-free. In some embodiments, the substrate is xeno-free laminin or a fragment thereof.

In some embodiments, the laminin or fragment thereof comprises an alpha chain, a beta chain, and a gamma chain. In some embodiments, the alpha chain is LAMA1, LAMA2, LAMA3, LAMA4, LAMA5, or a combination thereof. In some embodiments, the beta chain is LAMB1, LAMB2, LAMB3, LAMB4, or a combination thereof. In some embodiments, the gamma chain is LAMC1, LAMC2, LAMC3, or a combination thereof. In some embodiments, the laminin or a fragment thereof comprises any alpha, beta, and/or gamma chains as described in Aumailley, Cell Adh Migra (2013) 7(1):48-55 (see e.g., Table 1).

In some embodiments, the laminin or a fragment thereof is selected from the group consisting of: laminin 111, laminin 121, laminin 211, laminin 213, laminin 221, laminin 3A32, laminin 3B32, laminin 3A11, laminin 3A21, laminin 411, laminin 421, laminin 423, laminin 511, laminin 521, laminin 522, laminin 523, or a fragment of any of the foregoing. In some embodiments, the laminin is selected from laminin 521, laminin 111, laminin 511, and laminin 511-E8.

In some embodiments, the laminin or a fragment thereof comprises LAMA1, LAMB1, and LAMC1. In some embodiments, the laminin or a fragment thereof is laminin 111.

In some embodiments, the laminin or a fragment thereof comprises LAMA1, LAMB2, and LAMC1. In some embodiments, the laminin or a fragment thereof is laminin 121.

In some embodiments, the laminin or a fragment thereof comprises LAMA2, LAMB1, and LAMC1. In some embodiments, the laminin or a fragment thereof is laminin 211.

In some embodiments, the laminin or a fragment thereof comprises LAMA2, LAMB1, and LAMC3. In some embodiments, the laminin or a fragment thereof is laminin 213.

In some embodiments, the laminin or a fragment thereof comprises LAMA2, LAMB2, and LAMC1. In some embodiments, the laminin or a fragment thereof is laminin 221.

In some embodiments, the laminin or a fragment thereof comprises LAMA3A, LAMB3, and LAMC2. In some embodiments, the laminin or a fragment thereof is laminin 3A32.

In some embodiments, the laminin or a fragment thereof comprises LAMA3B, LAMB3, and LAMC2. In some embodiments, the laminin or a fragment thereof is laminin 3B32.

In some embodiments, the laminin or a fragment thereof comprises LAMA3A, LAMB1, and LAMC1. In some embodiments, the laminin or a fragment thereof is laminin 3A11.

In some embodiments, the laminin or a fragment thereof comprises LAMA3A, LAMB2, and LAMC1. In some embodiments, the laminin or a fragment thereof is laminin 3A21.

In some embodiments, the laminin or a fragment thereof comprises LAMA4, LAMB1, and LAMC1. In some embodiments, the laminin or a fragment thereof is laminin 411.

In some embodiments, the laminin or a fragment thereof comprises LAMA4, LAMB2, and LAMC1. In some embodiments, the laminin or a fragment thereof is laminin 421.

In some embodiments, the laminin or a fragment thereof comprises LAMA4, LAMB2, and LAMC3. In some embodiments, the laminin or a fragment thereof is laminin 423.

In some embodiments, the laminin or a fragment thereof comprises LAMA5, LAMB1, and LAMC1. In some embodiments, the laminin or a fragment thereof is laminin 511. In some embodiments, the laminin or a fragment thereof is a fragment of laminin 511. In some embodiments, the laminin or a fragment thereof comprises a fragment of LAMA5, a fragment of LAMB1, and a fragment of LAMC1. In some embodiments, the laminin or a fragment thereof comprises a truncated C-terminal fragment of LAMA5, a truncated, C-terminal fragment of LAMB1, and a truncated, C-terminal fragment of LAMC1. In some embodiments, the laminin or a fragment thereof comprises an E8 fragment of LAMA5, an E8 fragment of LAMB1, and an E8 fragment of LAMC1. In some embodiments, the laminin or a fragment thereof is laminin 511-E8 fragment. See Miyazaki et al., Nat Commun (2012) 3:1236.

In some embodiments, the laminin or a fragment thereof comprises LAMA5, LAMB2, and LAMC1. In some embodiments, the laminin or a fragment thereof is laminin 521.

In some embodiments, the laminin or a fragment thereof comprises LAMA5, LAMB2, and LAMC2. In some embodiments, the laminin or a fragment thereof is laminin 522.

In some embodiments, the laminin or a fragment thereof comprises LAMA5, LAMB2, and LAMC3. In some embodiments, the laminin or a fragment thereof is laminin 523.

In some embodiments, the substrate-coated culture vessel is exposed to poly-L-ornithine, optionally prior to being used for culturing cells. In some embodiments, the substrate-coated culture vessel is a 6-well or 24-well plate. In some embodiments, the substrate-coated culture vessel is a 6-well plate. In some embodiments, the substrate-coated culture vessel is a 24-well plate.

4. Adherent Culture

In some embodiments, the methods include performing a second incubation of the spheroid cells transferred to the substrate-coated culture vessel. In some embodiments, culturing the cells of the spheroid in the substrate-coated culture vessel under adherent conditions induces their differentiation into floor plate midbrain progenitor cells, determined dopamine (DA) neuron progenitor cells, and/or, dopamine (DA) neurons.

In some embodiments, the second incubation involves culturing cells of the spheroid in a culture vessel coated with a substrate including laminin, collagen, entactin, heparin sulfate proteoglycans, or a combination thereof, wherein beginning on day 7, the cells are exposed to (i) an inhibitor of BMP signaling and (ii) an inhibitor of GSK3β signaling; and beginning on day 11, the cells are exposed to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3); and (vi) an inhibitor of Notch signaling. In some embodiments, the method further includes harvesting the differentiated cells.

In some embodiments, the substrate-coated culture vessel is a culture vessel with a surface to which cells can attach. In some embodiments, the substrate-coated culture vessel is a culture vessel with a surface to which a substantial number of cells attach. In some embodiments, the substrate is a basement membrane protein. In some embodiments, the substrate is laminin, collagen, entactin, heparin sulfate proteoglycans, or a combination thereof. In some embodiments, the substrate is laminin. In some embodiments, the substrate is collagen. In some embodiments, the substrate is entactin. In some embodiments, the substrate is heparin sulfate proteoglycans. In some embodiments, the substrate is a recombinant protein. In some embodiments, the substrate is recombinant laminin. In some embodiments, the substrate is xeno-free. In some embodiments, the substrate is xeno-free laminin or a fragment thereof.

In some embodiments, the laminin or fragment thereof comprises an alpha chain, a beta chain, and a gamma chain. In some embodiments, the alpha chain is LAMA1, LAMA2, LAMA3, LAMA4, LAMA5, or a combination thereof. In some embodiments, the beta chain is LAMB1, LAMB2, LAMB3, LAMB4, or a combination thereof. In some embodiments, the gamma chain is LAMC1, LAMC2, LAMC3, or a combination thereof. In some embodiments, the laminin or a fragment thereof comprises any alpha, beta, and/or gamma chains as described in Aumailley, Cell Adh Migra (2013) 7(1):48-55 (see e.g., Table 1).

In some embodiments, the laminin or a fragment thereof is selected from the group consisting of: laminin 111, laminin 121, laminin 211, laminin 213, laminin 221, laminin 3A32, laminin 3B32, laminin 3A11, laminin 3A21, laminin 411, laminin 421, laminin 423, laminin 511, laminin 521, laminin 522, laminin 523, or a fragment of any of the foregoing. In some embodiments, the laminin is selected from laminin 521, laminin 111, laminin 511, and laminin 511-E8.

In some embodiments, the laminin or a fragment thereof comprises LAMA1, LAMB1, and LAMC1. In some embodiments, the laminin or a fragment thereof is laminin 111.

In some embodiments, the laminin or a fragment thereof comprises LAMA1, LAMB2, and LAMC1. In some embodiments, the laminin or a fragment thereof is laminin 121.

In some embodiments, the laminin or a fragment thereof comprises LAMA2, LAMB1, and LAMC1. In some embodiments, the laminin or a fragment thereof is laminin 211.

In some embodiments, the laminin or a fragment thereof comprises LAMA2, LAMB1, and LAMC3. In some embodiments, the laminin or a fragment thereof is laminin 213.

In some embodiments, the laminin or a fragment thereof comprises LAMA2, LAMB2, and LAMC1. In some embodiments, the laminin or a fragment thereof is laminin 221.

In some embodiments, the laminin or a fragment thereof comprises LAMA3A, LAMB3, and LAMC2. In some embodiments, the laminin or a fragment thereof is laminin 3A32.

In some embodiments, the laminin or a fragment thereof comprises LAMA3B, LAMB3, and LAMC2. In some embodiments, the laminin or a fragment thereof is laminin 3B32.

In some embodiments, the laminin or a fragment thereof comprises LAMA3A, LAMB1, and LAMC1. In some embodiments, the laminin or a fragment thereof is laminin 3A11.

In some embodiments, the laminin or a fragment thereof comprises LAMA3A, LAMB2, and LAMC1. In some embodiments, the laminin or a fragment thereof is laminin 3A21.

In some embodiments, the laminin or a fragment thereof comprises LAMA4, LAMB1, and LAMC1. In some embodiments, the laminin or a fragment thereof is laminin 411.

In some embodiments, the laminin or a fragment thereof comprises LAMA4, LAMB2, and LAMC1. In some embodiments, the laminin or a fragment thereof is laminin 421.

In some embodiments, the laminin or a fragment thereof comprises LAMA4, LAMB2, and LAMC3. In some embodiments, the laminin or a fragment thereof is laminin 423.

In some embodiments, the laminin or a fragment thereof comprises LAMA5, LAMB1, and LAMC1. In some embodiments, the laminin or a fragment thereof is laminin 511. In some embodiments, the laminin or a fragment thereof is a fragment of laminin 511. In some embodiments, the laminin or a fragment thereof comprises a fragment of LAMA5, a fragment of LAMB1, and a fragment of LAMC1. In some embodiments, the laminin or a fragment thereof comprises a truncated C-terminal fragment of LAMA5, a truncated, C-terminal fragment of LAMB1, and a truncated, C-terminal fragment of LAMC1. In some embodiments, the laminin or a fragment thereof comprises an E8 fragment of LAMA5, an E8 fragment of LAMB1, and an E8 fragment of LAMC1. In some embodiments, the laminin or a fragment thereof is laminin 511-E8 fragment. See Miyazaki et al., Nat Commun (2012) 3:1236.

In some embodiments, the laminin or a fragment thereof comprises LAMA5, LAMB2, and LAMC1. In some embodiments, the laminin or a fragment thereof is laminin 521.

In some embodiments, the laminin or a fragment thereof comprises LAMA5, LAMB2, and LAMC2. In some embodiments, the laminin or a fragment thereof is laminin 522.

In some embodiments, the laminin or a fragment thereof comprises LAMA5, LAMB2, and LAMC3. In some embodiments, the laminin or a fragment thereof is laminin 523.

In some embodiments, the substrate-coated culture vessel is exposed to poly-L-ornithine. In some embodiments, the substrate-coated culture vessel is exposed to poly-L-ornithine prior to being used for cell culture.

In some embodiments, the non-adherent culture vessel is a plate, a dish, a flask, or a bioreactor. In some embodiments, the non-adherent culture vessel is a plate, such as a multi-well plate. In some embodiments, the non-adherent culture vessel is a plate. In some embodiments, the non-adherent culture vessel is a 6-well or 24-well plate. In some embodiments, the non-adherent culture vessel is a dish. In some embodiments, the non-adherent culture vessel is a flask. In some embodiments, the non-adherent culture vessel is a bioreactor.

In some embodiments, the substrate-coated culture vessel allows for a monolayer cell culture. In some embodiments, cells derived from the cell aggregate (e.g., spheroid) produced by the first incubation are cultured in a monolayer culture on the substrate-coated plates. In some embodiments, cells derived from the cell aggregate (e.g., spheroid) produced by the first incubation are cultured to produce a monolayer culture of cells positive for one or more of LMX1A, FOXA2, EN1, CORIN, and combinations thereof. In some embodiments, cells derived from the cell aggregate (e.g., spheroid) produced by the first incubation are cultured to produce a monolayer culture of cells, wherein at least some of the cells are positive for EN1 and CORIN. In some embodiments, cells derived from the cell aggregate (e.g., spheroid) produced by the first incubation are cultured to produce a monolayer culture of cells, wherein at least some of the cells are TH+. In some embodiments, at least some cells are TH+ by or on about day 25. In some embodiments, cells derived from the cell aggregate (e.g., spheroid) produced by the first incubation are cultured to produce a monolayer culture of cells, wherein at least some of the cells are TH+FOXA2+. In some embodiments, at least some cells are TH+FOXA2+ by or on about day 25.

In the methods provided herein, the second incubation involves culturing cells of the spheroid in a substrate-coated culture vessel under conditions to induce neural differentiation of the cells. In some embodiments, the cells of the spheroid are plated on the substrate-coated culture vessel on about day 7.

In some embodiments, the number of cells plated on the substrate-coated culture vessel is between about 0.1×10⁶ cells/cm² and about 2×10⁶ cells/cm², between about 0.1×10⁶ cells/cm² and about 1×10⁶ cells/cm², between about 0.1×10⁶ cells/cm² and about 0.8×10⁶ cells/cm², between about 0.1×10⁶ cells/cm² and about 0.6×10⁶ cells/cm², between about 0.1×10⁶ cells/cm² and about 0.4×10⁶ cells/cm², between about 0.1×10⁶ cells/cm² and about 0.2×10⁶ cells/cm², between about 0.2×10⁶ cells/cm² and about 2×10⁶ cells/cm², between about 0.2×10⁶ cells/cm² and about 1×10⁶ cells/cm², between about 0.2×10⁶ cells/cm² and about 0.8×10⁶ cells/cm², between about 0.2×10⁶ cells/cm² and about 0.6×10⁶ cells/cm², between about 0.2×10⁶ cells/cm² and about 0.4×10⁶ cells/cm², between about 0.4×10⁶ cells/cm² and about 2×10⁶ cells/cm², between about 0.4×10⁶ cells/cm² and about 1×10⁶ cells/cm², between about 0.4×10⁶ cells/cm² and about 0.8×10⁶ cells/cm², between about 0.4×10⁶ cells/cm² and about 0.6×10⁶ cells/cm², between about 0.6×10⁶ cells/cm² and about 2×10⁶ cells/cm², between about 0.6×10⁶ cells/cm² and about 1×10⁶ cells/cm², between about 0.6×10⁶ cells/cm² and about 0.8×10⁶ cells/cm², between about 0.8×10⁶ cells/cm² and about 2×10⁶ cells/cm², between about 0.8×10⁶ cells/cm² and about 1×10⁶ cells/cm², or between about 1.0×10⁶ cells/cm² and about 2×10⁶ cells/cm². In some embodiments, the number of cells plated on the substrate-coated culture vessel is between about 0.4×10⁶ cells/cm² and about 0.8×10⁶ cells/cm².

In some embodiments, the second incubation is from about day 7 until harvesting of the cells. In some embodiments, the cells are harvested on about day 16 or later. In some embodiments, the cells are harvested between about day 16 and about day 30. In some embodiments, the cells are harvested between about day 19 and about day 24. In some embodiments, the cells are harvested between about day 18 and about day 25. In some embodiments, the cells are harvested on about day 18. In some embodiments, the cells are harvested on about day 19. In some embodiments, the cells are harvested on about day 20. In some embodiments, the cells are harvested on about day 21. In some embodiments, the cells are harvested on about day 22. In some embodiments, the cells are harvested on about day 23. In some embodiments, the cells are harvested on about day 24. In some embodiments, the cells are harvested on about day 25. In some embodiments, the second incubation is from about day 7 until about day 18. In some embodiments, the second incubation is from about day 7 until about day 25.

In some embodiments, the second incubation involves culturing cells derived from the cell aggregate (e.g., spheroid) in a culture media (“media”).

In some embodiments, the second incubation involves culturing the cells in the media from about day 7 until harvest or collection. In some embodiments, cells are cultured in the media to produce determined dopamine (DA) neuron progenitor cells, or dopamine (DA) neurons.

In some embodiments, the media is also supplemented with a serum replacement containing minimal non-human-derived components (e.g., KnockOut™ serum replacement). In some embodiments, the media is supplemented with the serum replacement from about day 7 through about day 10. In some embodiments, the media is supplemented with about 2% (v/v) of the serum replacement. In some embodiments, the media is supplemented with about 2% (v/v) of the serum replacement from about day 7 through about day 10.

In some embodiments, the media is further supplemented with small molecules. In some embodiments, the small molecules are selected from among the group consisting of: a Rho-associated protein kinase (ROCK) inhibitor, an inhibitor of bone morphogenetic protein (BMP) signaling, an inhibitor of glycogen synthase kinase 3β (GSK3β) signaling, and combinations thereof.

In some embodiments the media is supplemented with a Rho-associated protein kinase (ROCK) inhibitor on one or more days when cells are passaged. In some embodiments the media is supplemented with a ROCK inhibitor each day that cells are passaged. In some embodiments the media is supplemented with a ROCK inhibitor on day 7, day 16, day 20, or a combination thereof. In some embodiments the media is supplemented with a ROCK inhibitor on day 7. In some embodiments the media is supplemented with a ROCK inhibitor on day 16. In some embodiments the media is supplemented with a ROCK inhibitor on day 20. In some embodiments the media is supplemented with a ROCK inhibitor on day 7 and day 16. In some embodiments the media is supplemented with a ROCK inhibitor on day 16 and day 20. In some embodiments the media is supplemented with a ROCK inhibitor on day 7, day 16, and day 20.

In some embodiments, cells are exposed to the ROCK inhibitor at a concentration of between about 1 μM and about 20 μM, between about 5 μM and about 15 μM, or between about 8 μM and about 12 μM. In some embodiments, cells are exposed to the ROCK inhibitor at a concentration of between about 1 μM and about 20 μM. In some embodiments, cells are exposed to the ROCK inhibitor at a concentration of between about 5 μM and about 15 μM. In some embodiments, cells are exposed to the ROCK inhibitor at a concentration of between about 8 μM and about 12 μM. In some embodiments, cells are exposed to the ROCK inhibitor at a concentration of about 10 μM.

In some embodiments, the ROCK inhibitor is Fasudil, Ripasudil, Netarsudil, RKI-1447, Y-27632, GSK429286A, Y-30141, or a combination thereof. In some embodiments, the ROCK inhibitor is a small molecule. In some embodiments, the ROCK inhibitor selectively inhibits p160ROCK. In some embodiments, the ROCK inhibitor is Y-27632, having the formula:

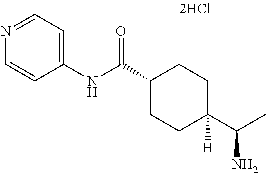 [SMILES: C[C@@H](N)[C@H]1CC[C@H](C(=O)Nc2ccncc2)CC1]

In some embodiments, cells are exposed to Y-27632 at a concentration of about 10 μM. In some embodiments, cells are exposed to Y-27632 at a concentration of about 10 μM on day 7, day 16, day 20, or a combination thereof. In some embodiments, cells are exposed to Y-27632 at a concentration of about 10 μM on day 7. In some embodiments, cells are exposed to Y-27632 at a concentration of about 10 μM on day 16. In some embodiments, cells are exposed to Y-27632 at a concentration of about M on day 20. In some embodiments, cells are exposed to Y-27632 at a concentration of about 10 μM on day 7 and day 16. In some embodiments, cells are exposed to Y-27632 at a concentration of about 10 μM on day 16 and day 20. In some embodiments, cells are exposed to Y-27632 at a concentration of about 10 μM on day 7, day 16, and day 20.

In some embodiments the media is supplemented with an inhibitor of BMP signaling. In some embodiments the media is supplemented with an inhibitor of BMP signaling from about day 7 up to about day 11 (e.g., day 10 or day 11). In some embodiments the media is supplemented with an inhibitor of BMP signaling from about day 7 through day 10, each day inclusive.

In some embodiments, cells are exposed to the inhibitor of BMP signaling at a concentration of between about 0.01 μM and about 5 μM, between about 0.05 μM and about 1 μM, or between about 0.1 μM and about 0.5 μM, each inclusive. In some embodiments, cells are exposed to the inhibitor of BMP signaling at a concentration of between about 0.01 μM and about 5 μM. In some embodiments, cells are exposed to the inhibitor of BMP signaling at a concentration of between about 0.05 μM and about 1 μM. In some embodiments, cells are exposed to the inhibitor of BMP signaling at a concentration of between about 0.1 μM and about 0.5 μM. In some embodiments, cells are exposed to the inhibitor of BMP signaling at a concentration of about 0.1 μM.

In some embodiments, the inhibitor of BMP signaling is a small molecule. In some embodiments, the inhibitor of BMP signaling is LDN193189 or K02288. In some embodiments, the inhibitor of BMP signaling is capable of inhibiting “Small Mothers Against Decapentaplegic” SMAD signaling. In In some embodiments, the inhibitor of BMP signaling inhibits ALK1, ALK2, ALK3, ALK6, or combinations thereof. In some embodiments, the inhibitor of BMP signaling inhibits ALK1, ALK2, ALK3, and ALK6. In some embodiments, the inhibitor of BMP signaling inhibits BMP2, BMP4, BMP6, BMP7, and Activin cytokine signals and subsequently SMAD phosphorylation of Smad1, Smad5, and Smad8. In some embodiments, the inhibitor of BMP signaling is LDN193189. In some embodiments, the inhibitor of BMP signaling is LDN193189 (e.g., IUPAC name 4-(6-(4-(piperazin-1-yl)phenyl)pyrazolo[1,5-a]pyrimidin-3-yl)quinoline, with a chemical formula of C25H22N6), having the formula:

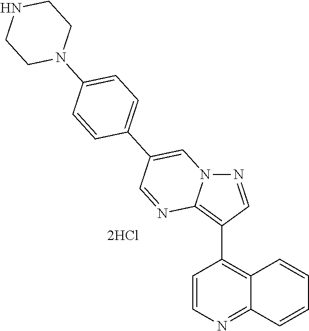 [SMILES: c1ccc2c(-c3cnn4cc(-c5ccc(N6CCNCC6)cc5)cnc34)ccnc2c1]

In some embodiments, cells are exposed to LDN193189 at a concentration of about 0.1 μM. In some embodiments, cells are exposed to LDN193189 at a concentration of about 0.1 μM from about day 7 up to about day 11 (e.g., day 10 or day 11). In some embodiments, cells are exposed to LDN193189 at a concentration of about 0.1 μM from about day 7 through about day 10, inclusive of each day.

In some embodiments the media is supplemented with an inhibitor of GSK3β signaling. In some embodiments the media is supplemented with an inhibitor of GSK3β signaling from about day 7 up to about day 13 (e.g., day 12 or day 13). In some embodiments the media is supplemented with an inhibitor of GSK3β signaling from about day 7 through day 12, each day inclusive.

In some embodiments, cells are exposed to the inhibitor of GSK3β signaling at a concentration of between about 0.1 μM and about 10 μM, between about 0.5 μM and about 8 μM, or between about 1 μM and about 4 μM, or between about 2 μM and about 3 μM, each inclusive. In some embodiments, cells are exposed to the inhibitor of GSK3β signaling at a concentration of between about 0.1 μM and about 10 μM. In some embodiments, cells are exposed to the inhibitor of GSK3β signaling at a concentration of between about 0.5 μM and about 8 μM. In some embodiments, cells are exposed to the inhibitor of GSK3β signaling at a concentration of between about 1 μM and about 4 μM. In some embodiments, cells are exposed to the inhibitor of GSK3β signaling at a concentration of between about 2 μM and about 3 μM. In some embodiments, cells are exposed to the inhibitor of GSK3β signaling at a concentration of about 2 μM.

In some embodiments, the inhibitor of GSK3β signaling is selected from lithium ion, valproic acid, iodotubercidin, naproxen, famotidine, curcumin, olanzapine, CHIR99012, or a combination thereof. In some embodiments, the inhibitor of GSK3β signaling is a small molecule. In some embodiments, the inhibitor of GSK3β signaling inhibits a glycogen synthase kinase 3β enzyme. In some embodiments, the inhibitor of GSK3β signaling inhibits GSK3α. In some embodiments, the inhibitor of GSK3β signaling modulates TGF-β and MAPK signaling. In some embodiments, the inhibitor of GSK3β signaling is an agonist of wingless/integrated (Wnt) signaling. In some embodiments, the inhibitor of GSK3β signaling has an IC50=6.7 nM against human GSK3β. In some embodiments, the inhibitor of GSK3β signaling is CHIR99021 (e.g., “3-[3-(2-Carboxyethyl)-4-methylpyrrol-2-methylidenyl]-2-indolinone” or IUPAC name 6-(2-(4-(2,4-dichlorophenyl)-5-(4-methyl-1H-imidazol-2-yl)pyrimidin-2-ylamino)ethylamino)nicotinonitrile), having the formula:

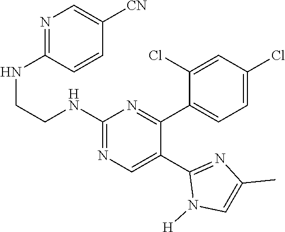 [SMILES: Cc1c[nH]c(-c2cnc(NCCNc3ccc(C#N)cn3)nc2-c2ccc(Cl)cc2Cl)n1]

In some embodiments, cells are exposed to CHIR99021 at a concentration of about 2.0 μM. In some embodiments, cells are exposed to CHIR99021 at a concentration of about 2.0 μM from about day 7 up to about day 13 (e.g., day 12 or day 13). In some embodiments, cells are exposed to CHIR99021 at a concentration of about 2.0 μM from about day 7 through about day 12, inclusive of each day.

In some embodiments the media is supplemented with brain-derived neurotrophic factor (BDNF). In some embodiments the media is supplemented with BDNF beginning on about day 11. In some embodiments the media is supplemented with BDNF from about day 11 until harvest or collection. In some embodiments the media is supplemented with BDNF from about day 11 through day 18. In some embodiments the media is supplemented with BDNF from about day 11 through day 25.

In some embodiments, cells are exposed to BDNF at a concentration of between about 1 ng/mL and 100 ng/mL, between about 5 ng/mL and about 50 ng/mL, between about 10 ng/mL and about 30 ng/mL. In some embodiments, cells are exposed to BDNF at a concentration of between about 10 ng/mL and about 30 ng/mL. In some embodiments, cells are exposed to BDNF at a concentration of about 20 ng/mL.

In some embodiments, the media is supplemented with about 20 ng/mL BDNF beginning on about day 11. In some embodiments the media is supplemented with 20 ng/mL BDNF from about day 11 until harvest or collection. In some embodiments the media is supplemented with about 20 ng/mL BDNF from about day 11 through day 18. In some embodiments the media is supplemented with about 20 ng/mL BDNF from about day 11 through day 25.

In some embodiments the media is supplemented with glial cell-derived neurotrophic factor (GDNF). In some embodiments the media is supplemented with GDNF beginning on about day 11. In some embodiments the media is supplemented with GDNF from about day 11 until harvest or collection. In some embodiments the media is supplemented with GDNF from about day 11 through day 18. In some embodiments the media is supplemented with GDNF from about day 11 through day 25.

In some embodiments, cells are exposed to GDNF at a concentration of between about 1 ng/mL and 100 ng/mL, between about 5 ng/mL and about 50 ng/mL, between about 10 ng/mL and about 30 ng/mL. In some embodiments, cells are exposed to GDNF at a concentration of between about 10 ng/mL and about 30 ng/mL. In some embodiments, cells are exposed to GDNF at a concentration of about 20 ng/mL.

In some embodiments, the media is supplemented with about 20 ng/mL GDNF beginning on about day 11. In some embodiments the media is supplemented with 20 ng/mL GDNF from about day 11 until harvest or collection. In some embodiments the media is supplemented with about 20 ng/mL GDNF from about day 11 through day 18. In some embodiments the media is supplemented with about 20 ng/mL GDNF from about day 11 through day 25.

In some embodiments the media is supplemented with ascorbic acid. In some embodiments the media is supplemented with ascorbic acid beginning on about day 11. In some embodiments the media is supplemented with ascorbic acid from about day 11 until harvest or collection. In some embodiments the media is supplemented with ascorbic acid from about day 11 through day 18. In some embodiments the media is supplemented with ascorbic acid from about day 11 through day 25.

In some embodiments, cells are exposed to ascorbic acid at a concentration of between about 0.05 mM and 5 mM, between about 0.1 mM and about 1 mM, between about 0.2 mM and about 0.5 mM, each inclusive. In some embodiments, cells are exposed to ascorbic acid at a concentration of between about 0.05 mM and about 5 mM, each inclusive. In some embodiments, cells are exposed to ascorbic acid at a concentration of between about 0.1 mM and about 1 mM, each inclusive. In some embodiments, cells are exposed to ascorbic acid at a concentration of about 0.2 mM.

In some embodiments, the media is supplemented with about 0.2 mM ascorbic acid beginning on about day 11. In some embodiments the media is supplemented with 0.2 mM ascorbic acid from about day 11 until harvest or collection. In some embodiments the media is supplemented with about 0.2 mM ascorbic acid from about day 11 through day 18. In some embodiments the media is supplemented with about 0.2 mM ascorbic acid from about day 11 through day 25.

In some embodiments the media is supplemented with dibutyryl cyclic AMP (dbcAMP). In some embodiments the media is supplemented with dbcAMP beginning on about day 11. In some embodiments the media is supplemented with dbcAMP from about day 11 until harvest or collection. In some embodiments the media is supplemented with dbcAMP from about day 11 through day 18. In some embodiments the media is supplemented with dbcAMP from about day 11 through day 25.

In some embodiments, cells are exposed to dbcAMP at a concentration of between about 0.05 mM and 5 mM, between about 0.1 mM and about 3 mM, between about 0.2 mM and about 1 mM, each inclusive. In some embodiments, cells are exposed to dbcAMP at a concentration of between about 0.1 mM and about 3 mM, each inclusive. In some embodiments, cells are exposed to dbcAMP at a concentration of between about 0.2 mM and about 1 mM, each inclusive. In some embodiments, cells are exposed to dbcAMP at a concentration of about 0.5 mM.

In some embodiments, the media is supplemented with about 0.5 mM dbcAMP beginning on about day 11. In some embodiments the media is supplemented with 0.5 mM dbcAMP from about day 11 until harvest or collection. In some embodiments the media is supplemented with about 0.5 mM dbcAMP from about day 11 through day 18. In some embodiments the media is supplemented with about 0.5 mM dbcAMP from about day 11 through day 25.

In some embodiments the media is supplemented with transforming growth factor beta 3 (TGFβ3). In some embodiments the media is supplemented with TGFβ3 beginning on about day 11. In some embodiments the media is supplemented with TGFβ3 from about day 11 until harvest or collection. In some embodiments the media is supplemented with TGFβ3 from about day 11 through day 18. In some embodiments the media is supplemented with TGFβ3 from about day 11 through day 25.

In some embodiments, cells are exposed to TGFβ3 at a concentration of between about 0.1 ng/mL and 10 ng/mL, between about 0.5 ng/mL and about 5 ng/mL, or between about 1.0 ng/mL and about 2.0 ng/mL. In some embodiments, cells are exposed to TGFβ3 at a concentration of between about 1.0 ng/mL and about 2.0 ng/mL, each inclusive. In some embodiments, cells are exposed to TGFβ3 at a concentration of about 1 ng/mL.

In some embodiments, the media is supplemented with about 1 ng/mL TGFβ3 beginning on about day 11. In some embodiments the media is supplemented with 1 ng/mL TGFβ3 from about day 11 until harvest or collection. In some embodiments the media is supplemented with about 1 ng/mL TGFβ3 from about day 11 through day 18. In some embodiments the media is supplemented with about 1 ng/mL TGFβ3 from about day 11 through day 25.

In some embodiments the media is supplemented with an inhibitor of Notch signaling. In some embodiments the media is supplemented with an inhibitor of Notch signaling beginning on about day 11. In some embodiments the media is supplemented with an inhibitor of Notch signaling from about day 11 until harvest or collection. In some embodiments the media is supplemented with an inhibitor of Notch signaling from about day 11 through day 18. In some embodiments the media is supplemented with an inhibitor of Notch signaling from about day 11 through day 25.

In some embodiments, an inhibitor of Notch signaling is selected from cowanin, PF-03084014, L685458, LY3039478, DAPT, or a combination thereof. In some embodiments, the inhibitor of Notch signaling inhibits gamma secretase. In some embodiments, the inhibitor of Notch signaling is a small molecule. In some embodiments, the inhibitor of Notch signaling is DAPT, having the following formula:

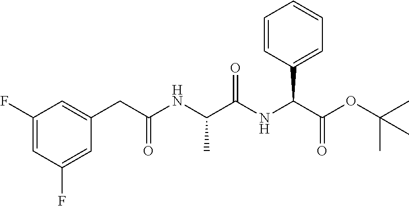 [SMILES: C[C@H](NC(=O)Cc1cc(F)cc(F)c1)C(=O)N[C@H](C(=O)OC(C)(C)C)c1ccccc1]

In some embodiments, cells are exposed to DAPT at a concentration of between about 1 μM and about 20 μM, between about 5 μM and about 15 μM, or between about 8 μM and about 12 μM. In some embodiments, cells are exposed to DAPT at a concentration of between about 1 μM and about 20 μM. In some embodiments, cells are exposed to DAPT at a concentration of between about 5 μM and about 15 μM. In some embodiments, cells are exposed to DAPT at a concentration of between about 8 μM and about 12 μM. In some embodiments, cells are exposed to DAPT at a concentration of about 10 μM.

In some embodiments, the media is supplemented with about 10 μM DAPT beginning on about day 11. In some embodiments the media is supplemented with 10 μM DAPT from about day 11 until harvest or collection. In some embodiments the media is supplemented with about 10 μM DAPT from about day 11 through day 18. In some embodiments the media is supplemented with about 10 μM DAPT from about day 11 through day 25.

In some embodiments, beginning on about day 11, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about day 11 until harvest or collection, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about day 11 until day 18, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT. In some embodiments, from about day 11 until day 25, the media is supplemented with about 20 ng/mL BDNF, about 20 ng/mL GDNF, about 0.2 mM ascorbic acid, about 0.5 mM dbcAMP, about 1 ng/mL TGFβ3, and about 10 μM DAPT.

In some embodiments, a serum replacement is provided in the media from about day 7 through about day 10. In some embodiments, the serum replacement is provided at 2% (v/v) in the media on day 7 through day 10.

In some embodiments, from day about 7 to about day 16, at least about 50% of the media is replaced daily. In some embodiments, from about day 7 to about day 16, about 50% of the media is replaced daily, every other day, or every third day. In some embodiments, from about day 7 to about day 16, about 50% of the media is replaced daily. In some embodiments, beginning on about day 17, at least about 50% of the media is replaced daily, every other day, or every third day. In some embodiments, beginning on about day 17, at least about 50% of the media is replaced every other day. In some embodiments, beginning on about day 17, about 50% of the media is replaced daily, every other day, or every third day. In some embodiments, beginning on about day 17, about 50% of the media is replaced every other day. In some embodiments, the replacement media contains small molecules about twice as concentrated as compared to the concentration of the small molecules in the media on day 0.

In some embodiments, the second incubation involves culturing cells derived from the cell aggregate (e.g., spheroid) in a “basal induction media.” In some embodiments, the second incubation involves culturing cells derived from the cell aggregate (e.g., spheroid) in a “maturation media.” In some embodiments, the second incubation involves culturing cells derived from the cell aggregate (e.g., spheroid) in the basal induction media, and then in the maturation media.

In some embodiments, the second incubation involves culturing the cells in the basal induction media from about day 7 through about day 10. In some embodiments, the second incubation involves comprises culturing the cells in the maturation media beginning on about day 11. In some embodiments, the second incubation involves culturing the cells in the basal induction media from about day 7 through about day 10, and then in the maturation media beginning on about day 11. In some embodiments, cells are cultured in the maturation media to produce determined dopamine (DA) neuron progenitor cells, or dopamine (DA) neurons.

In some embodiments, the basal induction media is formulated to contain Neurobasal™ media and DMEM/F12 media at a 1:1 ratio, supplemented with N-2 and B27 supplements, non-essential amino acids (NEAA), GlutaMAX™, L-glutamine, β-mercaptoethanol, and insulin. In some embodiments, the basal induction media is further supplemented with any of the molecules described in Section II.

In some embodiments, the maturation media is formulated to contain Neurobasal™ media, supplemented with N-2 and B27 supplements, non-essential amino acids (NEAA), and GlutaMAX™. In some embodiments, the maturation media is further supplemented with any of the molecules described in Section II.

In some embodiments, the cells are cultured in the basal induction media from about day 7 up to about day 11 (e.g., day 10 or day 11). In some embodiments, the cells are cultured in the basal induction media from about day 7 through day 10, each day inclusive. In some embodiments, the cells are cultured in the maturation media beginning on about day 11. In some embodiments, the cells are cultured in the basal induction media from about day 7 through about day 10, and then the cells are cultured in the maturation media beginning on about day 11. In some embodiments, the cells are cultured in the maturation media from about day 11 until harvest or collection of the cells. In some embodiments, cells are harvested between day 16 and 27. In some embodiments, cells are harvested between day 18 and day 25. In some embodiments, cells are harvested on day 18. In some embodiments, cells are harvested on day 20. In some embodiments, cells are harvested on day 25.

5. Harvesting, Collecting, and Formulating Differentiated Cells

In embodiments of the provided methods, neurally differentiated cells produced by the methods provided herein can be harvested or collected, such as for formulation and use of the cells. In some embodiments, the provided methods for producing differentiated cells, such as for use as a cell therapy in the treatment of a neurodegenerative disease may include formulation of cells, such as formulation of differentiated cells resulting from the provided methods described herein. In some embodiments, the dose of cells comprising differentiated cells (e.g., determined DA neuron progenitor cells or DA neurons), is provided as a composition or formulation, such as a pharmaceutical composition or formulation. Such compositions can be used in accord with the provided methods, such as in the prevention or treatment of neurodegenerative disorders, including Parkinson's disease.

In some cases, the cells are processed in one or more steps for manufacturing, generating or producing a cell therapy and/or differentiated cells may include formulation of cells, such as formulation of differentiated cells resulting from the methods. In some cases, the cells can be formulated in an amount for dosage administration, such as for a single unit dosage administration or multiple dosage administration.

In certain embodiments, one or more compositions of differentiated cells are formulated. In particular embodiments, one or more compositions of differentiated cells are formulated after the one or more compositions have been produced. In some embodiments, the one or more compositions have been previously cryopreserved and stored, and are thawed prior to the administration.

In certain embodiments, the differentiated cells include determined DA neuron progenitor cells. In some embodiments, a formulated composition of differentiated cells is a composition enriched for determined DA neuron progenitor cells. In certain embodiments, the differentiated cells include DA neurons. In some embodiments, a formulated composition of differentiated cells is a composition enriched for DA neurons.

In certain embodiments, the cells are cultured for a minimum or maximum duration or amount of time. In certain embodiments, the cells are cultured for a minimum duration or amount of time. In certain embodiments, the cells are cultured for a maximum duration or amount of time. In some embodiments, the cells are differentiated for at least 16 days. In some embodiments, the cells are differentiated for between 16 day and 30 days. In some embodiments, the cells are differentiated for between 16 day and 27 days. In some embodiments, the cells are differentiated for between 18 and 25 day. In some embodiments, the cells are differentiated for about 18 days. In some embodiments, the cells are differentiated for about 20 days. In some embodiments, the cells are differentiated for about 25 days.

In certain embodiments, the cells are cultured for a minimum or maximum duration or amount of time. In certain embodiments, the cells are cultured for a minimum duration or amount of time. In certain embodiments, the cells are cultured for a maximum duration or amount of time. In some embodiments, the cells are harvested after at least 16 days of culture. In some embodiments, the cells are harvested after at least 18 days of culture. In some embodiments, the cells are harvested after at least 19 days of culture. In some embodiments, the cells are harvested after at least 20 days of culture. In some embodiments, the cells are harvested between 16 days and 30 days of culture. In some embodiments, the cells are harvested between 16 days and 27 days of culture. In some embodiments, the cells are harvested between 18 days and 25 days of culture. In some embodiments, the cells are harvested between 19 days and 24 days of culture. In some embodiments, the cells are harvested after about 18 days of culture. In some embodiments, the cells are harvested after about 19 days of culture. In some embodiments, the cells are harvested after about 20 days of culture. In some embodiments, the cells are harvested after about 21 days of culture. In some embodiments, the cells are harvested after about 22 days of culture. In some embodiments, the cells are harvested after about 23 days of culture. In some embodiments, the cells are harvested after about 24 days of culture. In some embodiments, the cells are harvested after about 25 days of culture.

In some embodiments, cells harvested after about 18 days of culture are determined dopaminergic (DA) neuron progenitor cells or DA neurons. In some embodiments, cells harvested after about 18 days of culture are determined dopaminergic (DA) neuron progenitor cells. In some embodiments, cells harvested after about 18 days of culture are DA neurons. In some embodiments, cells harvested after about 20 days of culture are determined dopaminergic (DA) neuron progenitor cells or DA neurons. In some embodiments, cells harvested after about 20 days of culture are determined dopaminergic (DA) neuron progenitor cells. In some embodiments, cells harvested after about 20 days of culture are DA neurons.

In some embodiments, the cells are formulated in a pharmaceutically acceptable buffer, which may, in some aspects, include a pharmaceutically acceptable carrier or excipient. In some embodiments, the processing includes exchange of a medium into a medium or formulation buffer that is pharmaceutically acceptable or desired for administration to a subject. In some embodiments, the processing steps can involve washing the differentiated cells to replace the cells in a pharmaceutically acceptable buffer that can include one or more optional pharmaceutically acceptable carriers or excipients. Exemplary of such pharmaceutical forms, including pharmaceutically acceptable carriers or excipients, can be any described below in conjunction with forms acceptable for administering the cells and compositions to a subject. The pharmaceutical composition in some embodiments contains the cells in amounts effective to treat or prevent the neurodegenerative condition or disease (e.g., Parkinson's disease), such as a therapeutically effective or prophylactically effective amount.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as carbidopa-levodopa (e.g., Levodopa), dopamine agonists (e.g., pramipexole, ropinirole, rotigotine, and apomorphine), MAO B inhibitors (e.g., selegiline, rasagiline, and safinamide), catechol O-methyltransferase (COMT) inhibitors (e.g., entacapone and tolcapone), anticholinergics (e.g., benztropine and trihexylphenidyl), amantadine, etc.

Compositions in some embodiments are provided as sterile liquid preparations, e.g., isotonic aqueous solutions, suspensions, emulsions, dispersions, or viscous compositions, which may in some aspects be buffered to a selected pH. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

In embodiments, the formulation buffer contains a cryopreservative. In some embodiments, the cells are formulated with a cyropreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the processing steps can involve washing the differentiated cells to replace the cells in a cryopreservative solution. In some embodiments, the cells are frozen, e.g., cryopreserved or cryoprotected, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryopreserved or cryoprotected, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA.

In particular embodiments, the composition of differentiated cells are formulated, cryopreserved, and then stored for an amount of time. In certain embodiments, the formulated, cryopreserved cells are stored until the cells are released for administration. In particular embodiments, the formulated cryopreserved cells are stored for between 1 day and 6 months, between 1 month and 3 months, between 1 day and 14 days, between 1 day and 7 days, between 3 days and 6 days, between 6 months and 12 months, or longer than 12 months. In some embodiments, the cells are cryopreserved and stored for, for about, or for less than 1 days, 2 days, 3 days, 4 days, 5 days, 6 days, or 7 days. In certain embodiments, the cells are thawed and administered to a subject after the storage.

In some embodiments, the formulation is carried out using one or more processing step including washing, diluting or concentrating the cells. In some embodiments, the processing can include dilution or concentration of the cells to a desired concentration or number, such as unit dose form compositions including the number of cells for administration in a given dose or fraction thereof. In some embodiments, the processing steps can include a volume-reduction to thereby increase the concentration of cells as desired. In some embodiments, the processing steps can include a volume-addition to thereby decrease the concentration of cells as desired. In some embodiments, the processing includes adding a volume of a formulation buffer to differentiated cells. In some embodiments, the volume of formulation buffer is from or from about 1 μL to 5000 μL, such as at least or about at least or about or 5 μL, 10 μL, 20 μL, 50 μL, 100 μL, 200 μL, 300 μL, 400 μL, 500 μL, 1000 μL, 2000 μL, 3000 μL, 4000 μL, or 5000 μL.

A container may generally contain the cells to be administered, e.g., one or more unit doses thereof. The unit dose may be an amount or number of the cells to be administered to the subject or twice the number (or more) of the cells to be administered. It may be the lowest dose or lowest possible dose of the cells that would be administered to the subject.

In some embodiments, such cells produced by the method, or a composition comprising such cells, are administered to a subject for treating a neurodegenerative disease or condition. In some embodiments, the cells that have undergone stable integration of the DNA sequence encoding GBA1 as described in Section II and differentiation as described in Section III are referred to as “overexpressing cells.”

B. Exemplary Processes

As described by the methods provided herein, pluripotent stem cells may be differentiated into lineage specific cell populations, including determined DA progenitors cells and DA neurons. These cells may then be used in cell replacement therapy. As described by the methods here, in some embodiments, the pluripotent stem cells are differentiated into floor plate midbrain progenitor cells, and the resulting spheroid cells are further differentiated into determined dopamine (DA) neuron progenitor cells, and/or dopamine (DA) neurons. In some embodiments, the pluripotent stem cells are differentiated into determined DA neuron progenitor cells. In some embodiments, the pluripotent stem cells are differentiated into DA neurons. In some embodiments, pluripotent stem cells are embryonic stem cells. In some embodiments, pluripotent stem cells are induced pluripotent stem cells.

In some embodiments, embryonic stem cells are differentiated into floor plate midbrain progenitor cells, and then into determined dopamine (DA) neuron progenitor cells, and/or dopamine (DA) neurons. In some embodiments, embryonic stem cells are differentiated into determined DA neuron progenitor cells. In some embodiments, embryonic stem cells are differentiated into DA neurons.

In some embodiments, induced pluripotent stem cells are differentiated into floor plate midbrain progenitor cells, and then into determined dopamine (DA) neuron progenitor cells, and/or dopamine (DA) neurons. In some embodiments, induced pluripotent stem cells are differentiated into determined DA neuron progenitor cells. In some embodiments, induced pluripotent stem cells are differentiated into DA neurons.

In some embodiments, the method involves (a) performing a first incubation including culturing pluripotent stem cells in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and (b) performing a second incubation including culturing cells of the spheroid in a substrate-coated culture vessel under conditions to induce neural differentiation the cells.

In some embodiments, culturing the cells under conditions to induce neural differentiation of the cells involves exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3); and (vi) an inhibitor of Notch signaling.

In some embodiments, the method involves (a) performing a first incubation including culturing pluripotent stem cells in a plate having microwells under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; (iv) an inhibitor of glycogen synthase kinase 3β (GSK3β) signaling; and (v) a serum replacement; (b) dissociating the cells of the spheroid to produce a cell suspension; (c) transferring cells of the cell suspension to a laminin-coated culture vessel; (d) performing a second incubation including culturing cells of the spheroid in the laminin-coated culture vessel under conditions to induce neural differentiation of the cells; and (e) harvesting the neurally differentiated cells. In some embodiments, the second incubation involves culturing cells in the presence of a serum replacement. In some embodiments, culturing the cells under conditions to induce neural differentiation of the cells involves exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3); and (vi) an inhibitor of Notch signaling.

In some embodiments, the cells are exposed to the inhibitor of TGF-β/activin-Nodal (e.g., SB431542 or “SB”) from day 0 up to about day 7 (e.g., day 6 or day 7). In some embodiments, the cells are exposed to the inhibitor of TGF-β/activin-Nodal (e.g., SB431542 or “SB”) from day 0 through day 6, inclusive of each day. In some embodiments, the cells are exposed to the at least one activator of SHH signaling (e.g., SHH protein and purmorphamine, collectively “SHH/PUR”) from day 0 up to about day 7 (e.g., day 6 or day 7). In some embodiments, the cells are exposed to the at least one activator of SHH signaling (e.g., SHH protein and purmorphamine, collectively “SHH/PUR”) from day 0 through day 6, inclusive of each day. In some embodiments, the cells are exposed to the inhibitor of BMP signaling (e.g., LDN193189 or “LDN”) from day 0 up to about day 11 (e.g., day 10 or day 11). In some embodiments, the cells are exposed to the inhibitor of BMP signaling (e.g., LDN193189 or “LDN”) from day 0 through day 10, inclusive of each day. In some embodiments, the cells are exposed to the inhibitor of GSK3β signaling (e.g., CHIR99021 or “CHIR”) from day 0 up to about day 13 (e.g., day 12 or day 13). In some embodiments, the cells are exposed to the inhibitor of GSK3β signaling (e.g., CHIR99021 or “CHIR”) from day 0 through day 12.

In some embodiments, the cells are exposed to (i) an inhibitor of TGF-β/activin-Nodal signaling from day 0 up to about day 7 (e.g., day 6 or day 7); (ii) at least one activator of Sonic Hedgehog (SHH) signaling from day 0 up to about day 7 (e.g., day 6 or day 7); (iii) an inhibitor of bone morphogenetic protein (BMP) signaling from day 0 up to about day 11 (e.g., day 10 or day 11); and (iv) an inhibitor of glycogen synthase kinase 3β (GSK3β) signaling from day 0 up to about day 13 (e.g., day 12 or day 13). In some embodiments, the cells are exposed to (i) SB from day 0 up to about day 7 (e.g., day 6 or day 7); (ii) SHH/PUR from day 0 up to about day 7 (e.g., day 6 or day 8); (iii) LDN from day 0 up to about day 11 (e.g., day 10 or day 11); and (iv) CHIR from day 0 up to about day 13 (e.g., day 12 or day 13). In some embodiments, the cells are exposed to (i) an inhibitor of TGF-β/activin-Nodal signaling from day 0 through day 6, each day inclusive; (ii) at least one activator of Sonic Hedgehog (SHH) signaling from day 0 through day 6, each day inclusive; (iii) an inhibitor of bone morphogenetic protein (BMP) signaling from day 0 through day 10, each day inclusive; and (iv) an inhibitor of glycogen synthase kinase 3β (GSK3β) signaling from day 0 through day 12, each day inclusive. In some embodiments, the cells are exposed to (i) SB from day 0 through day 6, each day inclusive; (ii) SHH/PUR from day 0 through day 6, each day inclusive; (iii) LDN from day 0 through day 10, each day inclusive; and (iv) CHIR from day 0 through day 12, each day inclusive.

In some embodiments, the cells are exposed to brain-derived neurotrophic factor (BDNF) beginning on day 11. In some embodiments, the cells are exposed to ascorbic acid. In some embodiments, the cells are exposed to glial cell-derived neurotrophic factor (GDNF) beginning on day 11. In some embodiments, the cells are exposed to dibutyryl cyclic AMP (dbcAMP) beginning on day 11. In some embodiments, the cells are exposed to transforming growth factor beta-3 (TGFβ3) beginning on day 11. In some embodiments, the cells are exposed to the inhibitor of Notch signaling (e.g., DAPT) beginning on day 11. In some embodiments, beginning on day 11, the cells are exposed to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3); and (vi) the inhibitor of Notch signaling (e.g., DAPT) (collectively “BAGCT/DAPT”). In some embodiments, the cells are exposed to BAGCT/DAPT beginning on day 11 until harvest or collection. In some embodiments, the cells are exposed to BAGCT/DAPT from day 11 through day 18. In some embodiments, the cells are exposed to BAGCT/DAPT from day 11 through day 25.

In some embodiments, the cells are exposed to a Rho-associated protein kinase (ROCK) inhibitor on day 0. In some embodiments, the cells are exposed to a Rho-associated protein kinase (ROCK) inhibitor on day 7. In some embodiments, the cells are exposed to a Rho-associated protein kinase (ROCK) inhibitor on day 16. In some embodiments, the cells are exposed to a Rho-associated protein kinase (ROCK) inhibitor on day 20. In some embodiments, the cells are exposed to a Rho-associated protein kinase (ROCK) inhibitor on day 0, day 7, day 16, and day 20. In some embodiments, the cells are exposed to a ROCK inhibitor on the day on which the cells are passaged. In some embodiments, the cells are passaged on day 0, 7, 16, 20, or combinations thereof. In some embodiments, the cells are passaged on day 0, 7, 16, and 20.

In some embodiments, the cells are cultured in a basal induction medium comprising DMEM/F-12 and Neurobasal media (e.g., at a 1:1 ratio), supplemented with N2, B27, non-essential amino acids (NEAA), Glutamax, L-glutamine, β-mercaptoethanol, and insulin. In some embodiments, the cells are cultured in the basal induction media from about day 0 through about day 10. In some embodiments, the basal induction media is for differentiating pluripotent stem cells into floor plate midbrain progenitor cells.

In some embodiments, the cells are cultured in a maturation medium comprising Neurobasal media, supplemented with N2, B27, non-essential amino acids (NEAA), and Glutamax. In some embodiments, the cells are cultured in the basal induction media from about day 11 until harvest or collection. In some embodiments, the cells are cultured in the basal induction media from about day 11 through day 18. In some embodiments, the maturation media is for differentiating floor plate midbrain progenitor cells into determined dopamine (DA) neuron progenitor cells. In some embodiments, the cells are cultured in the basal induction media from about day 11 through day 25. In some embodiments, the maturation media is for differentiating floor plate midbrain progenitor cells into dopamine (DA) neurons.

In some embodiments, the media is supplemented with small molecules as described above, including SB, SHH/PUR, LDN, CHIR, BAGCT/DAPT, and ROCKi. In some embodiments, the media is changed every day or every other day. In some embodiments the media is changed every day. In some embodiments the media is changed every other day. In some embodiments, the media is changed every day from about day 0 up to about day 17 (e.g., day 16 or day 18). In some embodiments, the media is changed every other day from about day 18 until harvest or collection. In some embodiments, the media is changed every day from about day 0 up to about day 17 (e.g., day 16 or day 18), and then every other day from about day 18 until harvest or collection.

In some embodiments, a serum replacement is provided in the media from about day 0 up to about day 10 (e.g., day 9 or day 11). In some embodiments, the serum replacement is provided at 5% (v/v) in the media on day 0 and day 1. In some embodiments, the serum replacement is provided at 2% (v/v) in the media on day 2 through day 10. In some embodiments, the serum replacement is provided at 5% (v/v) in the media on day 0 and day 1 and at 2% (v/v) in the media on day 2 through day 10. In some embodiments, serum replacement is not provided in the media after day 10.

In some embodiments, at least about 50% or at least about 75% of the media is changed. In some embodiments, at least about 50% of the media is changed. In some embodiments, at least about 75% of the media is changed. In some embodiments about 100% of the media is changed.

In some embodiments, about 50% or about 75% of the media is changed. In some embodiments, about 50% of the media is changed. In some embodiments, about 75% of the media is changed. In some embodiments about 100% of the media is changed.

In some embodiments, the media is supplemented with small molecules selected from SB, SHH/PUR, LDN, CHIR, BAGCT/DAPT, ROCKi, or a combination thereof. In some embodiments, when about 50% of the media is changed, the concentration of each small molecule is doubled as compared to its concentration on day 0.

In some embodiments, cells are harvested between about day 16 and about day 30. In some embodiments, cells are harvested between about day 16 and about day 27. In some embodiments, cells are harvested between about day 18 and about day 25. In some embodiments, cells are harvested between about day 19 and about day 24. In some embodiments, cells are harvested on about day 18. In some embodiments, cells harvested on about day 18 are determined DA progenitor cells or DA neurons. In some embodiments, cells harvested on about day 18 are determined DA progenitor cells. In some embodiments, cells harvested on about day 18 are DA neurons. In some embodiments, cells are harvested on about day 20. In some embodiments, cells harvested on about day 20 are determined DA progenitor cells or DA neurons. In some embodiments, cells harvested on about day 20 are determined DA progenitor cells. In some embodiments, cells harvested on about day 20 are DA neurons. In some embodiments, cells are harvested on about day 25. In some embodiments, cells harvested on about day 25 are determined DA progenitor cells or DA neurons. In some embodiments, cells harvested on about day 25 are determined DA progenitor cells. In some embodiments, cells harvested on about day 25 are DA neurons. In some embodiments, compositions comprising cells generated by the methods provided herein are used for the treatment of a neurodegenerative disease or condition, such as Parkinson's disease. In some embodiments, a composition of cells generated by any of the methods described herein are administered to a subject who has Parkinson's disease. In some embodiments, a composition of cells generated by any of the methods described herein are administered by stereotactic injection, such as with a catheter. In some embodiments, a composition of cells generated by any of the methods described herein are administered to the striatum of a subject with Parkinson's disease.

Also provided herein is an exemplary method of differentiating neural cells, the method comprising: exposing the pluripotent stem cells to: (a) an inhibitor of bone morphogenetic protein (BMP) signaling; (b) an inhibitor of TGF-β/activin-Nodal signaling; and (c) at least one activator of Sonic Hedgehog (SHH) signaling. In some embodiments, during the exposing, the pluripotent stem cells are attached to a substrate. In some embodiments, during the exposing, the pluripotent stem cells are in a non-adherent culture vessel under conditions to produce a cellular spheroid.

In some embodiments, the method further comprises exposing the pluripotent stem cells to at least one inhibitor of GSK3β signaling. In some embodiments, during the exposing to the at least one inhibitor of GSK3β signaling, the pluripotent stem cells are attached to a substrate. In some embodiments, during the exposing to the at least one inhibitor of GSK3β signaling, the pluripotent stem cells are in a non-adherent culture vessel under conditions to produce a cellular spheroid.

In some embodiments, the inhibitor of TGF-β/activin-Nodal signaling is SB431542.

In some embodiments, the at least one activator of SHH signaling is SHH or purmorphamine. In some embodiments, the inhibitor of BMP signaling is LDN193189. In some embodiments, the at least one inhibitor of GSK3β signaling is CHIR99021.

In some embodiments, the exposing results in a population of differentiated neural cells. In some embodiments, the differentiated neural cells are floor plate midbrain progenitor cells, determined dopamine (DA) neuron progenitor cells, and/or, dopamine (DA) neurons.

The differentiated neural cells produced by any of the methods described herein are sometimes referred to as “overexpressing and differentiated cells.”

### IV. Compositions and Formulations

Also provided herein are populations of overexpressing (i.e., GBA1 overexpressing) cells, compositions containing overexpressing cells, and compositions enriched for overexpressing cells. The overexpressing cells are cells, e.g., PSCs, such as iPSCs, and cells differentiated therefrom, that have been introduced with (i) a deoxyribonucleic acid (DNA) sequence encoding GBA1 operably linked to a promoter; and (ii) a transposase or a nucleic acid sequence encoding a transposase by any of the methods described in Section II. In some embodiments, the overexpressing cells, the compositions containing overexpressing cells, and compositions enriched for overexpressing cells, are produced by the methods described herein, e.g., as described in Section II and Section III. In some embodiments, the population of overexpressing cells, the composition containing overexpressing cells, and the compositions enriched for overexpressing cells, include overexpressing cells that are differentiated neural cells, such as floor plate midbrain progenitor cells, determined dopamine (DA) neuron progenitor cells, and/or dopamine (DA) neurons, or glial cells, e.g., microglial cells, astrocytes, oligodendrocytes, or ependymocytes. In some embodiments, the population of overexpressing cells, the composition containing overexpressing cells, and the compositions enriched for overexpressing cells, include overexpressing cells that are macrophages. In some embodiments, the population of overexpressing cells, the composition containing overexpressing cells, and the compositions enriched for overexpressing cells, include overexpressing cells that are hematopoietic stem cells (HSCs). In some embodiments, the provided population of overexpressing cells is a population of the cell produced by any the methods described herein, e.g., as described in Section II and Section III. Accordingly, also provided herein is a population of the cell produced by any the methods described herein, e.g., as described in Section II and Section III, as well as compositions comprising the cell produced by any the methods described herein, e.g., as described in Section II and Section III, and compositions enriched for the cell produced by any the methods described herein, e.g., as described in Section II and Section IIII.

In some embodiments, the provided population of overexpressing cells, composition containing overexpressing cells, or composition enriched for overexpressing cells, include a cell population comprising cells that express (e.g., stably express) one or more transgene(s) containing GBA1, wherein a gene variant of GBA1 is associated with decreased GCase activity. In some embodiments, the GBA1 is the wildtype form or a functional form or portion thereof. In some embodiments, the gene variant is associated with PD. In some embodiments, the gene variant is associated with GD. In some embodiments, the provided population of overexpressing cells, composition containing overexpressing cells, or composition enriched for overexpressing cells, include a cell population comprising cells that express (e.g., stably express) one or more transgene(s) containing a wildtype version of GBA1, wherein a gene variant of GBA1 is associated with PD, such as a gene variant associated with PD that is within the human GBA1 locus. In some embodiments, at least 75%, 80%, 85%, 90%, 95%, 96%, 97%, 98%, or 99%, or 100 of the cells in the population of overexpressing cells, composition containing overexpressing cells, or composition enriched for overexpressing cells have been engineered to express (e.g., stably express) one or more transgene(s) containing GBA1. In some embodiments, at least 75%, 80%, 85%, 90%, 95%, 96%, 97%, 98%, or 99%, or 100 of the cells in the population of overexpressing cells, composition containing overexpressing cells, or composition enriched for overexpressing cells have been engineered to express (e.g., stably express) one or more transgene(s) containing a wildtype version of GBA1. In some embodiments, the cells have been introduced with one or more transgene(s) GBA1 by the methods described herein. In some embodiments, the cells have been introduced with one or more transgene(s) containing a wildtype version of GBA1 by the methods described herein. In some embodiments, the cells that have been introduced with the one or more transgene(s) containing GBA1 are less likely to cause, or contribute to, PD than the cells would be without the introducing. In some embodiments, the cells that have been introduced with the one or more transgene(s) containing a wildtype version of GBA1 are less likely to cause, or contribute to, PD than the cells would be without the introducing. In some embodiments, the cells that have been introduced with the one or more transgene(s) containing GBA1 are less likely to cause, or contribute to, GD than the cells would be without the introducing. In some embodiments, the cells that have been introduced with the one or more transgene(s) containing a wildtype version of GBA1 are less likely to cause, or contribute to, GD than the cells would be without the introducing. In some embodiments, the GBA1 is wildtype GBA1. In some embodiments, GBA1 is a functional GBA1 or a portion thereof.

A. Exemplary Features of Compositions

In some embodiments, the cells produced by any of the methods described herein comprise one or more stably integrated transgene(s) containing the GBA1 gene. In some embodiments, at least 10%, at least 20%, at least 30%, at least 40%, or at least 50% of the cells of any of the compositions described herein comprise one or more stably integrated transgene(s) containing the GBA1 gene. In some embodiments, at least 10% of the cells of any of the compositions described herein comprise one or more stably integrated transgene(s) containing the GBA1 gene. In some embodiments, at least 20% of the cells of any of the compositions described herein comprise one or more stably integrated transgene(s) containing the GBA1 gene. In some embodiments, at least 30% of the cells of any of the compositions described herein comprise one or more stably integrated transgene(s) containing the GBA1 gene. In some embodiments, at least 40% of the cells of any of the compositions described herein comprise one or more stably integrated transgene(s) containing the GBA1 gene. In some embodiments, at least 50% of the cells of any of the compositions described herein comprise one or more stably integrated transgene(s) containing the GBA1 gene. In some embodiments, at least 60% of the cells of any of the compositions described herein comprise one or more stably integrated transgene(s) containing the GBA1 gene. In some embodiments, at least 70% of the cells of any of the compositions described herein comprise one or more stably integrated transgene(s) containing the GBA1 gene. In some embodiments, at least 80% of the cells of any of the compositions described herein comprise one or more stably integrated transgene(s) containing the GBA1 gene. In some embodiments, at least 90% of the cells of any of the compositions described herein comprise one or more stably integrated transgene(s) containing the GBA1 gene.

In some embodiments, the cells produced by any of the methods described herein overexpress the GBA1 gene, such as compared to expression of the GBA1 gene in cells not produced by the methods described herein (i.e., cells not introduced with a DNA sequence encoding GBA1). In some embodiments, at least 10%, at least 20%, at least 30%, at least 40%, or at least 50% of the cells of any of the compositions described herein overexpress the GBA1 gene. In some embodiments, at least 10% of the cells of any of the compositions described herein overexpress the GBA1 gene. In some embodiments, at least 20% of the cells of any of the compositions described herein overexpress the GBA1 gene. In some embodiments, at least 30% of the cells of any of the compositions described herein overexpress the GBA1 gene. In some embodiments, at least 40% of the cells of any of the compositions described herein overexpress the GBA1 gene. In some embodiments, at least 50% of the cells of any of the compositions described herein overexpress the GBA1 gene. In some embodiments, at least 60% of the cells of any of the compositions described herein overexpress the GBA1 gene. In some embodiments, at least 70% of the cells of any of the compositions described herein overexpress the GBA1 gene. In some embodiments, at least 80% of the cells of any of the compositions described herein overexpress the GBA1 gene. In some embodiments, at least 90% of the cells of any of the compositions described herein overexpress the GBA1 gene.

In some embodiments, GBA1 is wildtype GBA1. In some embodiments, GBA1 is a functional GBA1 or a portion thereof. In some embodiments, GBA1 is a functional GBA1.

In some embodiments, the differentiated cells produced by any of the methods described herein are determined dopamine (DA) neuron progenitor cells. In some embodiments, the determined DA neuron progenitor cells are introduced with a DNA sequence encoding GBA1 (i.e., a GBA1-containing transgene) operably linked to a promoter. In some embodiments, the determined dopamine (DA) neuron progenitor cells comprise one or more stably integrated transgene(s) containing the wildtype GBA1 gene. In some embodiments the determined DA neuron progenitor cells introduced with the DNA sequence encoding the wildtype form of GBA1 overexpress the wild-type form of GBA1, such as compared to expression of GBA1 gene in cells not introduced with the DNA sequence.

In some embodiments, the overexpressing and/or differentiated cells (e.g., determined dopamine (DA) neuron progenitor cells) comprise a variant of human GBA1. In some embodiments, the variant is a single nucleotide polymorphism (SNP). In some embodiments, the SNP is rs76763715. In some of any such embodiments, the rs76763715 is a cytosine variant. In some of any such embodiments, the GBA1 comprising the SNP encodes a serine, rather than an asparagine, at amino acid position 370 (N370S). In some of any such embodiments, the SNP is rs421016. In some of any such embodiments, the rs421016 is a guanine variant. In some of any such embodiments, the GBA1 comprising the SNP encodes a proline, rather than a leucine, at amino acid position 444 (L444P). In some of any such embodiments, the SNP is rs2230288. In some of any such embodiments, the rs2230288 is a thymine variant. In some of any such embodiments, the GBA1 comprising the SNP encodes a lysine, rather than a glutamic acid, at position 326 (E326K).

In some embodiments, the differentiated cells produced by any of the methods described herein are capable of producing dopamine (DA). In some embodiments, the differentiated cells produced by any of the methods described herein do not produce or do not substantially produce norepinephrine (NE). Thus, in some embodiments, the differentiated cells produced by any of the methods described herein are capable of producing DA but do not produce or do not substantially produce NE

In some embodiments, the determined dopamine (DA) neuron progenitor cells express EN1. In some embodiments, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the total cells in the composition express EN1.

In some embodiments, the determined dopamine (DA) neuron progenitor cells express CORIN. In some embodiments, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the total cells in the composition express CORIN.

In some embodiments, the determined dopamine (DA) neuron progenitor cells express EN1 and CORIN. In some embodiments, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% of the total cells in the composition express EN1 and CORIN.

In some embodiments, less than 10% of determined dopamine (DA) neuron progenitor cells express TH. In some embodiments, the determined dopamine (DA) neuron progenitor cells express low levels of TH. In some embodiments, the determined dopamine (DA) neuron progenitor cells do not express TH. In some embodiments, the determined dopamine (DA) neuron progenitor cells express TH at lower levels than cells harvested or collected on other days. In some embodiments, some of the determined dopamine (DA) neuron progenitor cells express EN1 and CORIN and less than 10% of the cells express TH. In some embodiments, less than 10% of the determined dopamine (DA) neuron progenitor cells express TH, and at least about 20% of the cells express EN1. In some embodiments, less than 10% of the determined dopamine (DA) neuron progenitor cells express TH, and at least about 20% of the cells express CORIN. In some embodiments, less than 10% of the total determined dopamine (DA) neuron progenitor cells express TH, and at least about 20% of the cells express EN1 and CORIN.

In some embodiments, the differentiated cells produced by any of the methods described herein are dopamine (DA) neurons (e.g., midbrain fate DA neurons). In some embodiments, the midbrain fate dopamine (DA) neurons are FOXA2+/TH+ at the time of harvest. In some embodiments, the midbrain fate dopamine (DA) neurons are FOXA2+/TH+ by or on about day 18. In some embodiments, the midbrain fate dopamine (DA) neurons are FOXA2+/TH+ by or on about day 20. In some embodiments, the midbrain fate dopamine (DA) neurons are FOXA2+/TH+ by or on about day 25.

B. Compositions and Formulations

In some embodiments, the dose of cells comprising cells produced by any of the methods disclosed herein, is provided as a composition or formulation, such as a pharmaceutical composition or formulation. In some embodiments, the dose of cells comprises differentiated cells introduced with a DNA sequence encoding GBA1. In some embodiments, GBA1 is a functional GBA1 or a portion thereof. In some embodiments, GBA1 is a functional GBA1. In some embodiments, GBA1 is wildtype GBA1. In some embodiments, the dose of cells comprises differentiated cells introduced with a DNA sequence encoding the wildtype form of GBA1. Thus, in some embodiments, the dose of cells comprises overexpressing cells. In some embodiments, the dose of cells comprises cells produced by any of the methods described in Section II. In some embodiments, the dose of cells comprises cells produced by any of the methods described in Section III. In some embodiments, the dose of cells comprises cells produced by a combination of (1) any of the methods described in Section II, and (2) any of the methods described in Section III. In some embodiments, the dose of cells comprises cells produced by a process comprising (1) any of the methods of stably integrating one or more GBA1-containing transgenes described in Section II, and (2) any of the methods for differentiating cells described in Section III.

Such compositions can be used in accord with the provided methods, articles of manufacture, and/or with the provided compositions, such as in the prevention or treatment of diseases, conditions, and disorders, such as neurodegenerative disorders.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell or agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being prevented or treated with the cells or agents, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as carbidopa-levodopa (e.g., Levodopa), dopamine agonists (e.g., pramipexole, ropinirole, rotigotine, and apomorphine), MAO B inhibitors (e.g., selegiline, rasagiline, and safinamide), catechol O-methyltransferase (COMT) inhibitors (e.g., entacapone and tolcapone), anticholinergics (e.g., benztropine and trihexylphenidyl), amantadine, etc. In some embodiments, the agents or cells are administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

The formulation or composition may also be administered in combination with another form of treatment useful for the particular indication, disease, or condition being prevented or treated with the cells or agents, where the respective activities do not adversely affect one another. Thus, in some embodiments, the pharmaceutical composition is administered in combination with deep brain stimulation (DBS).

The pharmaceutical composition in some embodiments contains agents or cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

The agents or cells can be administered by any suitable means, for example, by stereotactic injection (e.g., using a catheter). In some embodiments, a given dose is administered by a single bolus administration of the cells or agent. In some embodiments, it is administered by multiple bolus administrations of the cells or agent, for example, over a period of months or years. In some embodiments, the agents or cells can be administered by stereotactic injection into the brain, such as in the striatum. In some embodiments, the agents or cells can be administered by stereotactic injection into the striatum, such as in the putamen.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

The cells or agents may be administered using standard administration techniques, formulations, and/or devices. Provided are formulations and devices, such as syringes and vials, for storage and administration of the compositions. With respect to cells, administration can be autologous. For example, non-pluripotent cells (e.g., fibroblasts) can be obtained from a subject, and administered to the same subject following reprogramming and differentiation. When administering a therapeutic composition (e.g., a pharmaceutical composition containing a genetically reprogrammed and/or differentiated cell or an agent that treats or ameliorates symptoms of a disease or disorder, such as a neurodegenerative disorder), it will generally be formulated in a unit dosage injectable form (solution, suspension, emulsion). Formulations include those for stereotactic administration, such as into the brain (e.g., the striatum).

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent or cells in a solvent, such as in admixture with a suitable carrier, diluent, or excipient such as sterile water, physiological saline, glucose, dextrose, or the like.

The formulations to be used for in vivo administration are generally sterile. Sterility may be readily accomplished, e.g., by filtration through sterile filtration membranes.

### V. Methods of Treatment

The present disclosure relates to methods of increasing the activity of GCase and/or increasing the expression of GBA1, such as in a subject having decreased expression and/or a variant of GBA1 associated with Parkinson's Disease (PD), and methods of lineage specific differentiation of pluripotent stem cells (PSCs), including embryonic stem (ES) cells and induced pluripotent stem cells (iPSCs) into DA neuron progenitor cells, including those in which activity of GCase and/or expression of the wildtype form of GBA1 has been increased, for use in treating neurodegenerative diseases. Specifically, the methods, compositions, and uses thereof provided herein contemplate differentiation of pluripotent stem cells into DA neuron progenitors cells and increased activity or GCase and/or increased expression of GBA1. In some embodiments, the cells have one or more GBA1 variant(s) associated with GD and/or PD, e.g., as described in Section II. Thus, in some embodiments, the methods, compositions, and uses thereof provided herein contemplate differentiation of pluripotent stem cells into DA neuron progenitors cells and increased activity or GCase and/or increased expression of GBA1, wherein one or more GBA1 variants is associated with PD, e.g., as described in Section II, for administration to subjects exhibiting a loss of a certain type of neuron, e.g., dopamine (DA) neurons, including Parkinson's disease. The methods, compositions, and uses thereof provided herein contemplate differentiation of pluripotent stem cells into DA neuron progenitors cells and increased activity of GCase and/or increase expression of GBA1, wherein one or more GBA1 variants is associated with PD, e.g., as described in Section II, for administration to subjects exhibiting the one or more GBA1 variants associated with PD. In some embodiments, the method increases the activity of GCase. In some embodiments, the method increases the expression of GBA1.

Specifically, provided herein is a method of treatment, comprising administering to a subject a therapeutically effective amount of a therapeutic composition, e.g., any composition as described in Section IV, wherein cells of the subject exhibit reduced activity of GCase and/or decreased expression of GBA1, as compared to reference cells (e.g., cells of a subject without Parkinson's Disease). In some embodiments, prior to administration of the therapeutic composition to the subject, cells of the subject exhibit reduced activity of GCase. In some embodiments, prior to administration of the therapeutic composition to the subject, cells of the subject exhibit decreased expression of GBA1. In some embodiments, prior to administration of the therapeutic composition to the subject, cells of the subject exhibit reduced activity of GCase and decreased expression of GBA1. In some embodiments, the reference cells do not exhibit reduced GCase activity. In some embodiments, the references cells do not exhibit reduced GBA1 expression. In some embodiments, the references cells do not exhibit reduced GCase activity or GBA1 expression. In some embodiments, the reference cells are from a subject without a LBD. In some embodiments, the reference cells are from a subject without PD. In some embodiments, the reference cells are from a subject without GD. In some embodiments, the reference cells are from a subject without PD or GD.

Also provided herein is a method of treatment, comprising administering to a subject a therapeutically effective amount of a therapeutic composition, e.g., any composition as described in Section IV, wherein the subject has reduced activity of the GCase enzyme. Also provided herein is a method of treatment, comprising administering to a subject a therapeutically effective amount of a therapeutic composition, e.g., any composition as described in Section IV, wherein the subject has a gene variant, e.g., SNP, associated with PD, such as a gene variant in human GBA1. In some embodiments, the subject has PD. In some embodiments, the subject has GD.

In some embodiments, the subject has a gene variant in the GBA1 gene, e.g., a rs76763715 SNP, that results in an N370S amino acid change due to the presence of a serine, rather than an asparagine, at amino acid position 370 in the expressed GCase enzyme (e.g., with respect to SEQ ID NO:1). In some embodiments, the subject has a gene variant in the GBA1 gene, e.g., a rs421016 SNP, that results in an L444P amino acid change due to the presence of a proline, rather than a leucine, at position 444 in the expressed GCase enzyme (e.g., with respect to SEQ ID NO:1). In some embodiments, the subject has a gene variant in the GBA1 gene, e.g., a rs2230288 SNP, that results in an E326K amino acid change due to the presence of a lysine, rather than a glutamic acid, at position 326 in the expressed GCase enzyme (e.g., with respect to SEQ ID NO:1).

Also provided herein is a method of treatment, comprising administering to a subject a therapeutically effective amount of a therapeutic composition, e.g., any composition as described in Section IV, wherein the subject has one or more gene variant(s), e.g., SNP, associated with GD, such as a gene variant in human GBA1. In some embodiments, the subject has a homozygous mutation (variant) in GBA1. In some embodiments, the subject has biallelic mutations in GBA1 (i.e., the mutations in each allele are not necessarily the same).

In some embodiments, a subject has a neurodegenerative disease. In some embodiments, the neurodegenerative disease comprises the loss of dopamine neurons in the brain. In some embodiments, the subject has lost dopamine neurons in the substantia nigra (SN). In some embodiments, the subject has lost dopamine neurons in the substantia nigra pas compacta (SNc). In some embodiments, the subject exhibits rigidity, bradykinesia, postural reflect impairment, resting tremor, or a combination thereof. In some embodiments, the subject exhibits abnormal [18F]-L-DOPA PET scan. In some embodiments, the subject exhibits [18F]-DG-PET evidence for a Parkinson's Disease Related Pattern (PDRP).

In some embodiments, the neurodegenerative disease is a Lewy body disease (LBD). In some embodiments, such as Parkinson's disease, Parkinson's disease demenetia, or dementia with Lewy bodies (DLB). In some embodiments, the neurodegenerative disease is Parkinsonism. In some embodiments, the neurodegenerative disease is Parkinson's disease dementia. In some embodiments, the neurodegenerative disease is DLB. In some embodiments, the neurodegenerative disease is Parkinson's disease. In some embodiments, the neurodegenerative disease is idiopathic Parkinson's disease. In some embodiments, the neurodegenerative disease is a familial form of Parkinson's disease. In some embodiments, the subject has mild Parkinson's disease. In some embodiments, the subject has a Movement Disorder Society-Unified Parkinson's Disease Rating Scale (MDS-UPDRS) motor score of less than or equal to 32. In some embodiments, the subject has Parkinson's Disease. In some embodiments, the subject has moderate or advanced Parkinson's disease. In some embodiments, the subject has mild Parkinson's disease. In some embodiments, the subject has a MDS-UPDRS motor score of between 33 and 60.

In some embodiments, cells of the subject have a GBA1 gene that includes a gene variant associated with PD. In some embodiments, the GBA1 variant encodes a serine, rather than an asparagine, at position 370 (N370S). In some embodiments, the GBA1 variant encodes an amino acid sequence comprising the amino acid sequence set forth in SEQ ID NO: 3. In some embodiments, the GBA1 variant encodes a proline, rather than a leucine, at position 444 (L444P). In some embodiments, the GBA1 variant encodes an amino acid sequence comprising the amino acid sequence set forth in SEQ ID NO: 4. In some embodiments, the GBA1 variant encodes a lysine, rather than a glutamic acid, at position 326 (E326K). In some embodiments, the GBA1 variant encodes an amino acid sequence comprising the amino acid sequence set forth in SEQ ID NO: 5. In some embodiments, the GBA1 variant encodes an amino acid sequence comprising the amino acid sequence set forth in any one of SEQ ID NOs: 3, 4, and 5.

In some embodiments, the subject has a GBA1 variant associated with PD that is a variant of rs76763715. In some embodiments, the subject has a GBA1 variant associated with PD that is a variant of rs76763715 that encodes a serine, rather than an asparagine, at position 370 (N370S). In some embodiments, the subject has a GBA1 variant associated with PD that is a a cytosine variant of rs76763715.

In some embodiments, the subject has a GBA1 variant associated with PD that is a variant of rs421016. In some embodiments, the subject has a GBA1 variant associated with PD that is a variant of rs421016 that encodes a proline, rather than a leucine, at position 444 (L444P). In some embodiments, the subject has a GBA1e variant associated with PD that is a guanine variant of rs421016.

In some embodiments, the subject has a GBA1 variant associated with PD that is a variant of rs2230288. In some embodiments, the subject has a GBA1 variant associated with PD that is a variant of rs2230288 that encodes a lysine, rather than a glutamic acid, at position 326 (E326K). In some embodiments, the subject has a GBA1 variant associated with PD that is a thymine variant of rs2230288.

In some embodiments, cells of the subject have a GBA1 gene that includes a gene variant associated with PD (e.g., with respect to SEQ ID NO:2). In some embodiments, the GBA1 variant encodes the amino acid sequence set forth in any one of SEQ ID NOS:6-15. In some embodiments, the GBA1 variant encodes a methionine, rather than a threonine, at position 369 (T369M) (e.g., with respect to SEQ ID NO:1). In some embodiments, the GBA1 variant encodes an amino acid sequence comprising the amino acid sequence set forth in SEQ ID NO: 6. In some embodiments, the GBA1 variant encodes a serine, rather than a glycine, at position 377 (G377S) (e.g., with respect to SEQ ID NO:1). In some embodiments, the GBA1 variant encodes an amino acid sequence comprising the amino acid sequence set forth in SEQ ID NO: 7. In some embodiments, the GBA1 variant encodes a histidine, rather than an aspartic acid, at position 409 (D409H) (e.g., with respect to SEQ ID NO: 1). In some embodiments, the GBA1 variant encodes an amino acid sequence comprising the amino acid sequence set forth in SEQ ID NO: 8. In some embodiments, the GBA1 variant encodes a tryptophan, rather than an arginine, at position 120 (R120W) (e.g., with respect to SEQ ID NO: 1). In some embodiments, the GBA1 variant encodes an amino acid sequence comprising the amino acid sequence set forth in SEQ ID NO: 9. In some embodiments, the GBA1 variant encodes a leucine, rather than a valine, at position 394 (V394L) (e.g., with respect to SEQ ID NO:1). In some embodiments, the GBA1 variant encodes an amino acid sequence comprising the amino acid sequence set forth in SEQ ID NO: 10. In some embodiments, the GBA1 variant encodes a histidine, rather than an arginine at position 496 (R496H) (e.g., with respect to SEQ ID NO:1). In some embodiments, the GBA1 variant encodes an amino acid sequence comprising the amino acid sequence set forth in SEQ ID NO: 11. In some embodiments, the GBA1 variant encodes a threonine, rather than a lysine, at position 178 (K178T) (e.g., with respect to SEQ ID NO:1). In some embodiments, the GBA1 variant encodes an amino acid sequence comprising the amino acid sequence set forth in SEQ ID NO: 12. In some embodiments, the GBA1 variant encodes a cysteine, rather than an arginine, at position 329 (R329C) (e.g., with respect to SEQ ID NO:1). In some embodiments, the GBA1 variant encodes an amino acid sequence comprising the amino acid sequence set forth in SEQ ID NO: 13. In some embodiments, the GBA1 variant encodes an arginine, rather than a leucine, at position 444 (L444R) (e.g., with respect to SEQ ID NO:1). In some embodiments, the GBA1 variant encodes an amino acid sequence comprising the amino acid sequence set forth in SEQ ID NO: 14. In some embodiments, the GBA1 variant encodes a serine, rather than an asparagine, at position 188 (N188S) (e.g., with respect to SEQ ID NO:1). In some embodiments, the GBA1 variant encodes an amino acid sequence comprising the amino acid sequence set forth in SEQ ID NO: 15.

In some embodiments, the therapeutic composition comprising cells, e.g., iPSCs, having increased expression of GBA1 (overexpressing cells), is administered to treat a subject having a disease or disorder associated with reduced GCase activity. In some embodiments, the therapeutic composition comprising cells, e.g., iPSCs, having increased expression of GBA1 (overexpressing cells), is administered to treat a neurodegenerative disease. In some embodiments, the neurodegenerative disease is a LBD. In some embodiments, the neurodegenenerative disease is Parkinson's disease dementia. In some embodiments, the neurodegenenerative disease is Parkinson's DLB. In some embodiments, the neurodegenerative disease is PD. In some embodiments, the neurodegenative disease is GD. In some embodiments, the therapeutic composition comprising cells, e.g., iPSCs, having increased expression of the wildtype form of the GBA1 (overexpressing cells), is administered to treat a neurodegenerative disease, e.g., PD, using cells that exhibit increased expression of (i.e., overexpress) the wildtype form of GBA1. By administering a therapeutic composition comprising cells, e.g., iPSCs, exhibiting increased expression of (i.e., overexpressing) the wildtype form of GBA1, the risk of recurrence of the neurodegenerative disease, e.g., is reduced.

In some embodiments, a dose of cells overexpressing the wildtype form of GBA1, e.g., as described in Section II, that have been neurally differentiated, e.g., as described in Section III, is administered to subjects in accord with the provided methods, and/or with the provided articles of manufacture, and/or with the provided compositions, e.g., as described in Section IV. In some embodiments, the dose of cells is a dose of cells, e.g., DA neuron progenitor cells, overexpressing the GBA1, e.g., as described in Section II, that are differentiated from pluripotent stem cells, e.g., as described in Section III. In some embodiments, GBA1 is wildtype GBA1. In some embodiments, GBA1 is a functional GBA1 or a portion thereof. In some embodiments, GBA1 is a functional GBA1. In some embodiments, the dose of cells is differentiated from pluripotent stem cells, e.g., as described in Section III. In some embodiments, the dose of cells is a dose of cells, e.g., DA neuron progenitor cells, overexpressing the wildtype form of GBA1, e.g., as described in Section II, that are differentiated from pluripotent stem cells, e.g., as described in Section III. In some embodiments, the dose of cells is a dose of a composition of cells, e.g., as described in Section IV.

In some embodiments, the size or timing of the doses is determined as a function of the particular disease or condition in the subject. In some cases, the size or timing of the doses for a particular disease in view of the provided description may be empirically determined.

In some embodiments, the dose of cells is administered to the striatum (e.g., putamen) of the subject. In some embodiments, the dose of cells is administered to one hemisphere of the subject's striatum (e.g., putamen). In some embodiments, the dose of cells is administered to both hemispheres of the subject's striatum (e.g., putamen).

In some embodiments, the dose of cells comprises between at or about 250,000 cells per hemisphere and at or about 20 million cells per hemisphere, between at or about 500,000 cells per hemisphere and at or about 20 million cells per hemisphere, between at or about 1 million cells per hemisphere and at or about 20 million cells per hemisphere, between at or about 5 million cells per hemisphere and at or about 20 million cells per hemisphere, between at or about 10 million cells per hemisphere and at or about 20 million cells per hemisphere, between at or about 15 million cells per hemisphere and at or about 20 million cells per hemisphere, between at or about 250,000 cells per hemisphere and at or about 15 million cells per hemisphere, between at or about 500,000 cells per hemisphere and at or about 15 million cells per hemisphere, between at or about 1 million cells per hemisphere and at or about 15 million cells per hemisphere, between at or about 5 million cells per hemisphere and at or about 15 million cells per hemisphere, between at or about 10 million cells per hemisphere and at or about 15 million cells per hemisphere, between at or about 250,000 cells per hemisphere and at or about 10 million cells per hemisphere, between at or about 500,000 cells per hemisphere and at or about 10 million cells per hemisphere, between at or about 1 million cells per hemisphere and at or about 10 million cells per hemisphere, between at or about 5 million cells per hemisphere and at or about 10 million cells per hemisphere, between at or about 250,000 cells per hemisphere and at or about 5 million cells per hemisphere, between at or about 500,000 cells per hemisphere and at or about 5 million cells per hemisphere, between at or about 1 million cells per hemisphere and at or about 5 million cells per hemisphere, between at or about 250,000 cells per hemisphere and at or about 1 million cells per hemisphere, between at or about 500,000 cells per hemisphere and at or about 1 million cells per hemisphere, or between at or about 250,000 cells per hemisphere and at or about 500,00 cells per hemisphere.

In some embodiments, the dose of cells is between at or about 1 million cells per hemisphere and at or about 30 million cells per hemisphere. In some embodiments, the dose of cells is between at or about 5 million cells per hemisphere and at or about 20 million cells per hemisphere. In some embodiments, the dose of cells is between at or about 10 million cells per hemisphere and at or about 15 million cells per hemisphere.

In some embodiments, the dose of cells is between about about 3×10⁶ cells/hemisphere and 15×10⁶ cells/hemisphere. In some embodiments, the dose of cells is about about 3×10⁶ cells/hemisphere. In some embodiments, the dose of cells is about about 4×10⁶ cells/hemisphere. In some embodiments, the dose of cells is about about 5×10⁶ cells/hemisphere. In some embodiments, the dose of cells is about about 6×10⁶ cells/hemisphere. In some embodiments, the dose of cells is about about 7×10⁶ cells/hemisphere. In some embodiments, the dose of cells is about about 8×10⁶ cells/hemisphere. In some embodiments, the dose of cells is about about 9×10⁶ cells/hemisphere. In some embodiments, the dose of cells is about about 10×10⁶ cells/hemisphere. In some embodiments, the dose of cells is about about 11×10⁶ cells/hemisphere. In some embodiments, the dose of cells is about about 12×10⁶ cells/hemisphere. In some embodiments, the dose of cells is about about 13×10⁶ cells/hemisphere. In some embodiments, the dose of cells is about about 14×10⁶ cells/hemisphere. In some embodiments, the dose of cells is about about 15×10⁶ cells/hemisphere.

In some embodiments, the dose of cells is about about 5×10⁶ cells in each putamen. In some embodiments, the dose of cells is about about 10×10⁶ cells in each putamen.

In some embodiments, the number of cells administered to the subject is between about 0.25×10⁶ total cells and about 20×10⁶ total cells, between about 0.25×10⁶ total cells and about 15×10⁶ total cells, between about 0.25×10⁶ total cells and about 10×10⁶ total cells, between about 0.25×10⁶ total cells and about 5×10⁶ total cells, between about 0.25×10⁶ total cells and about 1×10⁶ total cells, between about 0.25×10⁶ total cells and about 0.75×10⁶ total cells, between about 0.25×10⁶ total cells and about 0.5×10⁶ total cells, between about 0.5×10⁶ total cells and about 20×10⁶ total cells, between about 0.5×10⁶ total cells and about 15×10⁶ total cells, between about 0.5×10⁶ total cells and about 10×10⁶ total cells, between about 0.5×10⁶ total cells and about 5×10⁶ total cells, between about 0.5×10⁶ total cells and about 1×10⁶ total cells, between about 0.5×10⁶ total cells and about 0.75×10⁶ total cells, between about 0.75×10⁶ total cells and about 20×10⁶ total cells, between about 0.75×10⁶ total cells and about 15×10⁶ total cells, between about 0.75×10⁶ total cells and about 10×10⁶ total cells, between about 0.75×10⁶ total cells and about 5×10⁶ total cells, between about 0.75×10⁶ total cells and about 1×10⁶ total cells, between about 1×10⁶ total cells and about 20×10⁶ total cells, between about 1×10⁶ total cells and about 15×10⁶ total cells, between about 1×10⁶ total cells and about 10×10⁶ total cells, between about 1×10⁶ total cells and about 5×10⁶ total cells, between about 5×10⁶ total cells and about 20×10⁶ total cells, between about 5×10⁶ total cells and about 15×10⁶ total cells, between about 5×10⁶ total cells and about 10×10⁶ total cells, between about 10×10⁶ total cells and about 20×10⁶ total cells, between about 10×10⁶ total cells and about 15×10⁶ total cells, or between about 15×10⁶ total cells and about 20×10⁶ total cells.

In certain embodiments, the cells, or individual populations of sub-types of cells, are administered to the subject at a range of about 5 million cells per hemisphere to about 20 million cells per hemisphere or any value in between these ranges. Dosages may vary depending on attributes particular to the disease or disorder and/or patient and/or other treatments.

In some embodiments, the patient is administered multiple doses, and each of the doses or the total dose can be within any of the foregoing values. In some embodiments, the dose of cells comprises the administration of from or from about 5 million cells per hemisphere to about 20 million cells per hemisphere, each inclusive.

In some embodiments, the dose of cells, e.g., overexpressing cells, is administered to the subject as a single dose or is administered only one time within a period of two weeks, one month, three months, six months, 1 year or more.

In the context of stem cell transplant, administration of a given “dose” encompasses administration of the given amount or number of cells as a single composition and/or single uninterrupted administration, e.g., as a single injection or continuous infusion, and also encompasses administration of the given amount or number of cells as a split dose or as a plurality of compositions, provided in multiple individual compositions or infusions, over a specified period of time, such as a day. Thus, in some contexts, the dose is a single or continuous administration of the specified number of cells, given or initiated at a single point in time. In some contexts, however, the dose is administered in multiple injections or infusions in a single period, such as by multiple infusions over a single day period.

Thus, in some aspects, the cells of the dose are administered in a single pharmaceutical composition. In some embodiments, the cells of the dose are administered in a plurality of compositions, collectively containing the cells of the dose.

In some embodiments, cells of the dose may be administered by administration of a plurality of compositions or solutions, such as a first and a second, optionally more, each containing some cells of the dose. In some aspects, the plurality of compositions, each containing a different population and/or sub-types of cells, are administered separately or independently, optionally within a certain period of time.

In some embodiments, the administration of the composition or dose, e.g., administration of the plurality of cell compositions, involves administration of the cell compositions separately. In some aspects, the separate administrations are carried out simultaneously, or sequentially, in any order.

In some embodiments, the subject receives multiple doses, e.g., two or more doses or multiple consecutive doses, of the cells. In some embodiments, two doses are administered to a subject. In some embodiments, multiple consecutive doses are administered following the first dose, such that an additional dose or doses are administered following administration of the consecutive dose. In some aspects, the number of cells administered to the subject in the additional dose is the same as or similar to the first dose and/or consecutive dose. In some embodiments, the additional dose or doses are larger than prior doses.

In some aspects, the size of the first and/or consecutive dose is determined based on one or more criteria such as response of the subject to prior treatment, e.g., disease stage and/or likelihood or incidence of the subject developing adverse outcomes, e.g., dyskinesia.

In some embodiments, the dose of cells is generally large enough to be effective in improving symptoms of the disease.

In some embodiments, the cells are administered at a desired dosage, which in some aspects includes a desired dose or number of cells or cell type(s) and/or a desired ratio of cell types. In some embodiments, the dosage of cells is based on a desired total number (or number per kg of body weight) of cells in the individual populations or of individual cell types (e.g., TH+ or TH−). In some embodiments, the dosage is based on a combination of such features, such as a desired number of total cells, desired ratio, and desired total number of cells in the individual populations.

Thus, in some embodiments, the dosage is based on a desired fixed dose of total cells and a desired ratio, and/or based on a desired fixed dose of one or more, e.g., each, of the individual sub-types or sub-populations.

In particular embodiments, the numbers and/or concentrations of cells refer to the number of TH-negative cells. In particular embodiments, the numbers and/or concentrations of cells refer to the number of TH-positive cells. In other embodiments, the numbers and/or concentrations of cells refer to the number or concentration of all cells administered.

In some embodiments, the cells are administered at a desired dosage, which in some aspects includes a desired dose or number of cells or cell type(s) and/or a desired ratio of cell types. Thus, the dosage of cells in some embodiments is based on a total number of cells and a desired ratio of the individual populations or sub-types In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

Thus, in some embodiments, the dosage is based on a desired fixed dose of total cells and a desired ratio, and/or based on a desired fixed dose of one or more, e.g., each, of the individual sub-types or sub-populations.

In particular embodiments, the numbers and/or concentrations of cells refer to the number of TH-negative cells. In particular embodiments, the numbers and/or concentrations of cells refer to the number of TH-positive cells. In other embodiments, the numbers and/or concentrations of cells refer to the number or concentration of all cells administered.

In some aspects, the size of the dose is determined based on one or more criteria such as response of the subject to prior treatment, e.g., disease type and/or stage, and/or likelihood or incidence of the subject developing toxic outcomes, e.g., dyskinesia.

### VI. Articles of Manufacture and Kits

Also provided are articles of manufacture, systems, apparatuses, and kits useful in performing the provided methods.

Also provided are articles of manufacture, including: (i) a deoxyribonucleic acid (DNA) sequence encoding GBA1 operably linked to a promoter; and (ii) instructions for use of the DNA sequence for performing any methods described herein.

Also provided are articles of manufacture, including: (i) a transposase or a nucleic acid sequence encoding a transposase; and (ii) instructions for use of the DNA sequence for performing any methods described herein.

Also provided are articles of manufacture, including: (i) a deoxyribonucleic acid (DNA) sequence encoding GBA1 operably linked to a promoter; (ii) a transposase or a nucleic acid sequence encoding a transposase; and (iii) instructions for use of the DNA sequence for performing any methods described herein.

In some of any such embodiments, the transposase is a Class II transposase. In some embodiments, wherein the transposase is selected from the group consisting of: Sleeping Beauty, piggyBac, TcBuster, Frog Prince, Tol2, Tcl/mariner, or a derivative thereof having transposase activity. In some embodiments, the transposase is Sleeping Beauty, PiggyBac, or TcBuster. In some embodiments, the transposase is Sleeping Beauty. In some embodiments, the transposase is PiggyBac. In some embodiments, the transposase is TcBuster.

In some embodiments, the DNA sequence encoding GBA1 is positioned between inverted terminal repeat (ITRs).

In some embodiments, the promoter is selected from the group consisting of: ubiquitin C (UBC promoter) cytomegalovirus (CMV) promoter, phosphoglycerate kinase (PGK) promoter, CMV early enhancer/chicken b actin (CAG) promoter, glial fibrilary acidic protein (GFAP) promoter, synapsin-1 promoter, and Neuron Specific Enolase (NSE) promoter. In some embodiments, the promoter is a PGK promoter or a UBC promoter. In some embodiments, the promoter is a PGK promoter. In some embodiments, the promoter is a UBC promoter.

In some embodiments, the DNA sequence encoding GBA1 is part of a plasmid. In some embodiments, the nucleic acid encoding a transposase is part of a plasmid. In some embodiments, the plasmid containing the DNA sequence encoding GBA1 and the plasmid containing the nucleic acid sequence encoding the transposase are different plasmids. In some embodiments, the plasmid containing the DNA sequence encoding GBA1 and the plasmid containing the nucleic acid sequence encoding the transposase are the same plasmid.

Also provided are articles of manufacture, including: (i) one or more reagents for differentiation of pluripotent stem cells into floor plate midbrain progenitor cells, determined dopaminergic (DA) neuron progenitor cells, and/or dopaminergic (DA) neurons; and (ii) instructions for use of the one or more reagents for performing any methods described herein.

Also provided are articles of manufacture, including: (i) a deoxyribonucleic acid (DNA) sequence encoding GBA1 operably linked to a promoter; (ii) one or more reagents for differentiation of pluripotent stem cells into floor plate midbrain progenitor cells, determined dopamine (DA) neuron progenitor cells, and/or dopamine (DA) neurons; and instructions for use of the DNA sequence and the one or more reagents for performing any methods described herein.

Also provided are articles of manufacture, including: (i) a transposase or a nucleic acid sequence encoding a transposase; (ii) one or more reagents for differentiation of pluripotent stem cells into floor plate midbrain progenitor cells, determined dopamine (DA) neuron progenitor cells, and/or dopamine (DA) neurons; and instructions for use of the transposase or the nucleic acid sequence encoding the transposase and the one or more reagents for performing any methods described herein.

Also provided are articles of manufacture, including: (i) a deoxyribonucleic acid (DNA) sequence encoding GBA1 operably linked to a promoter; (ii) a transposase or a nucleic acid sequence encoding a transposase; (iii) one or more reagents for differentiation of pluripotent stem cells into floor plate midbrain progenitor cells, determined dopamine (DA) neuron progenitor cells, and/or dopamine (DA) neurons; and instructions for use of the DNA sequence, the transpossae or nucleic acid sequence encoding a transposase, and the one or more reagents for performing any methods described herein.

In some of any such embodiments, the reagent for differentiation is or includes a small molecule, capable of inhibiting TGF-β/activin-Nodal signaling. In some of any such embodiments, the reagent for differentiation is or includes SB431542. In some of any such embodiments, the reagent for differentiation is or includes a small molecule, capable of activating SHH signaling. In some of any such embodiments, the reagent for activating SHH signaling is or includes SHH. In some of any such embodiments, the reagent for activating SHH signaling is or includes purmorphamine. In some of any such embodiments, the reagent for activating SHH signaling is or includes SHH and purmorphamine. In some of any such embodiments, the reagent for differentiation is or includes a small molecule, capable of inhibiting BMP signaling. In some of any such embodiments, the reagent for inhibiting BMP signaling is LDN193189. In some of any such embodiments, the reagent for differentiation is or includes a small molecule, capable of inhibiting GSK3β signaling. In some of any of such embodiments, the reagent is or includes CHIR99021. In some of any of such embodiments, the reagent for differentiation is or includes one or more of BDNF, GDNF, dbcAMP, ascorbic acid, TGFβ3, and DAPT. The reagents in the kit in one embodiment may be in solution, may be frozen, or may be lyophilized.

Also provided are articles of manufacture, including (i) any composition described herein; and (ii) instructions for administering the composition to a subject.

In some embodiments, the articles of manufacture or kits include one or more containers, typically a plurality of containers, packaging material, and a label or package insert on or associated with the container or containers and/or packaging, generally including instructions for use, e.g., instructions for reagents for differentiation of pluripotent cells, e.g., differentiation of iPSCs into floor plate midbrain progenitor cells, determined dopamine (DA) neuron progenitor cells, and/or dopamine (DA) neurons, and instructions to carry out any of the methods provided herein. In some aspects, the provided articles of manufacture contain reagents for differentiation and/or maturation of cells, for example, at one or more steps of the manufacturing process, such as any reagents described in any steps of Sections III and IV.

Also provided are articles of manufacture and kits containing overexpressing and differentiated cells, such as those generated using the methods provided herein, and optionally instructions for use, for example, instructions for administering. In some embodiments, the instructions provide directions or specify methods for assessing if a subject, prior to receiving a cell therapy, is likely or suspected of being likely to respond and/or the degree or level of response following administration of differentiated cells for treating a disease or disorder. In some aspects, the articles of manufacture can contain a dose or a composition of overexpressing and differentiated cells.

The articles of manufacture provided herein contain packaging materials. Packaging materials for use in packaging the provided materials are well known to those of skill in the art. See, for example, U.S. Pat. Nos. 5,323,907, 5,052,558 and 5,033,252, each of which is incorporated herein in its entirety. Examples of packaging materials include, but are not limited to, blister packs, bottles, tubes, inhalers, pumps, bags, vials, containers, syringes, disposable laboratory supplies, e.g., pipette tips and/or plastic plates, or bottles. The articles of manufacture or kits can include a device so as to facilitate dispensing of the materials or to facilitate use in a high-throughput or large-scale manner, e.g., to facilitate use in robotic equipment. Typically, the packaging is non-reactive with the compositions contained therein.

In some embodiments, the reagents and/or cell compositions are packaged separately. In some embodiments, each container can have a single compartment. In some embodiments, other components of the articles of manufacture or kits are packaged separately, or together in a single compartment.

### VII. EXEMPLARY EMBODIMENTS

Among the provided embodiments are:

1. A method of increasing expression of GBA1 in a cell, the method comprising:

(i) introducing, into a pluripotent stem cell, a deoxyribonucleic acid (DNA) sequence encoding GBA1 operably linked to a promoter, wherein the DNA sequence is positioned between inverted terminal repeats and is capable of integrating into DNA in the cell; and

(ii) introducing, into the cell, a transposase or a nucleic acid sequence encoding a transposase,

wherein the introducing in (i) and (ii) results in integration of the DNA sequence encoding GBA1 into the genome of the cell.

2. The method of embodiment 1, wherein the cell comprises a variant of GBA1 associated with Parkinson's disease.
3. A method of increasing expression of GBA1 in a cell, the method comprising:

(i) introducing, into a pluripotent stem cell, a deoxyribonucleic acid (DNA) sequence encoding GBA1 operably linked to a promoter, wherein the DNA sequence is positioned between inverted terminal repeats and is capable of integrating into DNA in the cell; and

(ii) introducing, into the cell, a transposase or a nucleic acid sequence encoding a transposase, wherein:

the cell comprises a variant of GBA1 associated with Parkinson's disease, and

the introducing in (i) and (ii) results in integration of the DNA sequence encoding GBA1 into the genome of the cell.

4. The method of any one of embodiments 1-3, wherein the DNA sequence encoding GBA1 is part of a plasmid.
5. The method of any one of embodiments 1-4, wherein the transposase is a Class II transposase.
6. The method of any one of embodiments 1-5, wherein the transposase is selected from the group consisting of: Sleeping Beauty, piggyBac, TcBuster, Frog Prince, Tol2, Tcl/mariner, or a derivative thereof having transposase activity.
7. The method of any one of embodiments 1-6, wherein the transposase is Sleeping Beauty, PiggyBac, or TcBuster.
8. The method of any one of embodiments 1-7, wherein the transposase is TcBuster.
9. The method of any one of embodiments 1-8, wherein the promoter is selected from the group consisting of: ubiquitin C (UBC) promoter, cytomegalovirus (CMV) promoter, phosphoglycerate kinase (PGK) promoter, CMV early enhancer/chicken b actin (CAG) promoter, glial fibrilary acidic protein (GFAP) promoter, synapsin-1 promoter, and Neuron Specific Enolase (NSE) promoter.
10. The method of any one of embodiments 1-9, wherein the promoter is a PGK promoter.
11. The method of any one of embodiments 1-10, wherein the nucleic acid sequence encoding the transposase and/or the DNA sequence encoding GBA1 are introduced into the cell by electrotransfer, optionally electroporation or nucleofection; chemotransfer; or nanoparticles.
12. The method of any one of embodiments 1-11, wherein the method comprises introducing, into the cell, a nucleic acid encoding a transposase.
13. The method of any one of embodiments 1-12, wherein the nucleic acid encoding a transposase is part of a plasmid.
14. The method of embodiment 13, wherein the nucleic acid encoding a transposase is ribonucleic acid (RNA).
15. The method of embodiment 13, wherein the nucleic acid encoding a transposase is DNA.
16. The method of any one of embodiments 1-15, wherein the plasmid containing the DNA sequence encoding GBA1 and the plasmid containing the nucleic acid sequence encoding the transposase are different plasmids.
17. The method of any one of embodiments 1-15, wherein the plasmid containing the DNA sequence encoding GBA1 and the plasmid containing the nucleic acid sequence encoding the transposase are the same plasmid.
18. The method of any of embodiments 1-11, wherein the method comprises introducing, into the cell, a transposase.
19. The method of any one of embodiments 1-18, wherein (i) the DNA sequence encoding GBA1 and the (ii) the transposase or the nucleic acid sequence encoding the transposase are introduced into the cell at the same time.
20. The method of any one of embodiments 1-19, wherein the DNA sequence encoding GBA1 is not introduced into an exon.
21. The method of any one of embodiments 1-20, wherein the DNA sequence encoding GBA1 is introduced into an intron.
22. The method of anyone of embodiments 1-21, wherein the cell exhibits decreased expression of GBA1 prior to being introduced with the DNA sequence encoding GBA1 and the transposase or the nucleic acid sequence encoding the transposase, as compared to a reference cell, optionally as compared to a cell from a subject without Parkinson's Disease.
23. The method of any one of embodiments 1-22, wherein the cell exhibits reduced activity of the β-Glucocerebrosidase (GCase) enzyme encoded by GBA1 prior to being introduced with the DNA sequence encoding GBA1 and the transposase or the nucleic acid sequence encoding the transposase, as compared to a reference cell, optionally as compared to a cell from a subject without Parkinson's Disease.
24. The method of any one of embodiments 1-23, wherein GBA1 is human GBA1.
25. The method of any one of embodiments 1-24, wherein the DNA sequence encoding GBA1 comprises a coding region of the sequence set forth in SEQ ID NO:2 or a codon-optimized version of a coding region of the sequence set forth in SEQ ID NO:2.
26. The method of any one of embodiments 1-25, wherein the DNA encoding GBA1 encodes an amino acid comprising the amino acid sequence set forth in SEQ ID NO:1.
27. The method of any one of embodiments 2-26, wherein the variant of GBA1 comprises a single nucleotide polymorphism (SNP) that is associated with Parkinson's disease.
28. The method of embodiment 27, wherein the SNP is rs76763715.
29. The method of embodiment 28, wherein the rs76763715 is a cytosine variant.
30. The method of any one of embodiments 27-29, wherein the variant of GBA1 comprising a SNP encodes a serine, rather than an asparagine, at amino acid position 370 (N370S).
31. The method of embodiment 29 or embodiment 30, wherein the wild-type form of GBA1 comprises a thymine instead of the cytosine variant.
32. The method of embodiment 27, wherein the SNP is rs421016.
33. The method of embodiment 32, wherein the rs421016 is a guanine variant.
34. The method of any one of embodiments 27, 32, and 33, wherein the variant of GBA1 comprising the SNP encodes a proline, rather than a leucine, at amino acid position 444 (L444P).
35. The method of embodiment 33 or embodiment 34, wherein the wild-type form of GBA1 comprises an adenine instead of the guanine variant.
36. The method of embodiment 27, wherein the SNP is rs2230288.
37. The method of embodiment 36, wherein the rs2230288 is a thymine variant.
38. The method of any one of embodiments 27, 36, and 37, wherein the variant of GBA1 comprising the SNP encodes a lysine, rather than a glutamic acid, at position 326 (E326K).
39. The method of embodiment 37 or embodiment 38, wherein the wild-type form of GBA1 comprises a cytosine instead of the thymine variant.
40. The method of any one of embodiments 1-39, wherein the cell is an induced pluripotent stem cell (iPSC).
41. The method of embodiment 40, wherein the iPSC is artificially derived from a non-pluripotent cell from a subject.
42. The method of embodiment 41, wherein the non-pluripotent cell is a fibroblast.
43. The method of embodiment 41 or embodiment 42, wherein the subject has Parkinson's disease or Gaucher's disease.
44. The method of any of embodiments 41-43, wherein the subject has Parkinson's disease.
45. The method of any one of embodiments 1-44, wherein, after the integration of the DNA sequence encoding GBA1 into the DNA of the cell, the method further comprises determining the location of the integrated DNA sequence in the genome of the cell.
46. A method of differentiating neural cells, the method comprising:

(a) performing a first incubation comprising culturing the cells produced by the method of any one of embodiments 1-45 in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and

(b) performing a second incubation comprising culturing cells of the spheroid in a substrate-coated culture vessel under conditions to neurally differentiate the cells.

47. The method of embodiment 46, wherein the cells are exposed to the inhibitor of TGF-β/activin-Nodal signaling up to a day at or before day 7.
48. The method of embodiment 46 or embodiment 47, wherein the cells are exposed to the inhibitor of TGF-β/activin-Nodal beginning at day 0 and through day 6, inclusive of each day.
49. The method of any one of embodiments 46-48, wherein the cells are exposed to the at least one activator of SHH signaling up to a day at or before day 7.
50. The method of any one of embodiments 46-49, wherein the cells are exposed to the at least one activator of SHH signaling beginning at day 0 and through day 6, inclusive of each day.
51. The method of any one of embodiments 46-50, wherein the cells are exposed to the inhibitor of BMP signaling up to a day at or before day 11.
52. The method of any one of embodiments 46-51, wherein the cells are exposed to the inhibitor of BMP signaling beginning at day 0 and through day 10, inclusive of each day.
53. The method of any one of embodiments 46-52, wherein the cells are exposed to the inhibitor of GSK3β signaling up to a day at or before day 13.
54. The method of any one of embodiments 46-53, wherein the cells are exposed to the inhibitor of GSK3b signaling beginning at day 0 and through day 12, inclusive of each day.
55. The method of any one of embodiments 46-54, wherein culturing the cells under conditions to neurally differentiate the cells comprises exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch signaling.
56. The method of embodiment 55, wherein the cells are exposed to BAGCT and the inhibitor of Notch signaling beginning on day 11.
57. The method of embodiment 55 or embodiment 56, wherein the cells are exposed to BAGCT and the inhibitor of Notch signaling beginning at day 11 and until harvest of the neurally differentiated cells.
58. The method of any one of embodiments 46-57, wherein the inhibitor of TGF-β/activin-Nodal signaling is SB431542.
59. The method of any one of embodiments 46-58, wherein the at least one activator of SHH signaling is SHH or purmorphamine.
60. The method of any one of embodiments 46-59, wherein the inhibitor of BMP signaling is LDN193189.
61. The method of any one of embodiments 46-60, wherein the inhibitor of GSK3β signaling is CHIR99021.
62. The method of any of embodiments 1-61, wherein the neurally differentiated cells are harvested between about day 18 and about day 25, optionally on day 18 or day 20.
63. The method of any of embodiments 1-62, wherein the neurally differentiated cells are harvested on about day 20.
64. The method of any of embodiments 1-63, wherein the neurally differentiated cells are cryopreserved.
65. The method of any one of embodiments 1-64, further comprising cryopreserving the neurally differentiated cells.
66. The method of embodiment 65, wherein the cryopreserving comprises formulating the neurally differentiated cell with a cryoprotectant.
67. A pluripotent stem cell produced by the method of any of embodiments 1-45.
68. A neurally differentiated cell produced by the method of any of embodiments 46-66.
69. A pluripotent stem cell (PSC) comprising an exogenous deoxyribonucleic acid (DNA) sequence encoding GBA1 integrated into its genome.
70. A neurally differentiated cell comprising an exogenous deoxyribonucleic acid (DNA) sequence encoding GBA1 integrated into its genome.
71. A microglial cell comprising an exogenous deoxyribonucleic acid (DNA) sequence encoding GBA1 integrated into its genome.
72. A macrophage comprising an exogenous deoxyribonucleic acid (DNA) sequence encoding GBA1 integrated into its genome.
73. A hematopoietic stem cell (HSC) comprising an exogenous deoxyribonucleic acid (DNA) sequence encoding GBA1 integrated into its genome.
74. The cell of any of embodiments 69-73, wherein the DNA sequence is operably linked to a promoter.
75. The cell of any of embodiments 69-74, wherein the DNA sequence was integrated into the genome of the cell by a transposon-based system.
76. The pluripotent stem cell of any one of embodiments 69, 74, and 75, wherein the pluripotent stem cell is an induced pluripotent stem cell.
77. The neurally differentiated cell of any one of embodiments 70, 74, and 75, wherein the neurally differentiated cell expresses EN1 and CORIN.
78. The neurally differentiated cell of any one of embodiments 70, 74, 75, and 77, wherein the neurally differentiated cell is a committed dopaminergic precursor cells.
79. The cell of any one of embodiments 69-78, wherein the cell comprises a variant of GBA1 associated with Parkinson's disease.
80. The cell of any one of embodiments 67-79, wherein the cell is formulated with a cryoprotectant.
81. A therapeutic composition comprising the cell(s) of any one of embodiments 68, 70-75, and 77-80.
82. The therapeutic composition of embodiment 81, wherein cells of the composition express EN1 and CORIN and less than 10% of the total cells in the composition express TH.
83. The therapeutic composition of embodiment 81 or embodiment 82, wherein less than 5% of the total cells in the composition express TH.
84. The therapeutic composition of any one of embodiments 81-83, comprising a cryoprotectant.
85. A method of treatment, comprising administering to a subject a therapeutically effective amount of the therapeutic composition of any one of embodiments 81-84.
86. The method of embodiment 85, wherein the cells of the therapeutic composition are autologous to the subject.
87. The method of embodiment 85 or embodiment 86, wherein the subject has a disease or disorder associated with reduced GCase activity.
88. The method of any one of embodiments 85-87, wherein the subject has Gaucher's disease.
89. The method of any one of embodiments 85-87, wherein the subject has a Lewy body disease (LBD).
90. The method of embodiment 89, wherein the LBD is Parkinson's disease, Parkinson's disease dementia, or dementia with Lewy bodies (DLB).
91. The method of any one of embodiments 85-87, 89, and 90, wherein the subject has Parkinson's disease.
92. The method of any one of embodiments 85-91, wherein the administering comprises delivering cells of a composition by stereotactic injection.
93. The method of any one of embodiments 85-92, wherein the administering comprises delivering cells of a composition through a catheter.
94. The method of embodiment 92 or embodiment 93, wherein the cells are delivered to the striatum of the subject.
95. Use of the composition of any one of embodiments 81-84, for the treatment of a disease or disorder associated with reduced GCase activity.
96. Use of the composition of any one of embodiments 81-84, for the treatment of Gaucher's disease.
97. Use of the composition of any one of embodiments 81-84, for the treatment of a Lewy body disease (LBD).
98. The use of embodiment 97, wherein the LBD is Parkinson's disease, Parkinson's disease dementia, or dementia with Lewy bodies (DLB).
99. Use of the composition of any one of embodiments 81-84, for the treatment of Parkinson's disease.
100. A transposon-based system for increasing expression of GBA1 in a cell, the system comprising:

(i) a deoxyribonucleic acid (DNA) sequence encoding GBA1, wherein the DNA sequence is positioned between at least two inverted terminal repeats and is capable of integrating into DNA in a cell; and

(ii) a transposase or a nucleic acid sequence encoding a transposase,

wherein the cell exhibits (i) reduced activity of the β-Glucocerebrosidase (GCase) enzyme encoded by GBA1 and/or (ii) reduced expression of GBA1 prior to being introduced with the DNA sequence encoding GBA1 and the transposase or the nucleic acid sequence encoding the transposase, optionally as compared to a reference cell from a subject without Parkinson's Disease.

101. The system of embodiment 100, wherein the cell comprises a variant of GBA1 associated with Parkinson's Disease.
102. The system of embodiment 101, wherein the variant of wherein the variant of GBA1 comprises a single nucleotide polymorphism (SNP) that is associated with Parkinson's disease.
103. The system of embodiment 102, wherein the SNP is rs76763715.
104. The system of embodiment 103, wherein the rs76763715 is a cytosine variant.
105. The system of any one of embodiments 100-104, wherein the variant of GBA1 comprising a SNP encodes a serine, rather than an asparagine, at amino acid position 370 (N370S).
106. The system of embodiment 104 or embodiment 105, wherein the wild-type form of GBA1 comprises a thymine instead of the cytosine variant.
107. The system of embodiment 102, wherein the SNP is rs421016.
108. The system of embodiment 107, wherein the rs421016 is a guanine variant.
109. The system of any one of embodiments 102, 107, and 108, wherein the variant of GBA1 comprising the SNP encodes a proline, rather than a leucine, at amino acid position 444 (L444P).
110. The system of embodiment 108 or embodiment 109, wherein the wild-type form of GBA1 comprises an adenine instead of the guanine variant.
111. The system of embodiment 102, wherein the SNP is rs2230288.
112. The system of embodiment 111, wherein the rs2230288 is a thymine variant.
113. The system of any one of embodiments 102, 111, and 112, wherein the variant of GBA1 comprising the SNP encodes a lysine, rather than a glutamic acid, at position 326 (E326K).
114. The system of embodiment 112 or embodiment 113, wherein the wild-type form of GBA1 comprises a cytosine instead of the thymine variant.
115. The system of any one of embodiments 100-114, wherein the cell is a pluripotent stem cell (PSC), optionally an induced pluripotent stem cell (iPSC).
116. The system of any one of embodiments 100-115, wherein a plurality of the PSC, optionally the iPSCs are neurally differentiated by a method comprising:

(a) performing a first incubation comprising culturing the PSCs in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and

(b) performing a second incubation comprising culturing cells of the spheroid in a substrate-coated culture vessel under conditions to neurally differentiate the cells.

117. The system of embodiment 116, wherein the PSCs are exposed to the inhibitor of TGF-β/activin-Nodal signaling and the at least one activator of SHH signaling up to a day at or before day 7.
118. The system of embodiment 116 or embodiment 117, wherein the PSCs are exposed to the inhibitor of BMP signaling up to a day at or before day 11.
119. The system of any one of embodiments 116-118, wherein the PSCs are exposed to the inhibitor of GSK3β signaling up to a day at or before day 13.
120. The system of any one of embodiments 116-119, wherein culturing the cells under conditions to neurally differentiate the cells comprises exposing the cells to (i) brain-derived neurotrophic factor (BDNF); (ii) ascorbic acid; (iii) glial cell-derived neurotrophic factor (GDNF); (iv) dibutyryl cyclic AMP (dbcAMP); (v) transforming growth factor beta-3 (TGFβ3) (collectively, “BAGCT”); and (vi) an inhibitor of Notch signaling.

### VIII. EXAMPLES

The following examples are included for illustrative purposes only and are not intended to limit the scope of the invention.

### Example 1: Differentiation of iPSCs into Dopaminergic Neuron Progenitors

A. Generation of iPSCs

Fibroblasts from a human donor (“Donor 1”) having Parkinson Disease (PD) were obtained, and single nucleotide polymorphism (SNP) analysis was performed to confirm the donor carried a PD risk variant identified as SNP rs76763715 caused by the presence of a cytosine in place of a thymine (C>T), which causes an amino acid substitution of asparagine to serine at position 370 (N370S) in the beta-glucocerebrosidase (GCase) enzyme encoded by the beta-glucocerebrosidase (GBA1) gene. The presence of this SNP in the GBA1 gene reduces activity of the GCase enzyme, which may disrupt lysosomal homeostasis. The genomic sequence of the Donor 1 cell line was also analyzed to determine the presence of any other known SNP variant(s) that might contribute to PD, other than the rs76763715n SNP. No other known SNP variant(s) that might contribute to PD were identified in the Donor 1 cell line.

The Donor 1 cell line was reprogrammed into induced pluripotent stem cells (iPSCs) using CytoTune™-iPS 2.0 Sendai Reprogramming Kit (ThermoFisher), and RNA sequencing information was used to confirm the pluripotency of the cells using PluriTest™.

Donor 1 iPSCs were subjected to an exemplary dopaminergic (DA) neuronal differentiation protocol. Briefly, iPSCs were maintained by plating in 6-well plates (e.g., laminin-coated plates) at 200,000 cells per cm². The cells were cultured without feeder cells in mTeSR™1-based media until they reached approximately 90% confluence (day 0). The iPSCs were then washed with sterile PBS and detached from the 6-well plates by enzymatic dissociation with Accutase™. The collected iPSCs were then used in a subsequent differentiation protocol.

B. Differentiation Protocol

The collected iPSCs were re-suspended in “basal induction media” (see below) and seeded under non-adherent conditions using 6-well or 24-well AggreWell™ plates. The cells were seeded under conditions to achieve the following concentrations: 500 cells/spheroid; 1,000 cells/spheroid, 2,000 cells/spheroid; 3,000 cells/spheroid; 10,000 cells/spheroid; or 15,000 cells/spheroid. Following seeding of the cells, the 6-well or 24-well plates were immediately centrifuged at 200×g or 100×g for 3 minutes, respectively. Beginning at day 0, the media was supplemented with various small molecules as described below. The cells were cultured for 7 days, with media replacement as detailed below, to form spheroids. On day 7, the resulting spheroids were dissociated into single cells by enzymatic dissociation with Accutase™. The cells were plated as monolayers at a concentration of 600,000 cells/cm² on substrate-coated 6-well plates (e.g., laminin-coated plates) for the remainder of culture, and were further supplemented with nutrients and small molecules as described below.

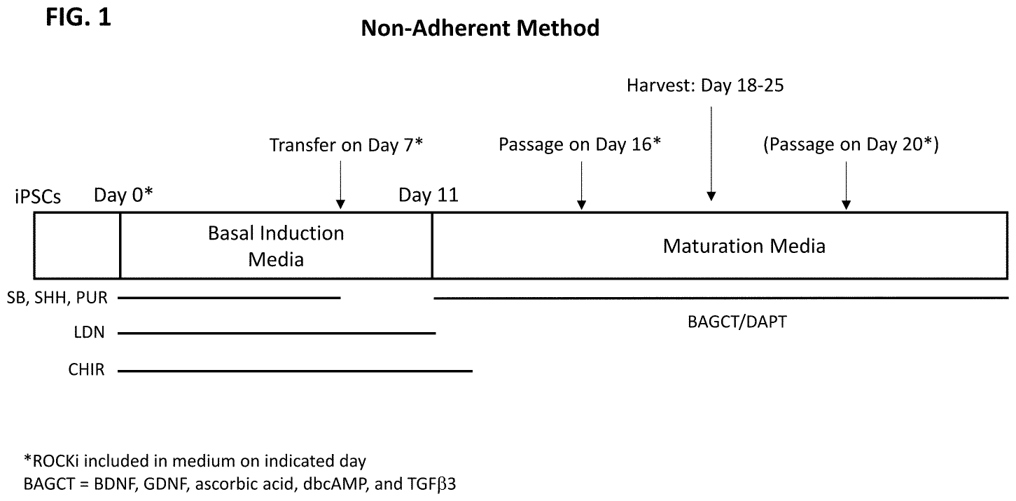

A schematic of the exemplary non-adherent differentiation protocol is shown in FIG. 1 and Table E1, which depict the small molecule compounds that were added at various days during the differentiation method. From days 0 to 10, cells were cultured in the basal induction media, which was formulated to contain Neurobasal™ media and DMEM/F12 media at a 1:1 ratio (and with N-2 and B27 supplements, non-essential amino acids (NEAA), GlutaMAX™, L-glutamine, β-mercaptoethanol, and insulin), and was supplemented with the appropriate small molecule compound(s). From days 11 to 25, cells were cultured in a “maturation media” (Neurobasal™ media containing N-2 and B27 supplements, non-essential amino acids (NEAA), and GlutaMAX™), and were supplemented with the appropriate small molecule compound(s). The basal induction media also included a serum replacement.

TABLE E1
Differentiation Protocol

| Day | Media | Small Molecules | | | | | | | |
|---|---|---|---|---|---|---|---|---|---|
| 0* | Basal | 5% | LDN | SB | SHH | PUR | CHIR | ROCKi | |
| | Induction | S | | | | | | | |
| 1 | Basal | 5% | LDN | SB | SHH | PUR | CHIR | | |
| | Induction | S | | | | | | | |
| 2 | Basal | 2% | LDN | SB | SHH | PUR | CHIR | | |
| | Induction | S | | | | | | | |
| 3 | Basal | 2% | LDN | SB | SHH | PUR | CHIR | | |
| | Induction | S | | | | | | | |
| 4 | Basal | 2% | LDN | SB | SHH | PUR | CHIR | | |
| | Induction | S | | | | | | | |
| 5 | Basal | 2% | LDN | SB | SHH | PUR | CHIR | | |
| | Induction | S | | | | | | | |
| 6 | Basal | 2% | LDN | SB | SHH | PUR | CHIR | | |
| | Induction | S | | | | | | | |
| 7* | Basal | 2% | LDN | | | | CHIR | ROCKi | |
| | Induction | S | | | | | | | |
| 8 | Basal | 2% | LDN | | | | CHIR | | |
| | Induction | S | | | | | | | |
| 9 | Basal | 2% | LDN | | | | CHIR | | |
| | Induction | S | | | | | | | |
| 10 | Basal | 2% | LDN | | | | CHIR | | |
| | Induction | S | | | | | | | |
| 11 | Maturation | | BDNF | GDNF | ascorbic | dbcAMP | CHIR | TGFβ3 | DAPT |
| 12 | Maturation | | BDNF | GDNF | ascorbic | dbcAMP | CHIR | TGFβ3 | DAPT |
| 13 | Maturation | | BDNF | GDNF | ascorbic | dbcAMP | | TGFβ3 | DAPT |
| 14 | Maturation | | BDNF | GDNF | ascorbic | dbcAMP | | TGFβ3 | DAPT |
| 15 | Maturation | | BDNF | GDNF | ascorbic | dbcAMP | | TGFβ3 | DAPT |

Day 16: 1^{st} Passage

| | | | | | | | |
|---|---|---|---|---|---|---|---|
| 16* | Maturation | BDNF | GDNF | ascorbic | dbcAMP | TGFβ3 | DAPT |
| 17 | Maturation | BDNF | GDNF | ascorbic | dbcAMP | TGFβ3 | DAPT |
| 18 | Maturation | BDNF | GDNF | ascorbic | dbcAMP | TGFβ3 | DAPT |
| 19 | Maturation | BDNF | GDNF | ascorbic | dbcAMP | TGFβ3 | DAPT |

Day 20: 2^{nd} Passage

| | | | | | | | |
|---|---|---|---|---|---|---|---|
| 20* | Maturation | BDNF | GDNF | ascorbic | dbcAMP | TGFβ3 | DAPT |
| 21 | Maturation | BDNF | GDNF | ascorbic | dbcAMP | TGFβ3 | DAPT |
| 22 | Maturation | BDNF | GDNF | ascorbic | dbcAMP | TGFβ3 | DAPT |
| 23 | Maturation | BDNF | GDNF | ascorbic | dbcAMP | TGFβ3 | DAPT |
| 24 | Maturation | BDNF | GDNF | ascorbic | dbcAMP | TGFβ3 | DAPT |
| 25 | Maturation | BDNF | GDNF | ascorbic | dbcAMP | TGFβ3 | DAPT |

S: Serum replacement;
LDN: LDN193189;
SB: SB431542;
SHH: recombinant mouse Sonic Hedgehog (rmSHH);
PUR: Purmorphamine;
CHIR: CHI99021;
ROCKi: Y-27632;
BDNF: recombinant human brain-derived neurotrophic factor (rhBDNF);
GDNF: recombinant human glial cell-derived neurotrophic factor (rhGDNF);
TGFβ3: recombinant human transforming growth factor beta 3 (rhTGFβ3);
dbcAMP: dibutyryl cyclic AMP;
Ascorbic: ascorbic acid;
*Indicates media supplemented with ROCK inhibitor (Y-27632)

Specifically, on day 0, the basal induction media was formulated to contain: 5% serum replacement, 0.1 μM LDN, 10 μM SB, 0.1 μg/mL SHH, 2 μM PUR, 2 μM of the GSK3β inhibitor CHIR99021, and 10 μM of the ROCK inhibitor Y-27632. The media was completely replaced on day 1 to provide the same concentration of the small molecule compounds as on day 0, except that no ROCK inhibitor was added. From days 2 to 6, the same concentration of the small molecule compounds as on day 1 was provided daily but by 50% media exchange; the concentrations of small molecules in the basal induction media were doubled on days 2 to 6, to ensure the same total concentration of compounds was added to the cell cultures. Also, the media on days 2 to 6 was formulated with 2% serum replacement.

On day 7 when the cells were transferred to substrate-coated plates, the basal induction media was formulated to contain: 2% serum replacement, 0.1 μM LDN, 10 μM SB, 2 μM CHIR99021, and 10 μM Y-27632. The media was replaced daily from days 8 to 10, with basal induction media formulated to contain 2% serum replacement, 0.1 μM LDN and 2 μM CHIR99021.

Starting on day 11, the media was switched to maturation media formulated to contain: 20 ng/mL BDNF, 0.2 mM ascorbic acid, 20 ng/mL GDNF, 0.5 mM dbcAMP, and 1 ng/mL TGFβ3 (collectively, “BAGCT”), 10 μM DAPT, and 2 μM CHIR99021. The media was replaced on day 12 with the same media formulation containing the same concentrations of small molecule compounds as on day 11. From day 13 until harvest, the media was replaced either every day (days 13-17) or every other day (after day 17) with maturation media formulated to contain BAGCT and DAPT (collectively, “BAGCT/DAPT”) at the same concentrations as on days 11 and 12.

On day 16, the cells were passaged by enzymatic dissociation with dispase and collagenase. Cells were re-suspended as small clumps and re-plated in maturation media that was further supplemented with the ROCK inhibitor.

On day 18, the differentiated cells were harvested by enzymatic dissociation, and analyzed by immunohistochemistry for markers of midbrain DA neurons, including FOXA2 and tyrosine hydroxylase (TH), or cryofrozen for downstream use. In some embodiments, the differentiated cells were harvested on day 20. If differentiation past day 20 is desired (such as until day 25), the cells are passaged on day 20 by enzymatic dissociation with dispase and collagenase, re-suspended as small clumps, and re-plated in maturation media supplemented with the ROCK inhibitor.

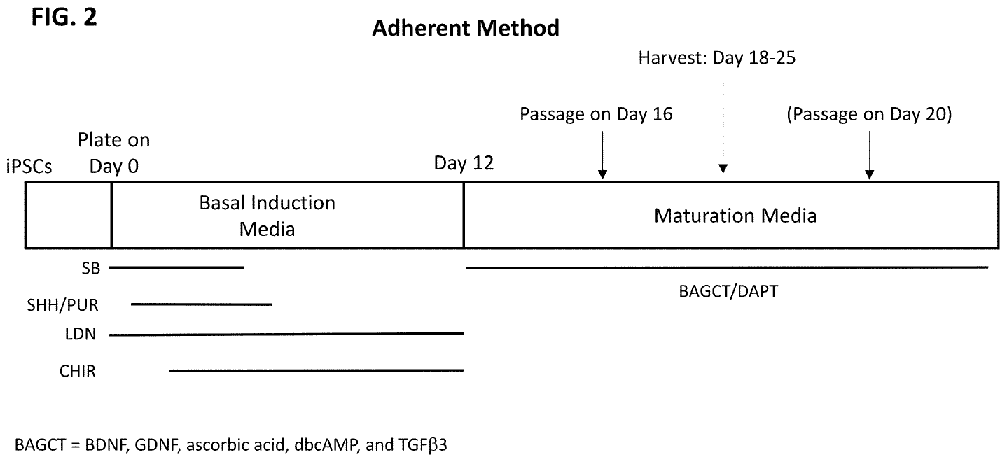

In some cases, differentiated cells were generated from iPSCs by an alternative method, in which the cells were initially plated in 6-well plates (e.g., laminin-coated plates) on day 0 and remained plated for the duration of the differentiation protocol (“adherent method”), such as until harvest at day 20. The adherent method also differed from the non-adherent method, in that the small molecules were added on different schedules (FIG. 2).

It was observed that mature DA neurons differentiated from donor iPSCs exhibited impaired lysosomal function, but mitochondrial function was not affected. Additionally, an increase in the uptake of alpha-synuclein preformed fibrils and a decrease in their lysosomal degradation was observed in the mature DA neurons.

### Example 2: Transposon-Based Modulation of GBA1

iPSCs obtained from Donor 1 were transfected with a transposon construct containing a human GBA1 transgene, in order to achieve stable overexpression of the wild-type GBA1 transcript and increase GCase activity in the cells. Transfected cells were subsequently differentiated via the exemplary adherent method as described in Example 1.

TcBuster transposon constructs were generated to contain a transgene encoding human GBA1 and green fluorescent protein (GFP) under a Ubiquitin C promoter (UBC-GBA-T2A-GFP) or a phosphoglycerate kinase 1 promoter (PGK-GBA-T2A-GFP). The sequences encoding GBA1 and GFP were separated by a sequence encoding a T2A self-cleaving peptide. iPSCs (day 0) were obtained from Donor 1 and transfected by nucleofection with (1) a plasmid encoding a transposase and (2) the UBC-GBA-T2A-GFP or PGK-GBA-T2A-GFP transposon construct. Following nucleofection, successfully transfected iPSCs were identified and selected based on GFP expression, and the integration site and copy number of the inserted transgene were analyzed. iPSC clones that were confirmed to have integrated the transposon construct at a non-disruptive site in the genome were expanded in culture and subsequently differentiated into dopaminergic (DA) neuron progenitors as described in Example 1. Differentiated cells were cultured until day 25, at which point the cells were harvested for analysis of promoter and GCase activity. Transfected iPSCs were also harvested on day 0 for comparison.

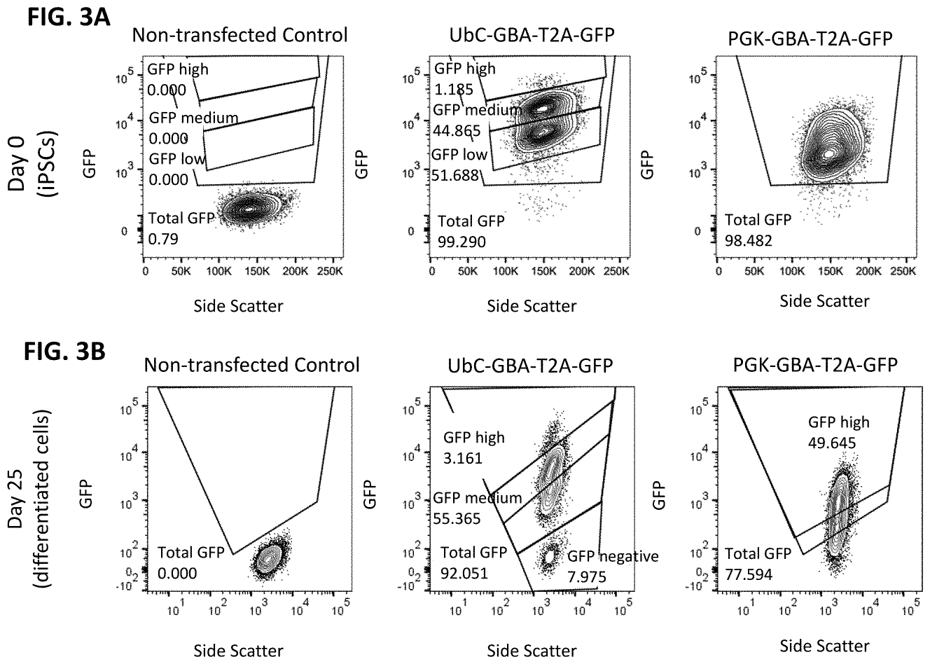

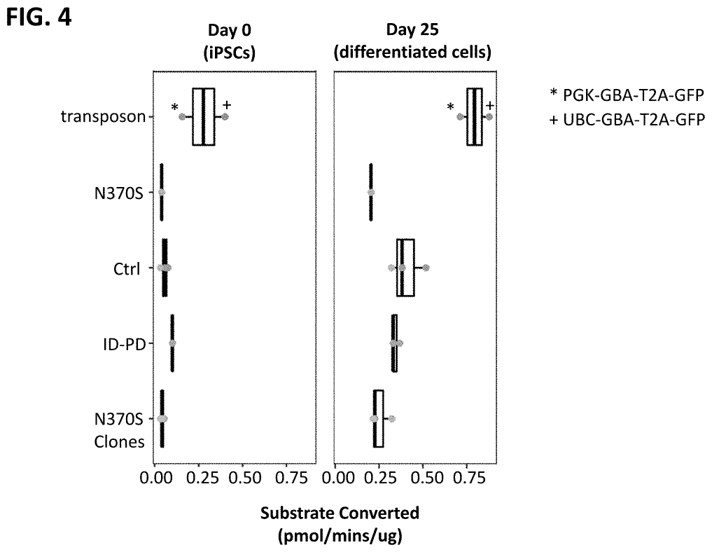

The activity of the UBC and PGK promoters was determined by analysis of GFP expression in day 0 iPSCs and day 25 differentiated cells. Flow cytometric analysis revealed that the majority of day 0 iPSCs transfected with either the UBC-GBA-T2A-GFP or the PGK-GBA-T2A-GFP construct expressed GFP (FIG. 3A). Among day 25 differentiated cells, activity of both promoters was observed to be heterogeneous (FIG. 3B), though the majority of cells transfected with either construct exhibited GFP expression. It was hypothesized that at least some of the heterogeneity of GFP expression observed in differentiated cells is attributable to differences in the integration site and copy number of the transposon construct.

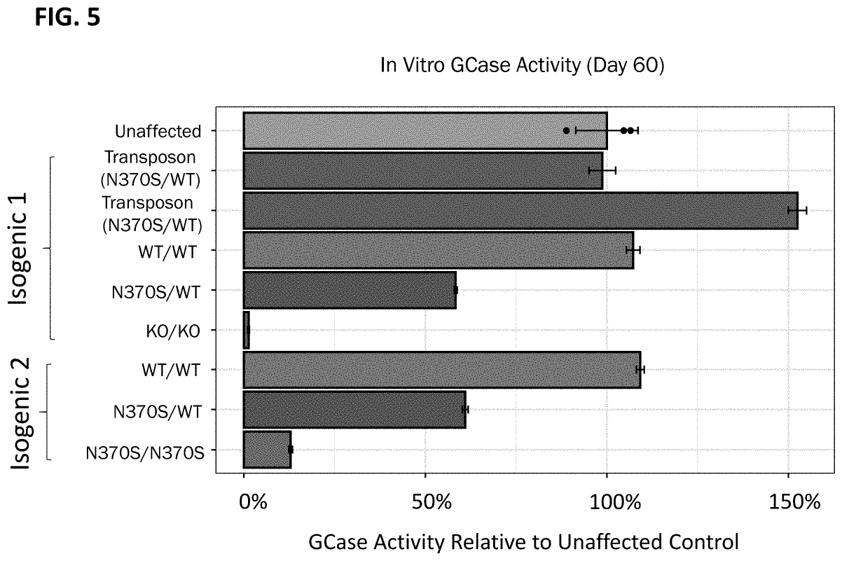

The GCase activity of transfected iPSCs (day 0), as well as of differentiated cells that were transfected (day 25), was assessed (FIG. 4; *PGK-GBA-T2A-GFP and +UBC-GBA-T2A-GFP). GCase activity was determined by an enzymatic activity reaction wherein protein isolated from cells was combined with the 4-methylumbelliferyl beta-D-glucopyranosidase (4-MBDG) substrate. Cleavage of the substrate by GCase yielded 4-methylumbelliferone (4-MU), the concentration of which was measured by comparing its fluorescent intensity to a standard curve. The GCase activity of Donor 1's transfected cells from a single clone (transposon) was compared to that of non-transfected cells from the same Donor 1 clone (N307S), cells from donors not having Parkinson's disease (Ctrl), cells from a donor having idiopathic Parkinson's Disease (ID-PD), and three different non-transfected clones derived from Donor 1 (N370S clones).

Expression levels of neuronal differentiation markers TH and FOXA2 were assessed in Donor 1 cells that were transfected with either the UBC-GBA-T2A-GFP or the PGK-GBA-T2A-GFP construct, differentiated, and harvested on day 25. Transfection with the constructs was not observed to affect the fate of differentiated cells, as TH and FOXA2 expression was present in non-transfected cells and transfected cells.

### Example 3: Further Assessment of GCase Activity

GCase activity was assessed in iPSC-derived, differentiated dopaminergic (DA) neurons generated from isogenic cell lines having different GBA1 genotypes. The first isogenic cell line (“Isogenic 1”) was generated to contain two wild-type GBA1 alleles (WT/WT); one wild-type GBA1 allele and one GBA1 N370S allele (N370S/WT); or complete knockout of the GBA1 locus (KO/KO) as a negative control. The second isogenic cell line (“Isogenic 2”) was generated to contain the WT/WT genotype, the N370S/WT genotype, or two GBA1 N370S alleles (N370S/N370S). Three cell lines from three different donors not having a GBA1 mutation served as positive control (“Unaffected”; each dot represents a different donor).

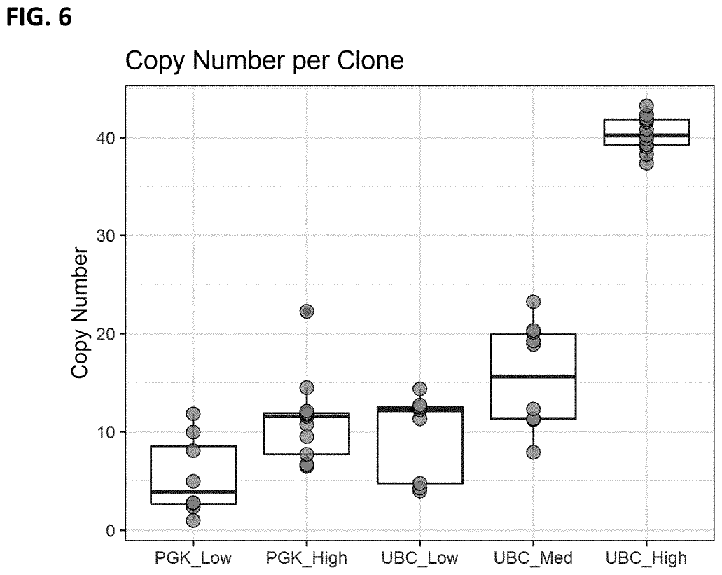

The isogenic iPSCs were differentiated via the exemplary adherent method as described in Example 1. The GCase activity of differentiated cells was assessed at day 60 by the enzymatic activity reaction as described in Example 2 (FIG. 5). The level of GCase activity in the Unaffected cell line was set to 100%. Cells having two alleles of wild-type GBA1 (WT/WT) exhibited similar GCase activity as compared to the unaffected cell line. Cells expressing one or two allele(s) of GBA1 N370S exhibited dose-wise decreases in GCase activity. However, cells having one GBA1 N370S allele and transfected with the PGK-GBA-T2A-GFP construct as described in Example 2 exhibited similar levels of GCase activity as compared to Unaffected cells and cells having two wild-type GBA1 alleles. These data are consistent with a finding that transposon-mediated overexpression of wild-type GBA1 can restore GCase activity in cells having reduced endogenous GCase activity, such as due to a N370S mutation in GBA1.

### Example 4: Assessment of Transgene Genomic Integration

iPSCs were transfected with UBC-GBA-T2A-GFP (“UBC”) or PGK-GBA-T2A-GFP (“PGK”) transposon constructs. The constructs were characterized as having promoters with low (“low”), medium (“med”), or high (“high”) activity (see FIG. 3). GBA1 copy number, integration location, and gene expression were assessed.

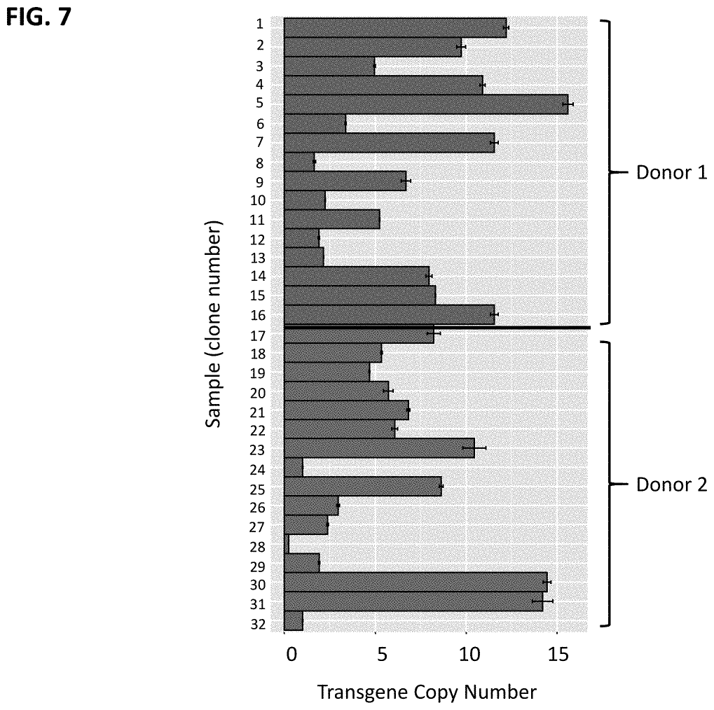

As shown in FIG. 6 (each dot represents a clone), iPSCs transfected with transposon constructs incorporating a PGK promoter tended to have fewer copies of the wild-type GBA1 transgene integrated, as compared to those transfected with transposon constructs incorporating a UBC promoter. The copy number was observed to approximately correlate with GFP expression.

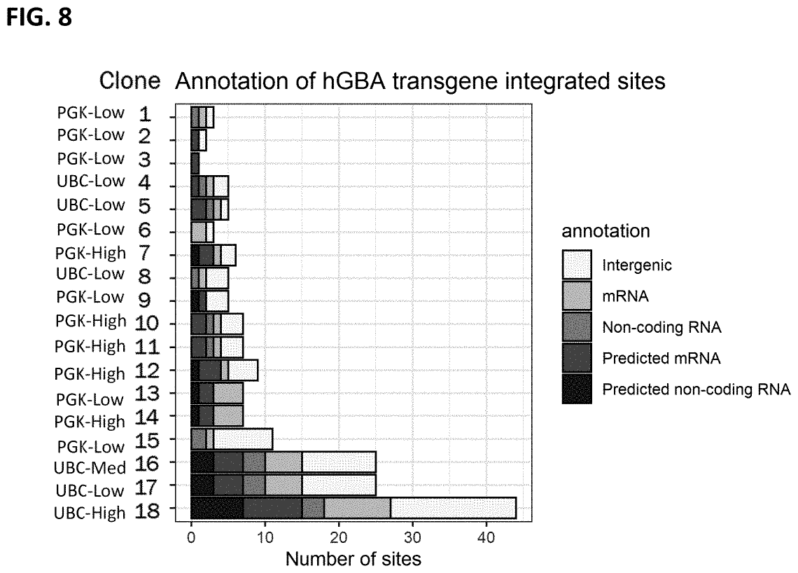

In a related experiment, the number of wild-type GBA1 transgene copies was assessed in iPSC clones derived from two different human donors and transfected with a low PGK-GBA-T2A-GFP transposon construct. Of the 32 total clones analyzed, 31 were found to have at least 1 copy of the transgene. Of the 31 clones containing at least 1 copy of the transgene, 14 were found to have 5 or fewer copies of the transgene (FIG. 7).

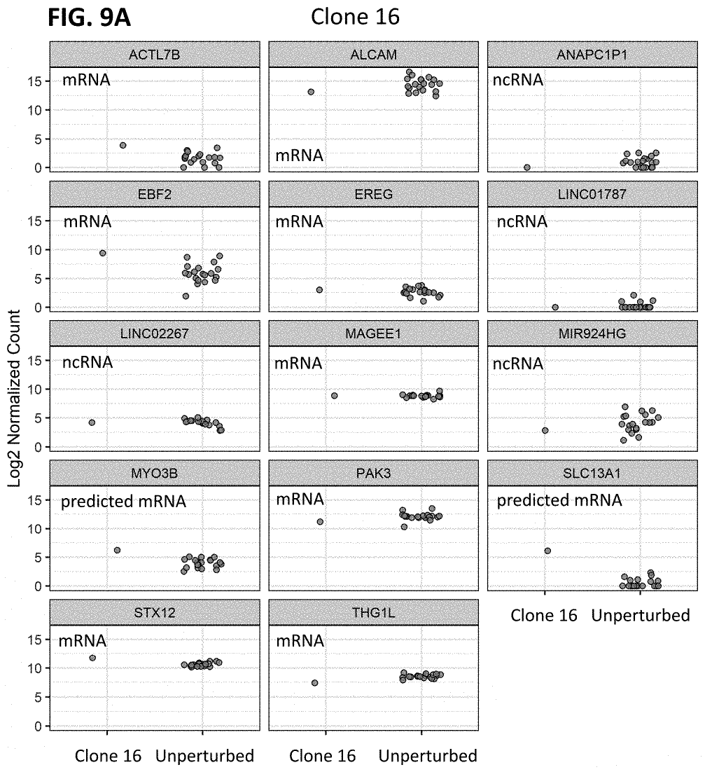

18 different iPSC clones transfected with a PGK-GBA-T2A-GFP or UBC-GBA-T2A-GFP transposon construct were selected for site integration analysis to determine if the wild-type GBA1 transgene was integrated in an intergenic region, mRNA, non-coding RNA (ncRNA), a predicted mRNA, or a predicted non-coding RNA site. 15 of the 18 clones had fewer than 10 copies of the wild-type GBA1 gene integrated, while 3 clones had greater than 10 copies of the wild-type GBA1 gene integrated. Among all of the clones, no integration sites were inside exons, no sites overlapped with oncogene regions (as assessed by TruSight® Assay, Illumina), and 3 clones exhibited no integration in mRNA (FIG. 8).

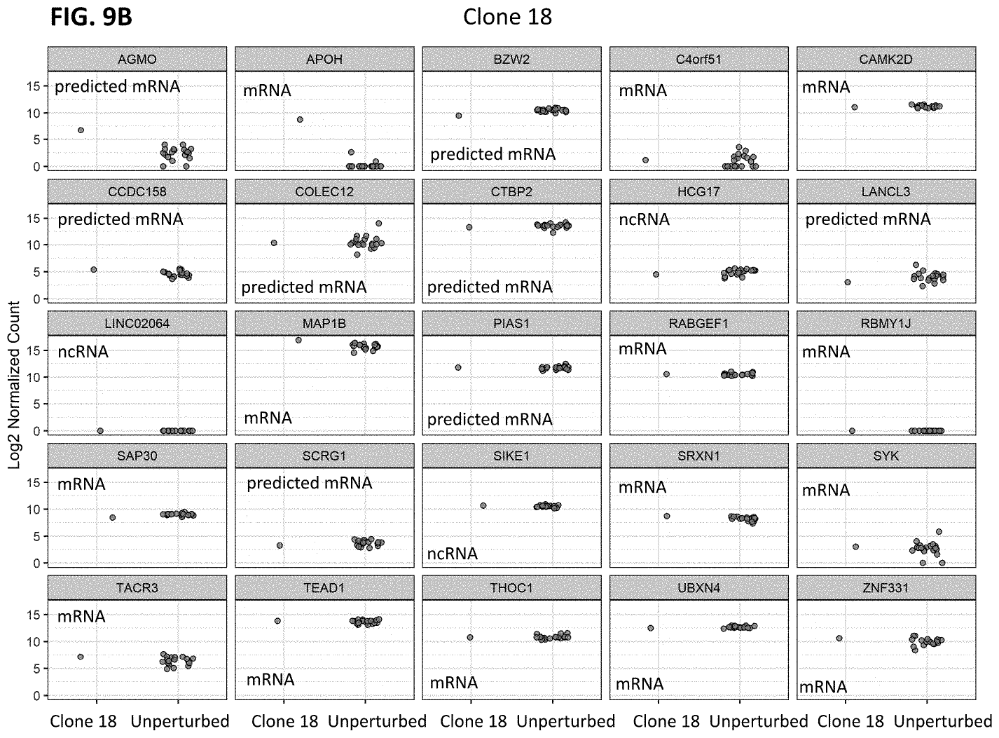

To determine whether site integration in introns affected gene expression, the expression of various genes was assessed in clones 16 and 18 (FIGS. 9A and 9B, respectively). Only one of 14 genes assessed for clone 16 (SLC13A1) showed increased expression, while none showed decreased expression. Only one of 25 genes assessed for clone 18 showed increased expression (APOH), while none showed decreased expression.

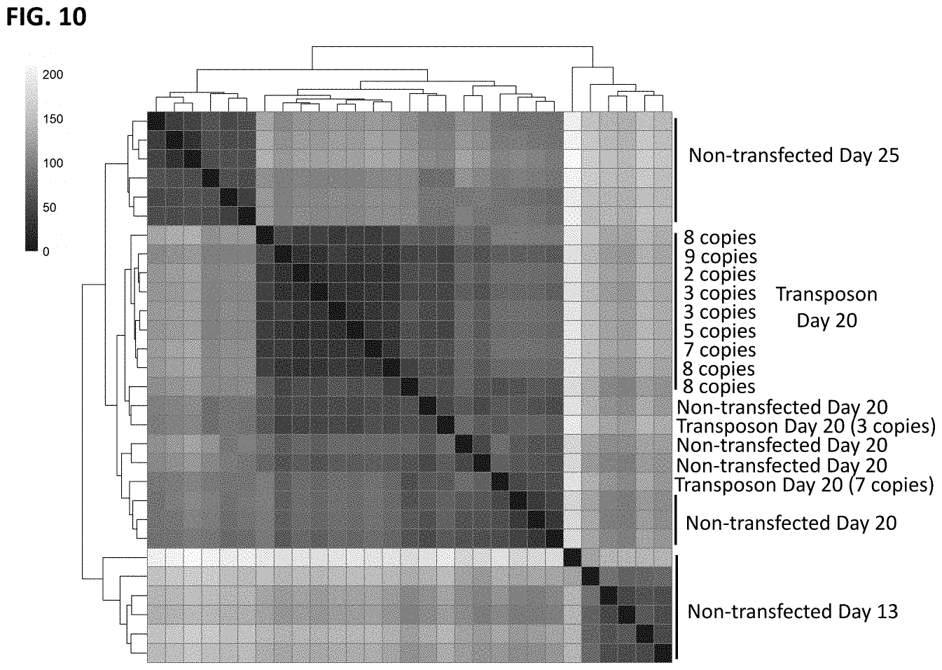

To determine whether transgene integration affects genome-wide transcription, iPSCs were transfected with a low PGK-GBA-T2A-GFP transposon construct, differentiated by the adherent method described in Example 1, and harvested at day 20. Transfected clones were observed to have between 2 and 9 copies of the wild-type GBA1 transgene. Non-transfected iPSCs were differentiated by the same method and harvested on day 13, 20, or 25. Genome-wide gene expression was compared among the different cells (FIG. 10; scale shows Euclidian distance between each sample pair). The transcriptome signature of transfected cells harboring the wild-type GBA1 transgene was observed to be similar to that of non-transfected, differentiated cells. Among the transfected cells, no effect of copy number on transcriptome signature was observed.

### Example 5: Assessment of Neuronal Differentiation Following Transgene Integration

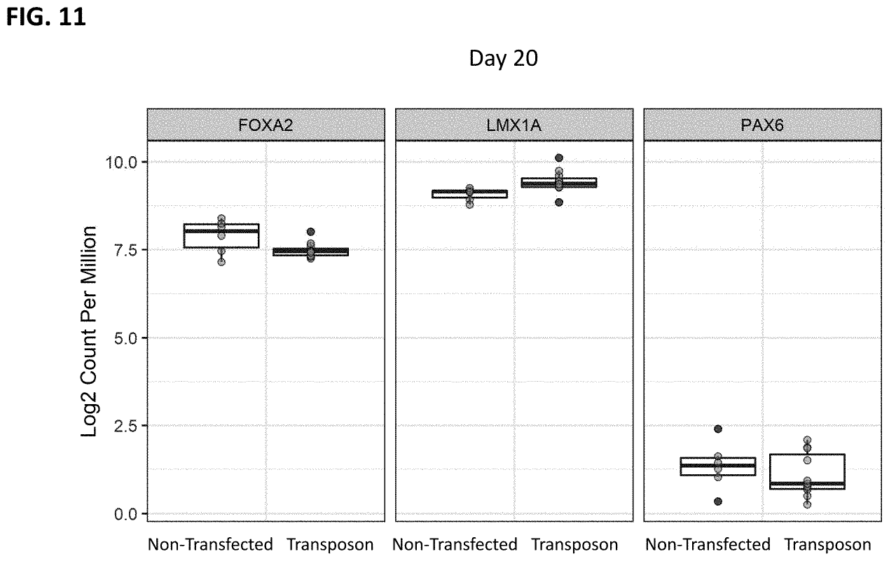

iPSCs were transfected with a PGK-GBA-T2A-GFP transposon construct substantially as described in Example 2, differentiated by the exemplary adherent method as described in Example 1, and harvested at day 20 for analysis of DA neuronal differentiation markers. For comparison, non-transfected iPSCs were differentiated by the same methods and harvested at day 20. Expression of the FOXA2, LMX1A, and PAX6 genes was observed to be similar between non-transfected and transfected cells (FIG. 11).

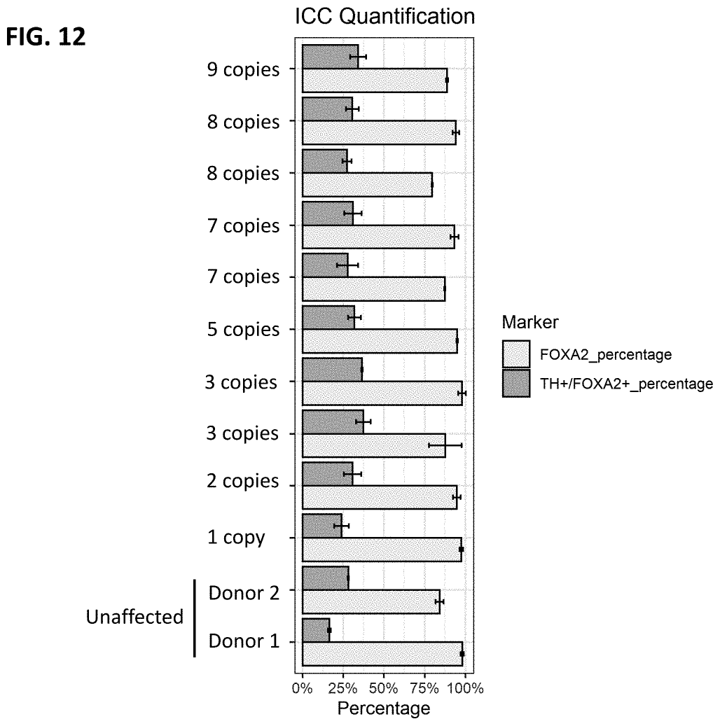

In a similar experiment, cells transfected with a PGK-GBA-T2A-GFP transposon construct and non-transfected cells were differentiated and harvested at day 35 for analysis of FOXA2 and TH expression by immunocytochemistry. The percentage of FOXA2+ and TH+FOXA2+ cells was determined from clones having integrated between 1 and 9 copies of the wild-type GBA1 transgene. Clones integrating the wild-type GBA1 transgene were observed to yield a comparable number of dopaminergic neurons as compared to non-transfected clones, independent of the number of GBA1 transgene copies (FIG. 12).

### Example 6: Assessment of GCase Expression and Activity

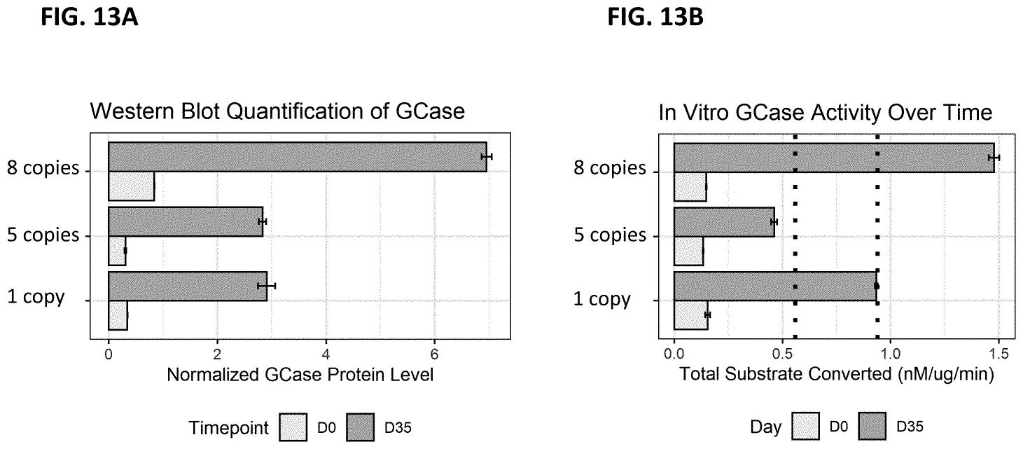

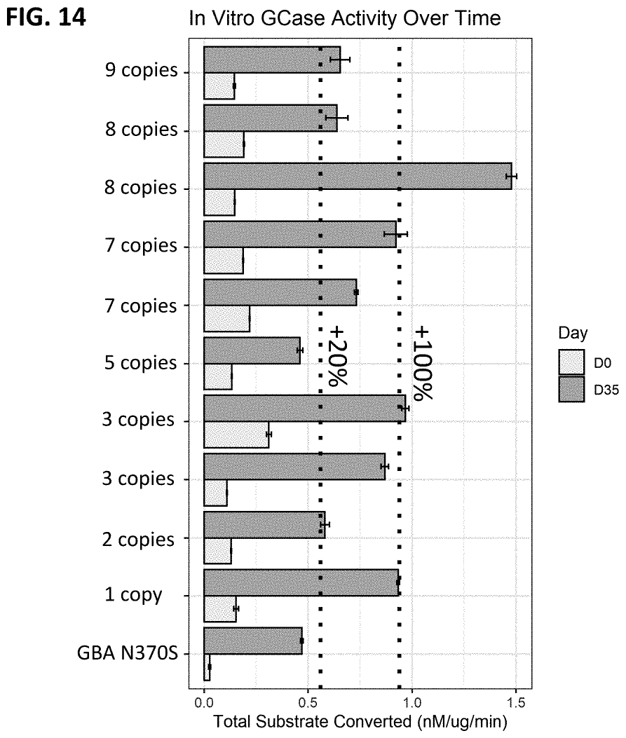

The protein expression and activity of the GCase enzyme were assessed in iPSCs (day 0) or neuronally differentiated cells (day 35) from clones introduced with the wild-type GBA1 transgene via transfection with a PGK-GBA-T2A-GFP transposon construct, substantially as described in Example 2. Cells from clones incorporating 1, 5, or 8 copies of the wild-type GBA1 transgene exhibited increased GCase protein expression and activity at day 35, as compared to day 0 (FIGS. 13A and 13B, respectively). Similar increases in GCase activity between day 0 and day 35 were observed for clones incorporating between 1 and 9 copies of the wild-type GBA1 transgene (FIG. 14). Further, in comparison to a clone having a GBA1 N370S mutation and not introduced with the wild-type GBA1 transposon construct, most clones incorporating the wild-type GBA1 transgene exhibited 20-100% greater GCase activity at day 35.

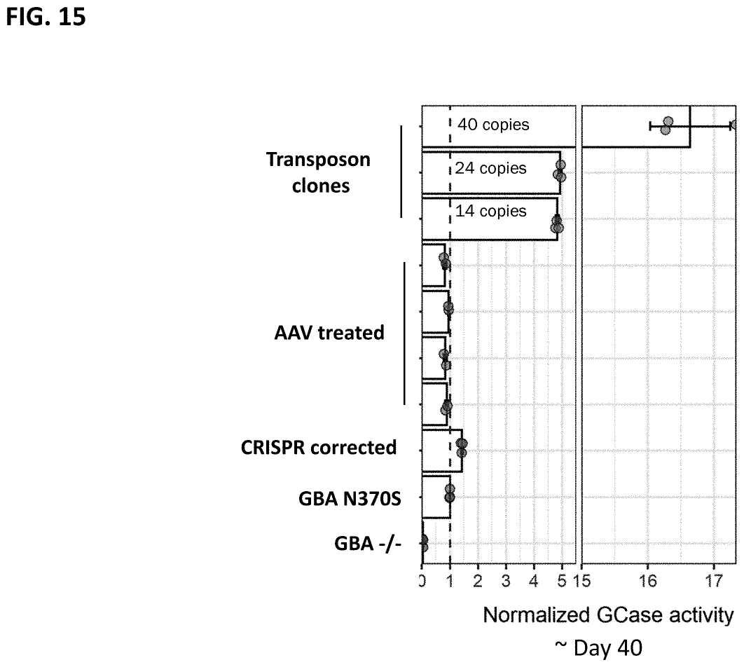

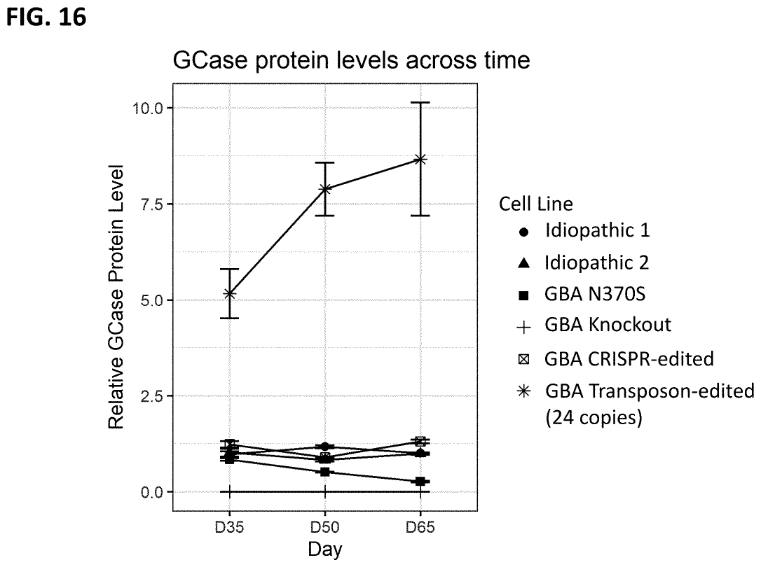

The ability of the PGK-GBA-T2A-GFP transposon construct-based method to increase GCase activity in differentiated cells at day 40 was also compared to either (1) an AAV-based method of overexpressing wild-type GBA1 or (2) a CRISPR/Cas-based method of correcting the N370S mutation. As controls, cells either completely knocked out for GBA1 or having a GBA1 N370S mutation were assessed. GCase activity for all cells was normalized to that of the GBA1 N370S cells. As shown in FIG. 15, clones derived from transposon-based overexpression of the wild-type GBA1 transgene were exhibited substantially increased GCase activity as compared to AAV- and CRISPR/Cas-modified cells. To further compare the transposon- and CRISPR/Cas-based methods, GCase protein levels were assessed in differentiated cells at days 35, 50, and 65. Cells modified by the transposon-based method had 24 integrated copies of the wild-type GBA1 transgene. As controls, differentiated cells derived from a donor having idiopathic Parkinson's disease (idiopathic), cells having a GBA N370S mutation, and cells completely knocked out for GBA1 were also included. GBA protein levels were observed to increase over time for cells modified by the transposon-based methods, whereas no significant increase in GCase protein levels were observed over time in any of the other cells (FIG. 16).

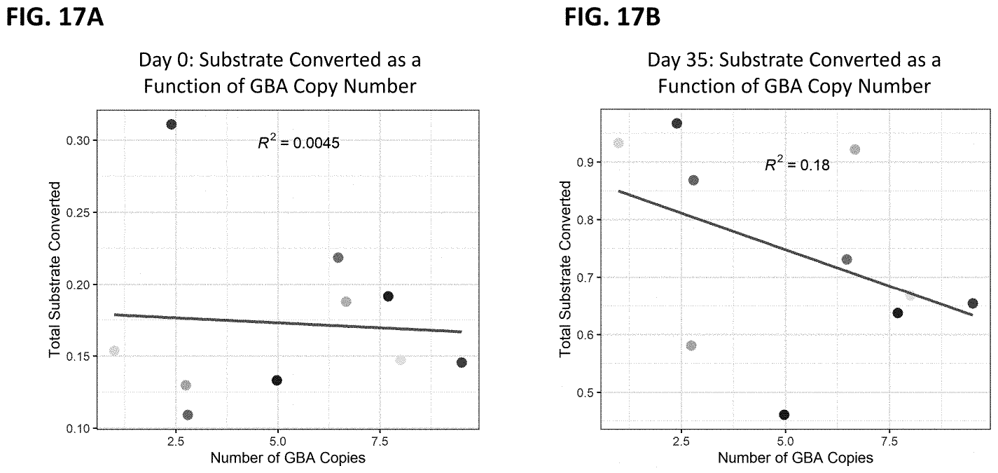

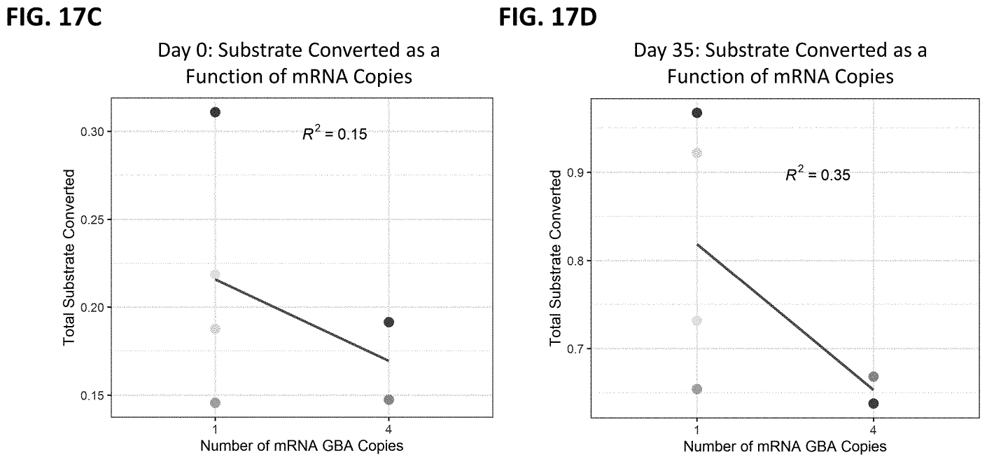

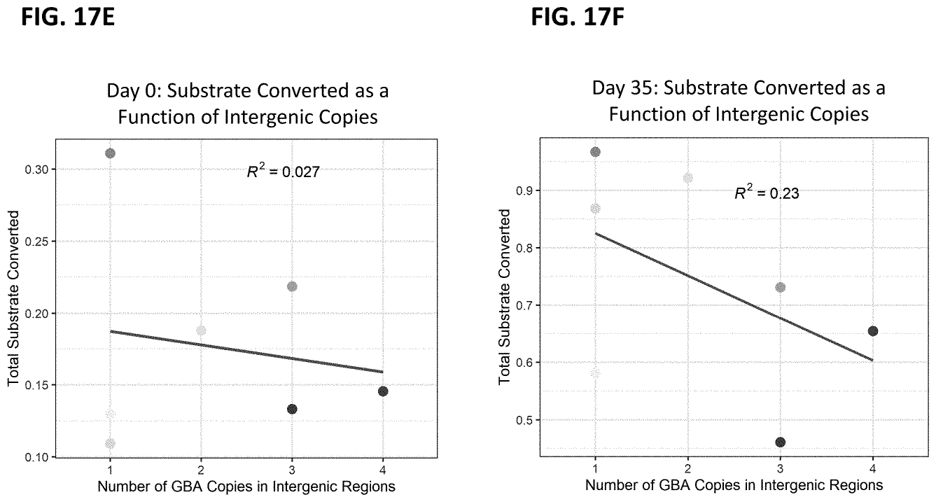

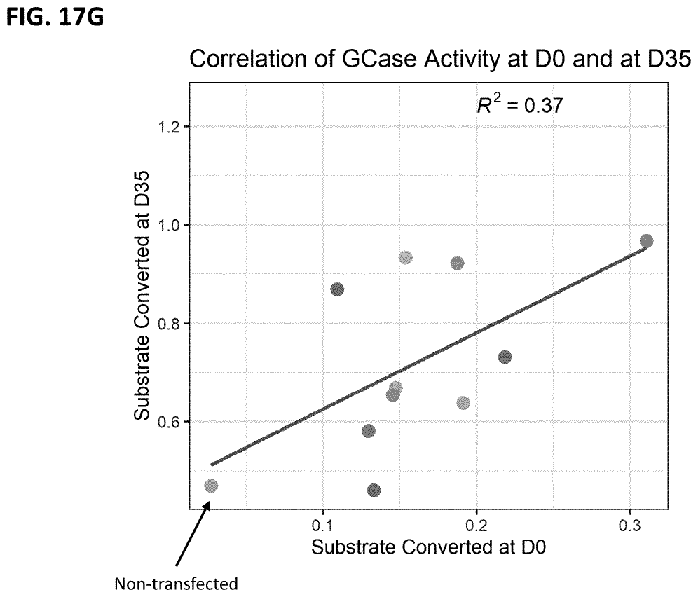

Additional experiments were carried out to identify potential correlates of GCase activity in differentiated cells modified to overexpress wild-type GBA1 by the PGK-GBA-T2A-GFP transposon construct-based method described in Example 2. No relationship was observed between GCase activity and total wild-type GBA1 transgene copy number in day 0 iPSCs or day 35 neuronally differentiated cells (FIGS. 17A and 17B, respectively). Similarly, no correlation was observed in day 0 iPSCs or day 35 neuronally differentiated cells between GCase activity and the number of wild-typeGBA1 transgene copies integrated in mRNA (FIGS. 17C and 17D, respectively) or in intergenic regions (FIGS. 17E and 17F, respectively). GCase activity in day 0 iPSCs was also observed not to correlate with GCase activity in day 35 neuronally differentiated cells (FIG. 17G). Each dot represents a different clone, unless noted otherwise.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### Sequences

| SEQ ID | | |
|---|---|---|
| NO | Sequence | Description |
| 1 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (wildtype) |
| | LHNFSLPEEDTKLKIPLIHRALQLAQRPVSLLASPWTSPTWLKTNGAV | amino acid |
| | NGKGSLKGQPGDIYHQTWARYFVKFLDAYAEHKLQFWAVTAENEPS | sequence |
| | AGLLSGYPFQCLGFTPEHQRDFIARDLGPTLANSTHHNVRLLMLDDQ | |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 2 | ACAGAAGTACAATGCTGATTATGATCTGTCAGCTCAGCAAGGGGC | Human |
| | AGACACCCTGGCCTTCATGTCTCTCCTGGAGGAGAAGTTGATCCCG | GBA1 |
| | GTGCTGGTGAGTGTGCCCAGACCTCCCAGCATCCATGGCCAGCCG | (wildtype) |
| | GGGAGGGGACGGGAACACACAGACCCACACAGAGACTCAGGAGA | nucleotide |
| | GCATGGAGGTCAGAAGCCCACCTTGAATCAGACAGGTGCACTGGC | sequence |
| | TCAGACCTGCCTGTTTCTTCCTGCCCACCCAATCCAGGTACATACT | |
| | TTTTGGATAGACACCAAGAACTACGTAGAAGTGACCCGGAAGTGG | |
| | TATGCAGAGGCTATGCCCTTTCCCCTCAACTTCTTCCTGCCTGGCC | |
| | GCATGCAGCGGCAGTACATGGAACGGCTACAGCTGCTGACTGGGG | |
| | AGCACAGGCCTGAGGACGAGGAAGAGCTGGAGAAGGAGGTAGCT | |
| | CTGAGACCGGGGGCTATTGTATGAGATGAGCCCCAAGGATGCTGG | |
| | CCAGGAATGGGAGTGCTTAGGTGCGGAGGTGGCACTGTTCCCGCA | |
| | GCTGCAAGCCTACCTGTGTCGCCCCTACAGCTGTACCGAGAGGCTC | |
| | GGGAGTGTCTGACCCTGCTCTCTCAGCGCCTGGGCTCTCAAAAGTT | |
| | CTTCTTTGGAGATGCGTGAGTCTGACTCCAAGAGGGTAATGGGTG | |
| | GCTTGGAAGAAGATACAGGTTCAGATGGAGCAGCTGGAGCTGGGG | |
| | CTGGGGCTGGGGCTGGCTCAGGCTCTGGATAGGAGGTCCCTGAGA | |
| | CAGATACTGGCCCTGGTGACAGTGGGGCTGTGCGTGGGGCCAGAG | |
| | CCTTCTCAGAGGTACAAAAGGGTAGGGTGGGAGGGCAGCCAGGCA | |
| | CAGGAAGGGCCTGAAGAGCTGTGGGGCACTGAGTGTGCCCTTTAT | |
| | GCAGCCCTGGGATAGAGCCCTATTCAGGGCCAGGCTGGCGCCACC | |
| | TGGGGATCTCTCCCCATACCAGGTCTAGAACTGTGTGTCCTGTCCT | |
| | TCCCTGGTGGCCGCCTGCTGCCCAGAGCCCACCTCCCAAGGCTGAC | |
| | TCTTCCTCCAGCTCCATCTTTACCCCTTCTACCCCAGTGGTTCTCCT | |
| | CCATCCCACCCTTCTCTCTCTGCTCCAGCCCTGCCTCCTTGGACGCC | |
| | TTCGTCTTCAGCTACTTGGCCCTGCTGCTGCAGGCAAAGCTGCCCA | |
| | GTGGGAAGCTGCAGGTCCACCTGCGTGGGCTGCACAACCTCTGTG | |
| | CCTATTGTACCCACATTCTCAGTCTCTACTTCCCCTGGGATGGAGG | |
| | TAAGGGGCAGATGGGAGGGGCAGCCCTGGGGAGAGTGGGCAGGG | |
| | ATCCAAGAACTAGTTCTCCTAACACACCTTCCTTCCTTGACCCTCA | |
| | GCTGAGGTACCACCGCAACGCCAGACACCAGCAGGCCCAGAGACT | |
| | GAGGAGGAGCCATACCGGCGCCGGAACCAGATCCTATCTGTGCTG | |
| | GCAGGACTGGCAGCCATGGTGGGCTACGCCTTGCTCAGCGGCATT | |
| | GTCTCCATCCAGCGGGCAACGCCTGCTCGGGCCCCAGGCACCCGG | |
| | ACCCTGGGCATGGCTGAGGAGGATGAAGAGGAATGATTTGTCCTC | |
| | ACGCTCCCAAGACTGGTTTTTCTACTCTCATGCATTCCAGAGGCCC | |
| | CCGTGCCTCCTCGTTGTTGGTACAGCCGGACACGGGGTGCTGCCAC | |
| | CCAGAATAAAGCCACTCACACTGACTGGGCTCAAACATTTTCTCCT | |
| | TTAAGAGCTGCCATTTTTCCTGGCTGGTGCCATAGGAATCATCTGG | |
| | GTGCCTGGGCACACCCGCTGCTGCTTTAAGGCTTCCGCCCTGATGC | |
| | TGACACTGCTGCTCCACGGGCCCAGTTCTGCATCTCCAGGAAAGAC | |
| | AAACAGTCTCCAGTTTTGGGCCCAGCTTTCCTAGTCTCTTCTTTTCC | |
| | TTACCCTCAGCCCTGATCTTGTGTTTGTACGGACAGTGAGCTCACC | |
| | CTAGGCCTGGACCCAGGCCCAGTTTCCAAAGCAAGCAGCACACAG | |
| | CGCATGTTCACATAAGCATGGGGGCTGGGGGGACACTGGGGCTTA | |
| | CTGATCTTTTTCTAGGGGCCTCCAGCCCCTGGCACCACCTAGAGGG | |
| | GAAAGTGAGTCACCCAAACCATTGCCCCTGGGCTTACGTCGCTGTA | |
| | AGCTCACACTGGCCCTGCTGTGCCCTCTTTAGTCACAGACAGCGTG | |
| | TGAGCTGACTCTGTCCCTTTAATGCCCAGGCTGAGCCCAGTGCCTC | |
| | CTTGAGTATCTGCTCCATCACTGGCGACGCCACAGGTAGGTGTGAA | |
| | TGGAGTAGCCAGGTGAGATTGTCTCCAGGAAGCCCACAGCAGGAT | |
| | CCTTGATGGTAAGAGGCACATCCTTAGAGGAGCTAGGGAGCAGGG | |
| | AGGAGAAGCTGAGAGTGTGATCCTGCCAAGGCCCCCAACGCTGTC | |
| | TTCAGCCCACTTCCCAGACCTCACCATTGCCCTCACCGGTTTAGCA | |
| | CGACCACAACAGCAGAGCCATCGGGATGCATCAGTGCCACTGCGT | |
| | CCAGGTCGTTCTTCTGACTGGCAACCAGCCCCACTCTCTGGGAGCC | |
| | CTCAGGAATGAACTTGCTGAATGTGGGAACAGATGGTCAGAGTCC | |
| | CTCGGGGTACCTCCCATGAAACCCTCATCTAAGAAGTCACCCACCC | |
| | ACGGACCCACCCCATAACTCCTGCAGAGGCTCTGCCCTGGCTCTCT | |
| | AGGCCTGGAGCCATGCTGCTGGGCACTGACCCTGCTTTTCTGCATC | |
| | GCAGTCCAGCCTCAGGCATTGGGGTTTTCTGTTGCTACCTAGTCAC | |
| | TTCCTGCCTCCATGGTGCAAAAGGGGATGGGTGTGCCTCTTCCGAG | |
| | GTTCCACCCTGAACACCTTCCTGCTCCCTCGTGGTGTAGAGTGATG | |
| | TAAGCCATCCGATGTAGGAGATGATAGGCCTGGTATGGAATGGGG | |
| | GTGCCCGCCCTCCACTCACCTGAAGTGGCCAAGGTGGTAGAACAT | |
| | GGGCTGTTTGTAAAACGTGTCCTTGGTGATGTCTACAATGATGGGA | |
| | CTGTCGACAAAGTTACGCACCCAATTGGGTCCTCCTTCGGGGTTCA | |
| | GGGCAAGGTTCCAGTCGGTCCAGCCGACCACATGGTACAGGAGGT | |
| | TCTAGGGTAAGGACAAAGGCAAAGAGACAAAGGCGCAACACTGG | |
| | GGGTCCCCAGAGAGTGTAGGTAAGGGTCACATGTGGGAGAGGCAG | |
| | CTGTGGGTAGGTCAGCCCTGTGAGGGGCACATTCCTTAGTAGCTAA | |
| | GGAGTTGGGGGTGTGAAGATCCAGGCATCTCAAGGGGAGCTGAGA | |
| | AGTCTGAGGCAGCTGCAAGTGCCTCAGTAGTTGCAAAAGGGGCAA | |
| | TGAGGTGTGCAGACCTGTGAAGGAAAGGGAAGATAGGGAATCAT | |
| | GGTTCCCCAGAGTTGCTCAAAAGGGCAGGCTAGCTGGGGAAAGCT | |
| | GGACAGGAAGGGCTTCTGTCAGTCTTTGGTGAAACTAGTAAGAGG | |
| | TCTGAGGTCTGCTTTGCAGGAAGGGAGACTGGGGTGGCTTACCGT | |
| | GATGATGCTGTGGCTGTACTGCATCCCTCGATCCCAGGAGCCTAGC | |
| | CGCACACTCTGCTCCCAGAACTTGGAGCCCACACAGGCCTCTGAG | |
| | GCAAAGAGCATGGTGTTGGGGAACAGGCGGTGTGTCTCCCCTAGG | |
| | GTGGCTTTGGCTGGAGCCAGAAAGTCCAGGTACCAATGTACAGCA | |
| | ATGCCATGAACATATTTAGCTGCTTCTGGGTCTGTCAGTACCTGCA | |
| | AAGGAAGAGCAACTGATCCTGGACCTTGCACACAGGCTTCTGGAA | |
| | CTTCTAGTTCCTGTTGTAGGAATCCTGGAGTTGGGTGACGGGAAGA | |
| | ATGCAACTAGAGAGGTTTGGGGAGATTTTTTTTTGTTTTTGAGACA | |
| | GGATGTCACTCTGTCACTCGGGCTGGAGTACAGTGGCGCAATCAC | |
| | GACTCACTGCAGCCTTGACTTCCTAGGGTCAACTGATCCTCCCACA | |
| | TTAGCCTCCTGAGTAGCTGGGACTACACGGGTGCCACACCCAGCT | |
| | AATTTGTGTGTGTATGTGTGTGTATGTATGTGTGTGTGTGTATATAT | |
| | ATATACATATAAACACATATATATGTATATACACATACATACATGA | |
| | ACCACCACCCCCAGCCTAGATAGTTTTGTTTTGTTTTGTTTCGAGAT | |
| | GGAGTCTCGCTCAGCCTCCCAGGCTGGAGTGCAGTGGCGCGATCT | |
| | CGGCTCACCGCAACCACCATCTCCCGGGTTCAACTGATTCTCCCTT | |
| | CTCAGCCTCCTGAGTAGCTGGGATTACAGACACCCACCATCATGCC | |
| | CAGATAATTTTTTTTTTTTGTATTTTAGTAGACACAGGGTTTCAACA | |
| | CGTTGGCCAGGGTGGTCTTAAACTCCTGACCTCAGGTGATCACCCG | |
| | CCTCGGCTTCCCAAAGTGCTGGGTTTGCATGAGTGAGCCACCTCGC | |
| | CCAGCCCCTAGAAAGGTTTCAAGCGACAACTGTGGGATCCATGGC | |
| | ACCCTGGAGGTCCAGGGGAATGGTGCTCTAGGAATCCATAGTTGG | |
| | GTAGAGAAATCGCTCTAAGTTTGGGAGCCAGTCATTTGGATGCTG | |
| | GATTTGAAGGTCACTGGAGCACCATGGAGGTCCAGGCCTTACCAC | |
| | CTTTGCCCAGTGGGGCAGCAGCAAGCGTTGGTCATCCAGCATGAG | |
| | TAGGCGGACATTGTGGTGAGTACTGTTGGCGAGGGTAGGACCTAG | |
| | GTCACGGGCAATGAAGTCTCGCTGATGTTCAGGGGTGAAGCCCAG | |
| | GCACTGGAAGGGGTATCCACTCAACAGCCCAGCAGAAGGCTCATT | |
| | TTCAGCTGTCACTGCCCAGAACTGTAACTTGTGCTCAGCATAGGCA | |
| | TCCAGGAACCTGGCAAGAGAAAGGTCATGAATGATCCGGCCAAGA | |
| | AAGTGGACCAGACCAGCTGGGTGTGGTGGCTCACACCTGTAATCC | |
| | CAGCACTTTGGGAAGCCGAGGCAGGTGGATCACTTGAGTTCAGGA | |
| | GTTCGAGAACAGCCTGGCGAAACCCCGTCTCTACTAAAAATAGAA | |
| | AAATCAGCTGGGCCTGGTGGCAGGCGCCTATAATCCCAGCTACTT | |
| | GGAAGGCTGAGGCAGGAGAATTGCTTGAACTCAGGAGGCAGAGG | |
| | TTACAGTGAGTGAAGATGGCGCCACAGCACTCCAGCCTGGGTGAC | |
| | AGAGAGAGAGACTCCTTCTCAAAAAAAAAAAAAAGAAGAAAAAT | |
| | AAAAAGAAAGTGGGCCAGACCGAGAGAACAGGAAGCCTGATGGA | |
| | GTGGGCAAGATTGACAGGCCCAAGGCTGAAAGGCCCAGAAGGTA | |
| | GAAAGGTGAGCTGAGGACAGGCAGATCTGGAAGTGGAACTAGGTT | |
| | GAGGGTTGGGACACAGATCAGCATGGCTAAATGGGAGGCCAGTCC | |
| | TGATCCCACATCCTTGCTGATCCCTTACTTCACAAAGTATCTGGCC | |
| | CAGGTCTGGTGGTAGATGTCTCCGGGCTGTCCCTTGAGTGACCCCT | |
| | TCCCATTCACCGCTCCATTGGTCTTGAGCCAAGTGGGTGATGTCCA | |
| | GGGGCTGGCAAGGAGTGAAACGGGACGCTGGGCCAACTGCAGGG | |
| | CTCGGTGAATCAGGGGTATCTAGAGACAAAGGTAGTGAAGAGAGA | |
| | AGCACCCAGAGTTGGAACACATACTAGCCCAACCAGTGCATCCGG | |
| | TTCAGCCATTAGCCTCCACCCTCCCACCCCCAGGACAAAACAGCA | |
| | GGGGACAAAATGTCTGTACAAGCAGACCTACCCTACAGTTTCTCA | |
| | ACCCCCAGACATCAGGGCCCTCAGGGCCTGAAAAAGCTAGAATGC | |
| | CTACCTTGAGCTTGGTATCTTCCTCTGGGAGGCTGAAGTTGTGCAA | |
| | CTGGAAATCATCAGGGGTGTCTGCATAGGTGTAGGTGCGGATGGA | |
| | GAAGTCACAGCTGGCCATGGGTACCCGGATGATGTTATATCCGATT | |
| | CCTACAGAAAAGGATGATCAAGATATGGTAGTCCGAGTCAATAGG | |
| | AGAGTATGGGACTCTGCTTATCACTTGCCAGTCCTAATAGTGTCTG | |
| | AGTCAGGGCCAAAGGGAACTTGGGCTCCTGGGTTGGAACCTGTGG | |
| | AGGCTGGCACCTGGGTGAAGCGCAGGCCTTTCTGAGCCTGAGTCC | |
| | GTAGCAGTTAGCAGATGATAGGCGGTGAAATCTTATTTCACAGGG | |
| | CATTAAAACAGGAACCAAATGTCAGGGATGGGCAGAAGTCAGGGT | |
| | CCAAAGAAAGGGCAAAGAAAAGTGTCAGTGGCTCACGCATGTAAT | |
| | CCCAGCACTTTGGGAGGCCGATGTGGGCAGATCACGAGGTCAGGA | |
| | GTTCGCAATCATCCTGGCCAACATAGTGAAACCCTGTCTCTACTAA | |
| | AAATACAAAAAATTAGCCGGGCGTGGTGGCAGGCACCTGTAATCC | |
| | CAGGTACTCGAGAGGCTGAGACAGGAGAATCACCTGAACCCGGGA | |
| | GGTGGAGGTTGCAGTGAGCTGAGATTGTGCCACTGCACTCCAGCC | |
| | TGGGTGACAGTGCGAGACTCTGTCTCAAAAAAAAAAAAAGAAAA | |
| | AAGAAAAGTGTCTGCTGGGCTCGGTGGCTCACACTTGTAATTCCAG | |
| | CACTTCGGGAGGCCAAGGCAGGTATATCATTTGAGGTCAGGAGTT | |
| | TAAGACTAGCCTGGTCAACATGATGAAACCCTGTCTCTACTAAAA | |
| | ATACAAAAATTAGCCAGGTGGTAGTGGCGCACGCCTATAATTCCA | |
| | GCTACTCAGGAGGCTGAGGCAGGAGAATCACTATAGCCTGGGAGG | |
| | CAGAGGTTGCTGTGAGGGGAGATCACACCACTGCACTCCTGTCTC | |
| | GCCGACAGAATGGGCAGAGTGAGATTCTGCCTCAAAAAAATTTTA | |
| | AAAAAAGAAAAGAAAAACGAAAAGTTTCAATGGCTCTATGTCATC | |
| | TTGTCCCCTTCCTCCTCACCTTCTTCAGAGAAGTACGATTTAAGTA | |
| | GCAAATTTTGGGCAGGGGGTGACAGGGCAAGGATGTTGAGAGCAG | |
| | CAGCATCTGTCATGGCCCCTCCAAATCCCTTCACTTTCTGGAACTT | |
| | CTGTTCTGGCTGCAGGGTCAGTAGCAGGCCTGAGGACATCCACAG | |
| | GGAATAAGGGTATCAGTACCCAGCGGGAAACTCCATGGTGATCAC | |
| | TGACACCATTTACCTCTAGGAGGACCCAGCCTGGCCCAGGGGGTG | |
| | AGGGGTGTAATGGTTACCTGTGCCCGTGTGATTAGCCTGGATGGGC | |
| | CCCATACTCAGCTCCATCCGTCGCCCACTGCGTGTACTCTCATAGC | |
| | GGCTGAAGGTACCAAGGGCAGGAAAGGTCGGGGGGTCAAAGGAG | |
| | TCACAGTATGTGGCATTGCAGACACACACCACCGAGCTGTAGCCG | |
| | AAGCTTTTAGGGATGCAGGGGCGGGCACCTGGGAGGGAGGGAGT | |
| | ACAAGCAGAGTGAGGTCTGATGAAGACATGGAGAATGGACACATC | |
| | TGCTAGGAGAGACTGAACACGGTTTCAAAATTCCTCACCCCTTGGC | |
| | CGGGCGCAGGGGCTCACACCTGTAATCCTAGCACTTTAGGAGGCC | |
| | GAGGTGGGCGGATCACCTGAGGTGAGGAGTTTGAGACCTGCCTTG | |
| | CCAGCATGGTGAAACCCCGTCTCTACTAAAAATACAAAAATTAGC | |
| | TGGGCGTAGTGGTGGCCGCCTGTAATCCCAGCTACCTGGGAAGCT | |
| | GAAGCAAGAGAATCGCTTGAATCTGGGAGGCAGAGGTTGGAATGA | |
| | GCCAAAATTGCACCACTGCACTCCAGCCCAGGCAACAGAGTAAGA | |
| | CTCTGTTTCAAAAGAAAAAAAACAAAATCCTCACCCCAAAGTTGG | |
| | TCTCAGTCACTCAAAAGGATTTATTGAGCACCTACTAAAAGTCTAC | |
| | CACCAGCTTACTGGAAGGCTACCAAAGGACTATGAGGCAGAAGGG | |
| | AGGCTCTGTGCTACCTCCCCACTGCCTTGACTCACTCACCTGATGC | |
| | CCACGACACTGCCTGAAGTAGAAGCAATCCTGTGAGGCTGCCAGC | |
| | CATGATGCTTACCCTACTCAAAGGCTTGGGACATTCCTGAGGACAG | |
| | AATGAGGAATGACTGAAAAGCAAGCCCCTCTCCACCCCTCAACTA | |
| | CTCTCCTGGGCAGGGCTTAGCTGCCTTTGGGTGCCCATGGCCCGGT | |
| | CTCCCACATTCATTAGGACAAGGCCCACAGAAACCTGGGTGCAGC | |
| | TCTCCCTGCAACCCTTCTGATGACAACTCTCTGCCCACCCCAAATC | |
| | AGGATGCTCCCCACCACTCTTCCCACCCATTTCAACTTCGACCCCT | |
| | CCCTCCATCTGTGCCTTGCTCAAAGAGCCATGATGGCCCTGGATTC | |
| | AAAGAGAGTCTGTCATTCATTAAATTCCAGTGCCAGGATTCCAGA | |
| | AGCACTGTGACAATGCTGATTGGGAGCTCTCTCTCTTACCTCTCTG | |
| | GAAGGACTTGAAAACTCCATCCCCTCAGGGTCATTAGATGAAGAG | |
| | AAGACCACAGGGGTTCCAGAGTCTCTGAAGGATAGAGGATCCACT | |
| | AAACAAAAACAAGGATGCAGGTACCTGCCTTAGCTATAGGCACTA | |
| | GGTTAGCCCTGCAAGTAATTCCGGCTTCCCGATGTGGATGGGTCAT | |
| | GTGATGACTAGGAGCGTCACATGACACAGGAAGTGAGGCAATCAC | |
| | AGCCATATTTCTAAAGGGCAATTGGCTTCCTCTCATCTGTTACAGA | |
| | TTATATGCCCTATAAAACTCTGGAGGGCATGTATGGGTGACAACTT | |
| | TAGGAAGAGCCTAGAACACAGATTTGCATGGAGAATGGGGTAGAG | |
| | GCTCCTAAATCCCAGAGGATGGGAACCACAGCAGGCACACTTCTT | |
| | TTTTGATAGAAATGTCAAAAAGGTACAAATAAAGCTGATATAATT | |
| | TAAAAAAAAAAACTTCACTGTGCAAAATACTGACACTACCAAGAT | |
| | GTTTAAGACAGTCTTTGCCCTATAGAGGTGTGTGAGGCATGGAAGT | |
| | CAGACACACAGATATTTACAGATAAAGACAGAAACGGTAACAGGT | |
| | GTGCATGGAGAGCTCTCTGAGATGAGGAGGGACCATTCGTGGGTG | |
| | GGGAATTATCCAGGATGGCTTCACAGAGGAAAAAGTGAGGGGGGT | |
| | TATTAGCCAGTGAAGTGCAGGGCACAAGAGGGTGGGACACTGGCA | |
| | GTGGGATGACAGGACTGGAGGGAGGGAGTGGTCAGTGCGGCTCCT | |
| | CTGCAGCGTCCCTTGTTTCATCATCAGATGCAGATGGTAATAACTG | |
| | TTCTTCCTTCCTCACAAGACAGGGAGGTTTGTAAAGTCGTTCAAAA | |
| | ACCAAAGTGTTGTACAAAGCCATATCCTCAGTGGACACAAGGAGG | |
| | AAGCTGTCCATGGTGTGGCCTCATGAACCACATCAAATGAGATTTA | |
| | GCGGGAGTGGCACACACAGTCATGACCTGACTAATCCCAGCTCTC | |
| | AGCCCATTTCCTTGCCTGGAAAATGGAGGCAATGCCACAACCTCA | |
| | AAGGGTGGTTACTGCAGTCAGTGAGGTAAGTGCAGTGCCTGACCA | |
| | CTTGGTAGGCACCTGGGAACTACTTGTCTCTTGTTTGTATTTTTTGT | |
| | TGTTGTTGTTTTTTGAGACAGAGTCTCACTCTGTTGCCCAGGCTGG | |
| | AGTGCAGTGGCGTGATCTTGGCTCACTGCAACCTCCACCTCCTGGG | |
| | TTCGAGTGATTCTCCTGCCTCAGCCTCCCAAGTAGCTGGGATTACA | |
| | GGTGCCCTCCAACATGCTCAGCTAATTTTTTTATTTTTAGTAGAGAT | |
| | GGGGTTTCACTATGTTGGCCAGGCTGGTCTGAAACTCTGGACCTCA | |
| | GGTGATCTGCTGCCTTGGTATCCGAAAGTGCTGGGATTACAGGCAT | |
| | GAGCCACTGCGCTGGACCCCAGCCATCCTTTTTGTTCTTTAAATGA | |
| | TCTCAGTGAAGTCTTTCTAGATACACTCCAAGCAAAATTGATCATT | |
| | TCTTCTTCTAGGTTCCTCCAGTAATTTTTTTTTTTTTGGTTTTGAGAC | |
| | AGAGTCTTGCCCTGTTGCCCAGGCTGGAGTGCAGTGTGATCTCGGC | |
| | TCAAGCAATTGCCCTGCCTCAGCCTCCTGAGTAGCTGGGATTACAG | |
| | GAGCCCACCACCATGCCCAGCTAATTTTTGTATTTTTAGTAGAGAT | |
| | GGGGTTTCACCATGTTGGCCAGGCTGGTCTCGAACCCCTGACCTCA | |
| | GGTGATCCACCAGCCTCAGCCTCCCAAGGTGTTGGGATTATAGCA | |
| | ATGAGCCACCACACCCGGTCCCTCCAGTAATTAAGTACAGTCTCAG | |
| | TGAGACAGCAAGTTTGGAATCCTGGCTACACCATTTACTGGCTGTT | |
| | TGACTTTCAATAAATCAATCACTCTAAGCCTCTGTTTCATCTATAA | |
| | AAGGGGAGTGATAACTCCTACCTCACAGAATTGTTGTGAGGTTTG | |
| | AGTGTGATAATGTGTCTCTAGTACACTGCTTGACACTAAACATTGC | |
| | AACATGTCAGGCCTCTGTTCCTGGAGTTCCTTGAAGGAATGTCTTA | |
| | TGCATTCTAAGTATCCTCGTAGATAGCCTGGCACAGGGGTAGCTGT | |
| | CAAGTTGTAGAATTGAACTAGTTGCTTCACTATGTCAGTAGCCACC | |
| | CCTTCCAGACTTCTCACTTTTCAAGGAAACATTGCACAGGATTTGT | |
| | TCTGGGTAGGGCAGGTAATATCTAGTACCTTACTTCCCTCAAGTTC | |
| | ATTCATCTCACAGATATTTCCTGAGCACATTCTACATTTGCCTCTCC | |
| | TGCTCTATTGAGTTTAGAAGTCCAAACATCTCCTTCCACTTCCCCTC | |
| | TGCATAGTGAGCCTCTTCTTTTTTGGCCAGGTACTGAGCAATTTTTT | |
| | GTTTGTTTGTTTGAGACAGGGTCTCCGTATGTTGCCCTGGATGGAG | |
| | TGTAGTAGCTTGATTTCGGCTCAGTGCAACCTCTGCCTCCAGAATA | |
| | AGCAATCCTCCCACCTCAGCCTCCAGAATAGCTGGGACTATAGGC | |
| | GCACACCACCACACCTGGCTAATTTCTGTATTTTTTGTAGAGACGG | |
| | GGTCTTACTGTGTTGCGCAGGTTGGTCTCAAACTCCTAAACTCAAA | |
| | GGATCCTCCCGCCTTGGCTTTGCAAAGTGCTGGGATTACAGGGTGA | |
| | GGCACTGCGCCCAGTCCGCTTGTTTTTTGTTTTGTTTTTTTTTTTGA | |
| | GACGGAGTATCTCTGTCACCCAGGCTGGAGTGCAGTGGCACGATC | |
| | TCGGTTCACTGCAACCTCCGCCTCCTAGGTTCAAGCGGAGGGTAGG | |
| | GACCAGTCCATCCTGGCACCCATCTGCAGCTCCAGTGCGAATCCCA | |
| | ACCCCGACGCTCGTCGCCGGGCTCCGTGAATGTTTGTCACATGTCT | |
| | GAAGAACGTATGAATTACATAACCTTCTTCCCACTCCACCCCTCAA | |
| | AAAGCAAGTGGATATAAAGACTTGAAGATTTTATAATCTCTTCTTC | |
| | ATTAGTAAAATCTGACCATCCTTACCGTTAAAAATAATAATGATGG | |
| | TTGGCCGGGCGCGGTGGCTCACGCCTATAATCACAGCTGTTTGGCA | |
| | GGCCGAGGCGGGCGGATCACGAGGTCAGGAATTGGAGACCAGCCT | |
| | GACCAACATGGTGAAACCCCGTCTCTACTAAAAGATACAAAAAAT | |
| | TAGCCGTGCGTGGTGGCGGGTGCCTGTAATCCCACGTACTTGGGA | |
| | GGCTGAGGTCGGAGAATCGCCTGAACCCGGGAGGCGGAGGTTGCA | |
| | GTGAGCCAAGAAGGCGCCATTACACTCCAGCCTGGGCGACAGGGC | |
| | GAGACTCCCTCAAAAAATAATAATAATAACAATAATAATAATAAT | |
| | GGTGAAAAAGGTTAAGTGCGAACGCAGGGAGGGGACAGCTAAGA | |
| | TCCAAAGGTCGAAATATTCATTACGCCTACCGTCGGCGAAGAGAA | |
| | ACAGCAGCCCCAACCGGAGGCAAAACGAAATCCCACCGCAGCCTG | |
| | CAAAGGCGCCTGGGCGGGACTGGAGACTGGGGCCCCGCGCAGTAA | |
| | GACTCTGAAGGCAGGATGCAGCCCGACCACCCGCAGCCGCGAGAA | |
| | AAGCAGCCCTGGGGAGTCGGGGCGGGACCTGGATTGGAAAAGAG | |
| | ACGGTCACTCATGCAGAGGCGGGACTCAGAGCCCTTCCTCAAGTC | |
| | TCATTGGTCAAGTTGAACGAACAAGTGTCGCTGGCGAGCCGGAGA | |
| | GAGAGAGAGAGCGAGAGCGAGAGAGCGAGCGAGAGAGAGCGAG | |
| | AGAGCGAGAGAGCGAGCGAGAGAGAGAGAGAGAGAGAGAGGAG | |
| | CCGGCGCGAGAACTACGCATGCGTGTCGGCGTTTTCCCGCCAGCA | |
| | CACTGTTGGTGGATGGGGGCGATTGAATTCCCACAGTGAGTCCAG | |
| | CCCACCGAAGCTCAGAGGATTCCTAACCTTCCTCTTTAGAGAGCCT | |
| | CAGGTTAGGGAACGTCCAGTGCGCAGAAGCTGCCCCGTGGGAATC | |
| | CCATTGTCCATCGCCTCCTAATGTTTCGTCCTGGCCGTGCCCTATCC | |
| | CTTCCTGAGGCTGGGTTGTTATGATGCTGAATTATTCAAGAAGTCT | |
| | TGCAGCCTGACGCCATCTCTGGGCAGTGCTCCTCCCACCTCCCTGT | |
| | CTTCCTTGGAGGGCACCACGTTGCCCCTACAAGCACAGGGTCCTGA | |
| | AGCTGTTTACAGGGCCCCACCCTGCCACTTTAGTATCTTAACGAAT | |
| | GTTTGTTGAGGACCTACTGAGTGTTAGACCCACTGCAATGAGCAA | |
| | GTTTCTGATCTTGGGACCTTAACATTCTATGGGACGACACAGATAT | |
| | ATAAACAAAAATAATAATTTTAGATAGTGATACATGCTGAAACCA | |
| | TGGGTATGTGGCCAGGCGCGGTGAGTCACACCTGTAATCCCAGCA | |
| | CTTTGGGAGACCGAGGCAGGCGGATTACTTGAGGTCAGGAGTTTG | |
| | AGACCCGCCTGGCCAACATGGTGAAACCCTATCTCTTCTAAAAATA | |
| | CAAAATTTAGCTGGGCGGGCATGGGGACGGGCGCCTGTAATCCCA | |
| | GCTACTCGGGAGGCTGAGGCAGGAGAATTGCTTGAACCCAGGAGG | |
| | TGGAAGGTTGCAGTGAGCCCAAATCACACCACTGCACTCCAGCCT | |
| | GGGTAACAAAGCAAGACCCTGTCTCAAAAAAAGACAAAATGAAA | |
| | CAAACAAACAAAAAAAAACATGGCTATGTGATAGAATGACTGCGA | |
| | TGGGGATTGGGTGCTCACTGAAATGGTGAGAATGGAGCTAAAACC | |
| | TGATATTAGGTGATAGCACCAGATATAGGGGCCAAGGGTTTCATG | |
| | CTTGGAAAACAGCAGGTGCAAACGTCCTTGTGCTGTGAGGAAATT | |
| | TGGCTGGAACAGAAGGTGGGCCAGATTGTAGCTGACCATAGTCTC | |
| | TGGGAGTTTGGAGATGATTCTAAATACAATGGGAAGCCATTAAAT | |
| | TGAGGCAGAGCCTTGATGTGACGTGCTTTATCATCATTTTGCTGAG | |
| | TGGAGAATGGATTGCTGGGAAAAGTGAAAGACTGATTCAGAGGTT | |
| | GTGGAGAGAGGATGGCTGGAGCTAGGTAGCTGGGGAGGTGGTGG | |
| | GATGTGGATGAATTCAGGATAGGATTGGTTGTGTTGGATATGGGA | |
| | AAAGAAGTCACCATGGTTCCTGAGCTTTTGGAATGTGCAAATGGG | |
| | TAATGGAGGTGCCATTGAGATGGGAACAGTGGAAAAATAACCGGT | |
| | GCTGGGGTGGGAATCAAGAGTTCAGTTGTGGCCATGTTGAATCTG | |
| | AAATGCCCATTAGACATCCAAGCAGACATGCTGAGTTGGATGGCG | |
| | CTCAT | |
| 3 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (N370S |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITSLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 4 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (L444P |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDPDAVALMHPDGSAVVVVLNRSSKDVPLT | |
| | IKDPAVGFLETISPGYSIHTYLWRRQ | |
| 5 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (E326K |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGKTHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 6 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (T369M |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIIMNLLYHVV | |
| | GWTDWNLALNPEGGPNWVRNFVDSPIIVDITKDTFYKQPMFYHLGHF | |
| | SKFIPEGSQRVGLVASQKNDLDAVALMHPDGSAVVVVLNRSSKDVPL | |
| | TIKDPAVGFLETISPGYSIHTYLWRRQ | |
| 7 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (G377S |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVS | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 8 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (D409H |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKHTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 9 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIWVPMASCDFSIRTYTYADTPDDFQ | (R120W |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 10 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (V394L |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWLRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 11 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (R496H |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFrPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRHQ | |
| 12 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (K178T |
| | LHNFSLPEEDTKLKIPLIHRALQLAQRPVSLLASTWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 13 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (R329C |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHCL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |
| 14 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (L444R |
| | LHNFSLPEEDTKLKIPLIHRALQLAQRPVSLLASPWTSPTWLKTNGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDRDAVALMHPDGSAVVVVLNRSSKDVPL | |
| | TIKDPAVGFLETISPGYSIHTYLWRRQ | |
| 15 | ARPCIPKSFGYSSVVCVCNATYCDSFDPPTFPALGTFSRYESTRSGRR | Human |
| | MELSMGPIQANHTGTGLLLTLQPEQKFQKVKGFGGAMTDAAALNIL | GBA1 |
| | ALSPPAQNLLLKSYFSEEGIGYNIIRVPMASCDFSIRTYTYADTPDDFQ | (N188S |
| | LHNFSLPEEDTKLKIPLIHRALQLAQRPVSLLASPWTSPTWLKTSGAV | mutant) |
| | NGKGSLKGQPGDIYHQTWARYFVKFLDAYAEHKLQFWAVTAENEPS | amino acid |
| | AGLLSGYPFQCLGFTPEHQRDFIARDLGPTLANSTHHNVRLLMLDDQ | sequence |
| | RLLLPHWAKVVLTDPEAAKYVHGIAVHWYLDFLAPAKATLGETHRL | |
| | FPNTMLFASEACVGSKFWEQSVRLGSWDRGMQYSHSIITNLLYHVVG | |
| | WTDWNLALNPEGGPNWVRNFVDSPIIVDITKDTFYKQPMFYHLGHFS | |
| | KFIPEGSQRVGLVASQKNDLDAVALMHPDGSAVVVVLNRSSKDVPLT | |
| | IKDPAVGFLETISPGYSIHTYLWRRQ | |

1. A method of increasing expression of GBA1 in a cell, the method comprising:
(i) introducing, into a pluripotent stem cell, a deoxyribonucleic acid (DNA) sequence encoding GBA1 operably linked to a promoter, wherein the DNA sequence is positioned between inverted terminal repeats and is capable of integrating into DNA in the cell; and
(ii) introducing, into the cell, a transposase or a nucleic acid sequence encoding a transposase,
wherein the introducing in (i) and (ii) results in integration of the DNA sequence encoding GBA1 into the genome of the cell.

2. The method of claim 1, wherein the cell comprises a variant of GBA1 associated with Parkinson's disease.

3. (canceled)

4. (canceled)

5. The method of claim 1, wherein the transposase is a Class II transposase.

6. The method of claim 1, wherein the transposase is selected from the group consisting of: Sleeping Beauty, piggyBac, TcBuster, Frog Prince, Tol2, Tcl/mariner, or a derivative thereof having transposase activity.

7. (canceled)

8. The method of claim 1, wherein the transposase is TcBuster.

9. The method of claim 1, wherein the promoter is selected from the group consisting of: ubiquitin C (UBC) promoter, cytomegalovirus (CMV) promoter, phosphoglycerate kinase (PGK) promoter, CMV early enhancer/chicken b actin (CAG) promoter, glial fibrilary acidic protein (GFAP) promoter, synapsin-1 promoter, and Neuron Specific Enolase (NSE) promoter.

10. The method of claim 1, wherein the promoter is a PGK promoter.

11. The method of claim 1, wherein the nucleic acid sequence encoding the transposase and/or the DNA sequence encoding GBA1 are introduced into the cell by electrotransfer.

12. The method of claim 1, wherein the method comprises introducing, into the cell, (a) a nucleic acid encoding a transposase, or (b) a transposase.

13. The method of claim 1, wherein the nucleic acid encoding a transposase is part of a plasmid; and/or the DNA sequence encoding GBA1 is part of a plasmid.

14. (canceled)

15. The method of claim 13, wherein the nucleic acid encoding a transposase is DNA.

16. The method of claim 1, wherein the plasmid containing the DNA sequence encoding GBA1 and the plasmid containing the nucleic acid sequence encoding the transposase are different plasmids.

17. (canceled)

18. (canceled)

19. The method of claim 1, wherein (i) the DNA sequence encoding GBA1 and the (ii) the transposase or the nucleic acid sequence encoding the transposase are introduced into the cell at the same time.

20. (canceled)

21. The method of claim 1, wherein the DNA sequence encoding GBA1 is introduced into an intron.

22. The method of claim 1, wherein:
the cell exhibits decreased expression of GBA1 prior to being introduced with the DNA sequence encoding GBA1 and the transposase or the nucleic acid sequence encoding the transposase, as compared to a reference cell, from a subject without Parkinson's Disease; or
the cell exhibits reduced activity of the β-Glucocerebrosidase (GCase) enzyme encoded by GBA1 prior to being introduced with the DNA sequence encoding GBA1 and the transposase or the nucleic acid sequence encoding the transposase, as compared to a reference cell from a subject without Parkinson's Disease.

23. (canceled)

24. The method of claim 1, wherein GBA1 is human GBA1.

25. The method of claim 1, wherein the DNA sequence encoding GBA1 comprises a coding region of the sequence set forth in SEQ ID NO:2 or a codon-optimized version of a coding region of the sequence set forth in SEQ ID NO:2.

26. The method of claim 1, wherein the DNA encoding GBA1 encodes an amino acid comprising the amino acid sequence set forth in SEQ ID NO:1.

27. The method of claim 2, wherein the variant of GBA1 comprises a single nucleotide polymorphism (SNP) that is associated with Parkinson's disease.

28. The method of claim 27, wherein the SNP is rs76763715.

29. The method of claim 28, wherein the rs76763715 is a cytosine variant.

30. The method of claim 27, wherein the variant of GBA1 comprising a SNP encodes a serine, rather than an asparagine, at amino acid position 370 (N370S).

31. The method of claim 30, wherein the wild-type form of GBA1 comprises a thymine instead of the cytosine variant.

32. The method of claim 27, wherein the SNP is rs421016.

33. The method of claim 32, wherein the rs421016 is a guanine variant.

34. The method of claim 27, wherein the variant of GBA1 comprising the SNP encodes a proline, rather than a leucine, at amino acid position 444 (L444P).

35. The method of claim 34, wherein the wild-type form of GBA1 comprises an adenine instead of the guanine variant.

36. The method of claim 27, wherein the SNP is rs2230288.

37. The method of claim 36, wherein the rs2230288 is a thymine variant.

38. The method of claim 27, wherein the variant of GBA1 comprising the SNP encodes a lysine, rather than a glutamic acid, at position 326 (E326K).

39. The method of claim 38, wherein the wild-type form of GBA1 comprises a cytosine instead of the thymine variant.

40. The method of claim 1, wherein the cell is an induced pluripotent stem cell (iPSC).

41. The method of claim 40, wherein the iPSC is artificially derived from a non-pluripotent cell from a subject.

42. (canceled)

43. The method of claim 41, wherein the subject has Parkinson's disease or Gaucher's disease.

44. The method of claim 41, wherein the subject has Parkinson's disease.

45. The method of claim 1, wherein, after the integration of the DNA sequence encoding GBA1 into the DNA of the cell, the method further comprises determining the location of the integrated DNA sequence in the genome of the cell.

46. A method of differentiating neural cells, the method comprising:
(a) performing a first incubation comprising culturing the cells produced by the method of claim 1 in a non-adherent culture vessel under conditions to produce a cellular spheroid, wherein beginning at the initiation of the first incubation (day 0) the cells are exposed to (i) an inhibitor of TGF-β/activin-Nodal signaling; (ii) at least one activator of Sonic Hedgehog (SHH) signaling; (iii) an inhibitor of bone morphogenetic protein (BMP) signaling; and (iv) an inhibitor of glycogen synthase kinase 3β (GSK3β) signaling; and
(b) performing a second incubation comprising culturing cells of the spheroid in a substrate-coated culture vessel under conditions to neurally differentiate the cells.

47-66. (canceled)

67. A pluripotent stem cell produced by the method of claim 1.

68. A neurally differentiated cell produced by the method of claim 46.

69. A pluripotent stem cell (PSC) comprising an exogenous deoxyribonucleic acid (DNA) sequence encoding GBA1 integrated into its genome.

70. A cell comprising an exogenous deoxyribonucleic acid (DNA) sequence encoding GBA1 integrated into its genome, wherein the cell is selected from the group consisting of a neurally differentiated cell, a microglial cell, a macrophage, and a hematopoietic stem cell (HSC).

71-84. (canceled)

85. A method of treatment, comprising administering to a subject a therapeutically effective amount of the therapeutic composition of claim 81.

86-99. (canceled)

100. A transposon-based system for increasing expression of GBA1 in a cell, the system comprising:
(i) a deoxyribonucleic acid (DNA) sequence encoding GBA1, wherein the DNA sequence is positioned between at least two inverted terminal repeats and is capable of integrating into DNA in a cell; and
(ii) a transposase or a nucleic acid sequence encoding a transposase,
wherein the cell exhibits (i) reduced activity of the β-Glucocerebrosidase (GCase) enzyme encoded by GBA1 and/or (ii) reduced expression of GBA1 prior to being introduced with the DNA sequence encoding GBA1 and the transposase or the nucleic acid sequence encoding the transposase, optionally as compared to a reference cell from a subject without Parkinson's Disease.

101-120. (canceled)
